(19) United States
(12) Patent Application Publication
(10) Pub. No.: US 2025/0123566 A1
(43) Pub. Date: Apr. 17, 2025

(54) ONIUM SALT TYPE MONOMER, POLYMER, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventors: Masahiro Fukushima, Joetsu-shi (JP); Jun Hatakeyama, Joetsu-shi (JP); Masaki Ohashi, Joetsu-shi (JP)

(73) Assignee: Shin-Etsu Chemical Co., Ltd., Tokyo (JP)

(21) Appl. No.: 18/909,136

(22) Filed: Oct. 8, 2024

(30) Foreign Application Priority Data
Oct. 17, 2023 (JP) 2023-178545

(51) Int. Cl. G03F 7/039; C07C 309/12; C07C 309/75; C07C 323/09; C07D 327/08; C08F 212/14; C08F 220/18; C08F 220/22; C08F 220/40; G03F 7/00; G03F 7/004

(52) U.S. Cl. CPC G03F 7/0397; C07C 309/12; C07C 309/75; C07C 323/09; C07D 327/08; C08F 212/24; C08F 220/1806; C08F 220/22; C08F 220/40; G03F 7/0046; G03F 7/70025; G03F 7/70033

(57) ABSTRACT

The onium salt type monomer for a chemically amplified resist composition has excellent solvent solubility and a high sensitivity and contrast, and is excellent in lithographic performance such as exposure tolerance (EL), LWR, CDU and depth of focus (DOF), and excellent in resistance to pattern collapse and etch resistance even in fine pattern formation. The onium salt type monomer has the following formula (a).

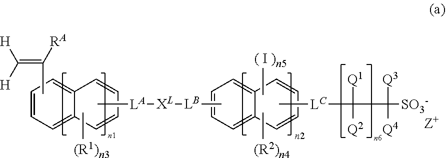

### CROSS-REFERENCE TO RELATED APPLICATIONS

This non-provisional application claims priority under 35 U.S.C. § 119(a) on Patent Application No. 2023-178545 filed in Japan on Oct. 17, 2023, the entire contents of which are hereby incorporated by reference.

### TECHNICAL FIELD

The present invention relates to an onium salt type monomer, a polymer, a chemically amplified resist composition, and a pattern forming process.

### BACKGROUND ART

To meet the demand for higher integration density and operating speed of LSIs, the effort to reduce the pattern rule is in rapid progress. The wide-spreading flash memory market and the demand for increased storage capacities drive forward the miniaturization technology. As the advanced miniaturization technology, manufacturing of microelectronic 65-nm mode devices by the ArF lithography has been implemented in a mass scale. Manufacturing of 45-nm node devices by the next generation ArF immersion lithography is approaching to the verge of high-volume application. The candidates for the next generation 32-nm node include ultra-high NA lens immersion lithography using a liquid having a higher refractive index than water in combination with a high refractive index lens and a high refractive index resist film, EUV lithography of wavelength 13.5 nm, and double patterning version of the ArF lithography, on which active research efforts have been made.

As the pattern feature size is reduced, approaching to the diffraction limit of light, light contrast lowers. In the case of positive resist film, a lowering of light contrast leads to reductions of resolution and focus margin of hole and trench patterns.

As the pattern feature size is reduced, the line width roughness (LWR) of line patterns and the critical dimension uniformity (CDU) of hole patterns are regarded significant. It is pointed out that these factors are affected by the segregation or agglomeration of a base polymer and acid generator and the diffusion of generated acid. There is a tendency that as the resist film becomes thinner, LWR becomes greater. A film thickness reduction to comply with the progress of size reduction causes a degradation of LWR, which becomes a serious problem.

The EUV lithography resist must meet high sensitivity, high resolution and low LWR at the same time. As the acid diffusion distance is reduced, LWR is reduced, but sensitivity becomes lower. For example, as the PEB temperature is lowered, the outcome is a reduced LWR, but a lower sensitivity. As the amount of quencher added is increased, the outcome is a reduced LWR, but a lower sensitivity. It is necessary to overcome the tradeoff relation between sensitivity and LWR.

Patent Document 1 discloses a resist compound comprising repeat units derived from an onium salt of a polymerizable unsaturated bond-containing sulfonic acid. The so called polymer-bound acid generator is capable of generating a polymer type sulfonic acid upon exposure and characterized by a very short distance of acid diffusion. Sensitivity may be enhanced by increasing a proportion of the acid generator. In the case of addition type acid generators, as the amount of acid generator added is increased, a higher sensitivity is achievable, but the acid diffusion distance is also increased. Since the acid diffusion is non-uniform, increased acid diffusion leads to degraded LWR and CDU. With respect to a balance of sensitivity, LWR and CDU, the polymer-bound acid generator has a high capability.

Iodine atoms absorb a very large amount of EUV having a wavelength 13.5 nm, and have been confirmed to be capable of generating secondary electrons during exposure, and attracted attention in the EUV lithography field. Patent Document 2 discloses a photoacid generator having iodine atoms in the anion, and Patent Document 3 discloses a polymerizable group-containing photoacid generator having iodine atoms in the anion. Iodine atoms have been confirmed to improve lithographic performance to some extent, but do not have high organic solvent solubility, and may be precipitated in the solvent.

### CITATION LIST

Patent Document 1: JP No. 4425776
Patent Document 2: JP No. 6720926
Patent Document 3: JP No. 6973274

### SUMMARY OF THE INVENTION

In the field of acid-catalyzed chemically amplified resist compositions, it is desired to have a resist composition having a higher sensitivity, improved LWR of line patterns, and improved CDU of hole patterns, and exhibiting excellent etch resistance after formation of patterns.

The present invention has been made in view of the above-described circumstances, and an object of the present invention is to provide an onium salt type monomer for a chemically amplified resist composition which is processed by photolithography using, in particular, high-energy radiation such as KrF excimer laser, ArF excimer laser, an electron beam (EB) or EUV, has excellent solvent solubility and a high sensitivity and contrast, and is excellent in lithographic performance such as exposure tolerance (EL), LWR, CDU and depth of focus (DOF), and excellent in resistance to pattern collapse and etch resistance even in fine pattern formation, a polymer comprising repeat units derived from the onium salt type monomer, a chemically amplified resist composition containing the polymer, and a pattern forming process using the chemically amplified resist composition.

The present inventors have extensively conducted studies for achieving the above-described object, and resultantly found that by using, as a polymer-bound acid generator, a polymer comprising repeat units derived from an onium salt having a styrene or vinyl naphthalene structure as a polymerizable group and containing a fluorosulfonic acid anion having an aromatic ring substituted with an iodine atom, a chemically amplified resist composition having a high sensitivity, improved LWR and CDU, high contrast, high resolution, and excellent etch resistance can be obtained.

The present invention provides the following onium salt type monomer, polymer, chemically amplified resist composition, and pattern forming process.
1. An onium salt type monomer having the following formula (a):

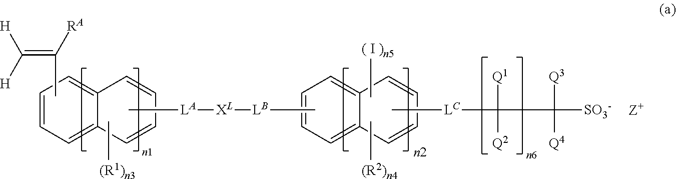

wherein n1 is 0 or 1, n2 is 0 or 1, n3 is an integer of 0 to 4, n4 is an integer of 0 to 4, n5 is an integer of 1 to 6, provided that the sum of n4+n5 is 1 or more and 4 or less when n2 is 0, and the sum of n4+n5 is 1 or more and 6 or less when n2 is 1, n6 is an integer of 0 to 4,
R^{A} is hydrogen atom, fluorine atom, methyl group, or trifluoromethyl group,
R¹ is a halogen atom, or a C₁-C₂₀ hydrocarbyl group which may contain a heteroatom, a plurality of R¹ moieties may bond together to form a ring with the carbon atom to which they are attached when n3 is 2, 3 or 4,
R² is a halogen atom other than an iodine atom, or a C₁-C₂₀ hydrocarbyl group which may contain a heteroatom, a plurality of R² moieties may bond together to form a ring with the carbon atom to which they are attached when n4 is 2, 3 or 4,
L^{A}, L^{B} and L^{C} are each independently a single bond, an ether bond, an ester bond, a sulfonic ester bond, a sulfonic amide bond, a carbonate bond or a carbamate bond,
X^{L} is a single bond, or a C₁-C₄₀ hydrocarbylene group which may contain a heteroatom,
Q¹ and Q² are each independently a hydrogen atom, a fluorine atom, or a C₁-C₆ fluorinated saturated hydrocarbyl group,
Q³ and Q⁴ are each independently a fluorine atom, or a C₁-C₆ fluorinated saturated hydrocarbyl group, and
Z⁺ is an onium cation.
2. The onium salt type monomer of 1 which has the following formula (a1):

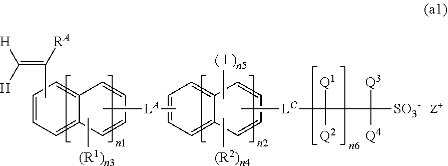

wherein n1 to n6, R^{A}, R¹, R², L^{A}, L^{C}, Q¹ to Q⁴, and Z⁺ are as defined above.
3. The onium salt type monomer of 2 which has the following formula (a2):

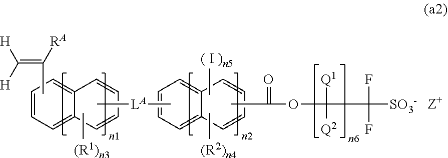

wherein n1 to n6, R^{A}, R¹, R², L^{A}, Q¹, Q², and Z⁺ are as defined above.
4. The onium salt type monomer of any one of 1 to 3, wherein Z⁺ is a sulfonium cation having the following formula (cation-1) or iodonium cation having the following formula (cation-2):

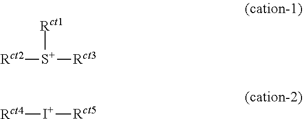

wherein R^{ct1} to R^{ct5} are each independently a halogen atom, or a C₁-C₃₀ hydrocarbyl group which may contain a heteroatom, and R^{ct1} and R^{ct2} may bond together to form a ring with the sulfur atom to which they are attached.
5. A polymer comprising repeat units derived from the onium salt type monomer of any one of 1 to 4.
6. The polymer of 5, further comprising repeat units having the following formula (b1) or (b2):

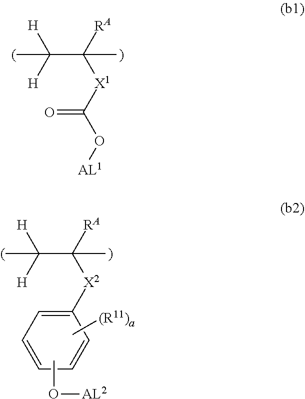

wherein R^{A} is each independently a hydrogen atom, a fluorine atom, a methyl group, or a trifluoromethyl group,
X¹ is a single bond, a phenylene group, a naphthylene group, *—C(═O)—O—X¹¹— or *—C(═O)—NH—X¹¹—, and the phenylene group or the naphthylene group may be substituted with a C₁-C₁₀ alkoxy group which may contain a fluorine atom, or a halogen atom, X¹¹ is a C₁-C₁₀ saturated hydrocarbylene group, a phenylene group, or a naphthylene group, and the saturated hydrocarbylene group may contain a hydroxy group, an ether bond, an ester bond or a lactone ring,
X² is a single bond, *—C(═O)—O— or *—C(═O)—NH—,
the asterisk (*) designates a point of attachment to the carbon atom in the backbone,
AL¹ and AL² are each independently an acid labile group,
R¹¹ is a halogen atom, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, and
a is an integer of 0 to 4.
7. The polymer of 5 or 6, further comprising repeat units having the following formula (c1):

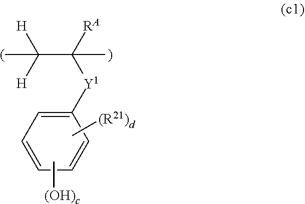

wherein R^{A} is a hydrogen atom, a fluorine atom, a methyl group, or a trifluoromethyl group,
Y¹ is a single bond, *—C(═O)—O— or *—C(═O)—NH—, the asterisk (*) designates a point of attachment to the carbon atom in the backbone,
R²¹ is a halogen atom, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom,
c is an integer of 1 to 4, and d is an integer of 0 to 3, provided that the sum of c+d is 1 or more and 5 or less.
8. The polymer of any one of 5 to 7, further comprising repeat units having the following formula (d1):

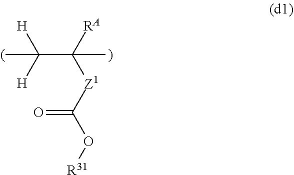

wherein R^{A} is a hydrogen atom, a fluorine atom, a methyl group, or a trifluoromethyl group,
Z¹ is a single bond, a phenylene group, a naphthylene group, *—C(═O)—O—Z¹¹— or *—C(═O)—NH—Z¹¹—, or the phenylene group or the naphthylene group may be substituted with a C₁-C₁₀ alkoxy group which may contain a fluorine atom, or a halogen atom, the asterisk (*) designates a point of attachment to the carbon atom in the backbone, Z¹¹ is a C₁-C₁₀ saturated hydrocarbylene group, a phenylene group, or a naphthylene group, and the saturated hydrocarbylene group may contain a hydroxy group, an ether bond, an ester bond or a lactone ring, and
R³¹ is a hydrogen atom, or a C₁-C₂₀ group containing at least one structure selected from a hydroxy group other than a phenolic hydroxy group, a cyano group, a carbonyl group, a carboxy group, an ether bond, an ester bond, a sulfonic ester bond, a carbonate bond, a lactone ring, a sultone ring, and a carboxylic anhydride (—C(═O)—O—C(═O)—).
9. A chemically amplified resist composition comprising (A) a base polymer containing the polymer of any one of 5 to 8.
10. The chemically amplified resist composition of 9, further comprising (B) an organic solvent.
11. The chemically amplified resist composition of 9 or 10, further comprising (C) a quencher.
12. The chemically amplified resist composition of any one of 9 to 11, further comprising (D) an acid generator.
13. The chemically amplified resist composition of any one of 9 to 12, further comprising (E) a surfactant.
14. The chemically amplified resist composition of any one of 9 to 13, further comprising (F) a dissolution inhibitor.
15. A pattern forming process comprising the steps of applying the chemically amplified resist composition of any one of 9 to 14 onto a substrate to form a resist film thereon, exposing the resist film to high-energy radiation, and developing the exposed resist film in a developer.
16. The pattern forming process of 15, wherein the high-energy radiation is ArF excimer laser having a wavelength 193 nm, KrF excimer laser having a wavelength 248 nm, EB, or EUV having a wavelength 3 to 15 nm.

### Advantageous Effects of Invention

A resist film containing a polymer comprising repeat units derived from an onium salt type monomer having the formula (a) has good solvent solubility, and a large amount of iodine atoms, and is characterized by a short distance of acid diffusion. Accordingly, a reduction of resolution due to blur by acid diffusion can be prevented, and LWR and CDU can be improved. Iodine atoms absorb a very large amount of EUV having a wavelength 13.5 nm, and generate secondary electrons during exposure, so that the sensitivity is increased. Accordingly, it is possible to construct a chemically amplified resist composition having a high sensitivity and improved LWR and CDU. The aromatic ring acts as a group exhibiting good etch resistance, and is suitable for fine pattern formation.

### DETAILED DESCRIPTION OF THE INVENTION

### [Onium Salt Type Monomer]

The inventive onium salt type monomer has the following formula (a).

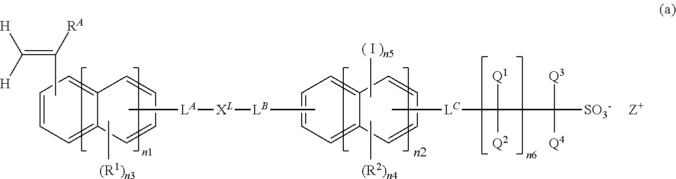

In formula (a), n1 is 0 or 1. The onium salt type monomer has a benzene ring when n1 is 0, and a naphthalene ring when n1 is 1, and n1 is preferably 0 from the aspect of solvent solubility. n2 is 0 or 1, The onium salt type monomer has a benzene ring when n2 is 0, and a naphthalene ring when n2 is 1, and n2 is preferably 0 from the aspect of solvent solubility. n3 is an integer of 0 to 4, n3 is preferably 0, 1 or 2, more preferably 0 or 1. n4 is an integer of 0 to 4, preferably 0, 1, 2 or 3, more preferably 0, 1 or 2, still more preferably 0 or 1. n5 is an integer of 1 to 6. As the number of iodine atoms in the anionic structure becomes larger, the amount of absorption of EUV increases, but precipitation in the resist composition may occur due to reduced solvent solubility. Therefore, n5 is preferably 1, 2 or 3, more preferably 1 or 2. Provided that the sum of n4+n5 is 1 or more and 4 or less when n2 is 0, and the sum of n4+n5 is 1 or more and 6 or less when n2 is 1, n6 is an integer of 0 to 4, preferably 0, 1, 2 or 3, more preferably 1.

In formula (a), R^{A} is hydrogen atom, fluorine atom, methyl group, or trifluoromethyl group. Of these, a hydrogen atom and a methyl group are preferred, and a hydrogen atom is more preferred.

In formula (a), the iodine atom in the aromatic ring of the anion is preferably bound in an ortho position with respect to a carbon atom to which L^{B} is attached. Since an iodine atom is an element having a large atomic radius, the rotation of the bond axis between the aromatic ring to which a polymerizable group is attached and the aromatic ring to which an iodine atom is attached is suppressed, leading to improved rigidity of the polymer.

In formula (a), R¹ is a halogen atom, or a C₁-C₂₀ hydrocarbyl group which may contain a heteroatom. The halogen atom is preferably a fluorine, chlorine, bromine or iodine atom, more preferably a fluorine or iodine atom. The hydrocarbyl group may be saturated or unsaturated, and may be linear, branched, or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icocyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, allyl, propenyl, butenyl and hexenyl groups; C₃-C₂₀ cyclic unsaturated hydrocarbyl groups such as a cyclohexenyl group; C₂-C₂₀ aryl groups such as phenyl and naphthyl groups; C₇-C₂₀ aralkyl groups such as benzyl, 1-phenylethyl and 2-phenylethyl groups; and combinations thereof. Some or all of hydrogen atoms of the hydrocarbyl group may be replaced by a group containing a heteroatom such as an oxygen atom, a sulfur atom, a nitrogen atom or a halogen atom, some constituent —CH₂— of the hydrocarbyl group may be replaced by a group containing a heteroatom such as an oxygen atom, a sulfur atom or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. Groups R¹ may be the same or different when n3 is 2, 3 or 4.

a plurality of R¹ moieties may bond together to form a ring with the aromatic ring carbon atom to which they are attached when n3 is 2, 3 or 4, Specific examples of the ring formed herein include cyclopropane, cyclobutane, cyclopentane, cyclohexane, norbornane and adamantane rings. Some or all of hydrogen atoms in the ring may be replaced by a group containing a heteroatom such as an oxygen atom, a sulfur atom, a nitrogen atom or a halogen atom, some constituent —CH₂— in the ring may be replaced by a group containing a heteroatom such as an oxygen atom, a sulfur atom or a nitrogen atom, and as a result, the hydrocarbyl group may contain a hydroxy group, a fluorine atom, a chlorine atom, a bromine atom, an iodine atom, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

In formula (a), R² is a halogen atom other than an iodine atom, a C₁-C₂₀ hydrocarbyl group which may contain a heteroatom. Specific examples of the halogen atom other than the iodine atom include a fluorine atom, a chlorine atom, a bromine atom, and an iodine atom. The hydrocarbyl group may be saturated or unsaturated, and may be linear, branched, or cyclic. Examples of the hydrocarbyl group are as exemplified above as a hydrocarbyl group R¹, but not limited thereto. Groups R² may be the same or different when n4 is 2, 3 or 4.

A plurality of R² moieties may bond together to form a ring with the aromatic ring carbon atom to which they are attached when n4 is 2, 3 or 4. The ring is preferably a five- or eight-membered ring.

In formula (a), L^{A}, L^{B} and L^{C} are each independently a single bond, an ether bond, an ester bond, a sulfonic ester bond, a sulfonic amide bond, a carbonate bond or a carbamate bond. Inter alia, L^{A} is preferably a single bond, an ether bond, an ester bond or a sulfonic ester bond, more preferably an ester bond or a sulfonic ester bond. L^{B} is preferably a single bond, an ether bond or an ester bond, more preferably a single bond. L^{C} is preferably a single bond, an ether bond, an ester bond or a sulfonic ester bond, more preferably an ether bond or an ester bond.

In formula (a), X^{L} is a single bond, or a C₁-C₄₀ hydrocarbylene group which may contain a heteroatom, The hydrocarbylene group may be straight, branched or cyclic, and specific examples thereof include an alkanediyl group, a cyclic saturated hydrocarbylene group, and an arylene group. Specific examples of the heteroatom include oxygen, nitrogen and sulfur atoms.

Specific examples of the C₁-C₄₀ hydrocarbylene group X^{L} which may contain a heteroatom are shown below, but not limited thereto. In the following formulae, the asterisk (*) is a point of attachment to L^{A} and L^{B}.

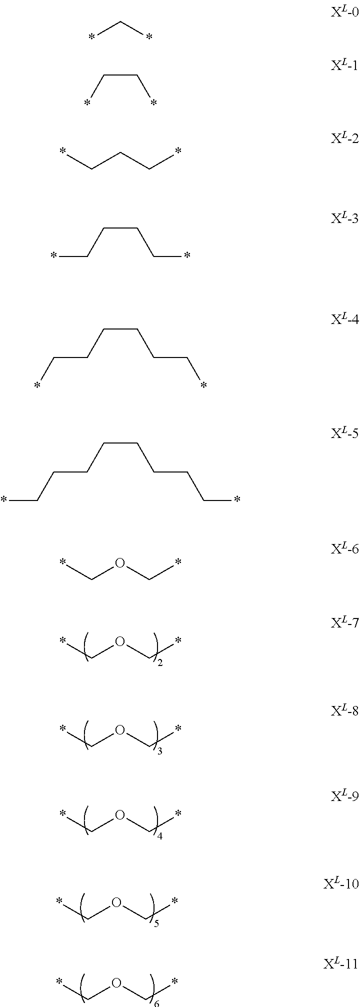

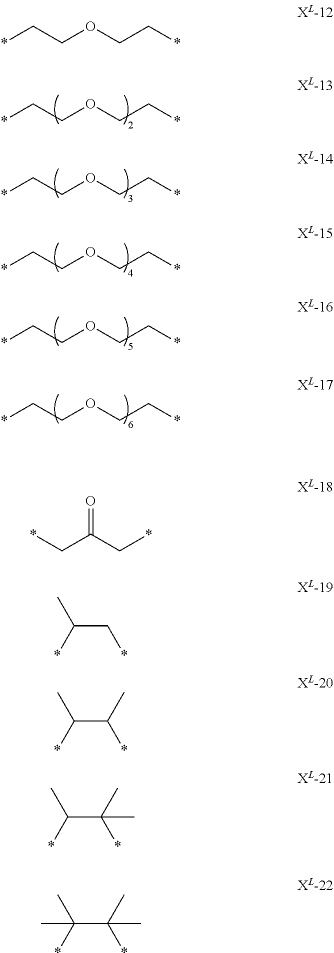

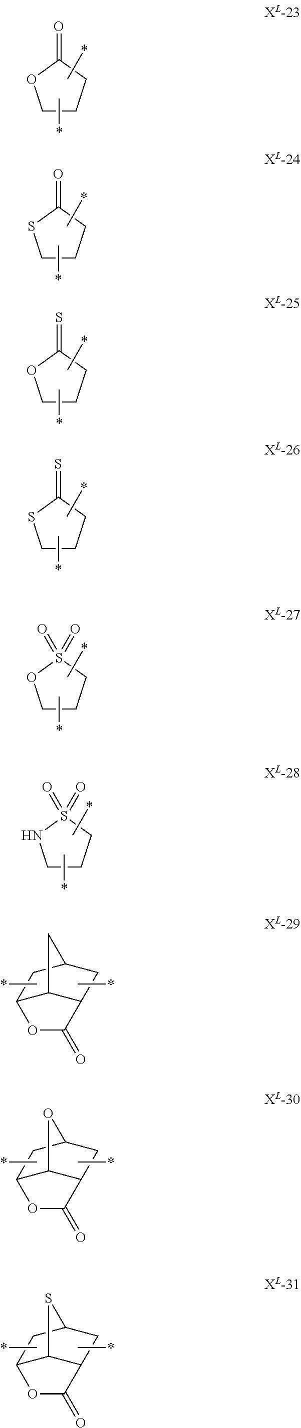

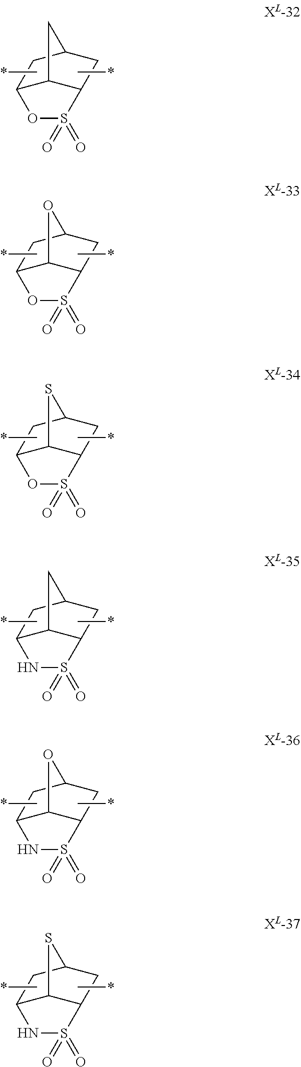

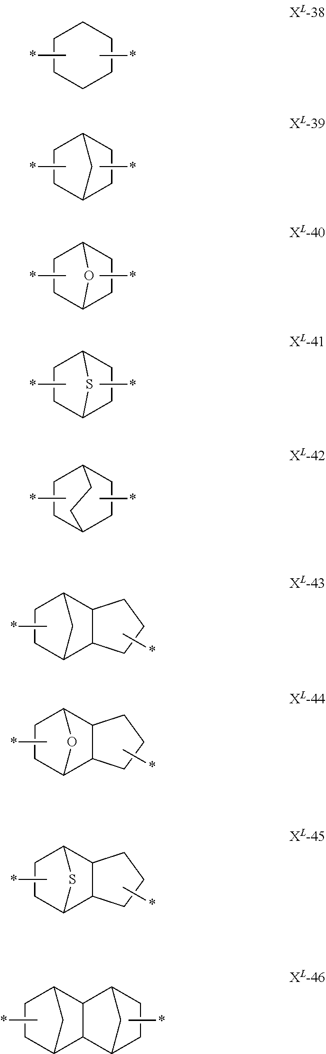

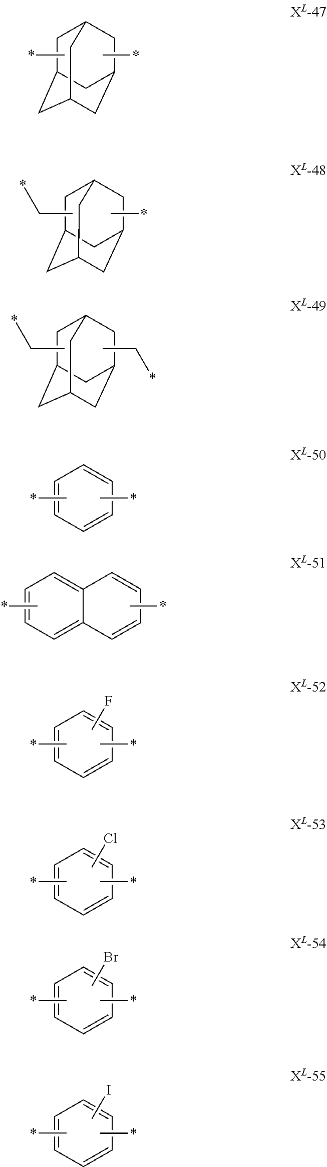

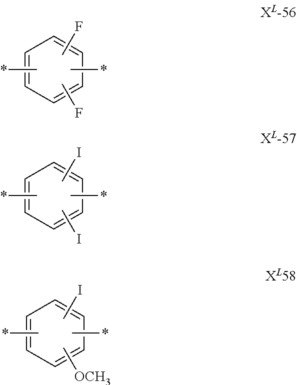

Of these, X^{L}-0 to X^{L}-22, X^{L}-29 to X^{L}-34, and X^{L}-47 to X^{L}-58 are preferred.
X^{L} is preferably a single bond from the viewpoint of rigidity of the polymer obtained.

In formula (a), Q¹ and Q² are each independently a hydrogen atom, a fluorine atom or a C₁-C₆ fluorinated saturated hydrocarbyl group. The C₁-C₆ fluorinated saturated hydrocarbyl group is preferably a trifluoromethyl group.

In formula (a), Q³ and Q⁴ are each independently a fluorine atom or a C1-C₆ fluorinated saturated hydrocarbyl group. The C₁-C₆ fluorinated saturated hydrocarbyl group is preferably a trifluoromethyl group. More preferably, Q³ and Q⁴ are fluorine atoms.

Preferred examples of the partial structure —[C(Q¹)(Q²)]ₙ₄-C(Q³)(Q⁴)-SO₃— in formula (a) are shown below, but the partial structure is not limited thereto. In the following formulae, the asterisk (*) designates a point of attachment to L^{C}.

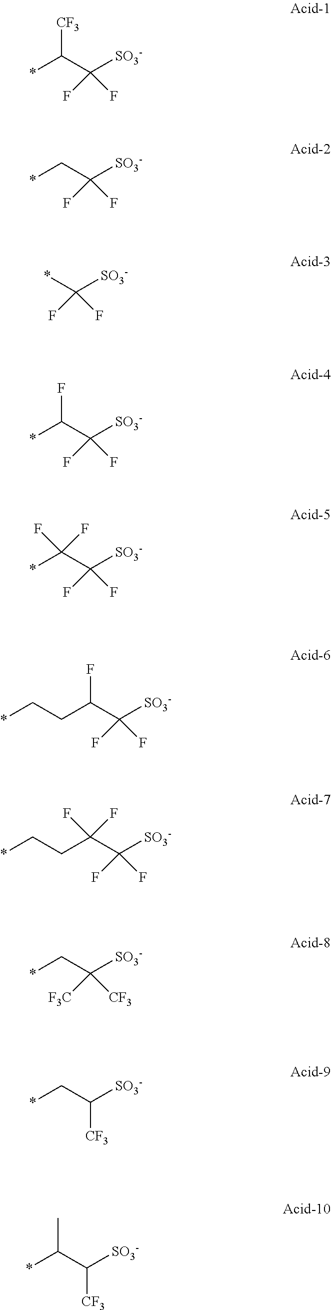

Of these, Acid-1 to Acid-7 are preferred, and Acid-1 to Acid-3, Acid-6 and Acid-7 are more preferred.

Of the onium salt type monomers of formula (a), a structure having formula (a1) is preferred.

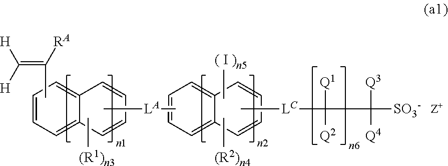

wherein n1 to n6, R^{A}, R¹, R², L^{A}, L^{C}, Q¹ to Q⁴, and Z⁺ are as defined above.

Of the onium salt type monomers of formula (a1), a structure having formula (a2) is preferred.

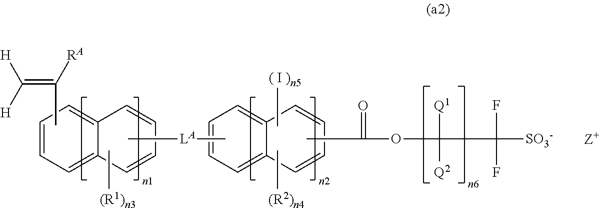

wherein n1 to n6, R^{A}, R¹, R², L^{A}, Q¹, Q², and Z⁺ are as defined above.

Examples of the anion in the onium salt type monomer having formula (a) are shown below, but not limited thereto. In the following formulae, R^{A} and Q¹ are as defined above, and Me is a methyl group. The bonding positions of the substituents on the aromatic ring may be interchanged.

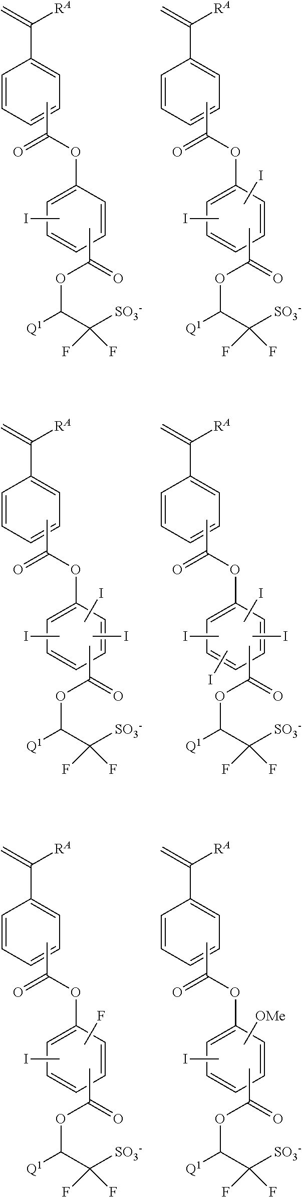

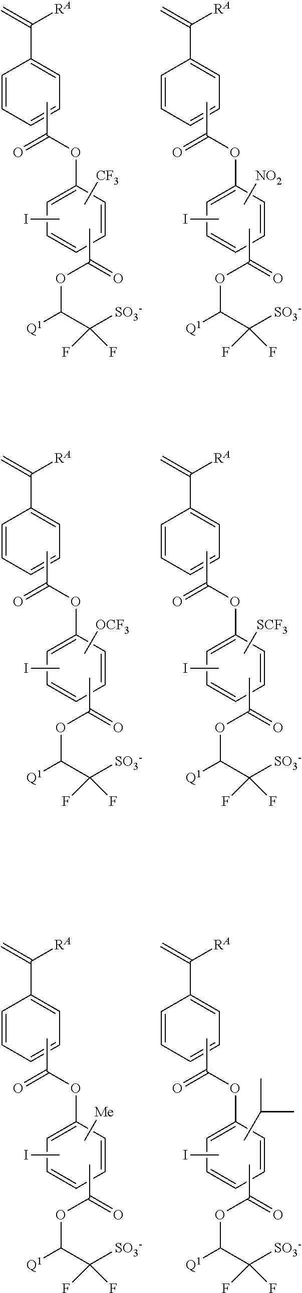

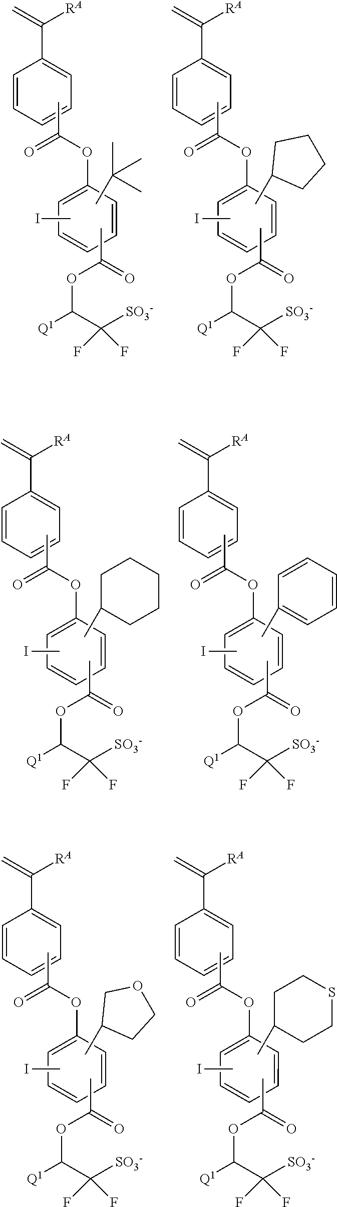

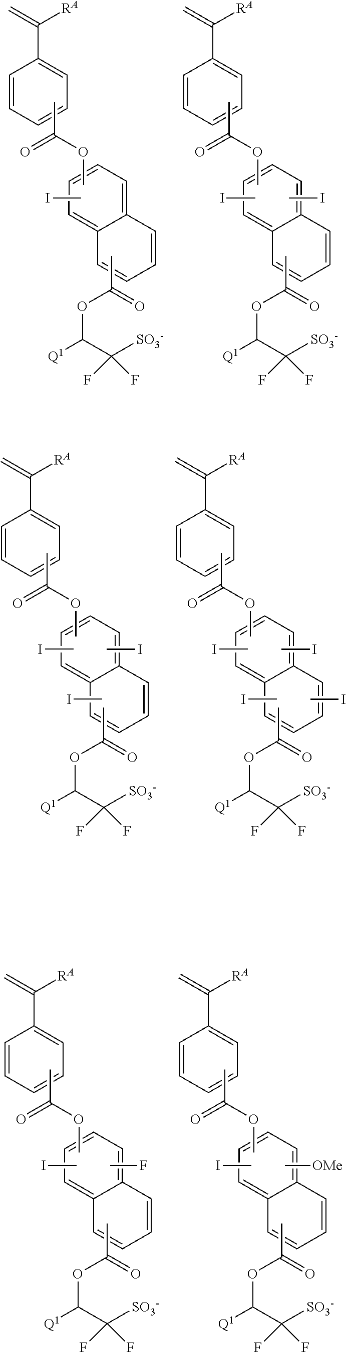

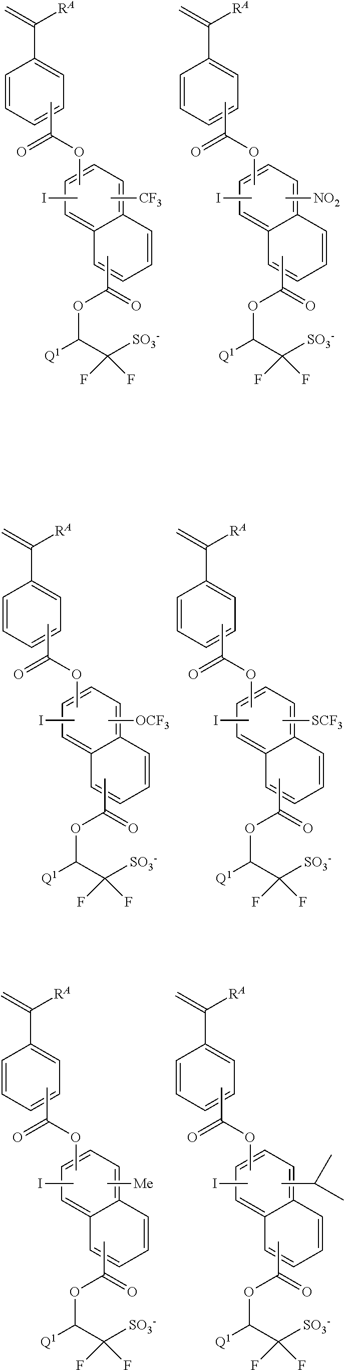

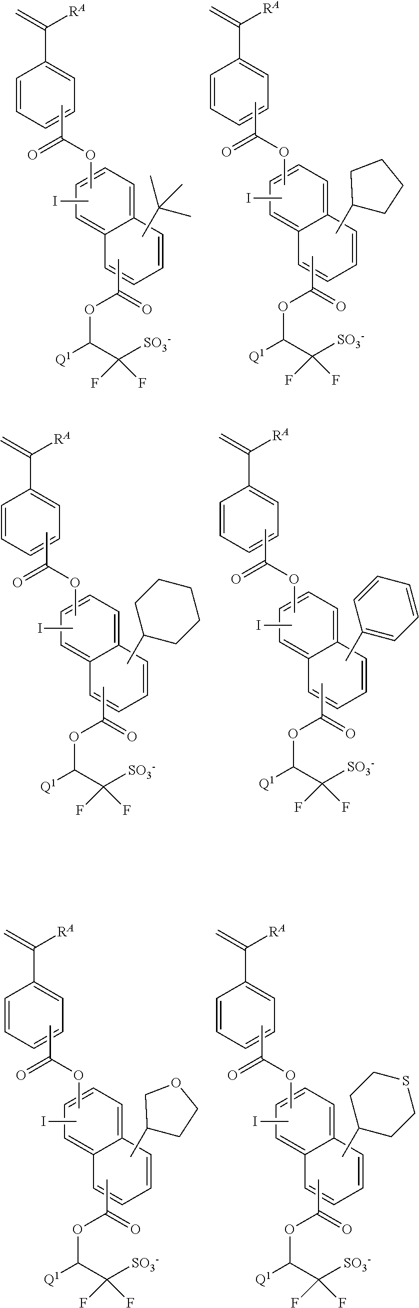

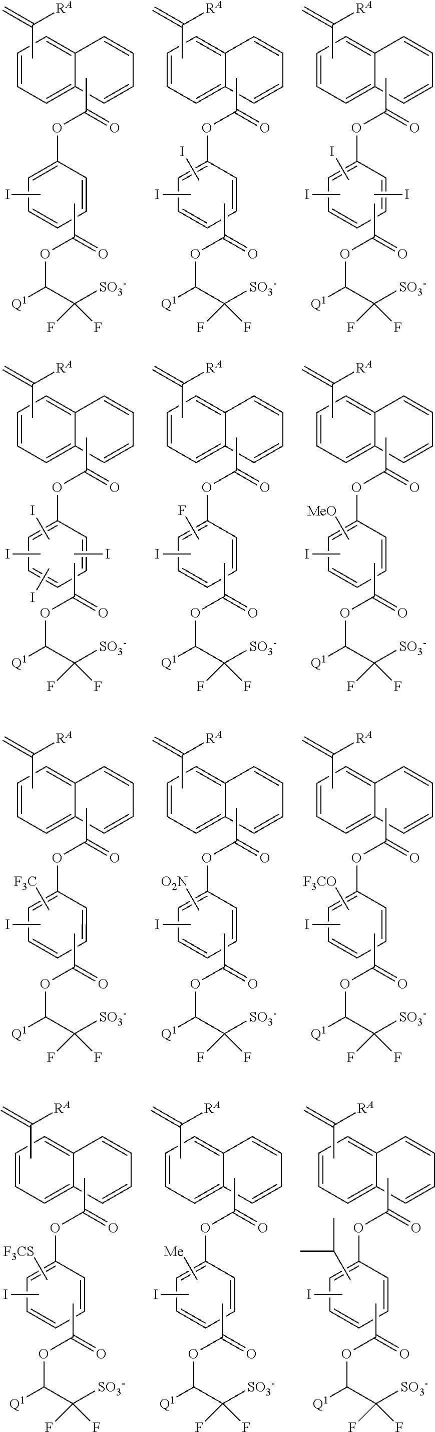

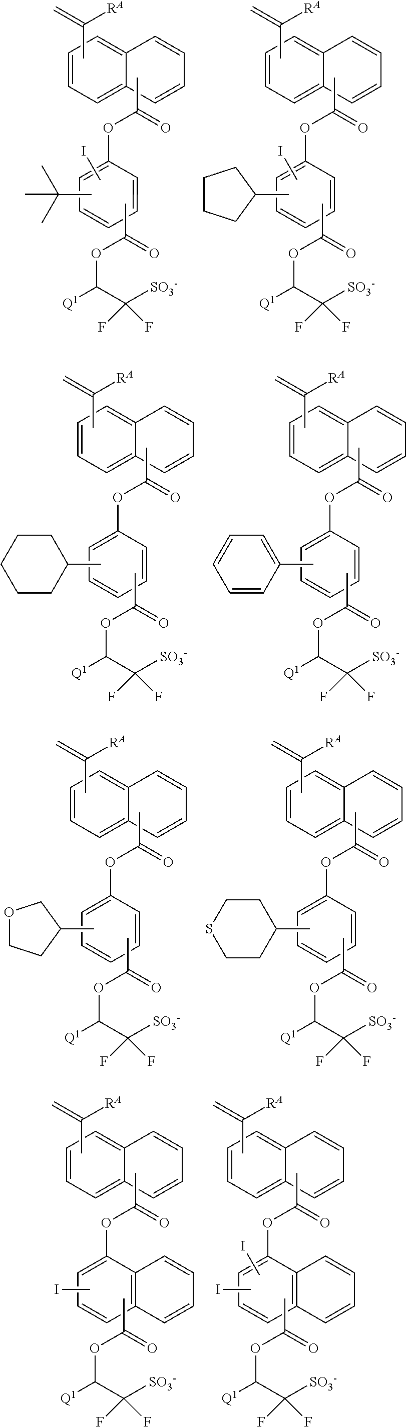

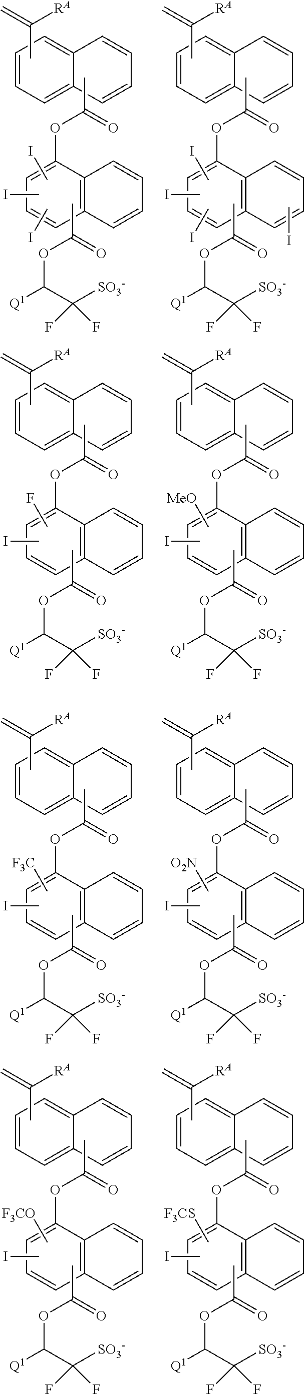

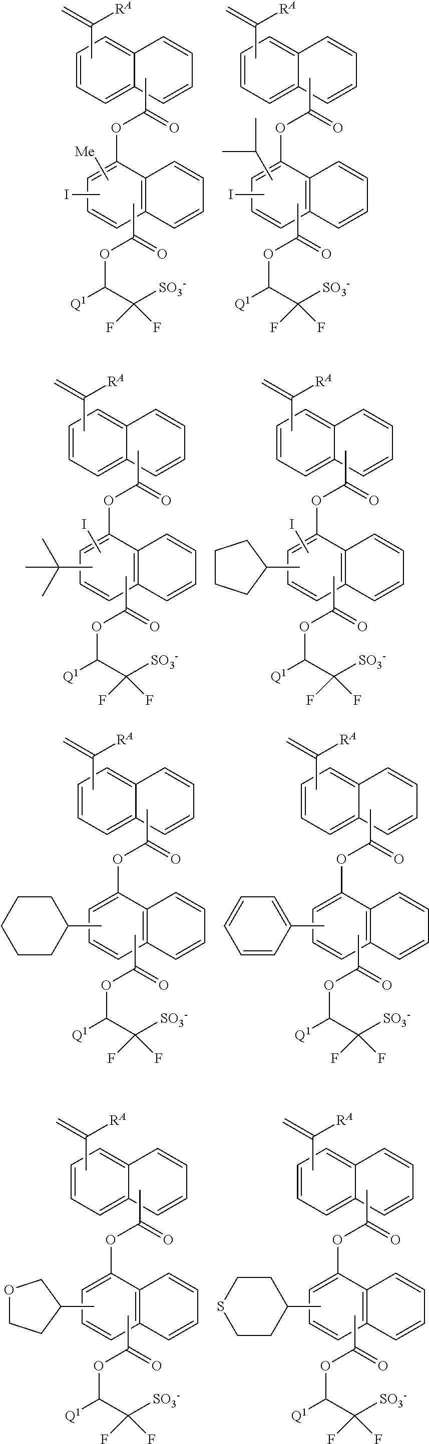

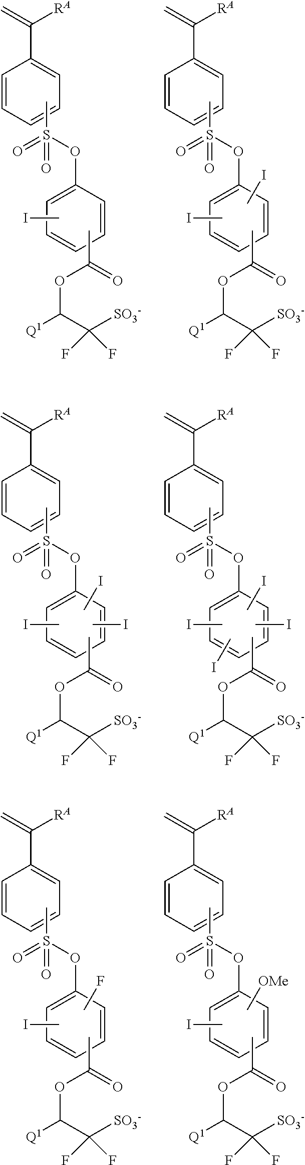

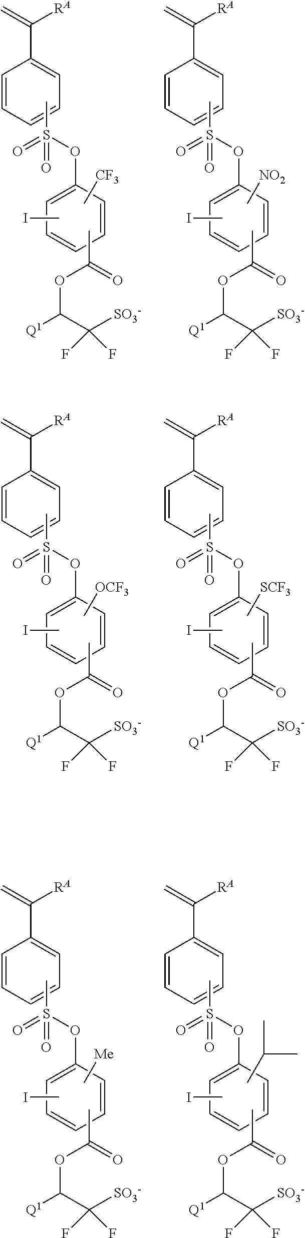

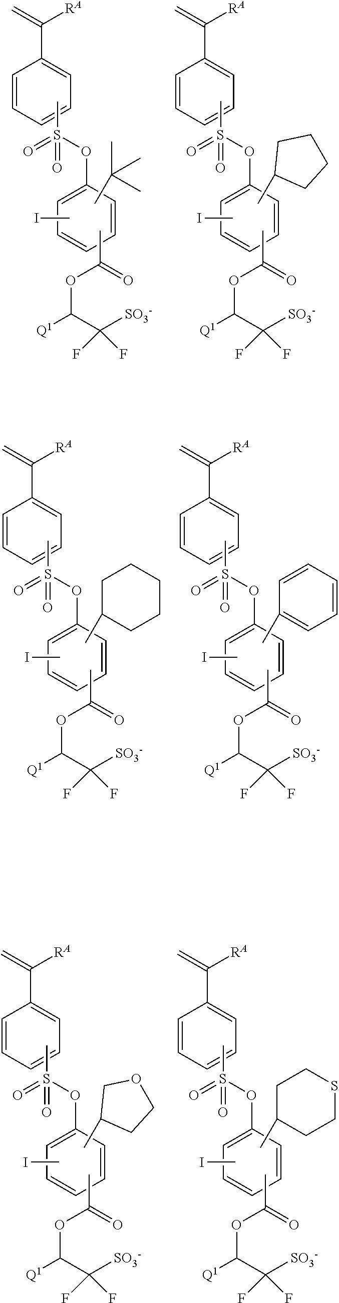

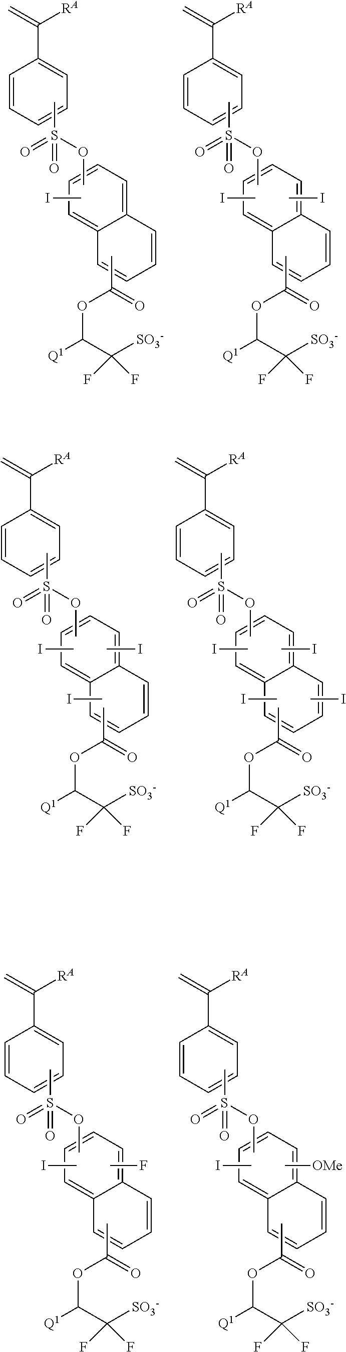

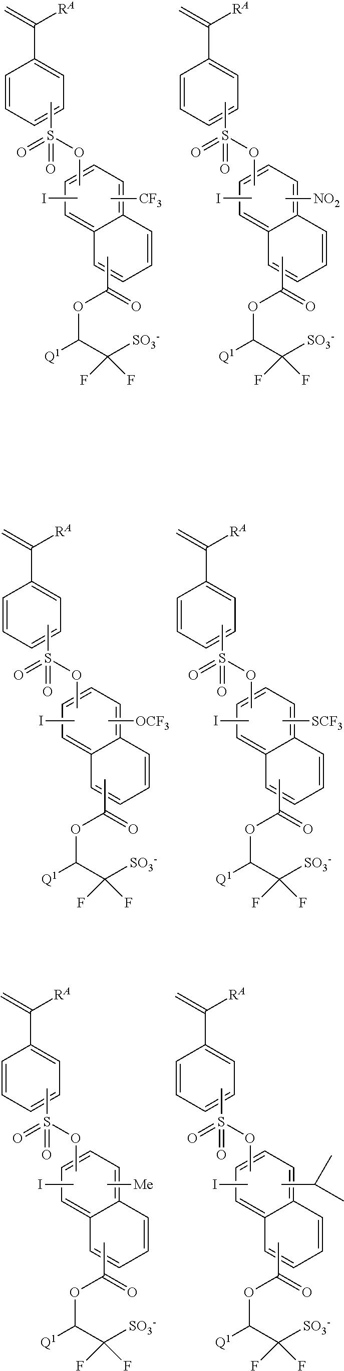

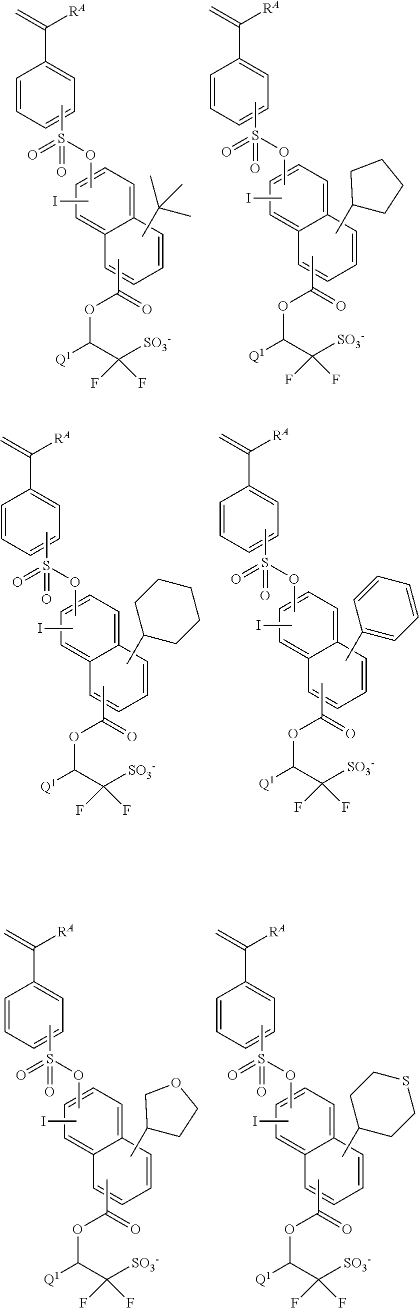

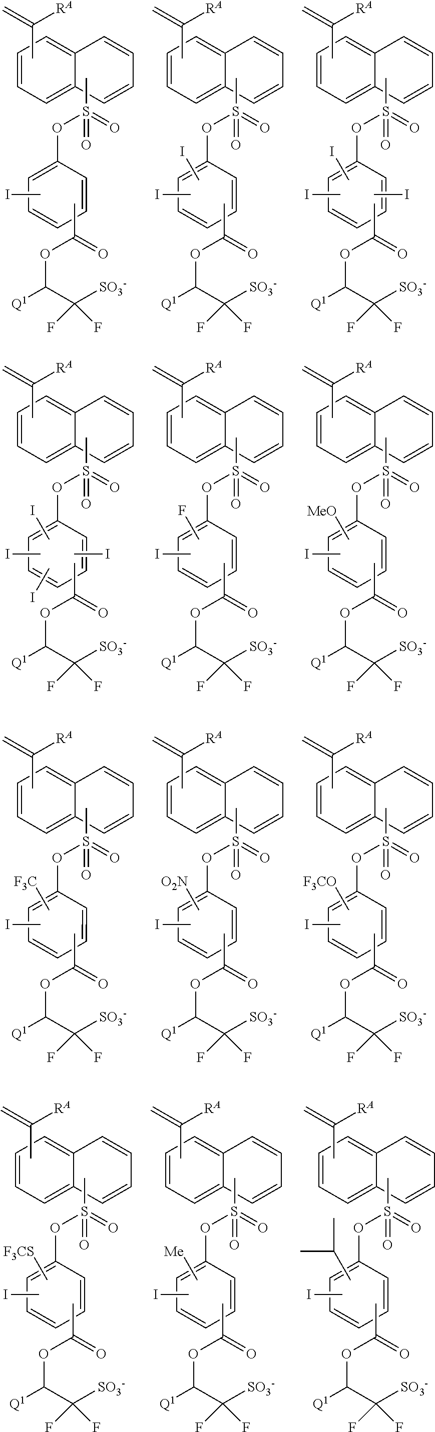

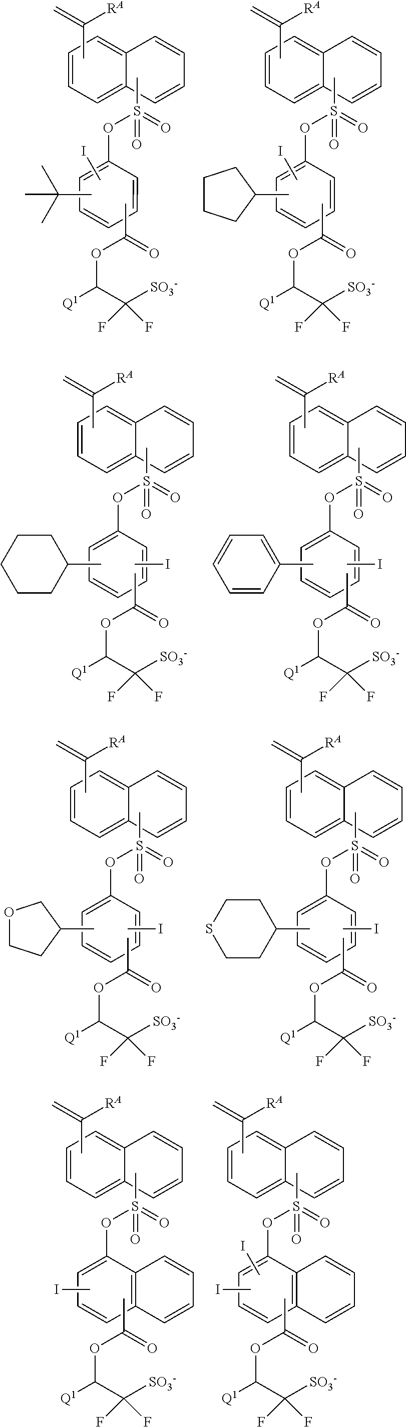

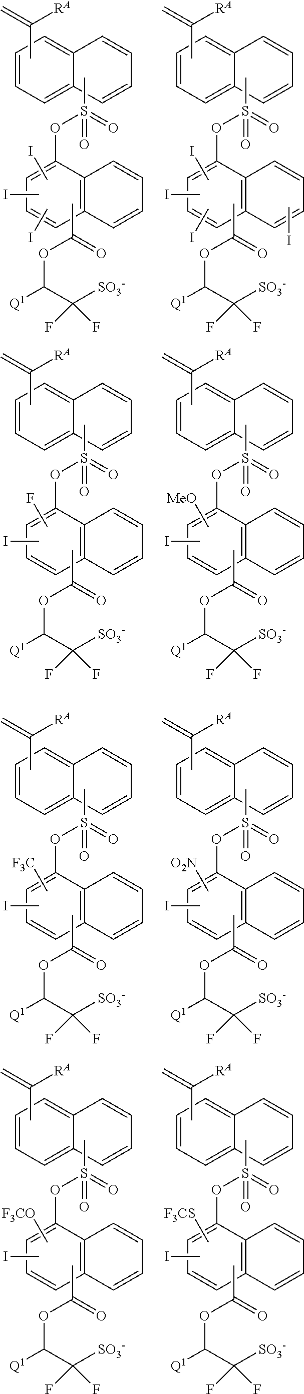

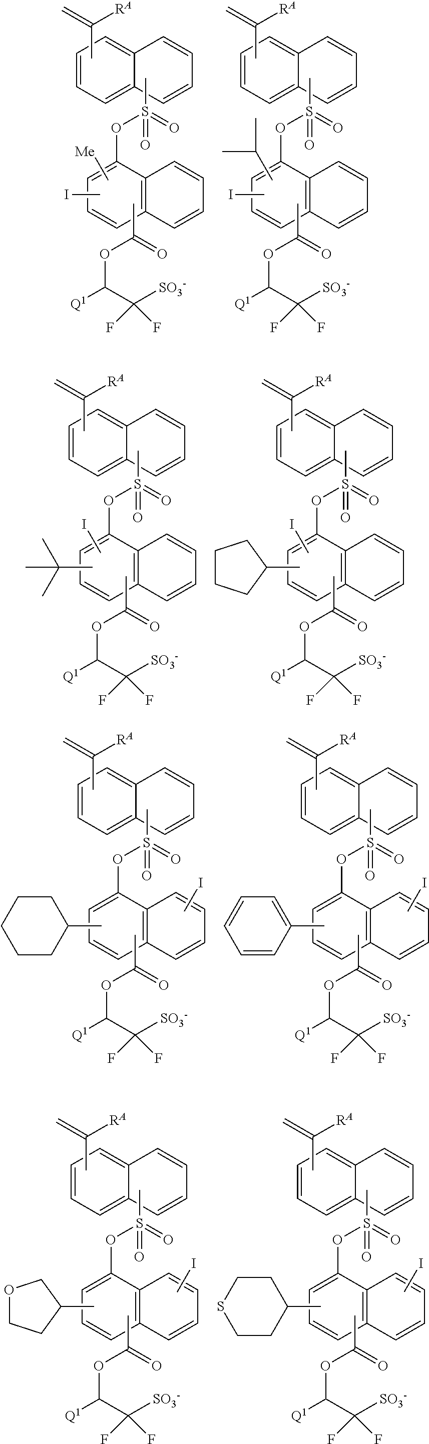

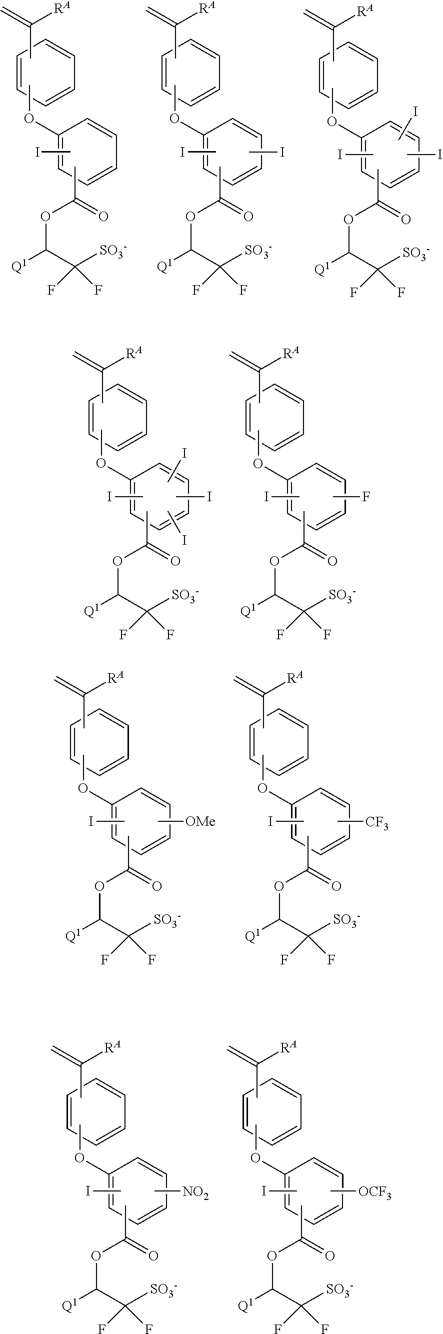

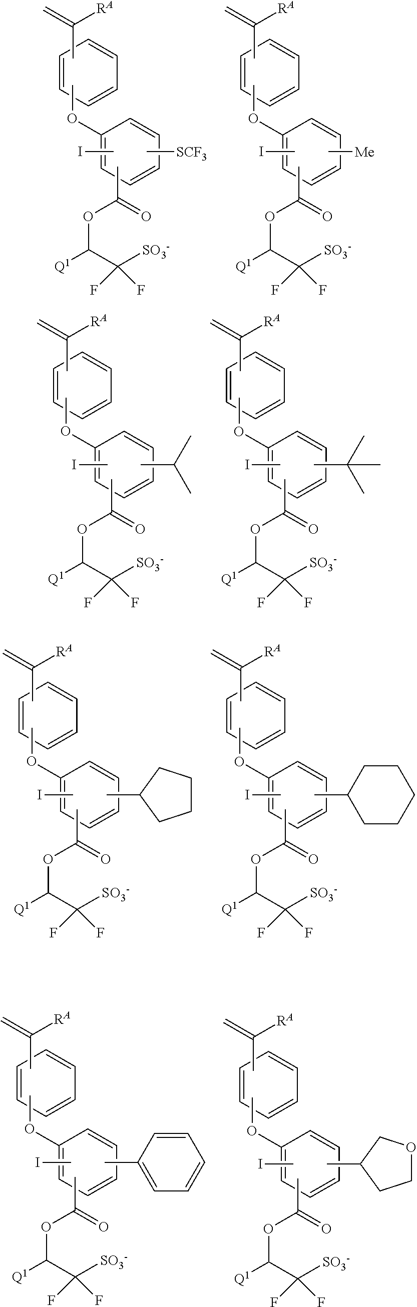

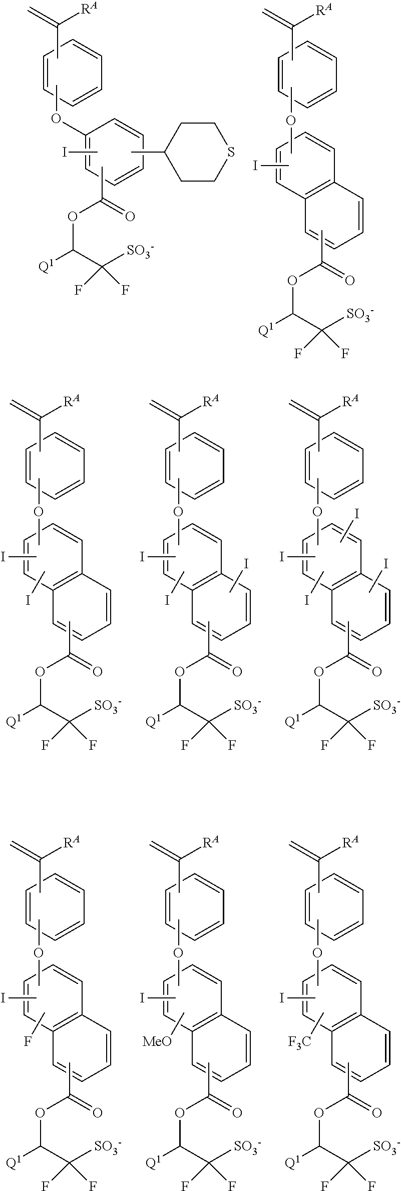

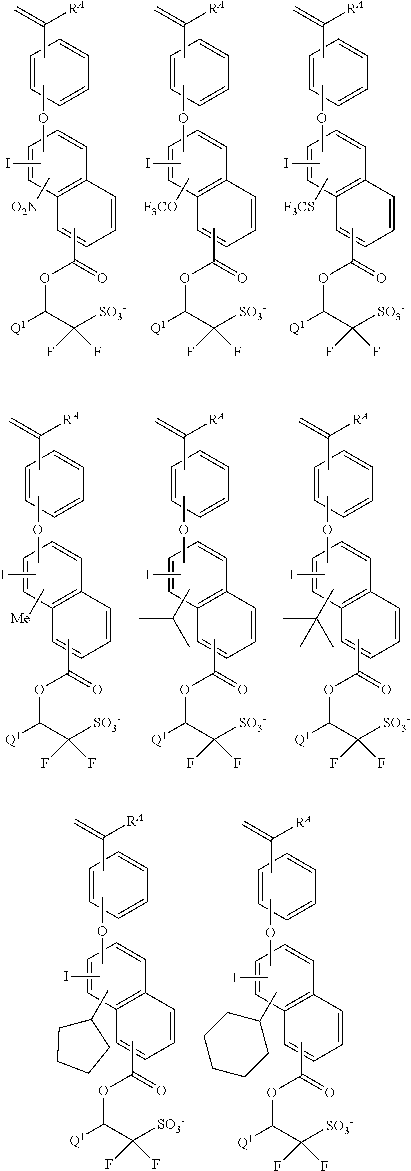

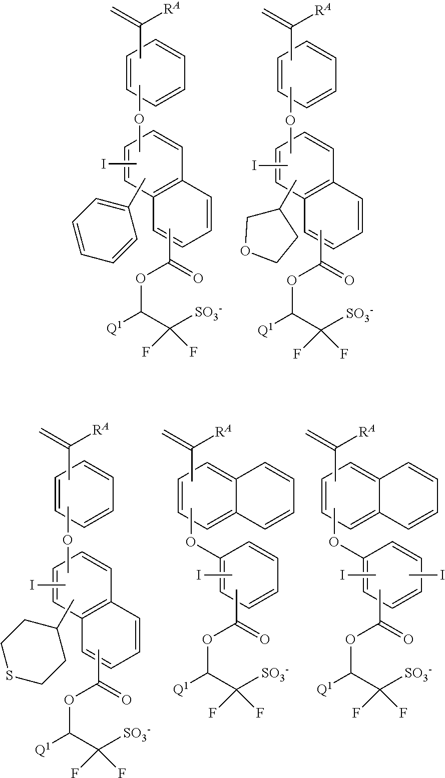

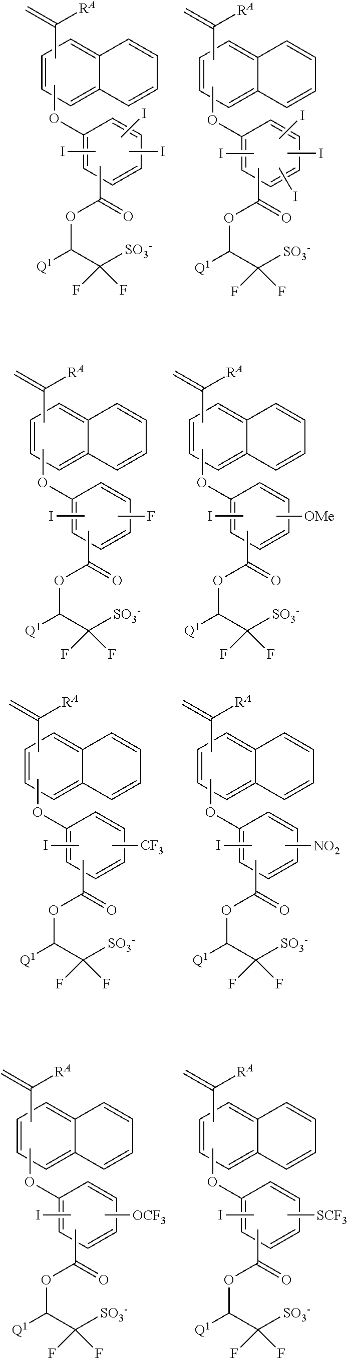

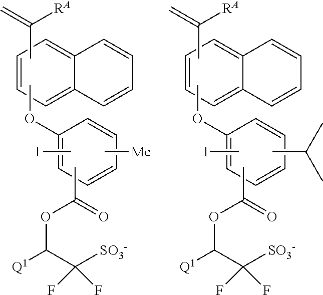

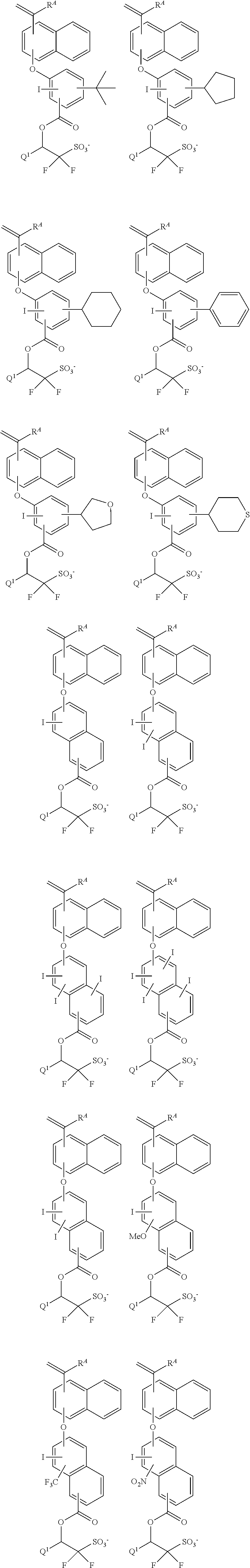

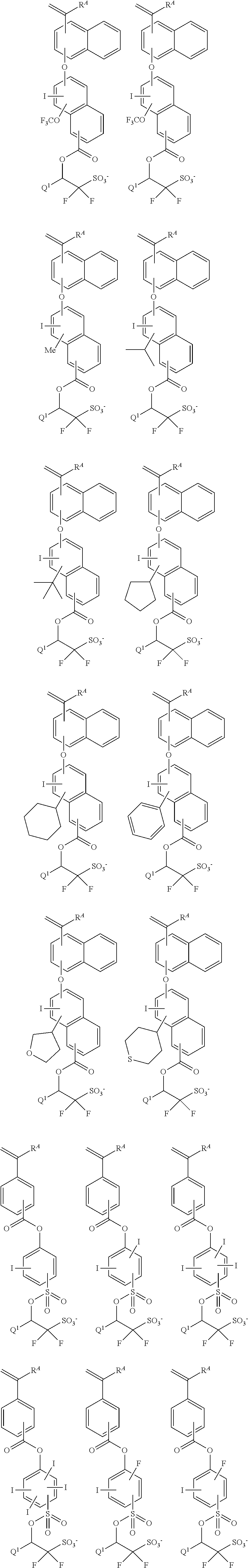

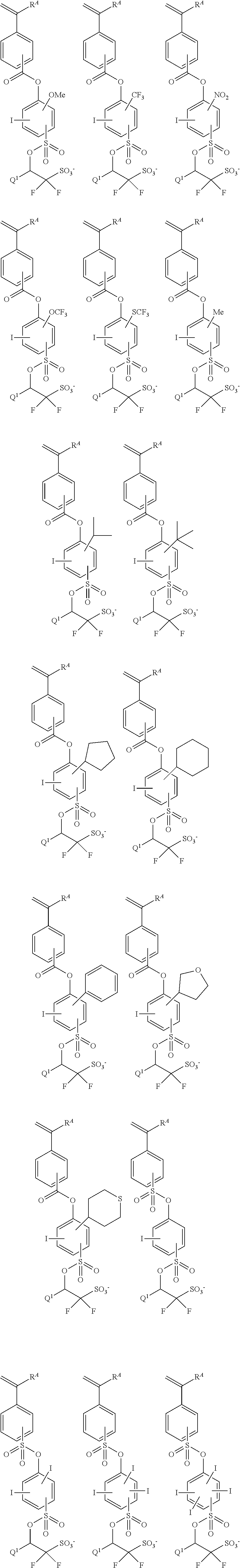

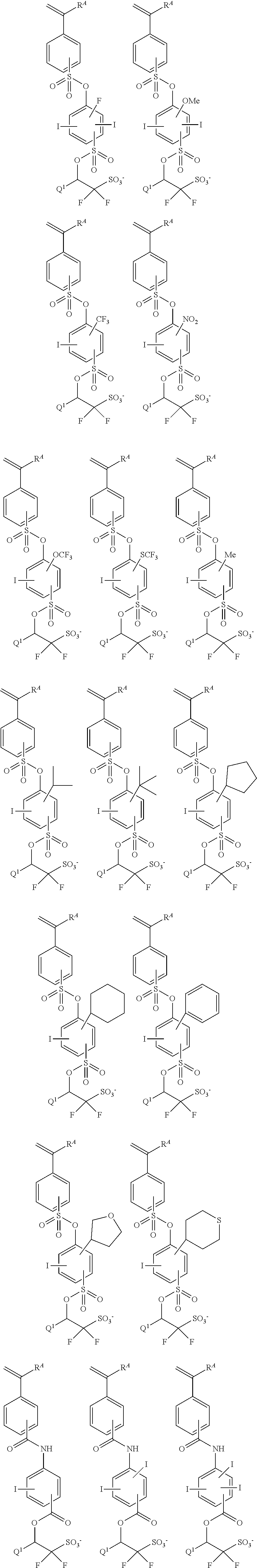

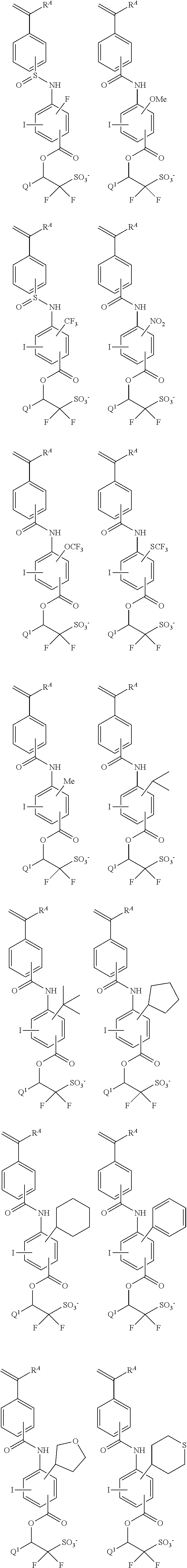

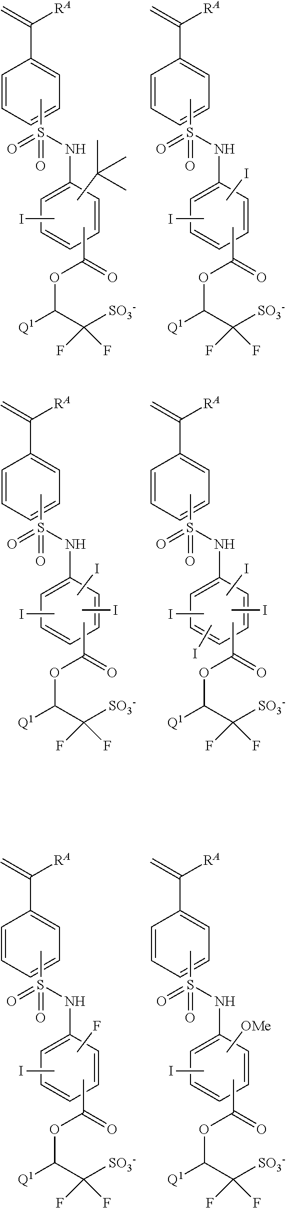

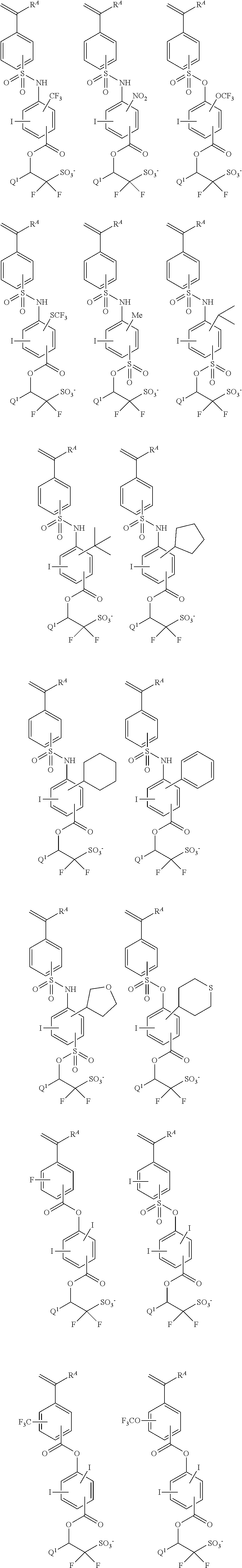

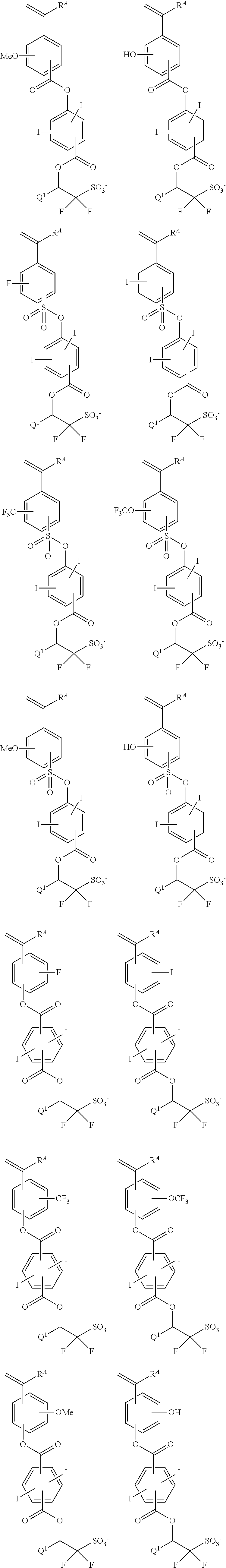

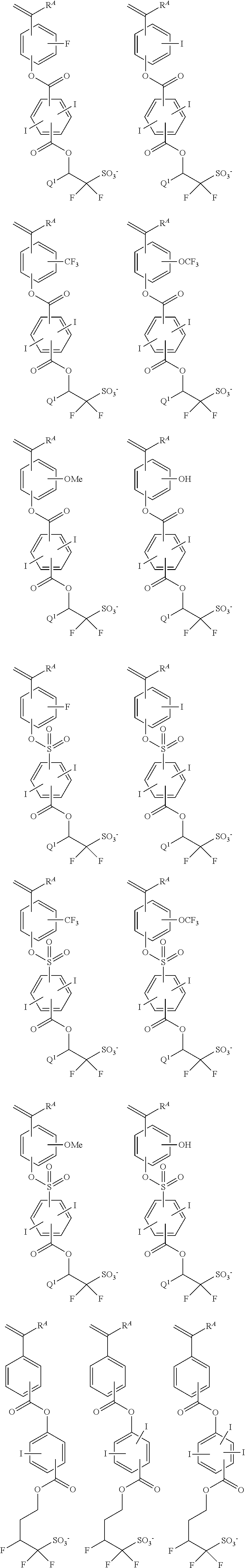

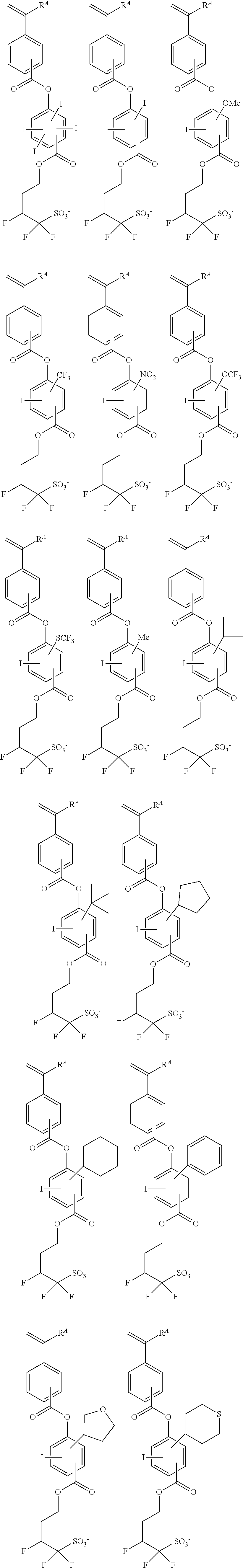

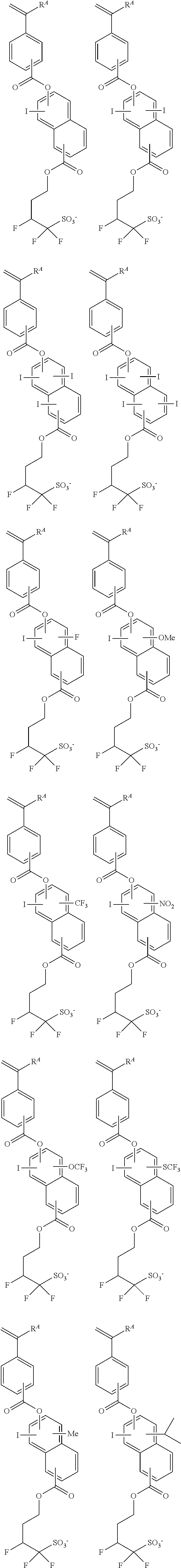

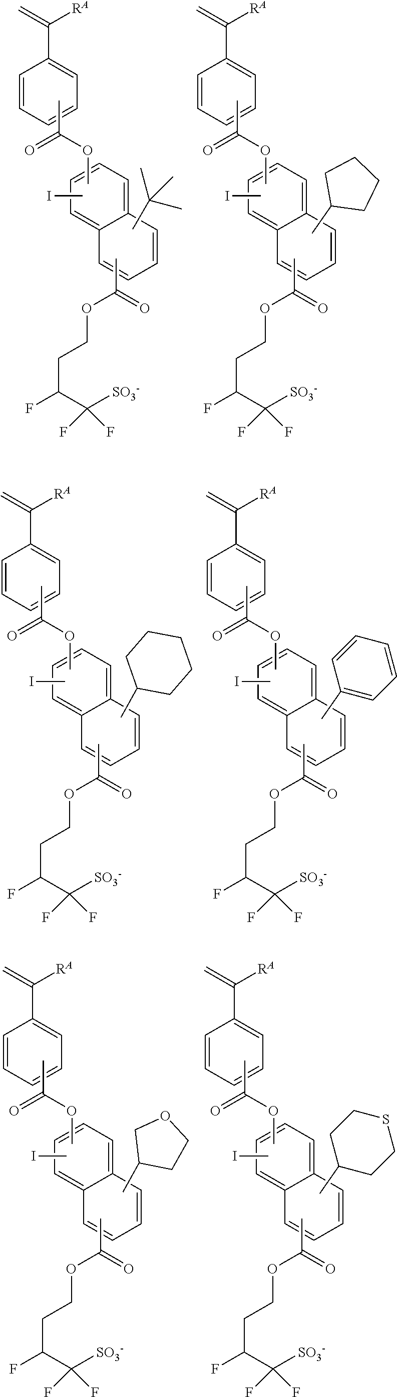

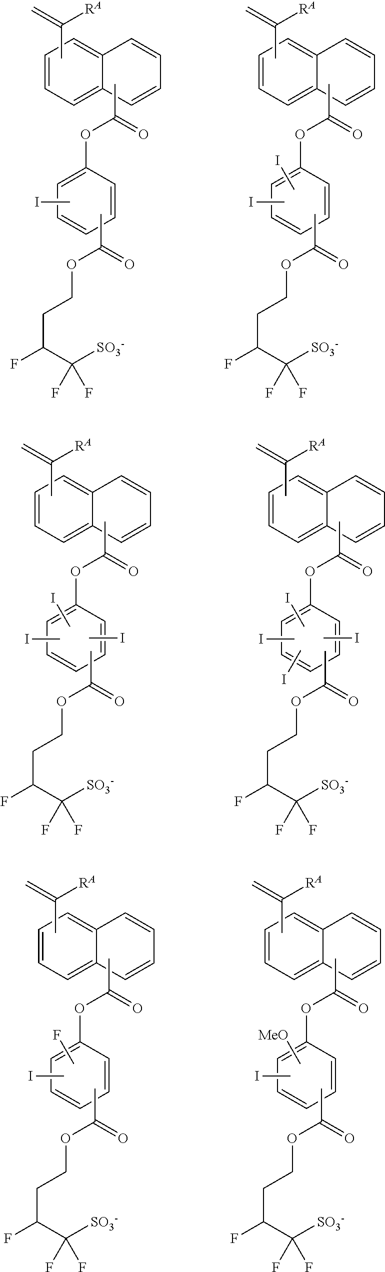

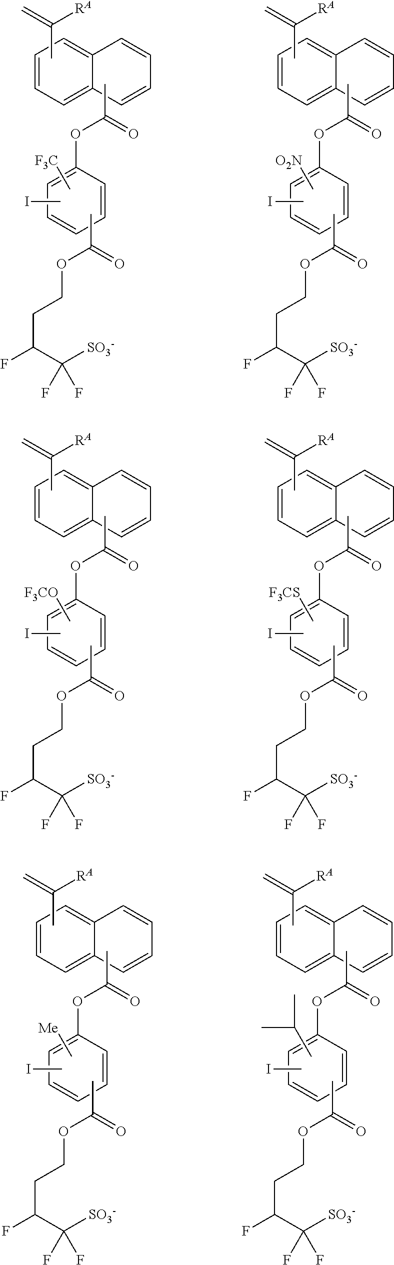

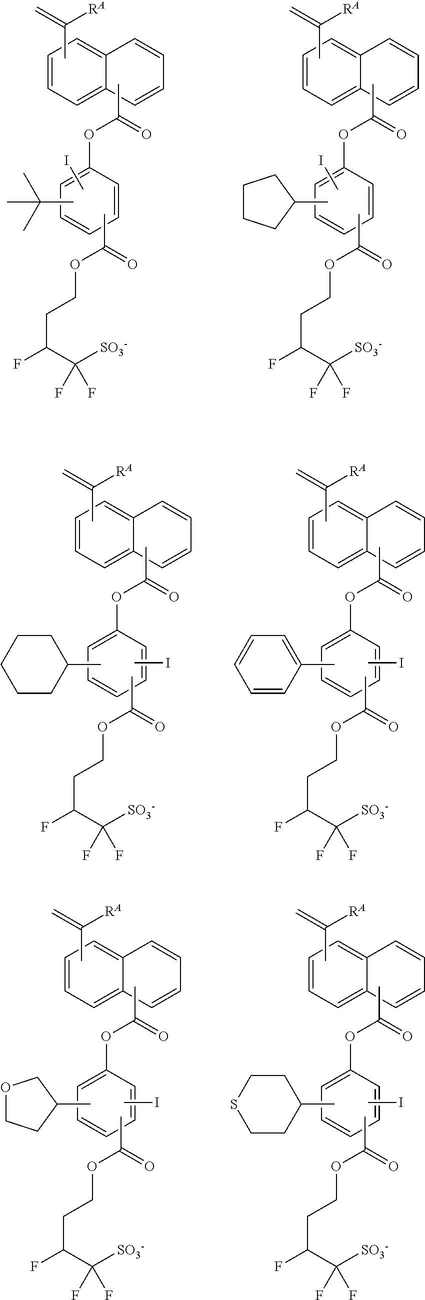

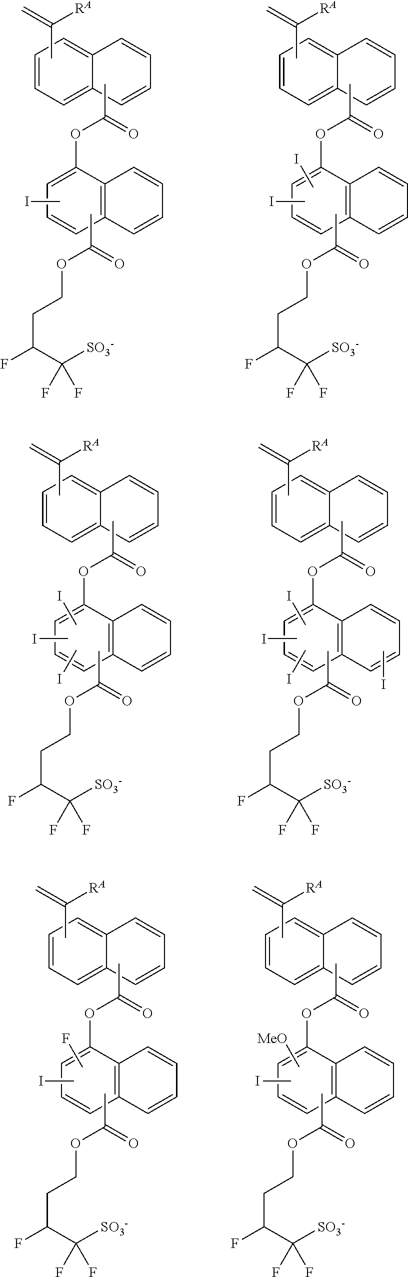

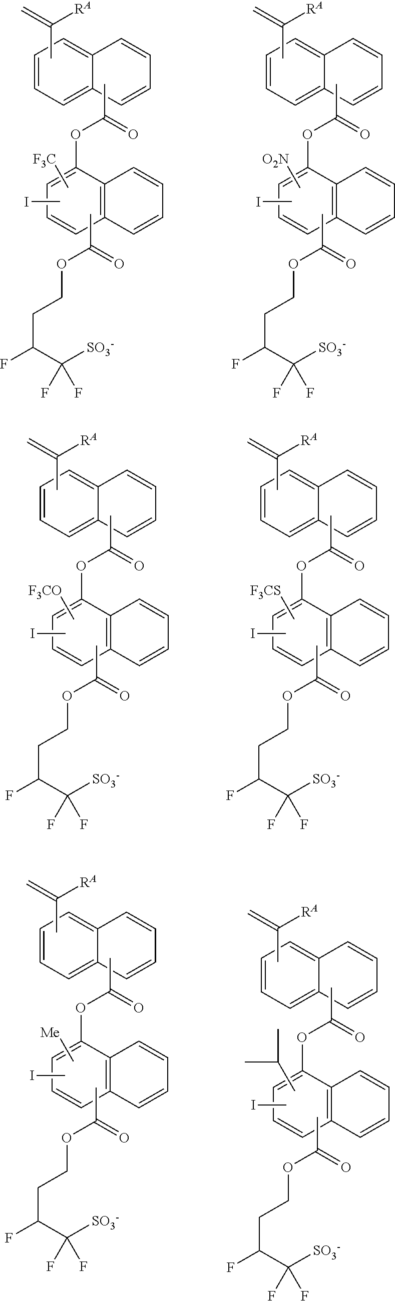

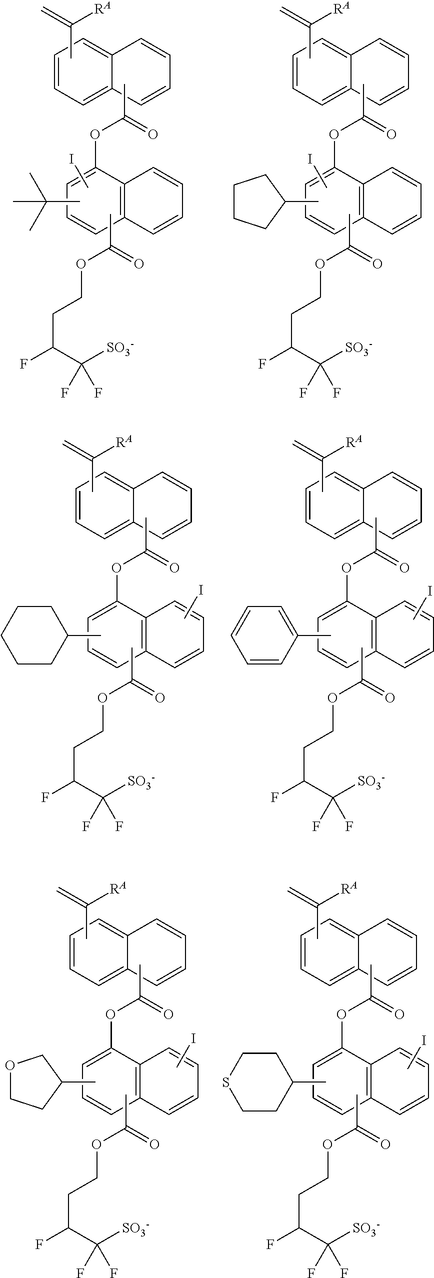

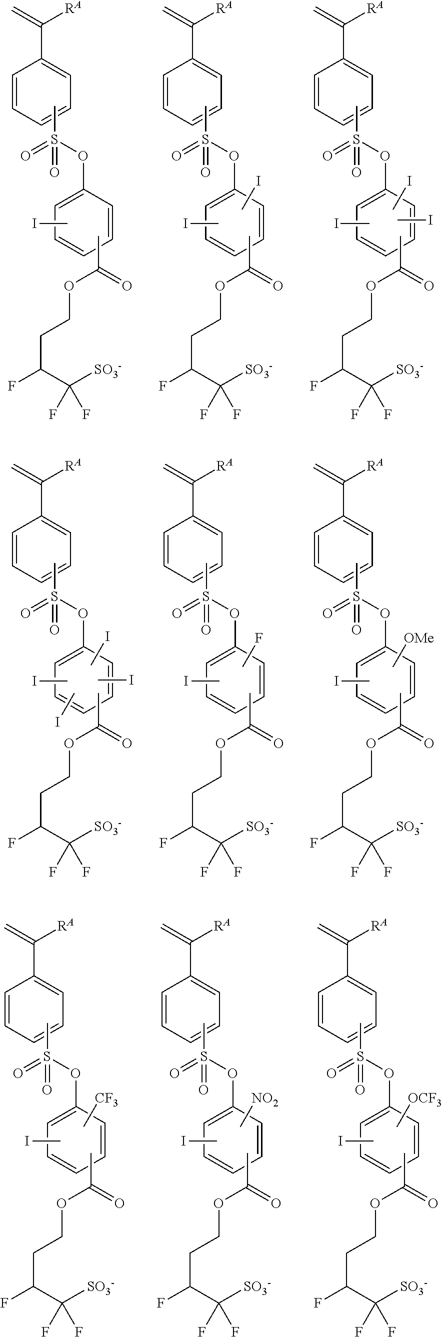

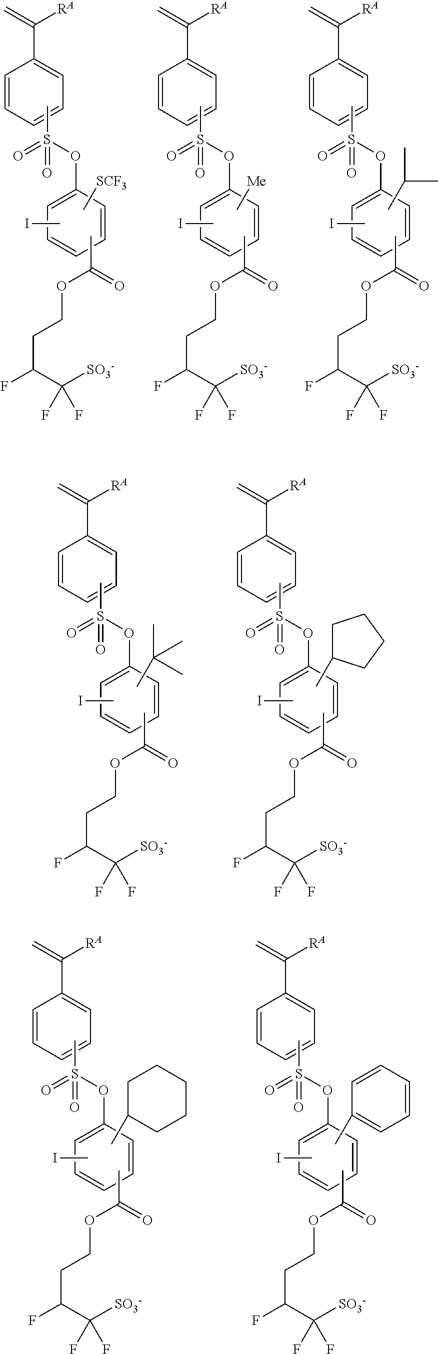

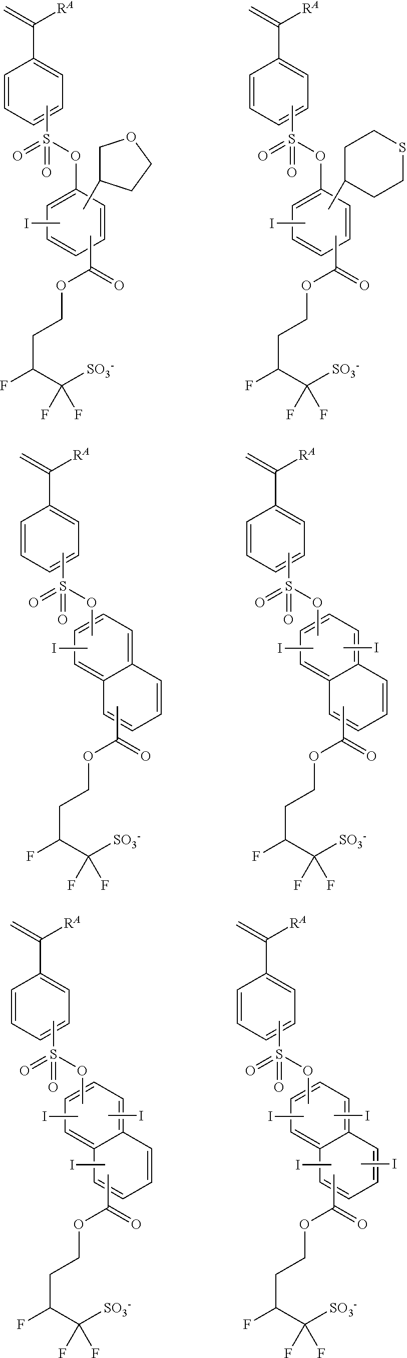

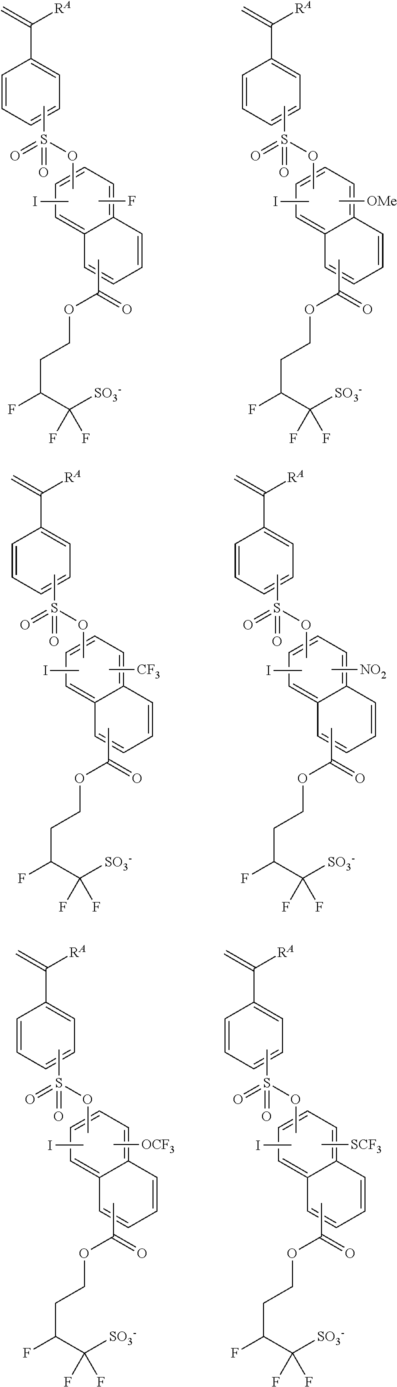

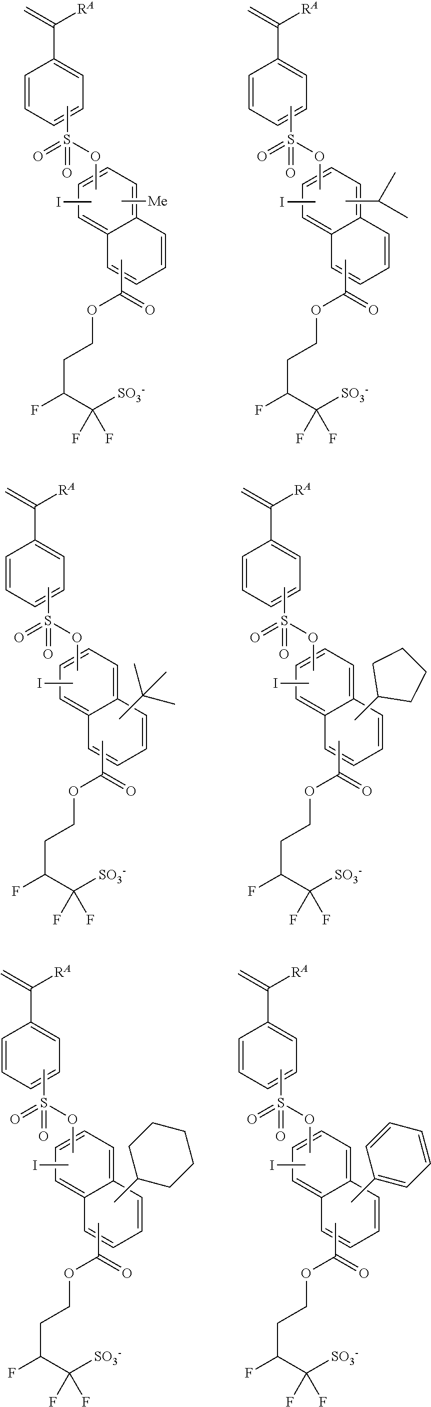

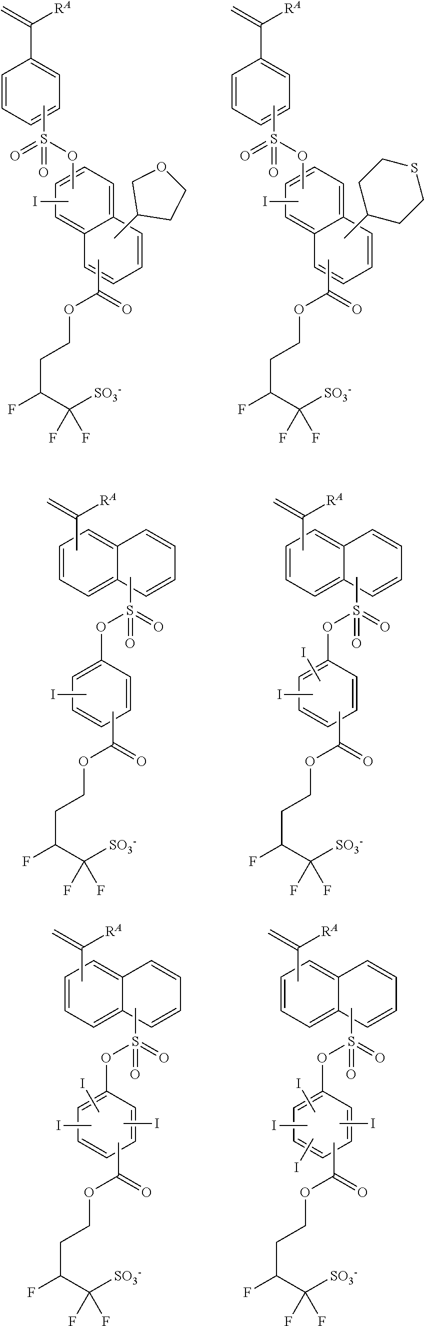

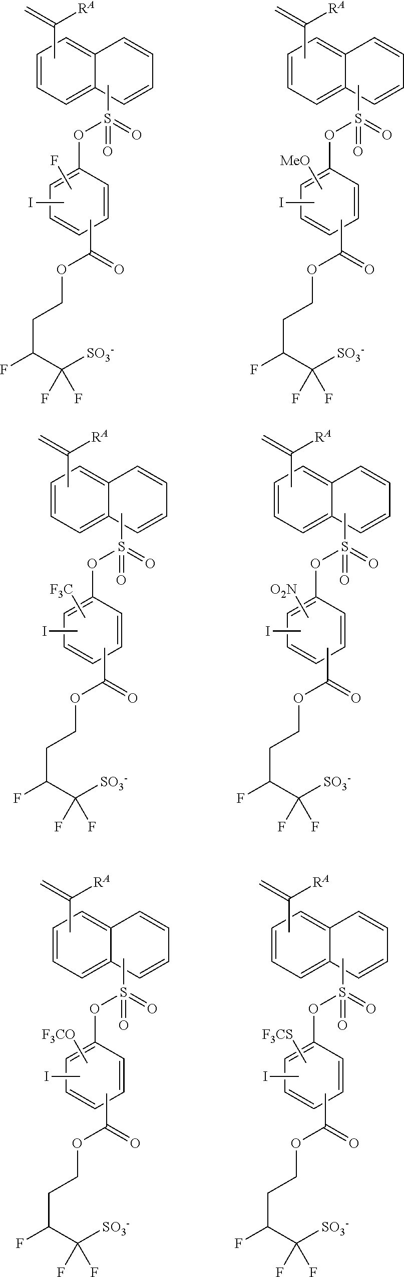

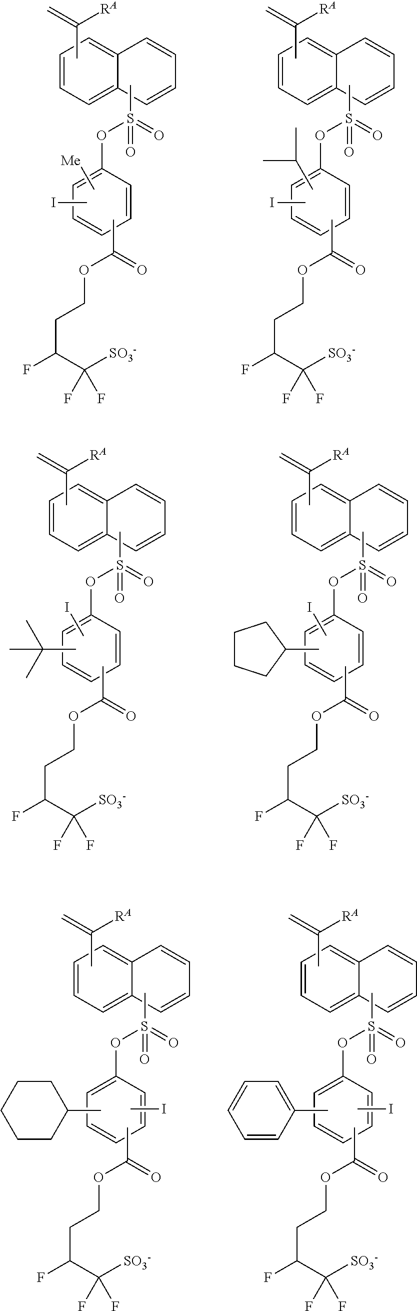

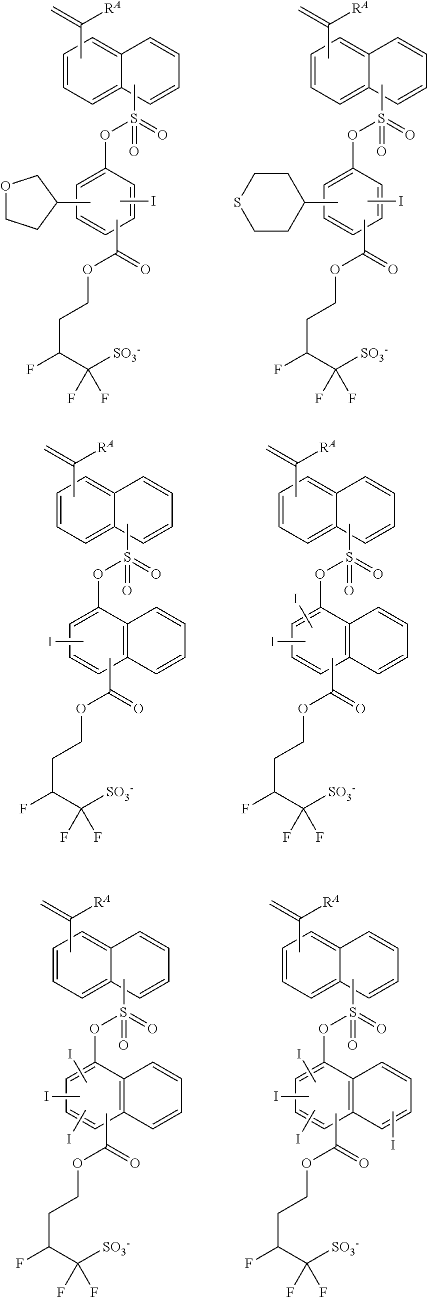

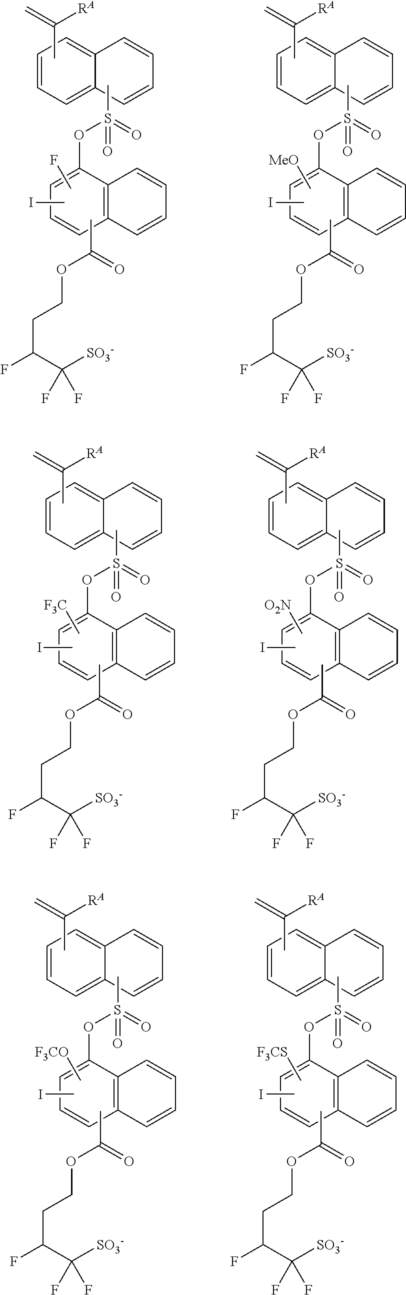

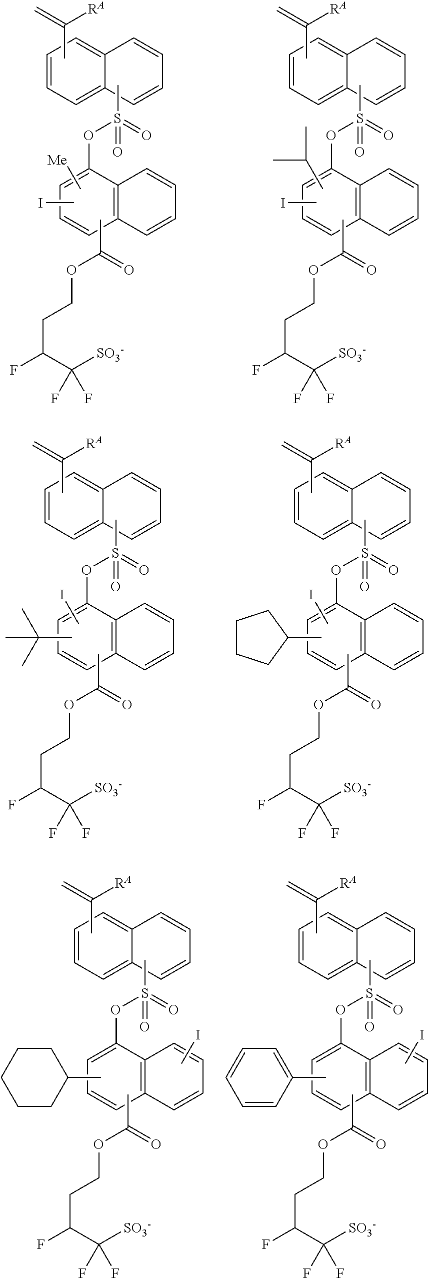

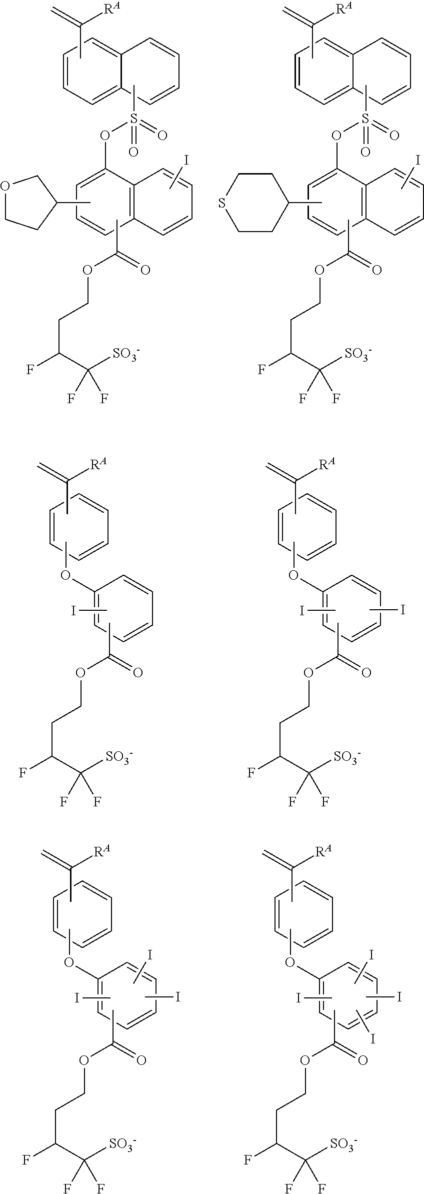

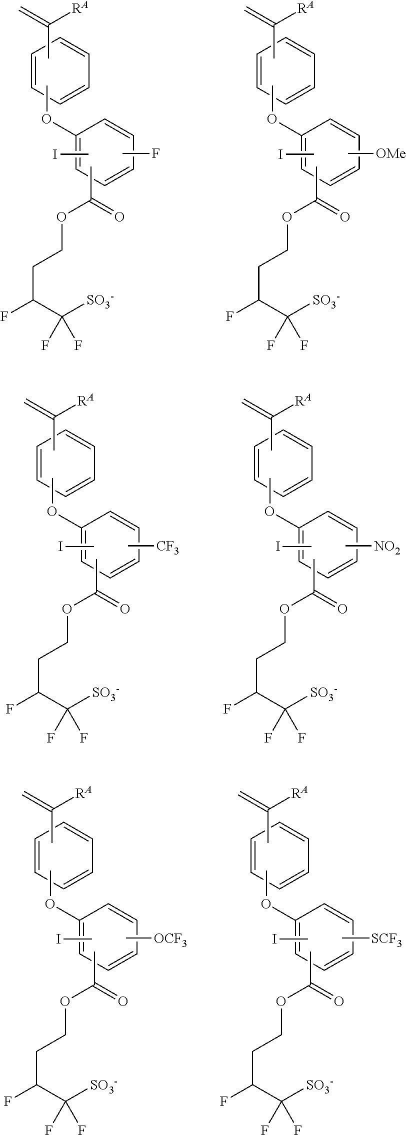

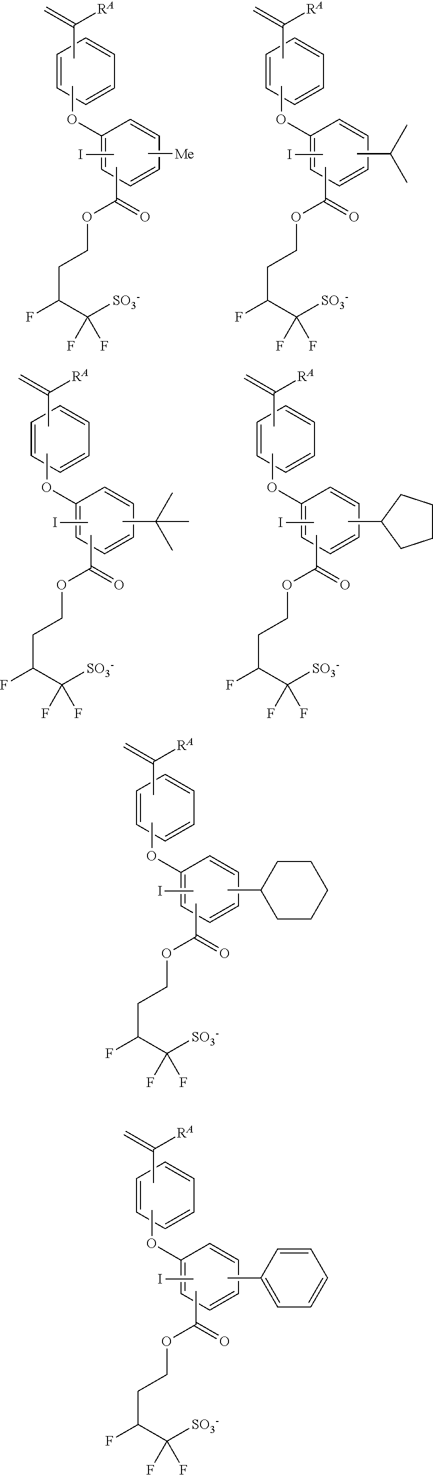

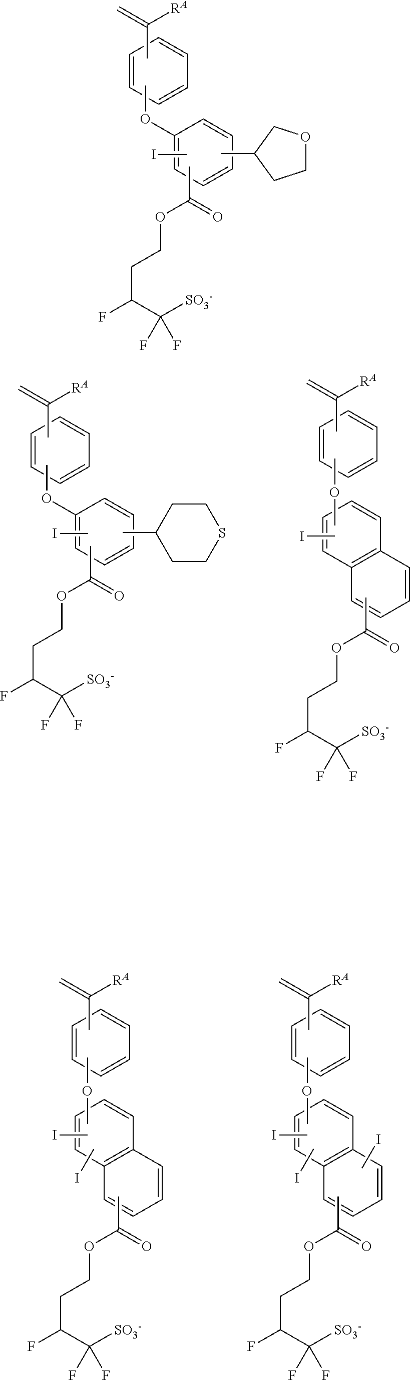

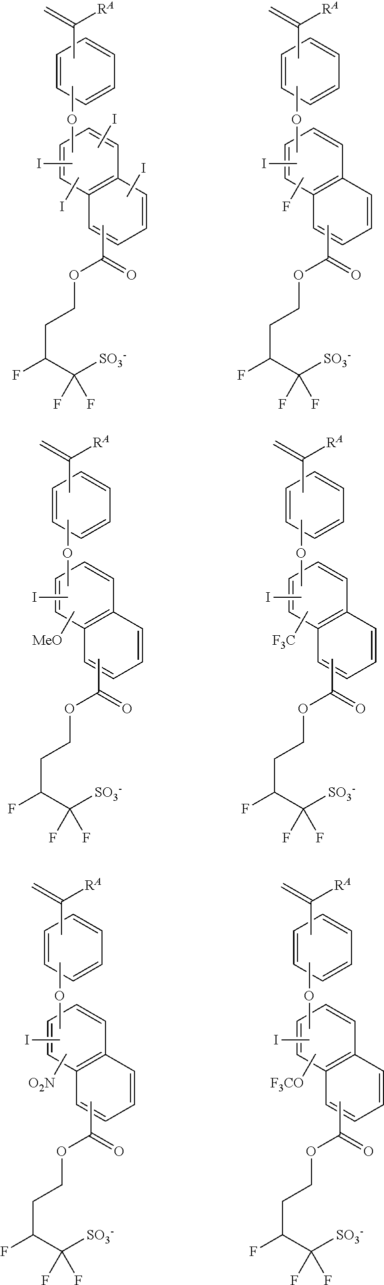

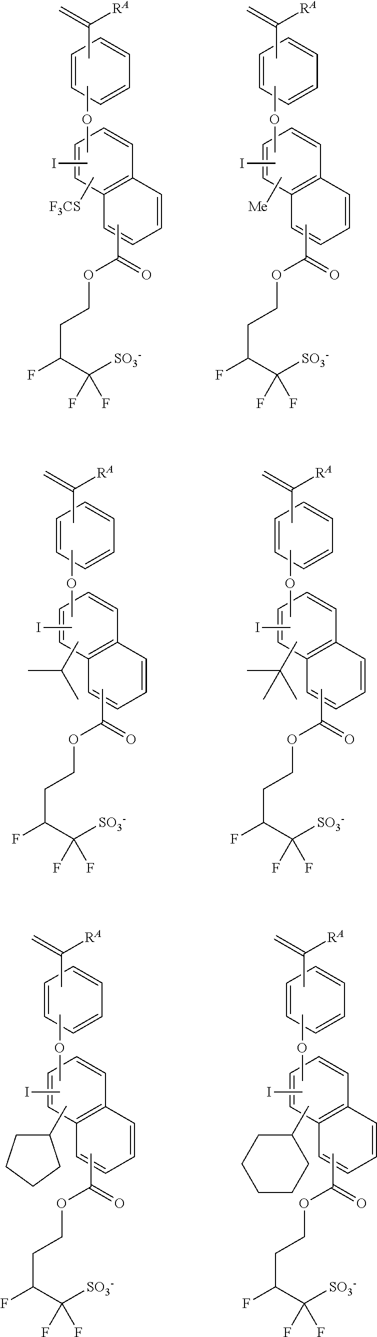

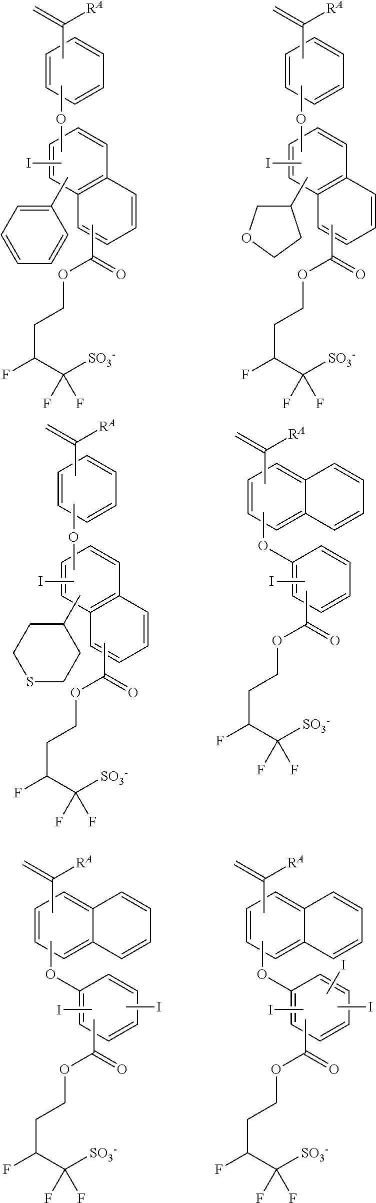

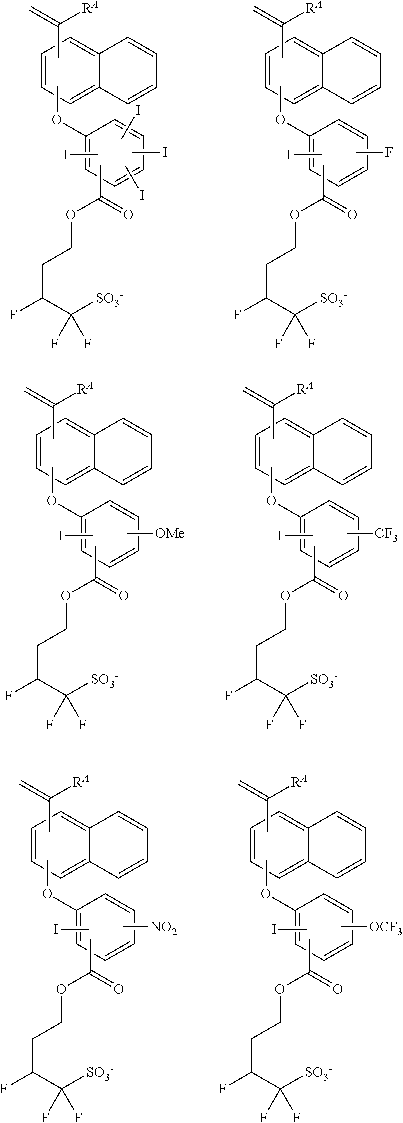

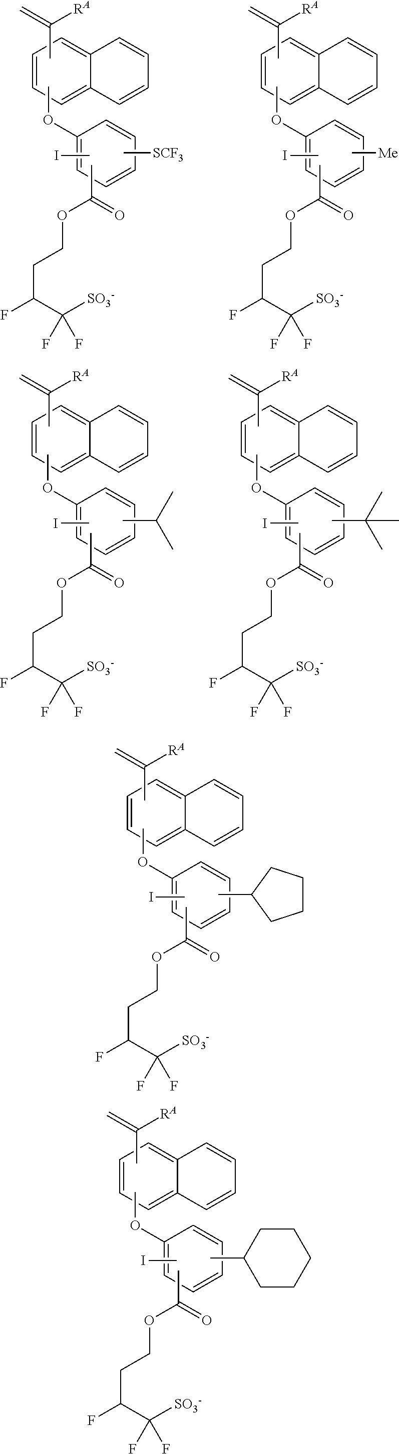

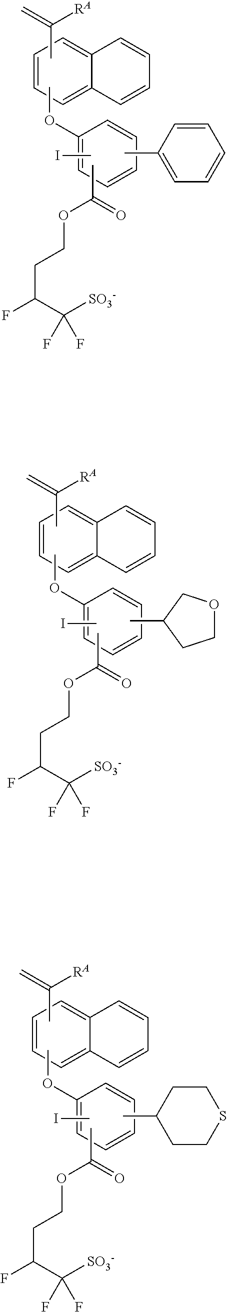

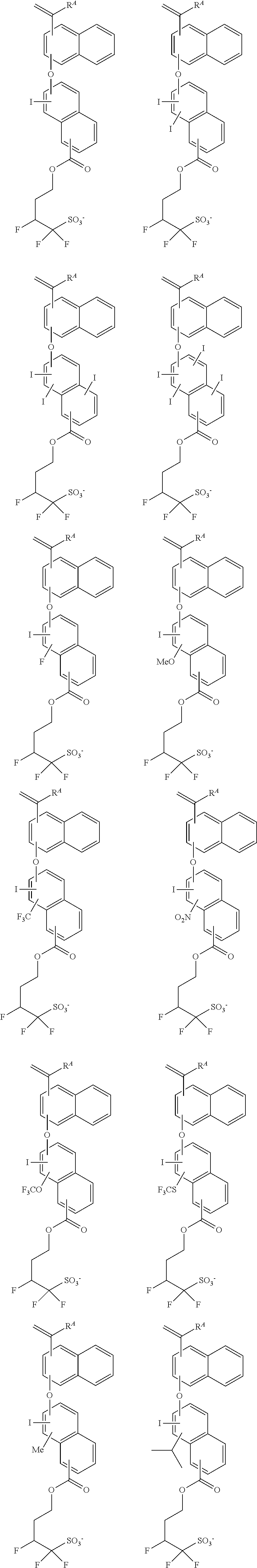

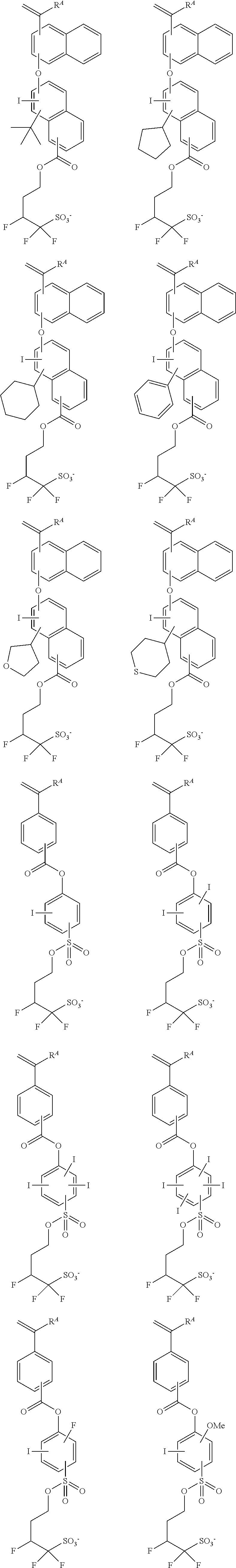

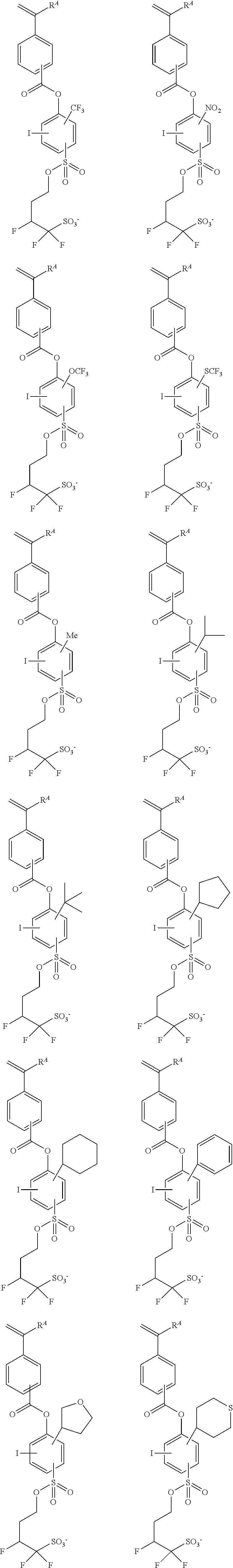

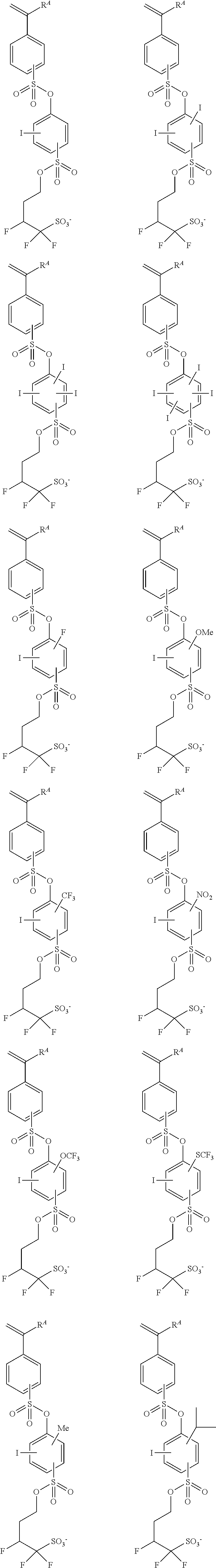

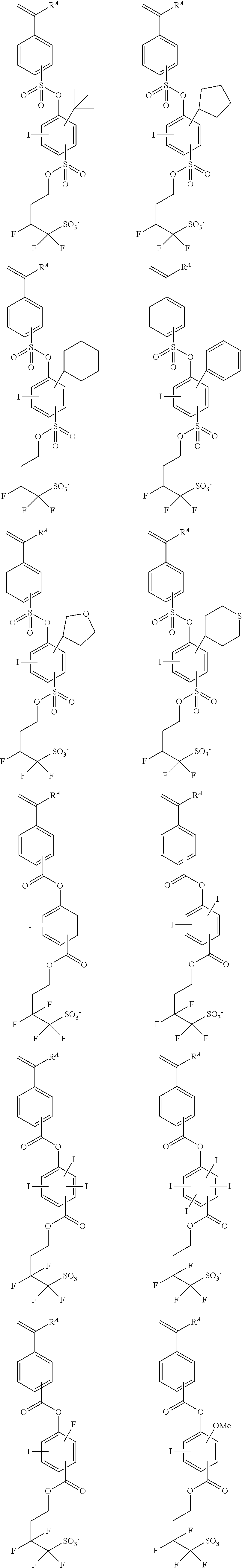

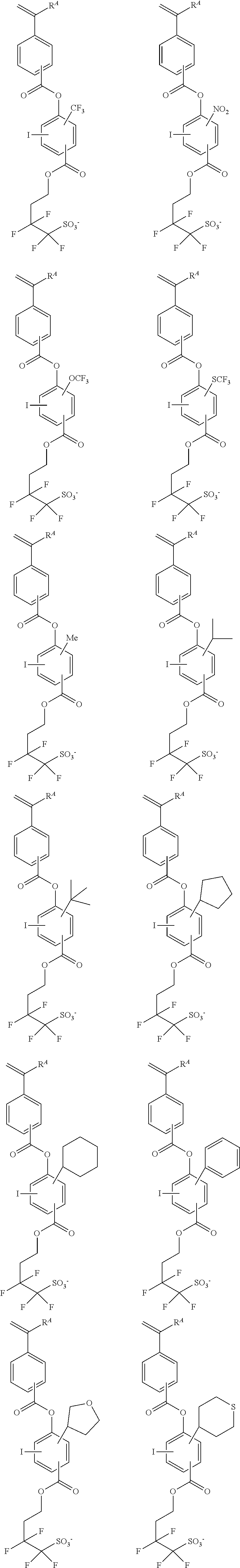

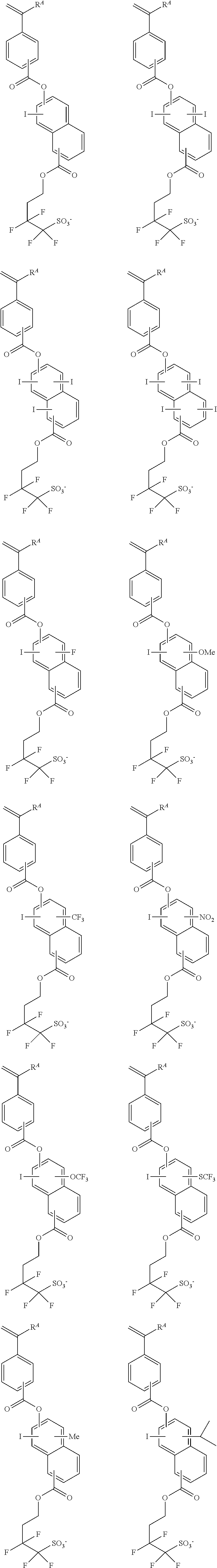

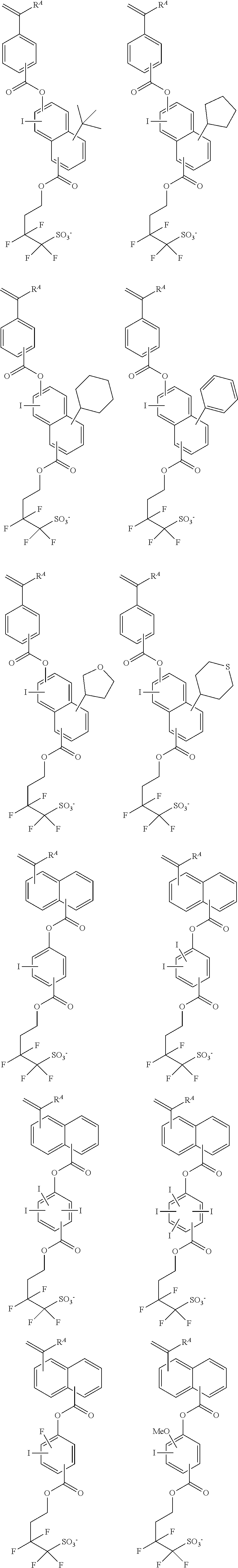

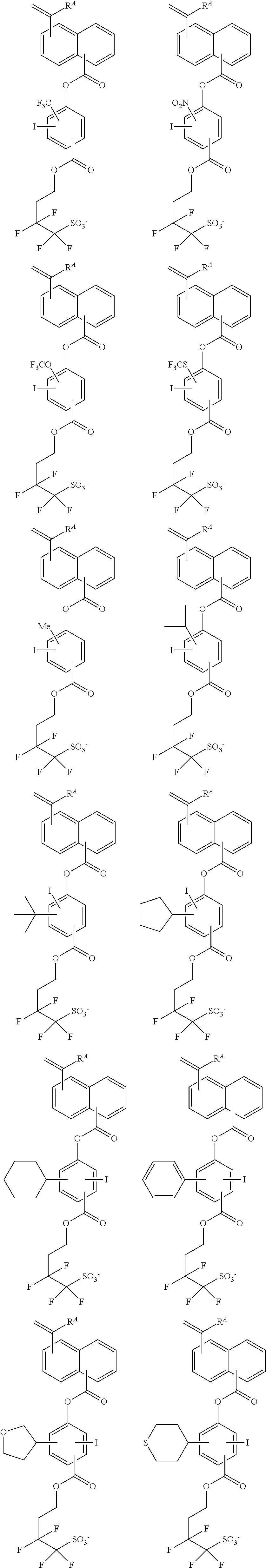

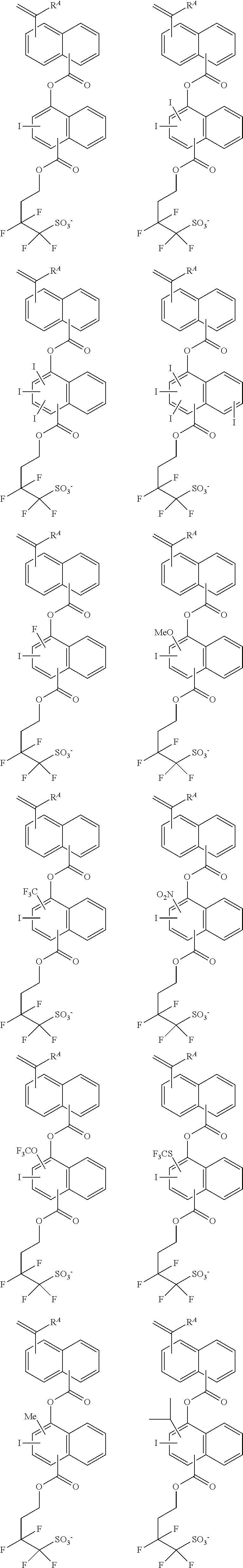

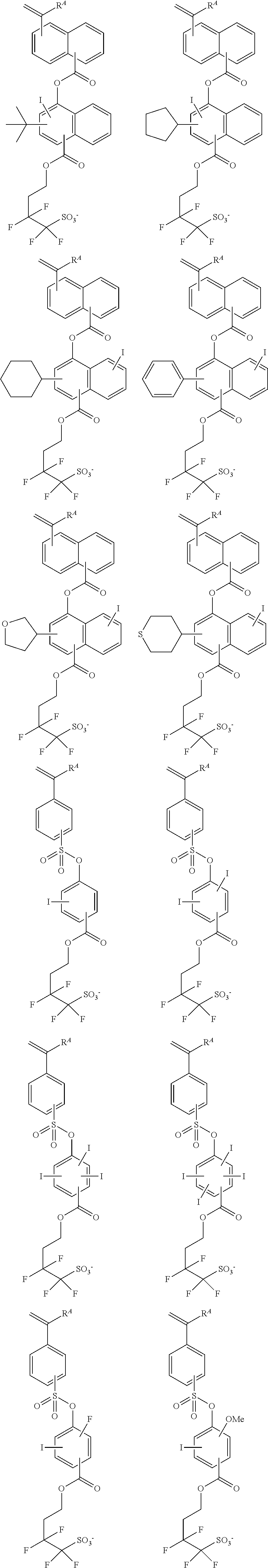

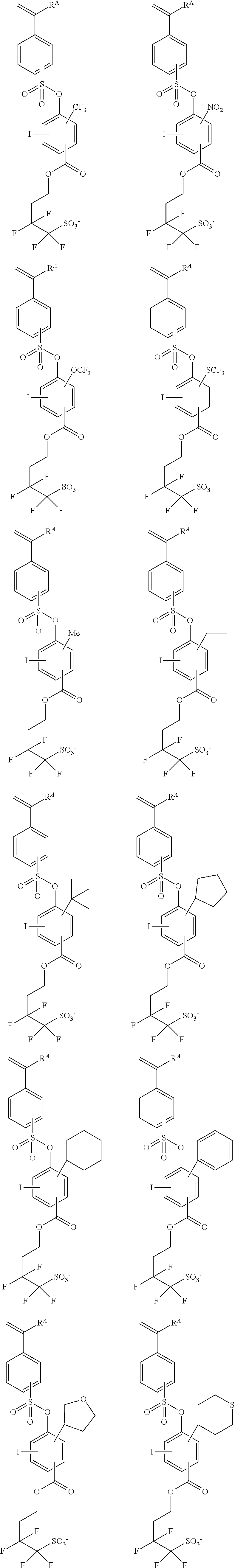

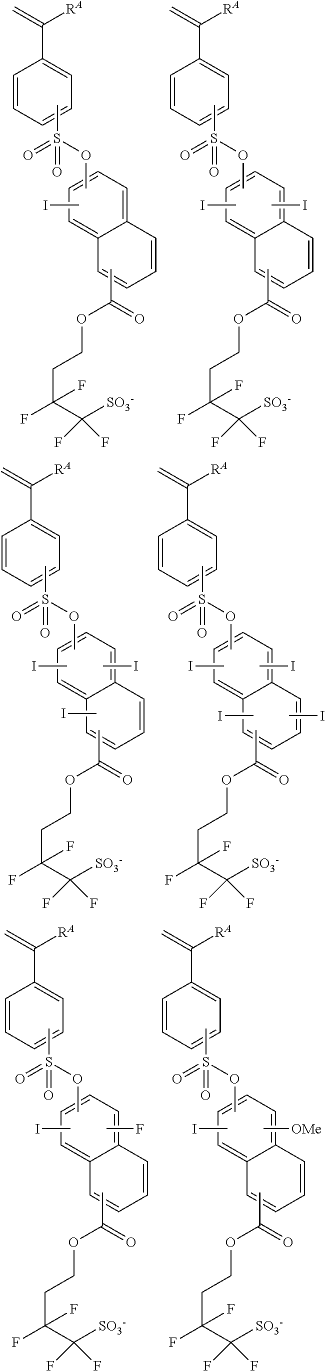

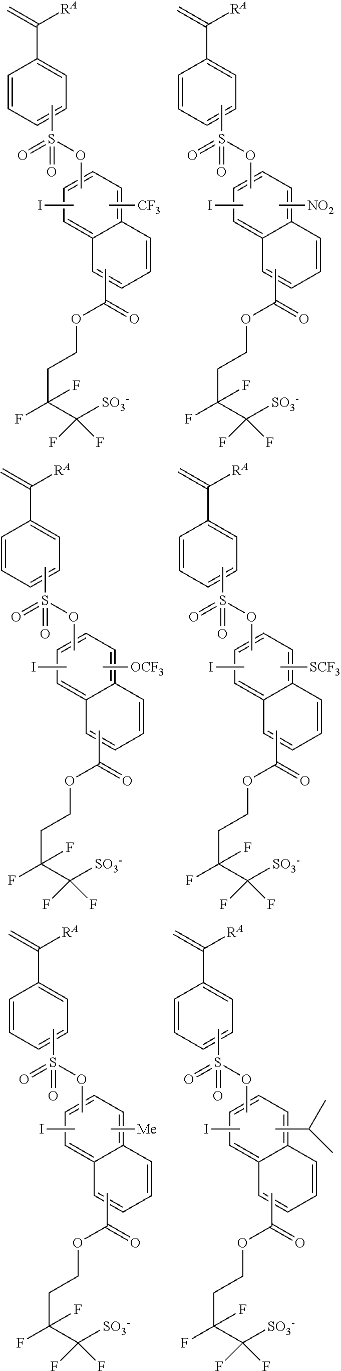

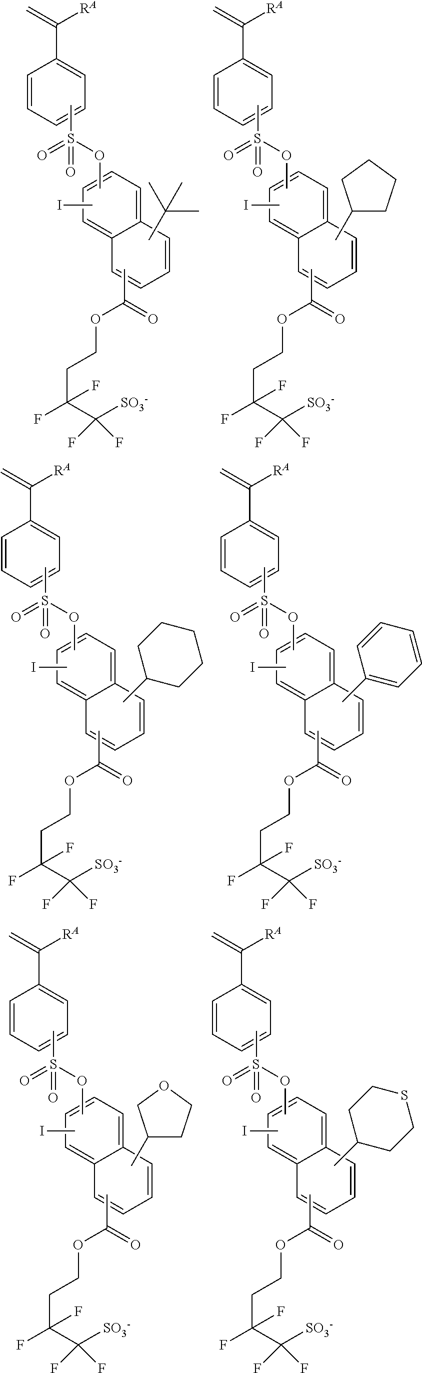

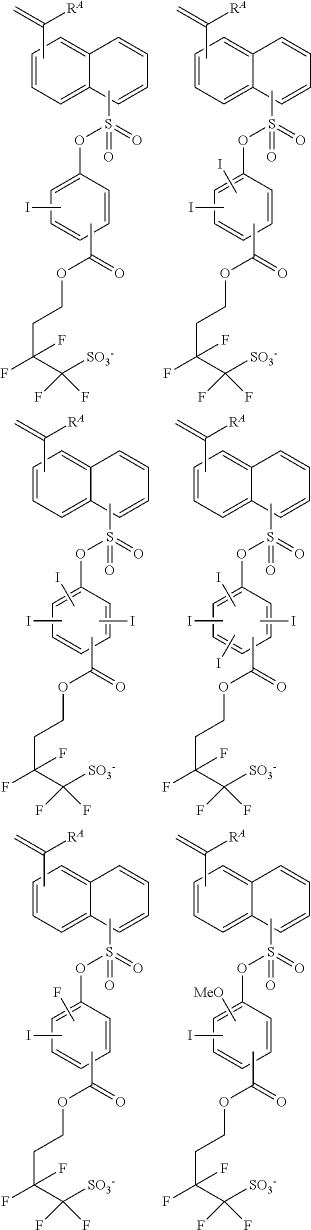

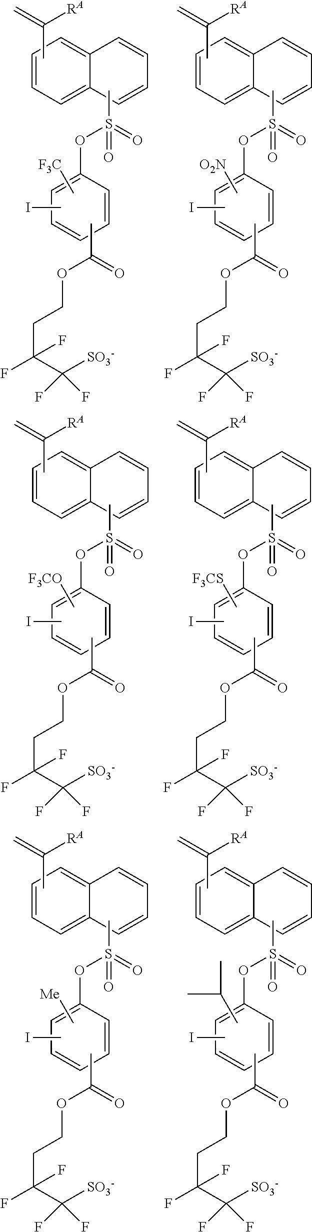

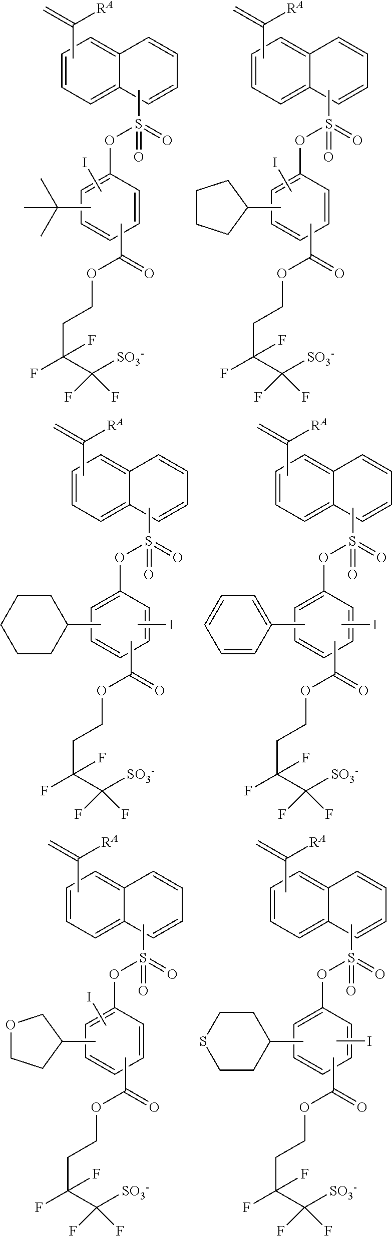

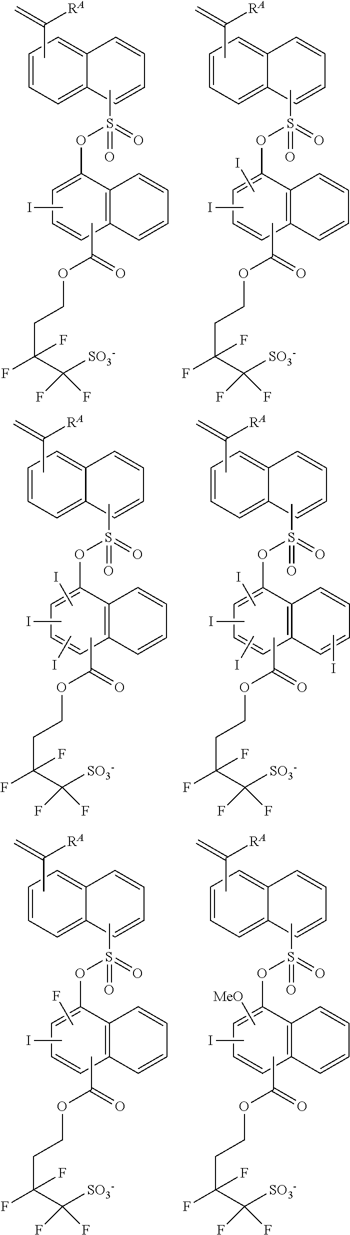

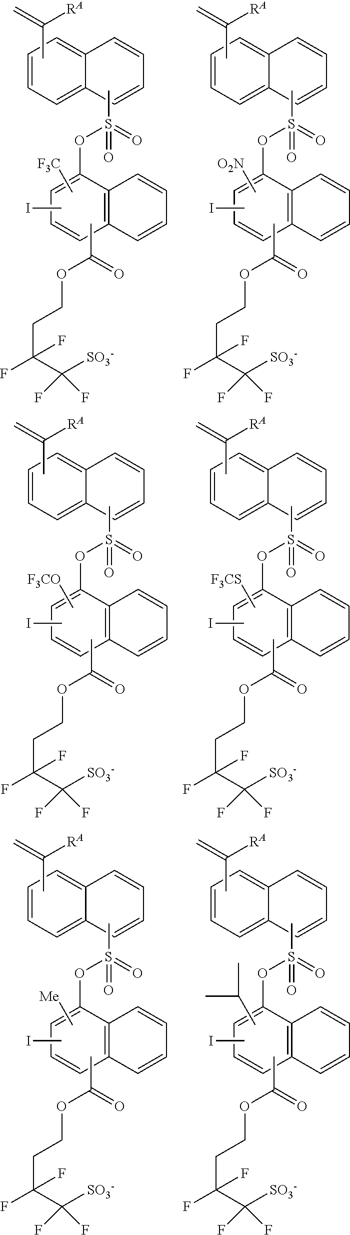

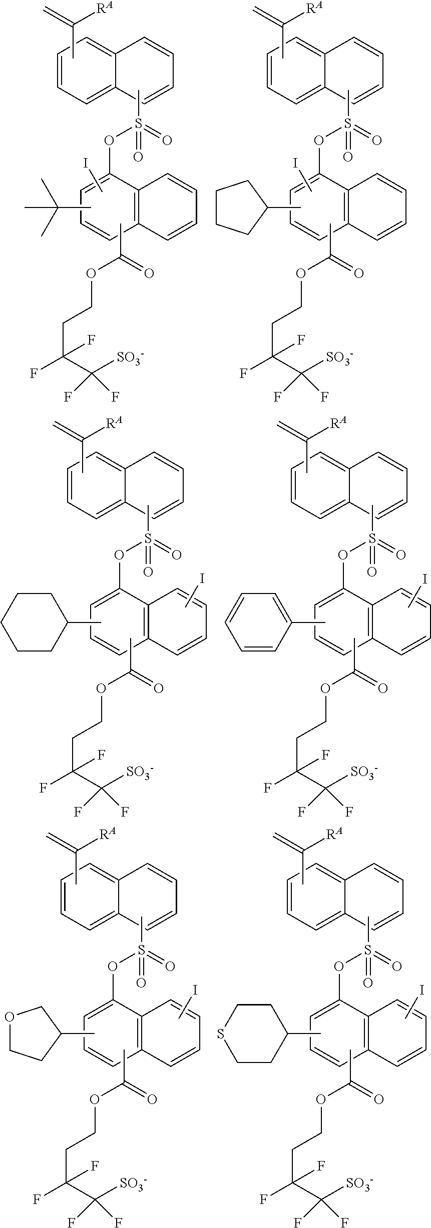

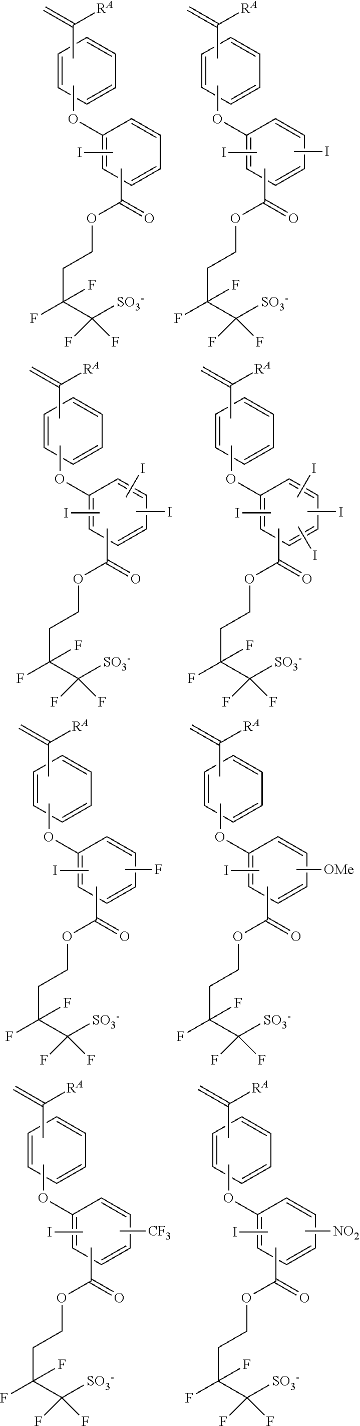

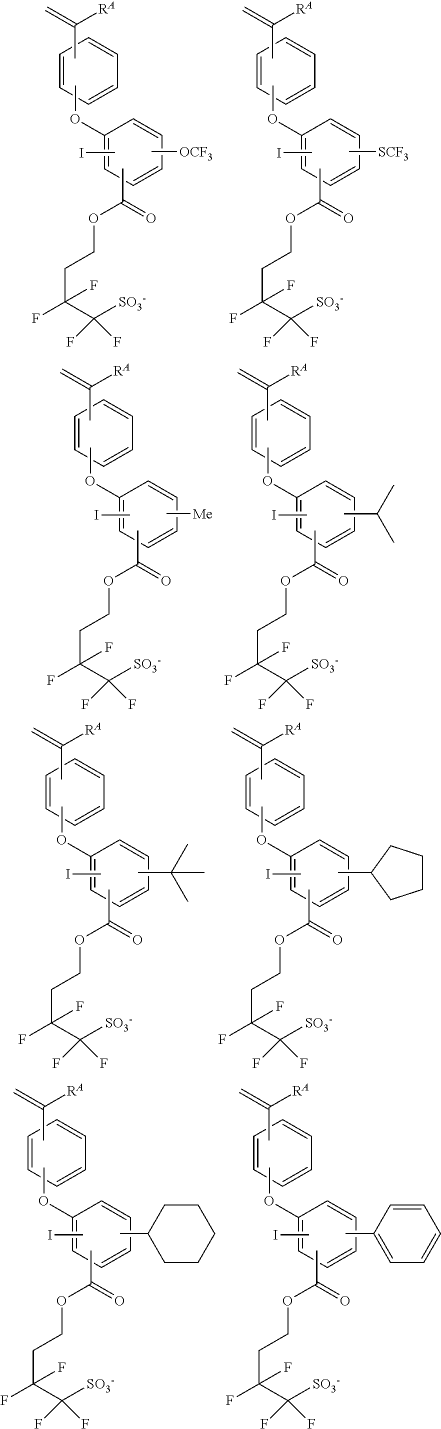

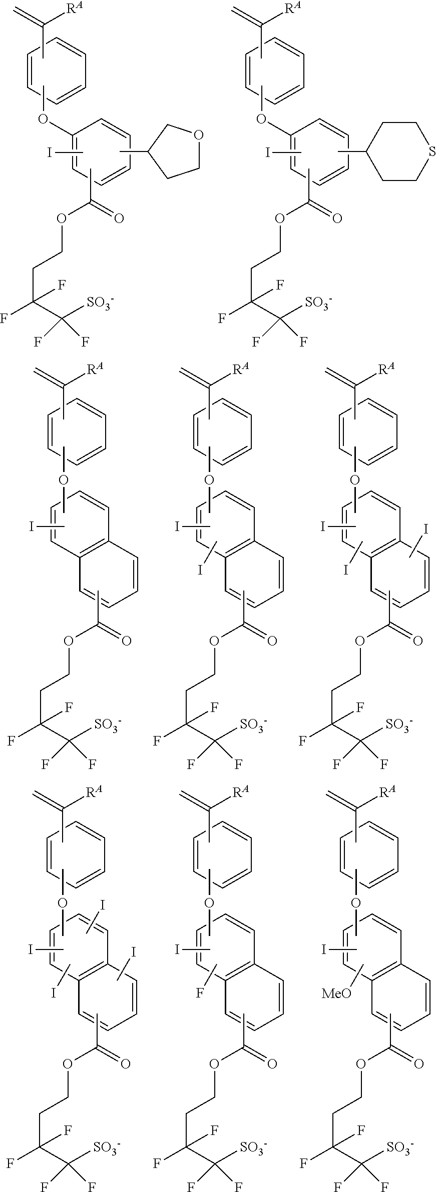

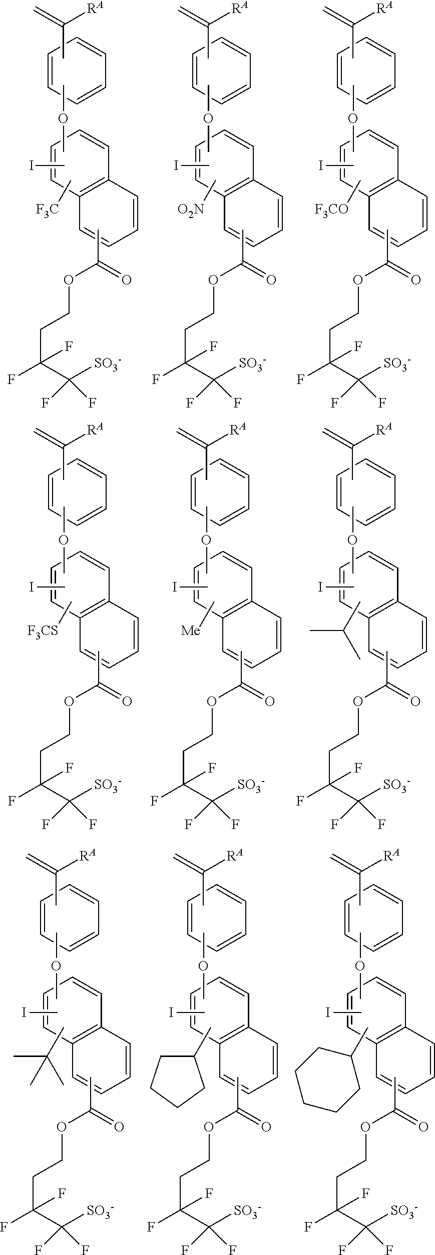

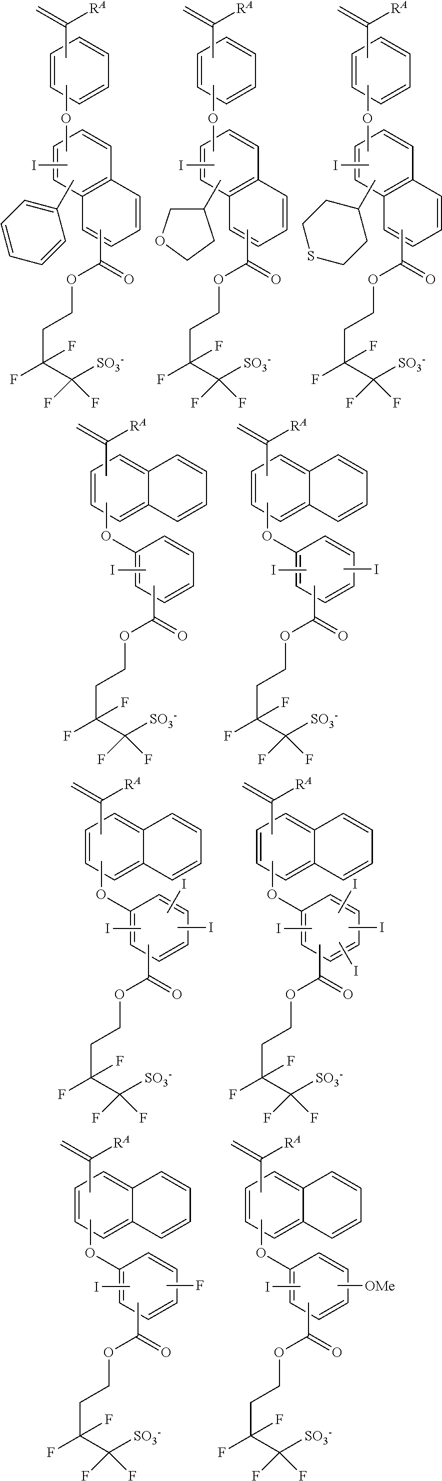

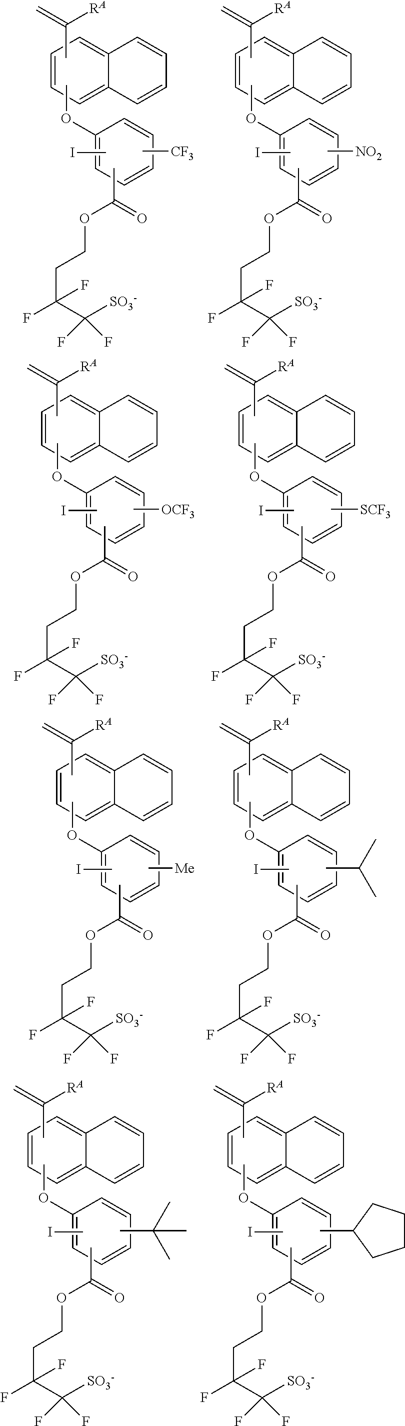

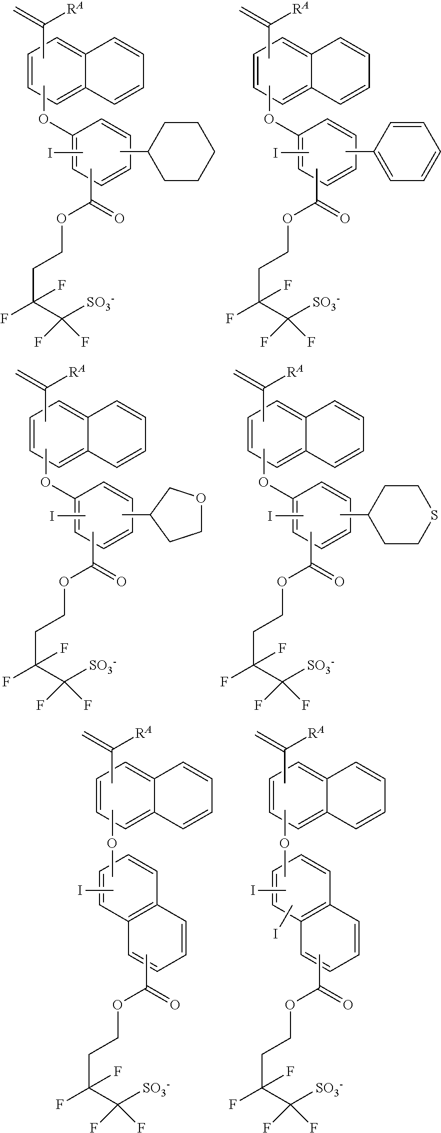

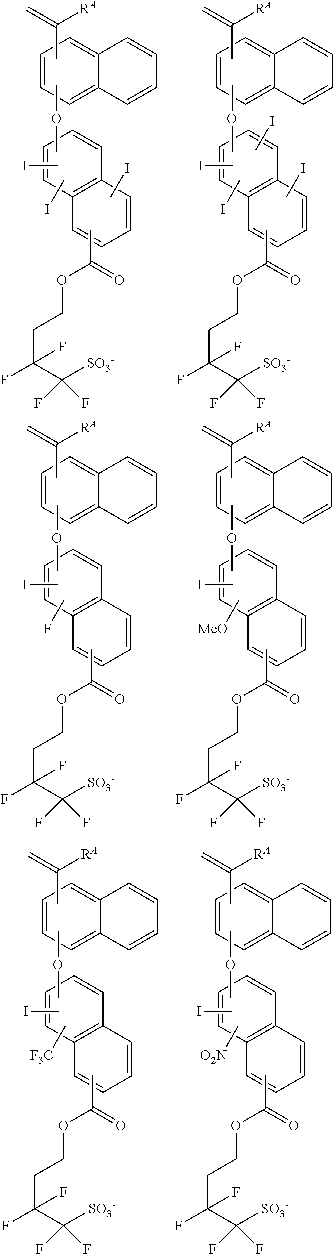

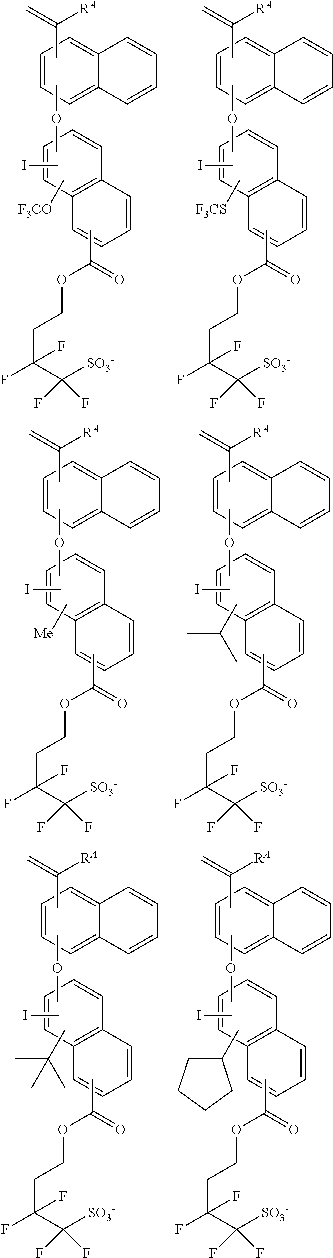

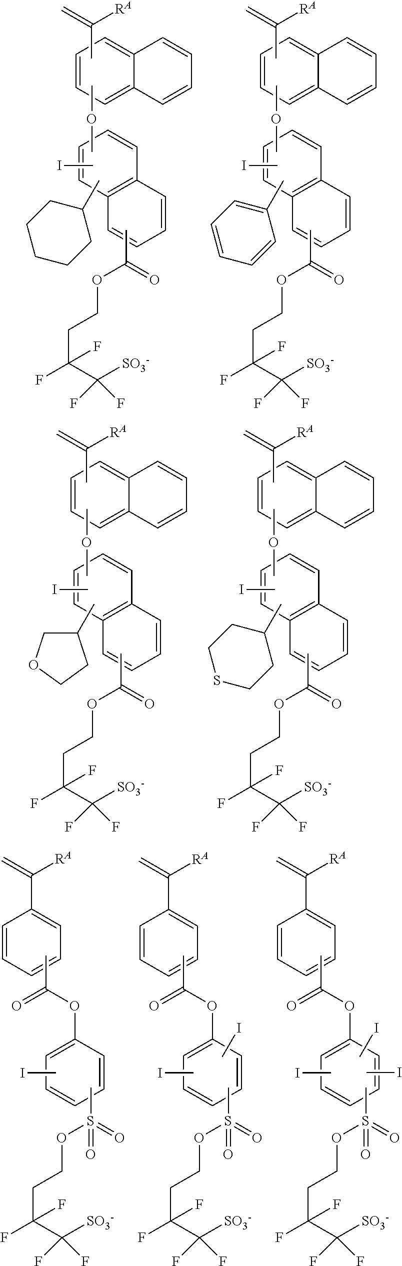

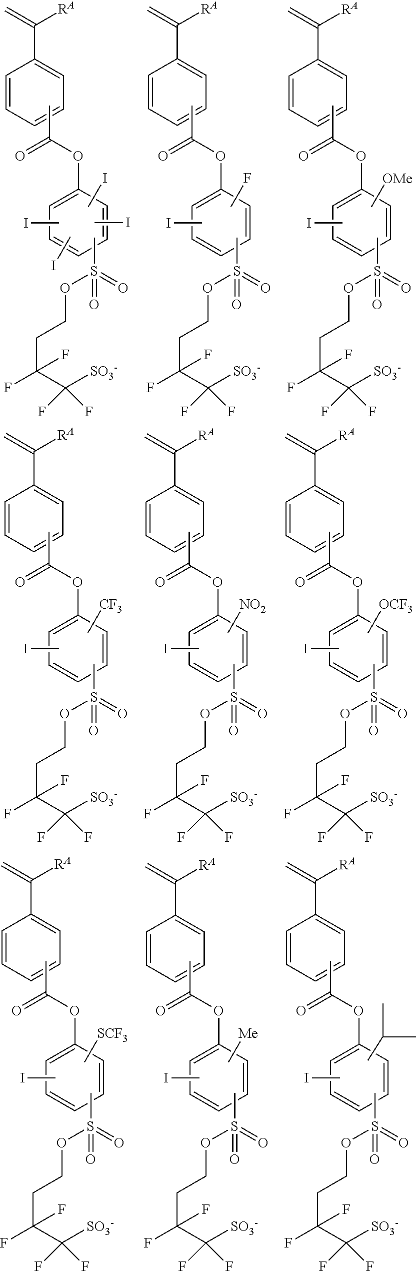

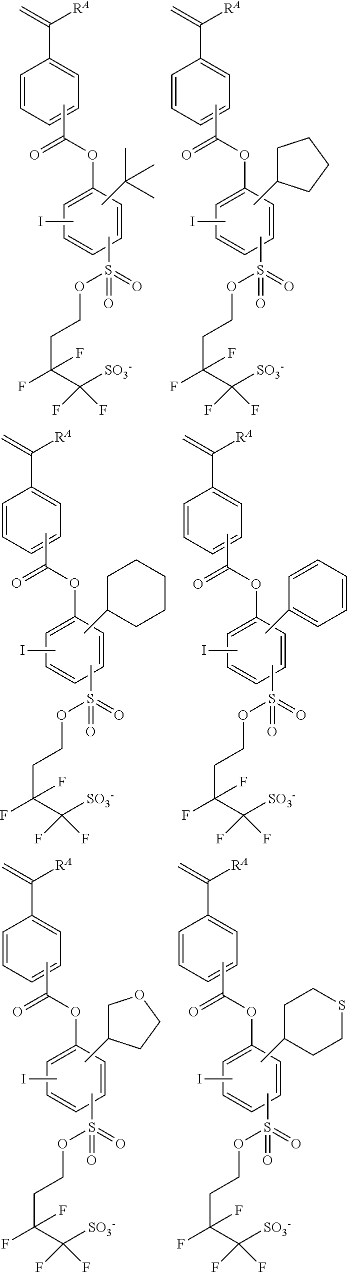

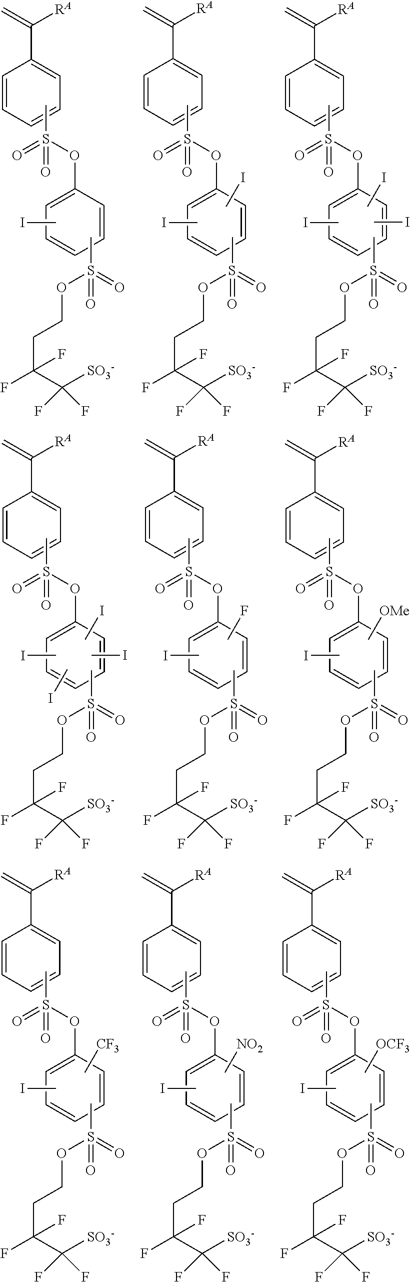

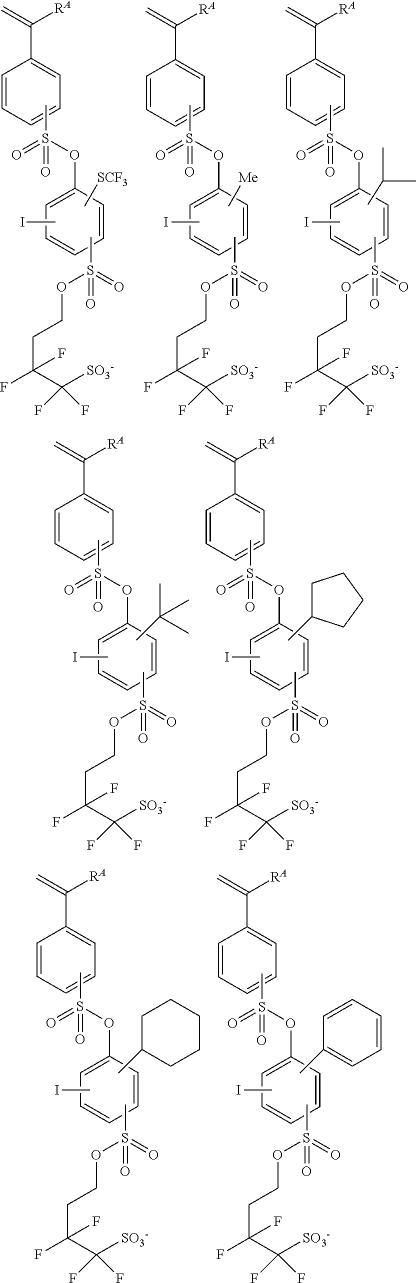

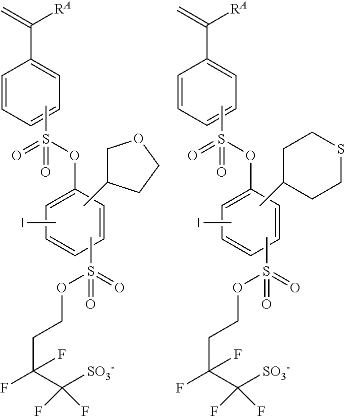

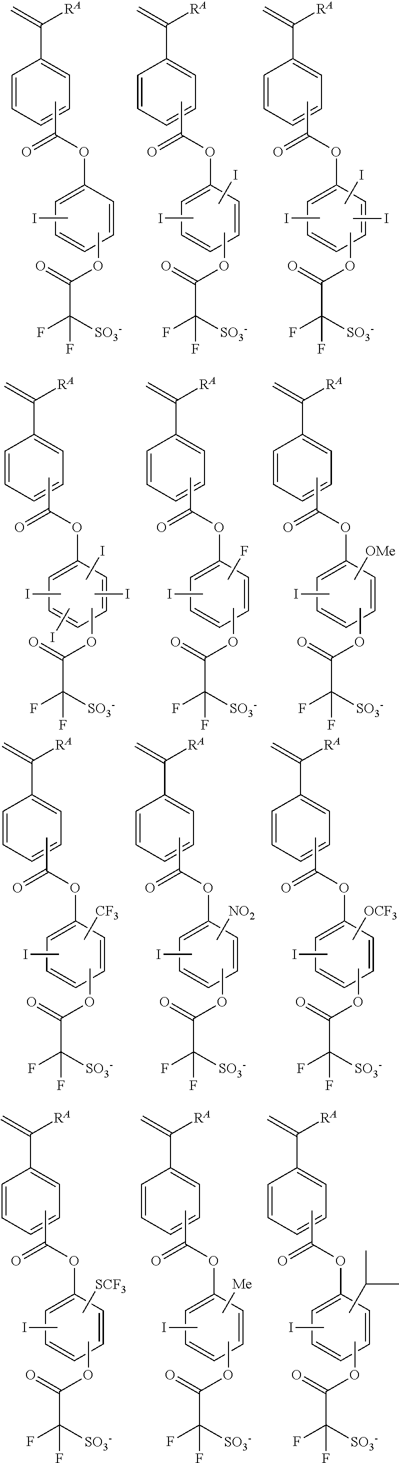

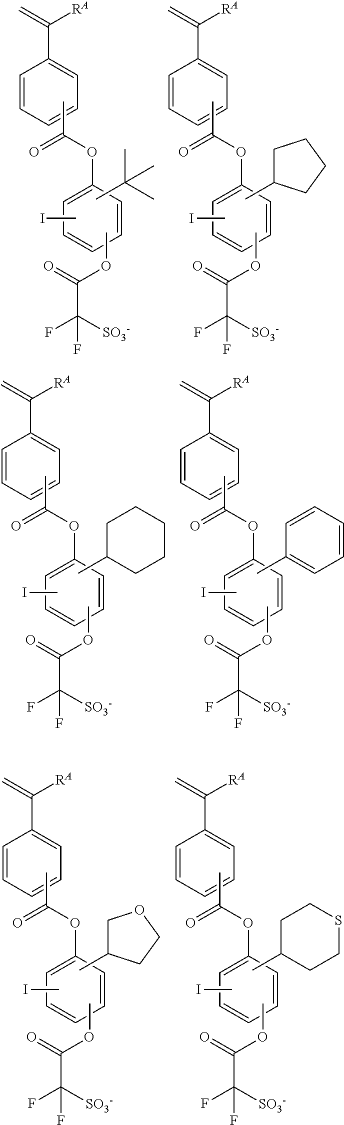

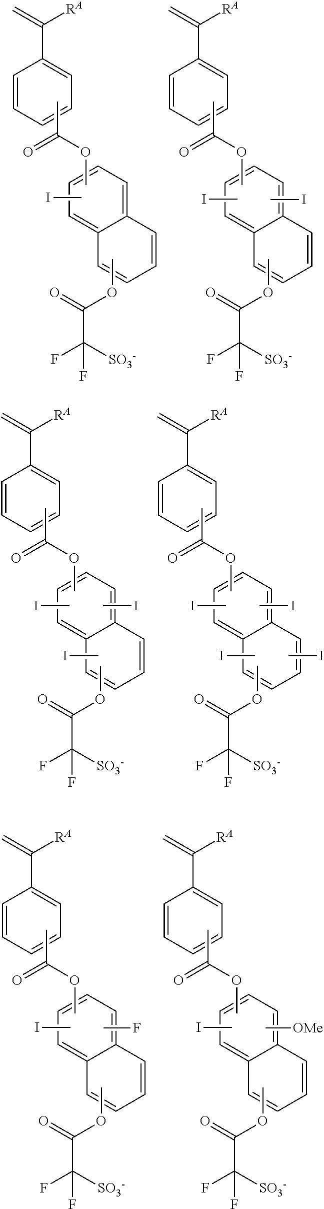

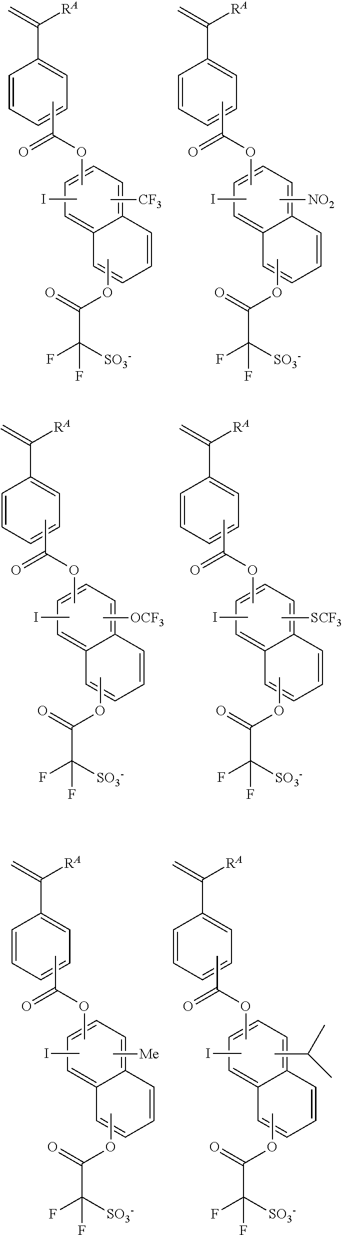

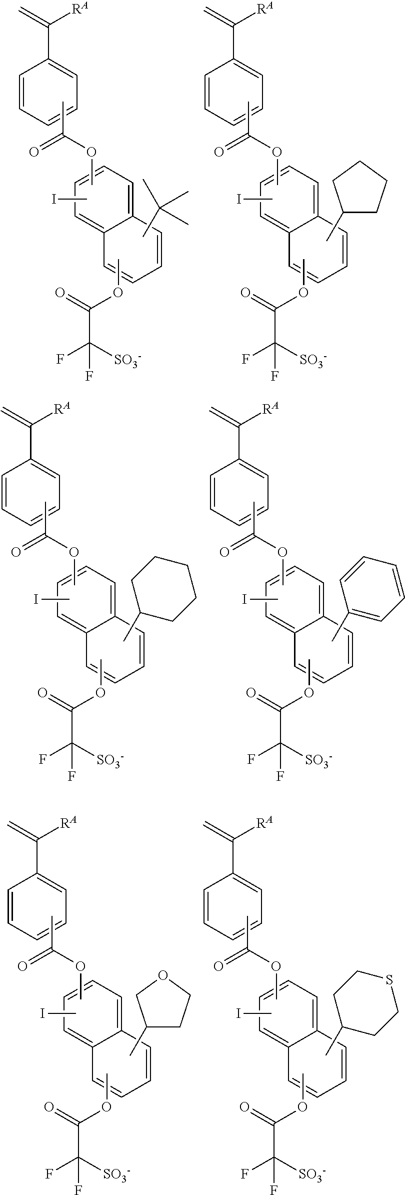

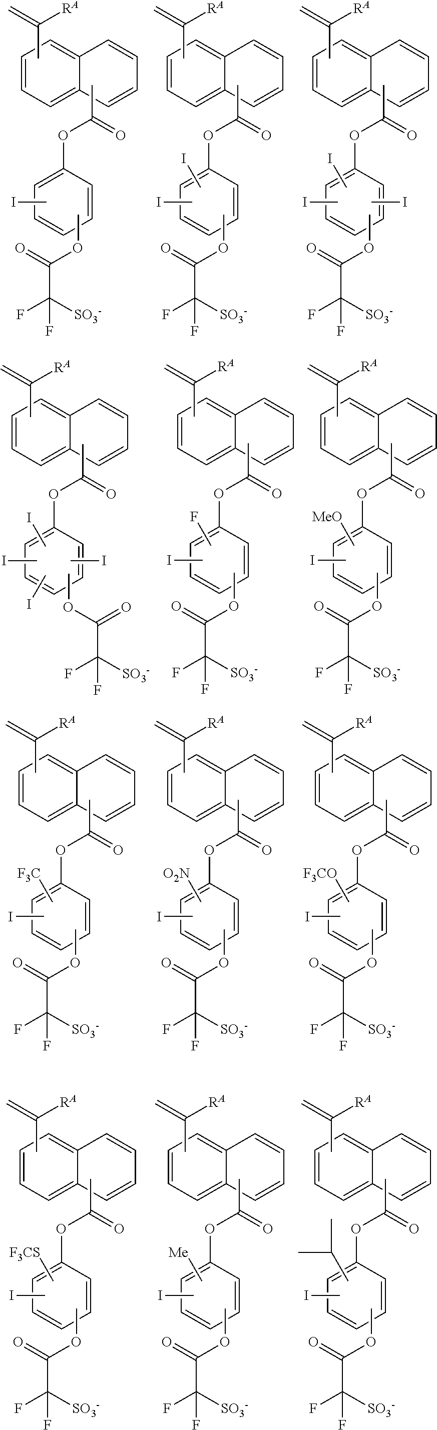

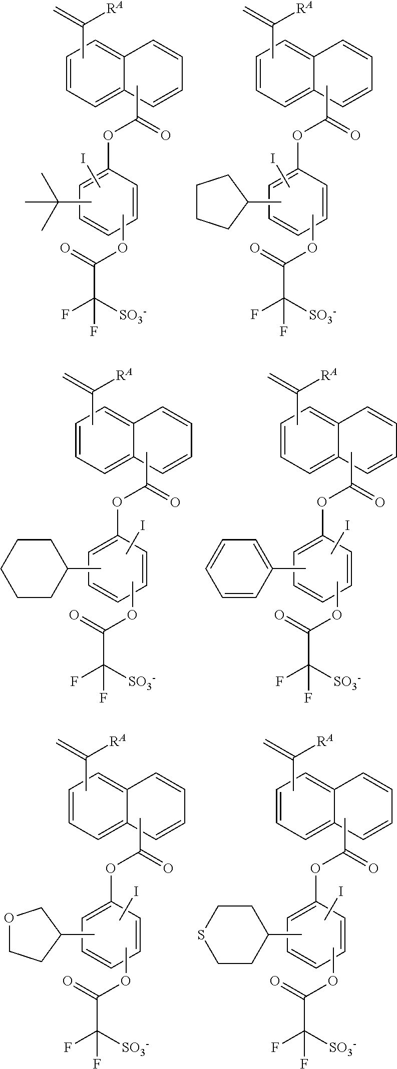

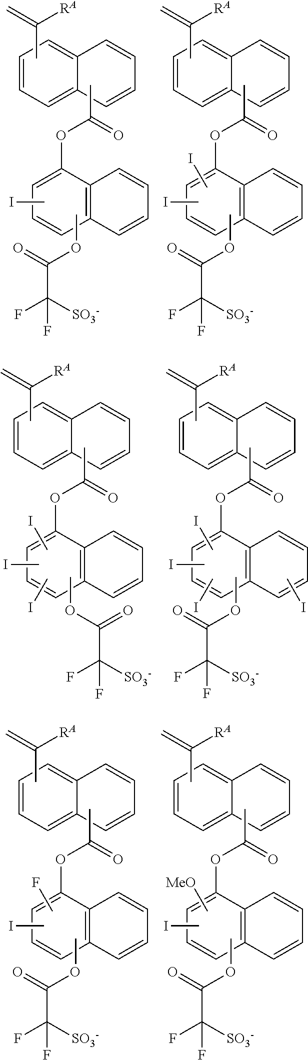

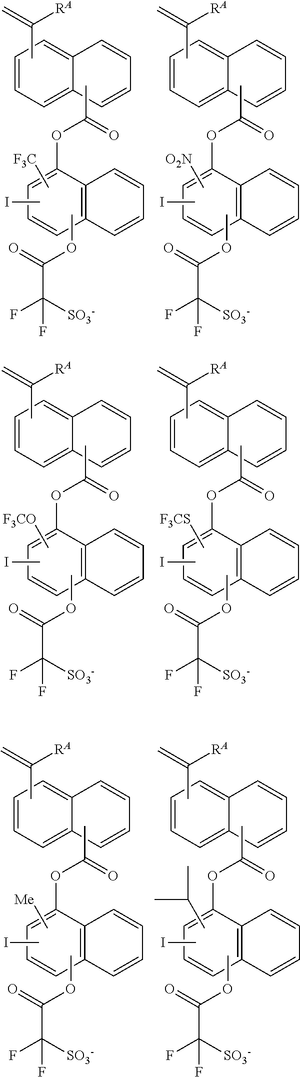

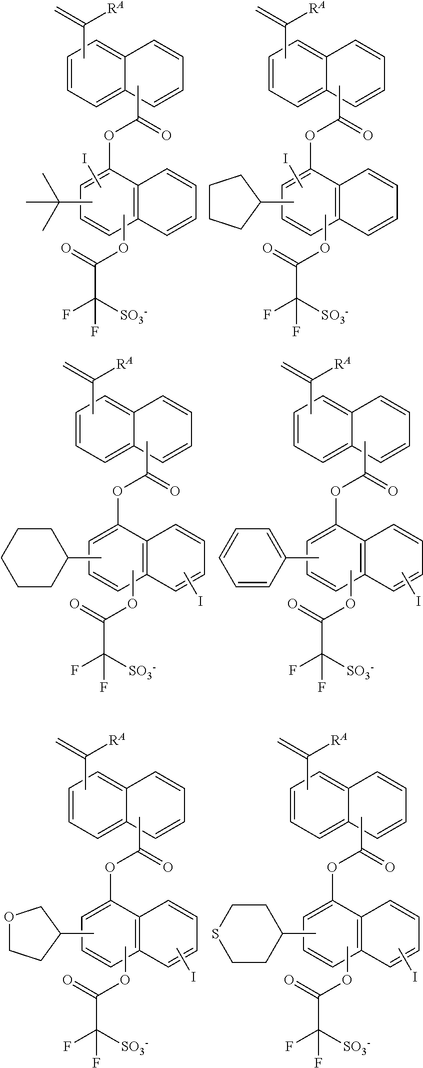

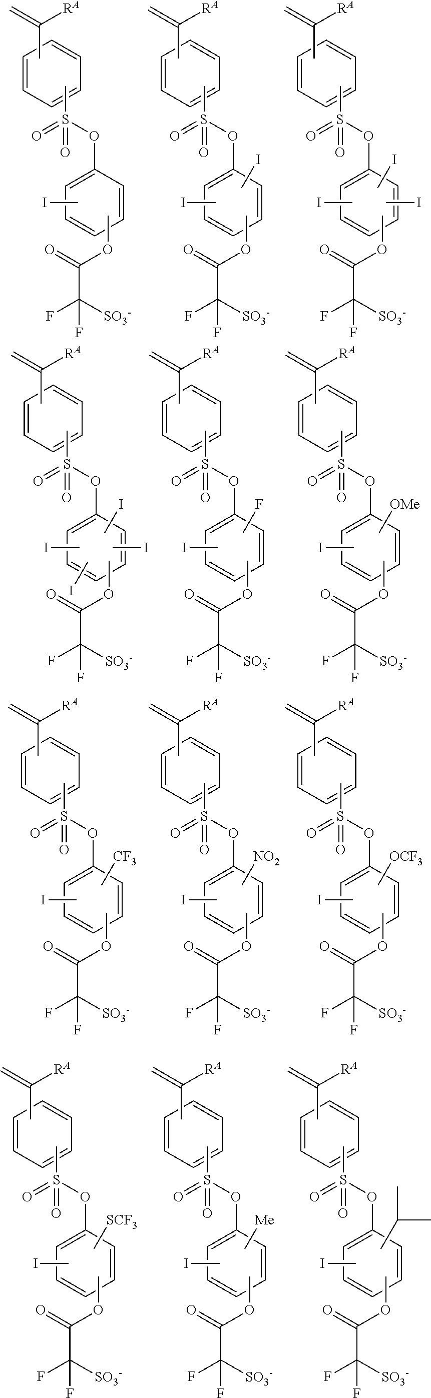

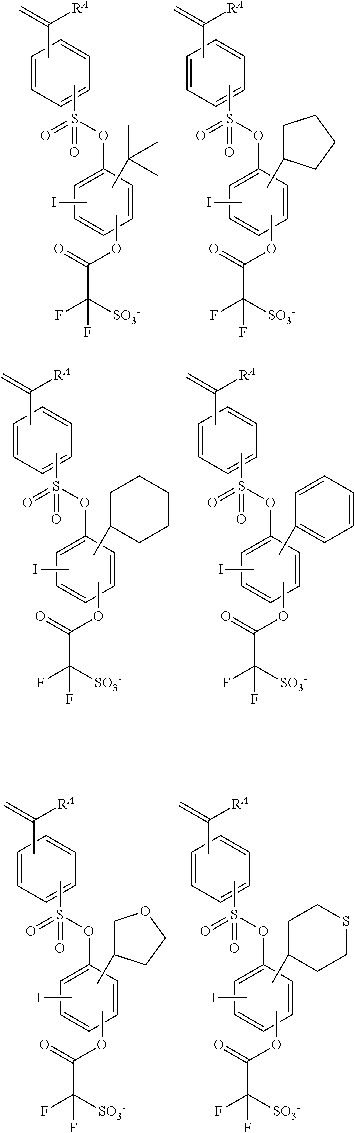

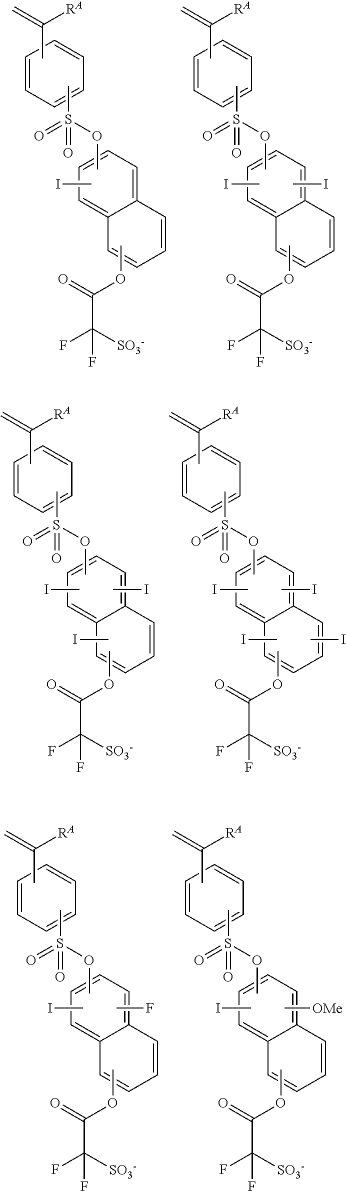

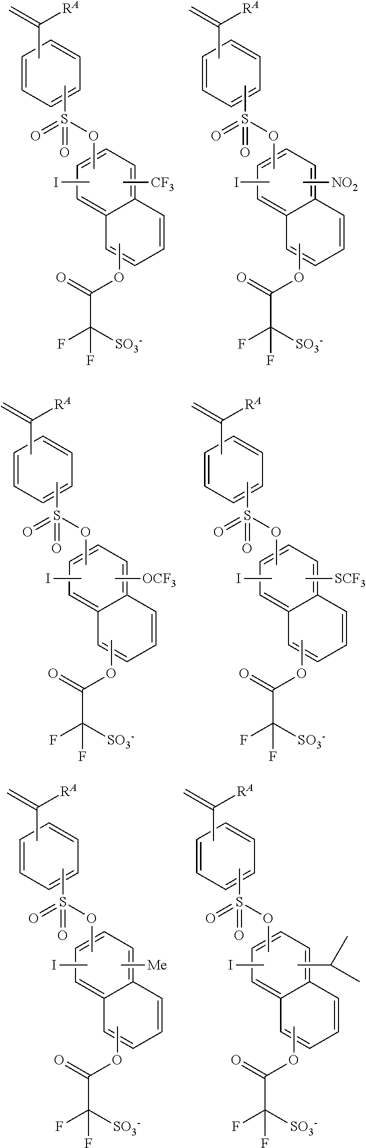

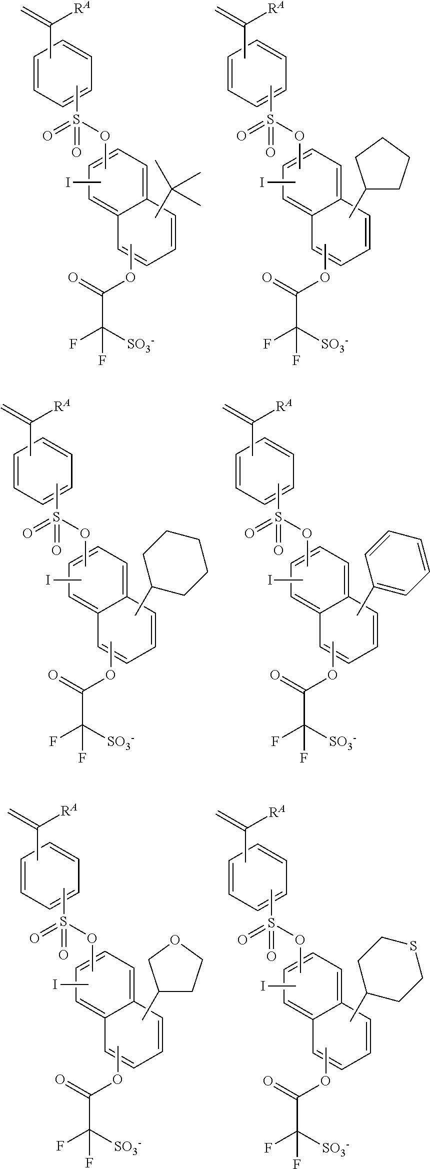

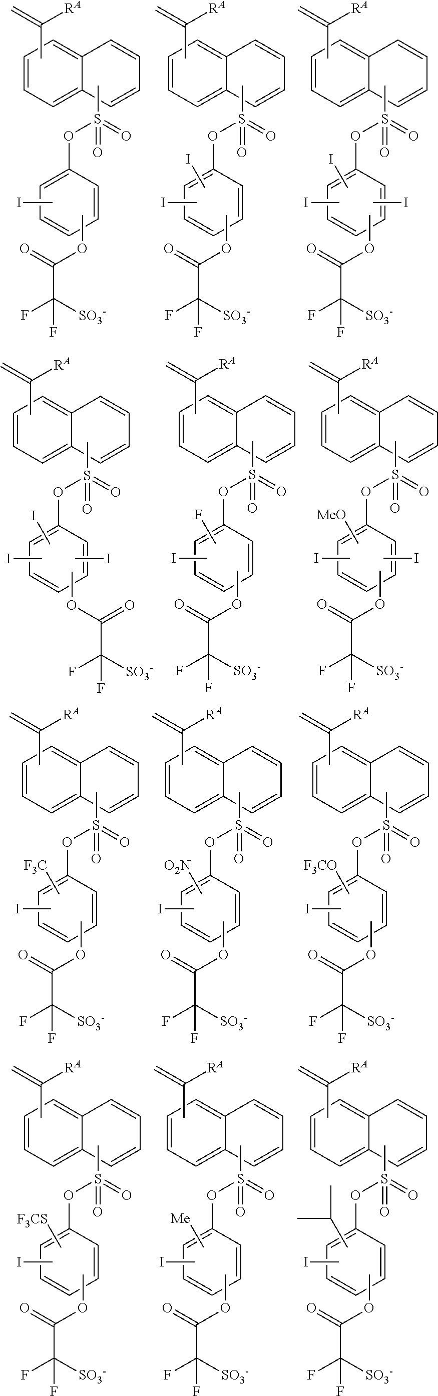

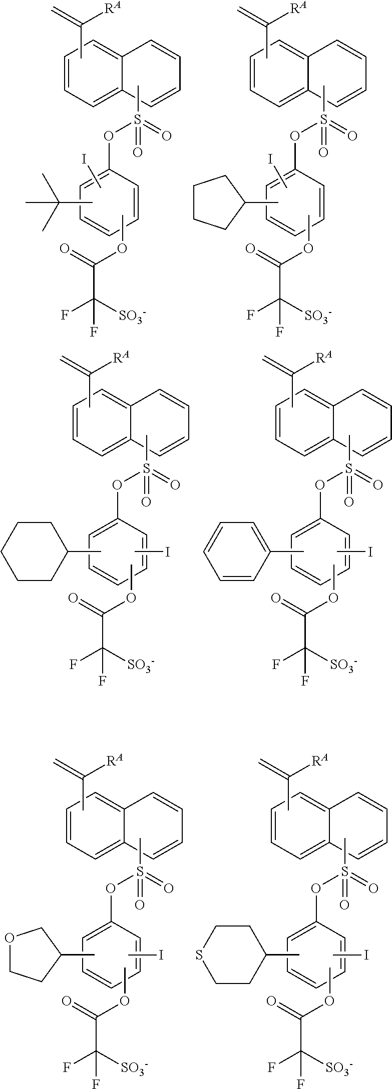

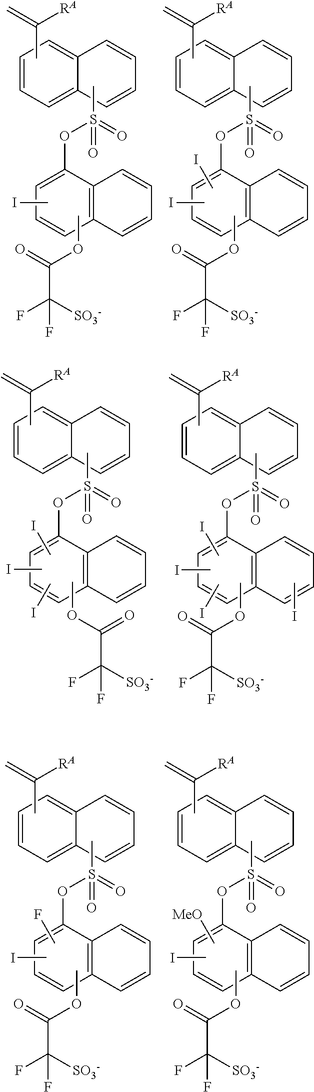

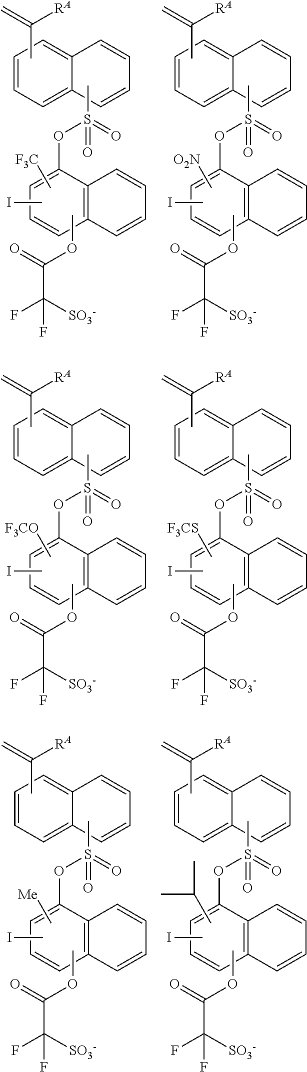

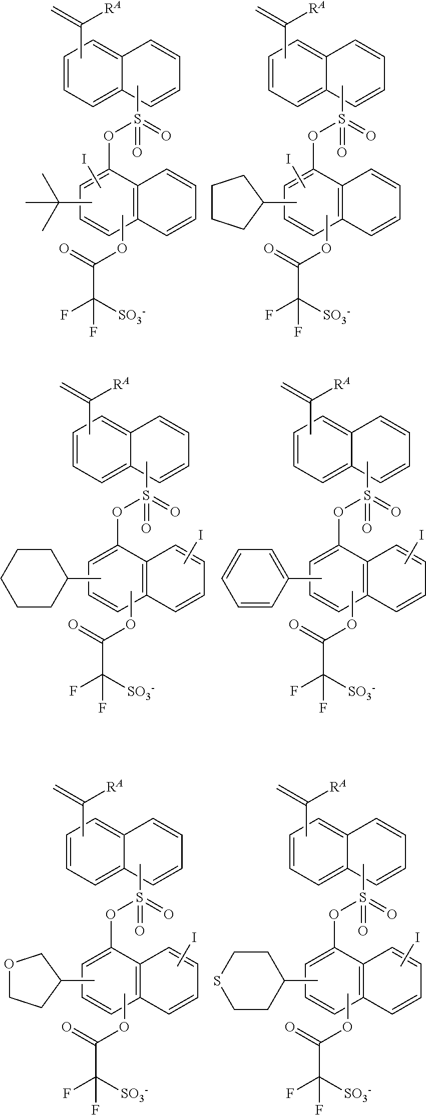

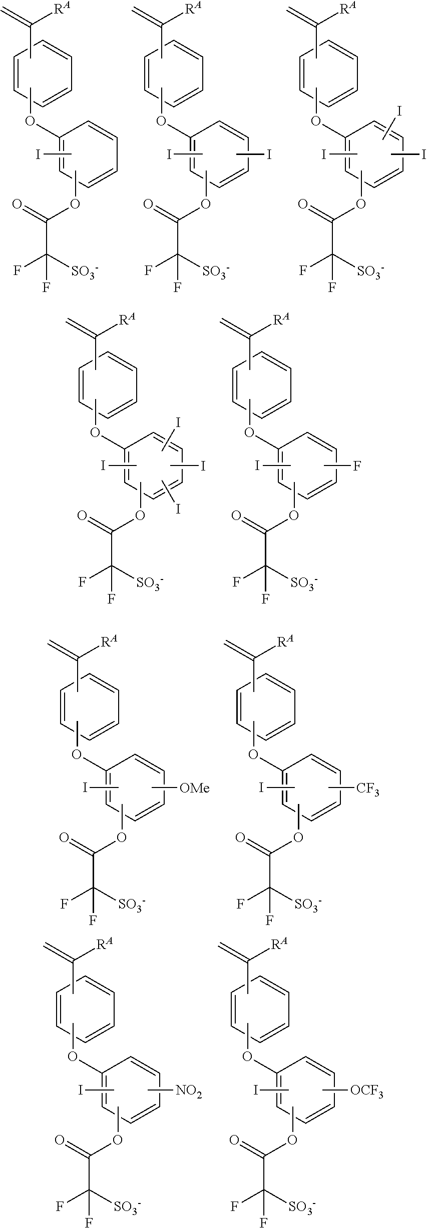

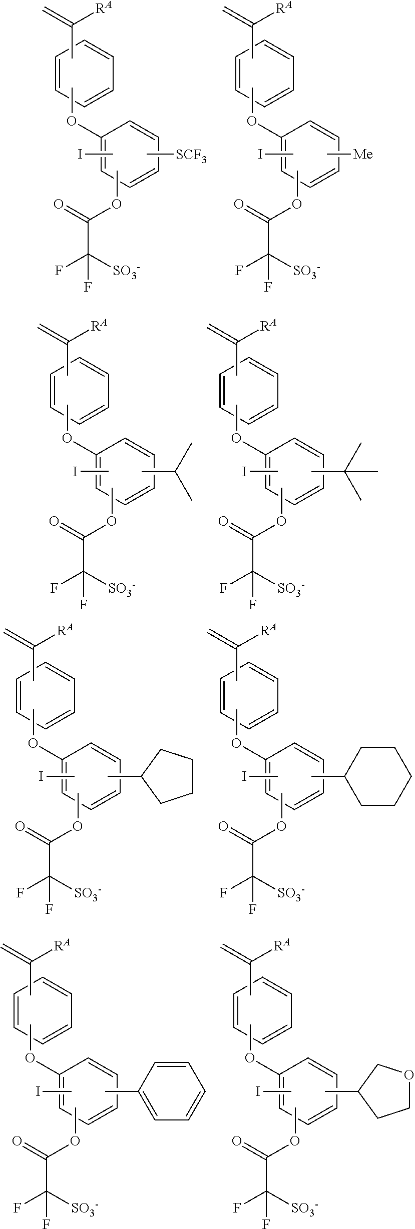

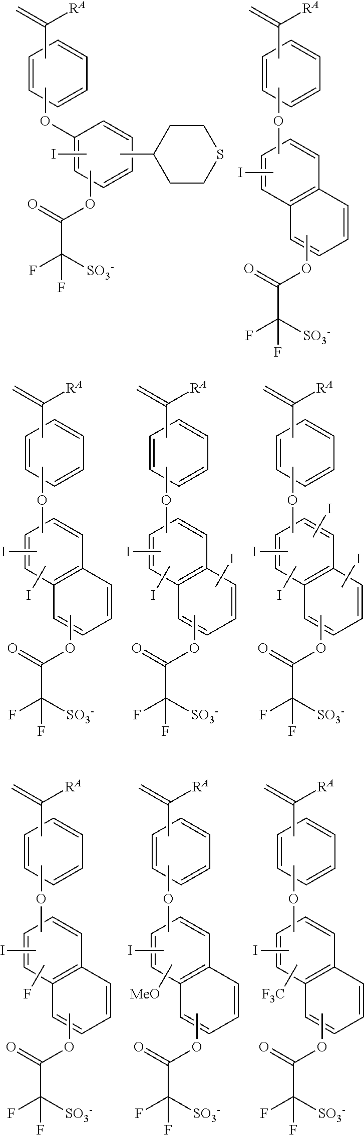

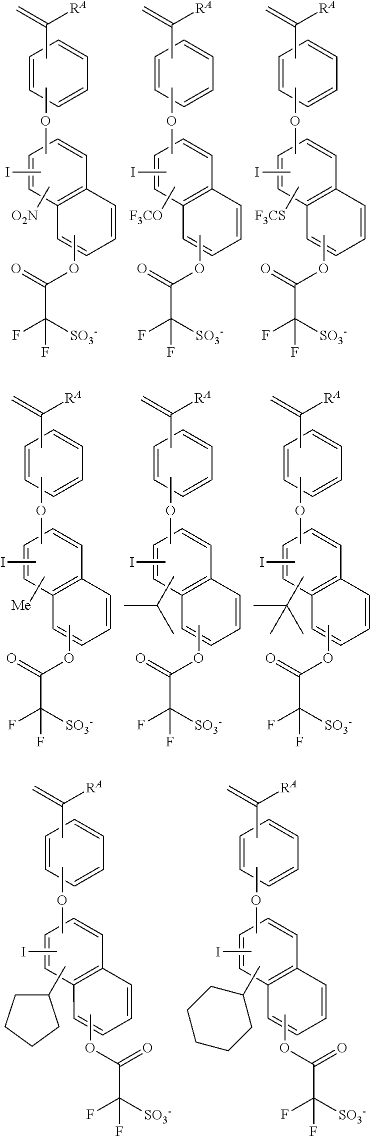

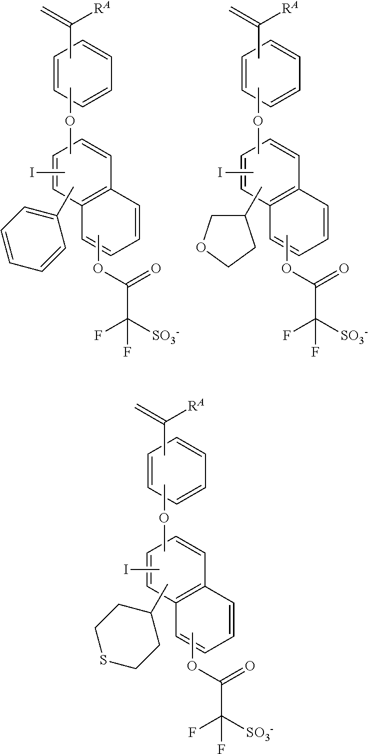

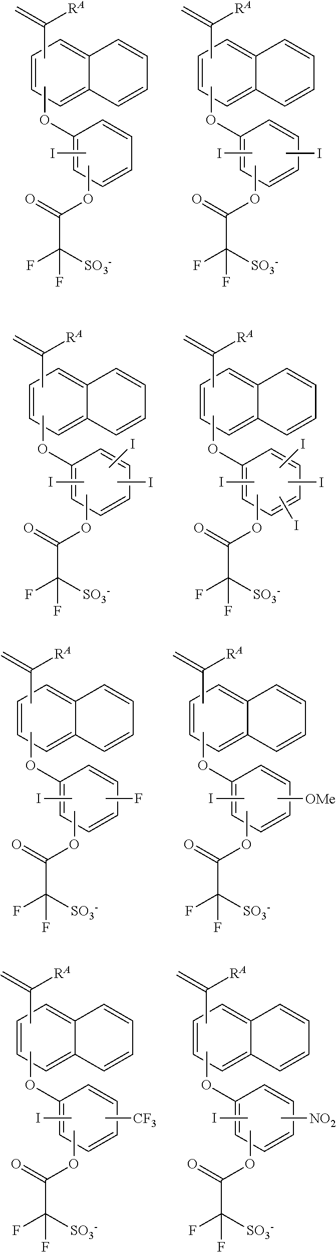

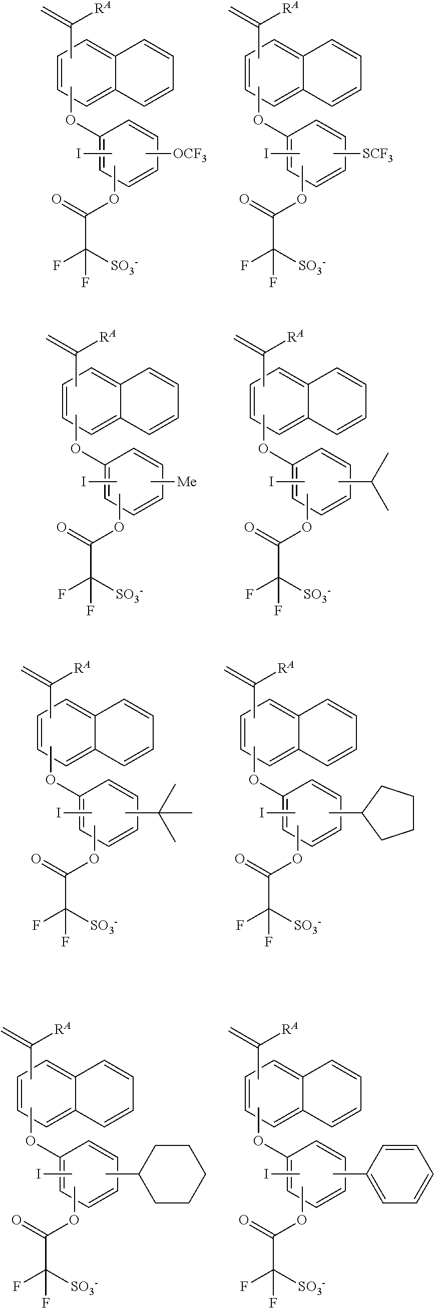

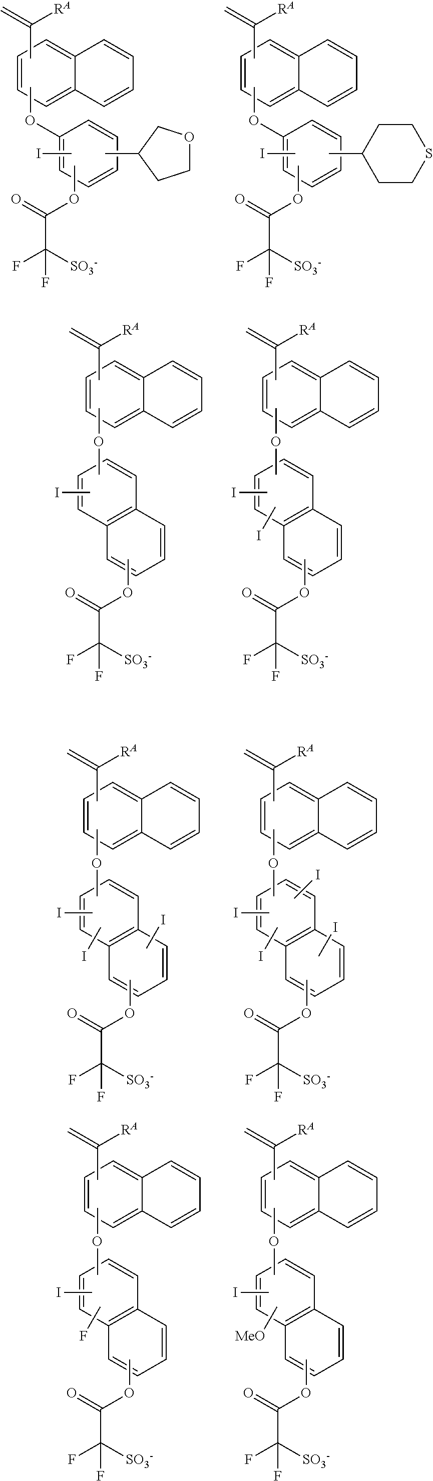

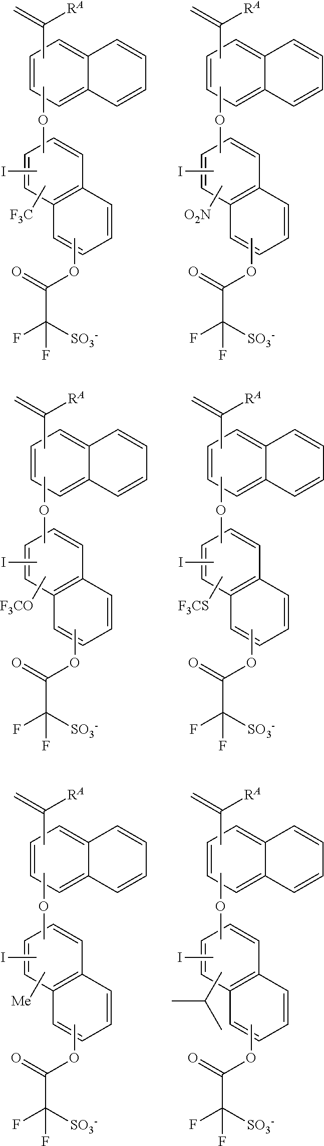

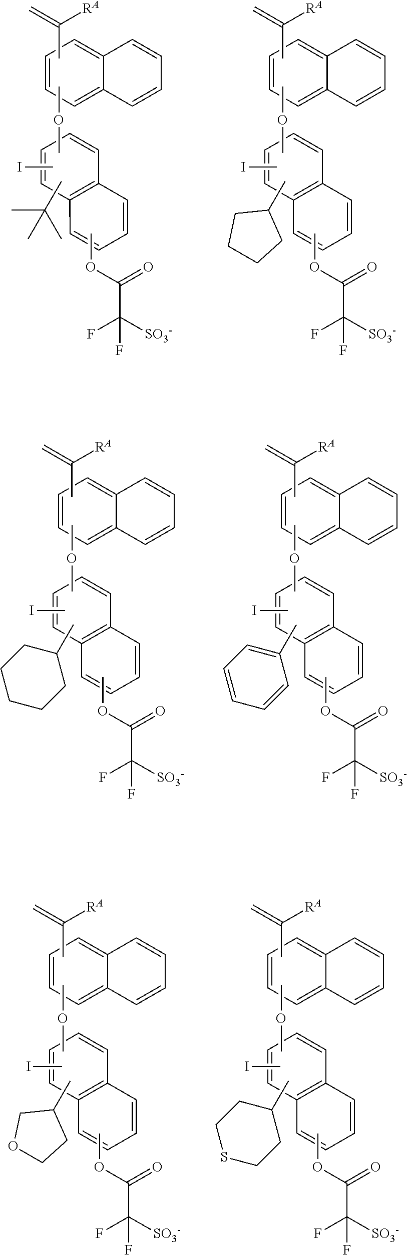

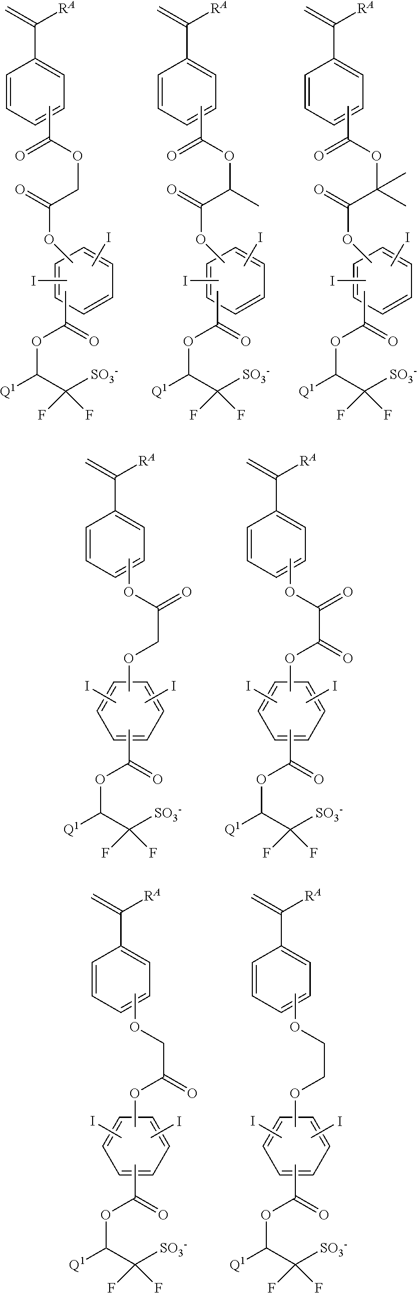

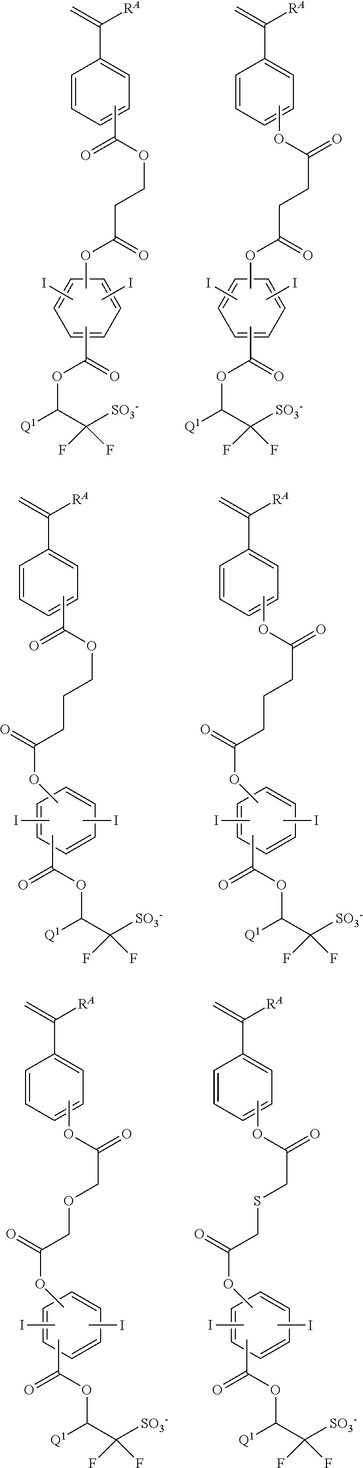

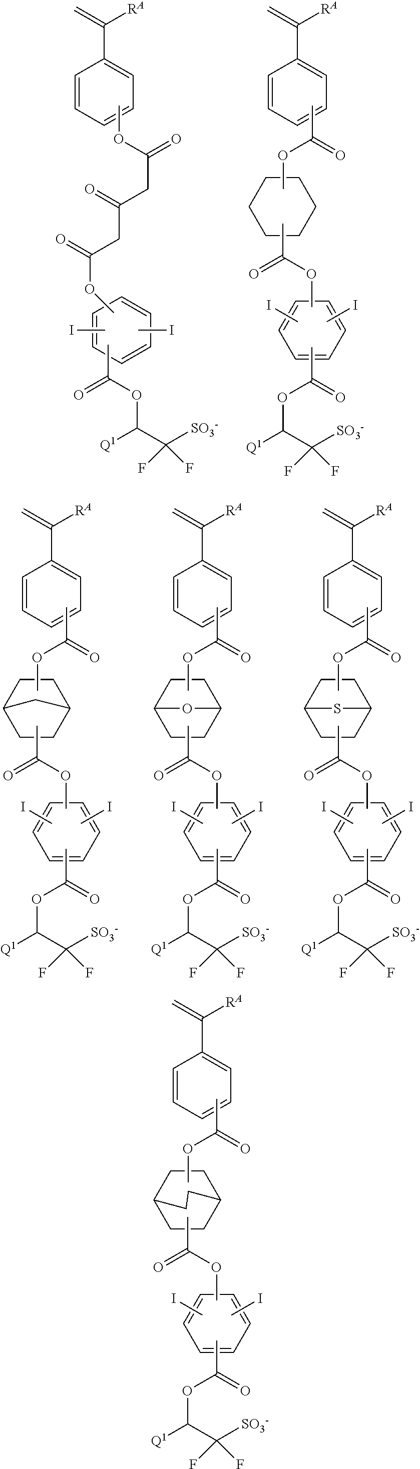

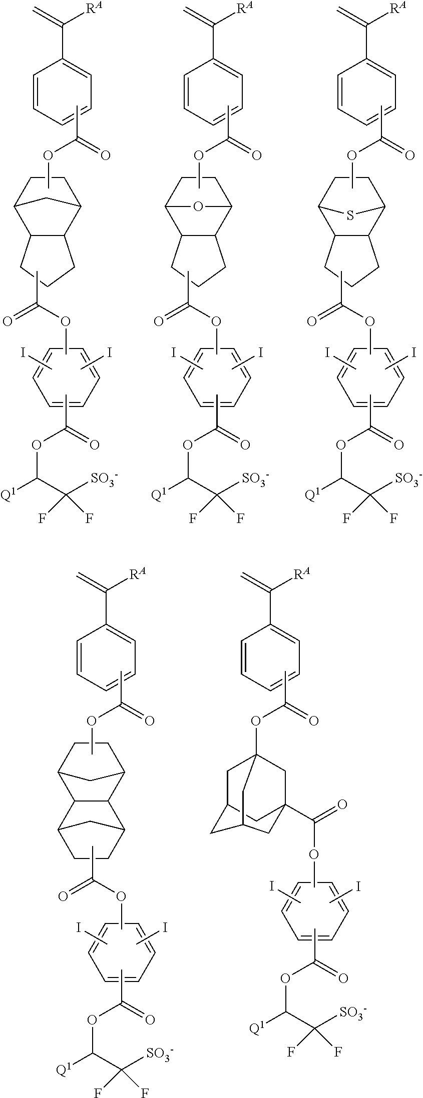

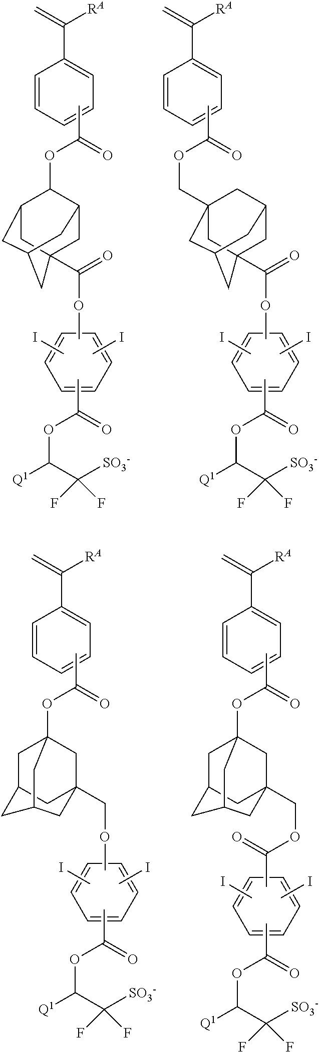

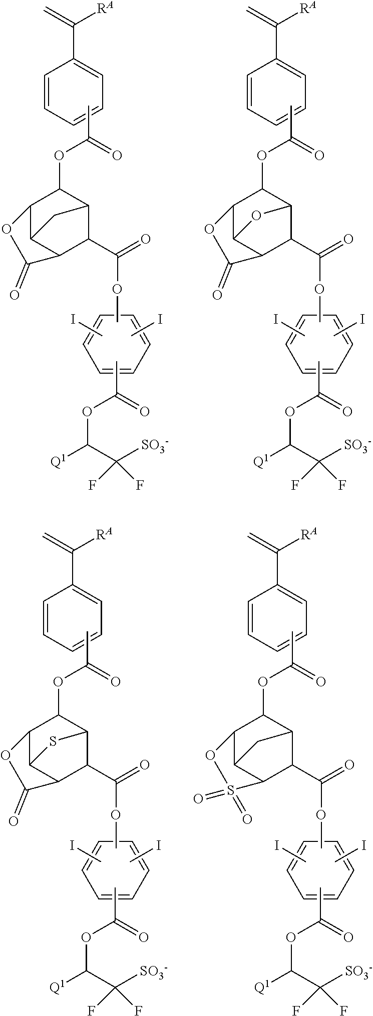

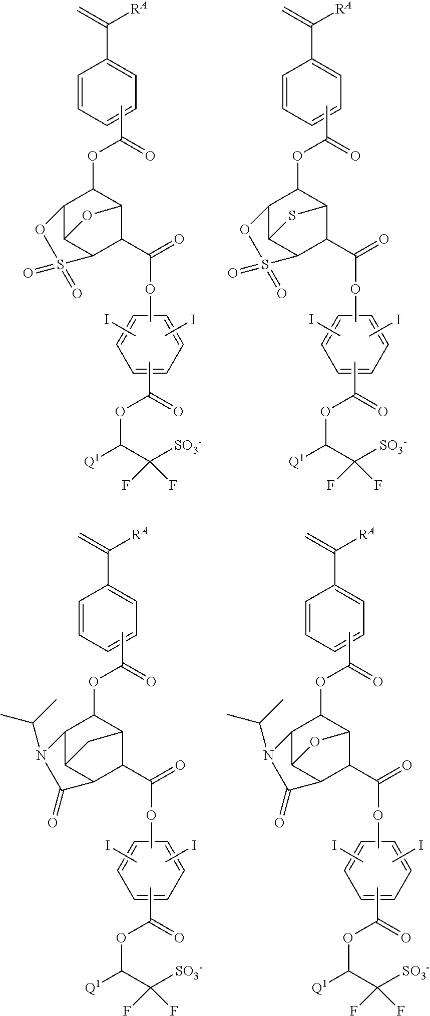

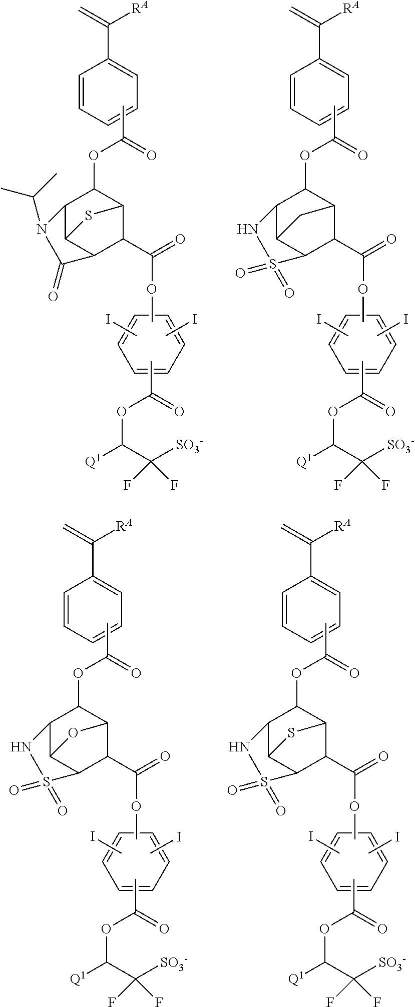

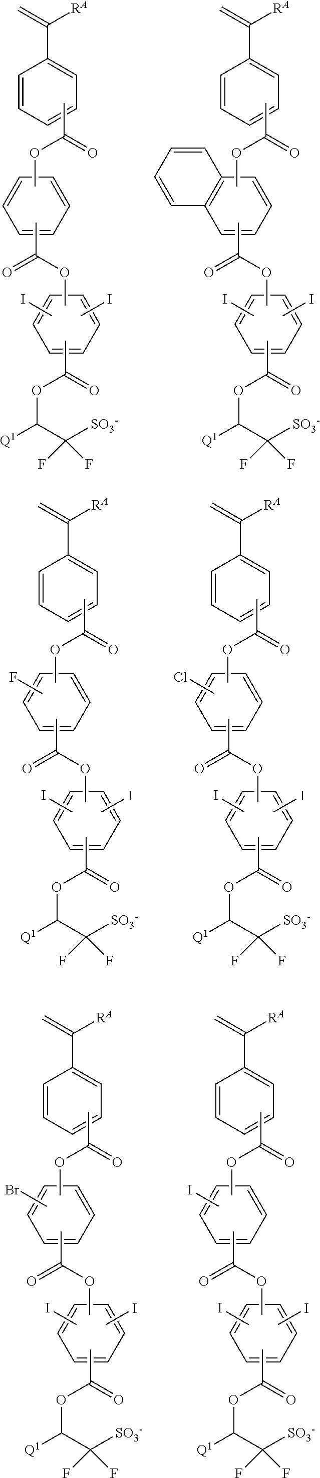

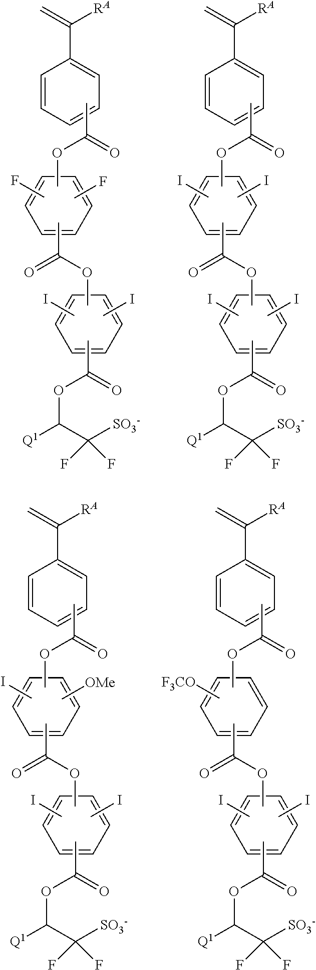

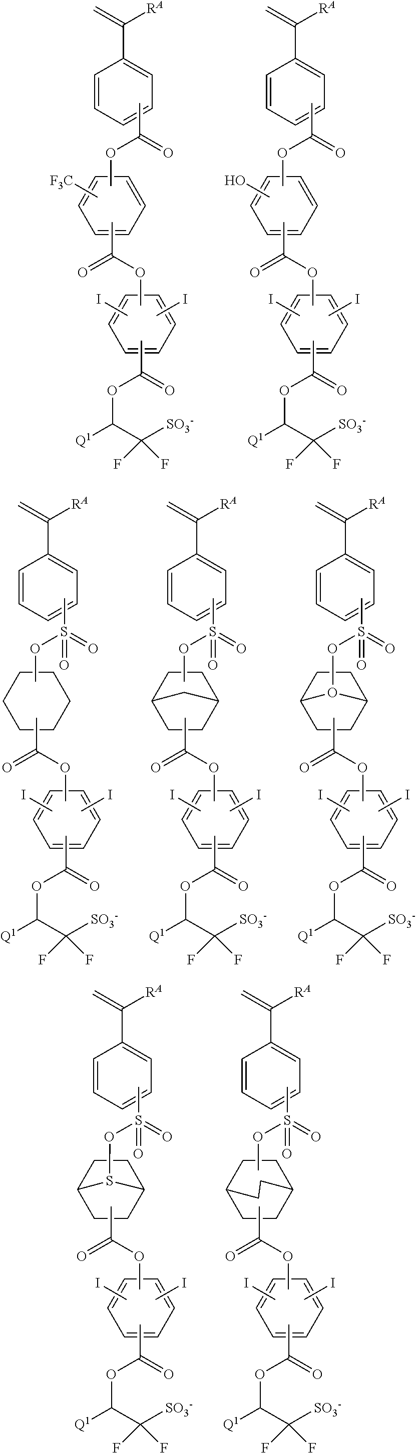

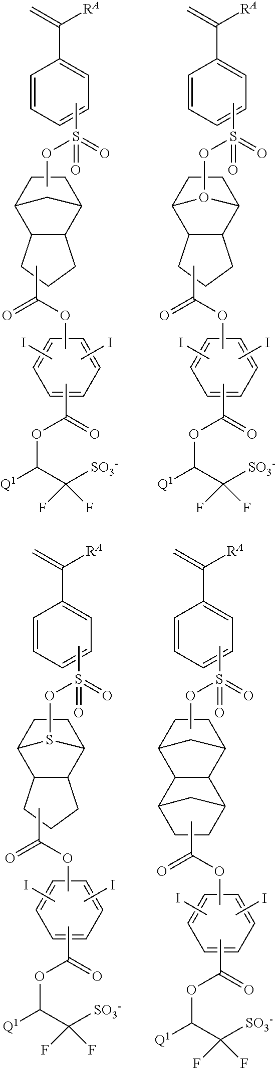

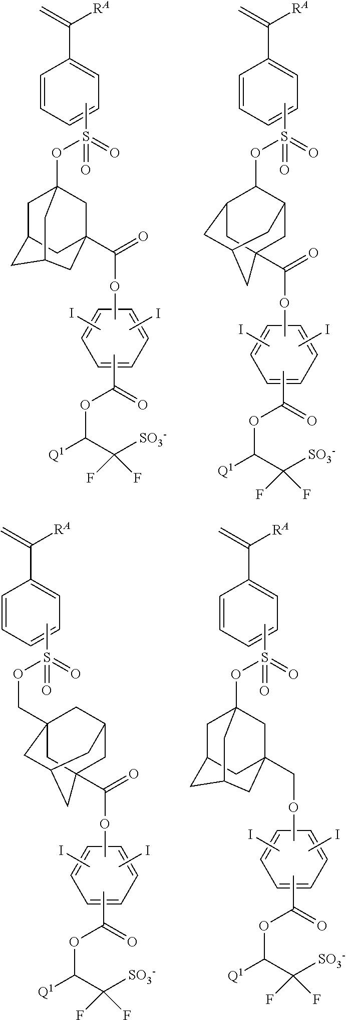

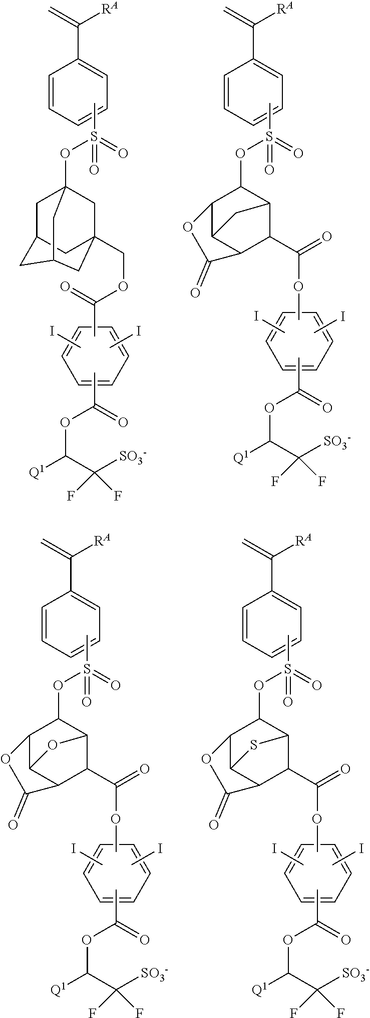

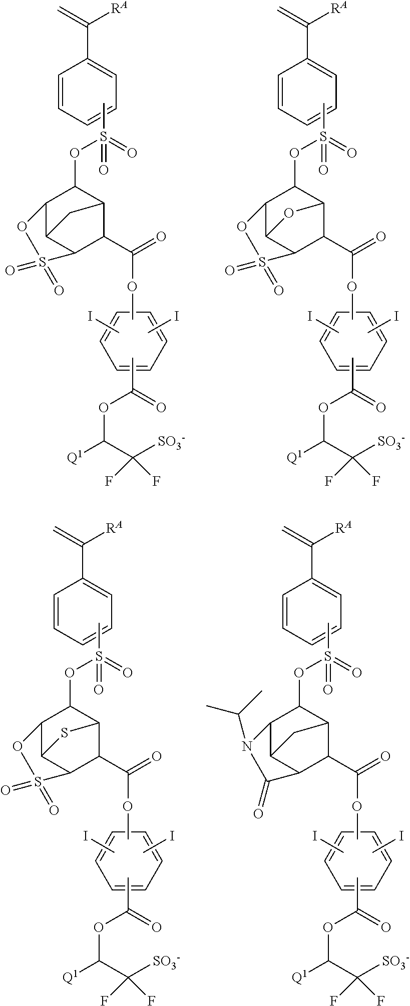

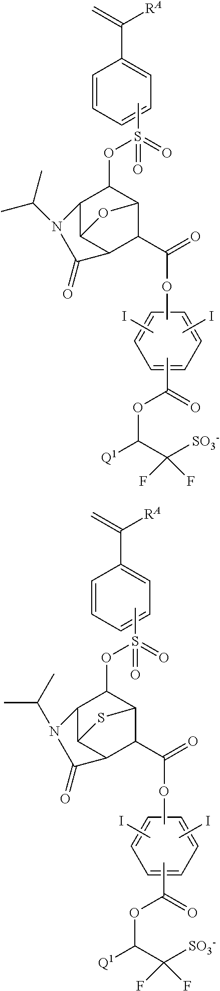

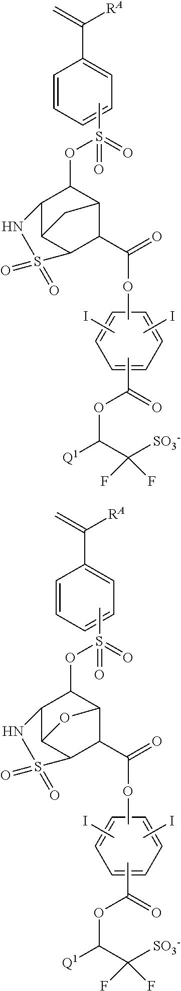

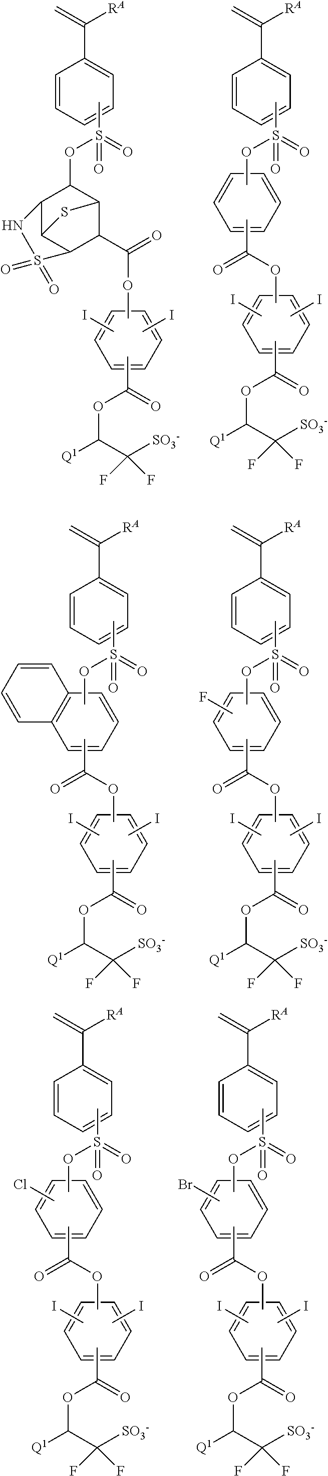

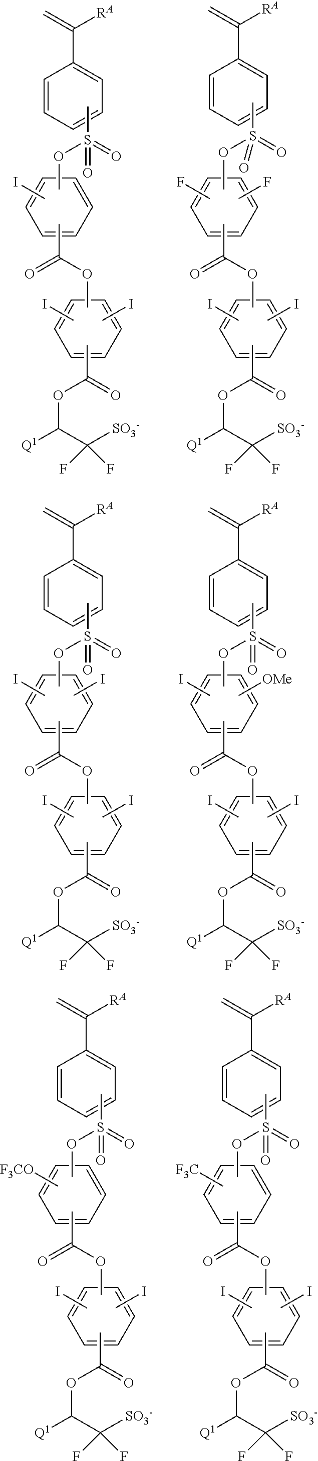

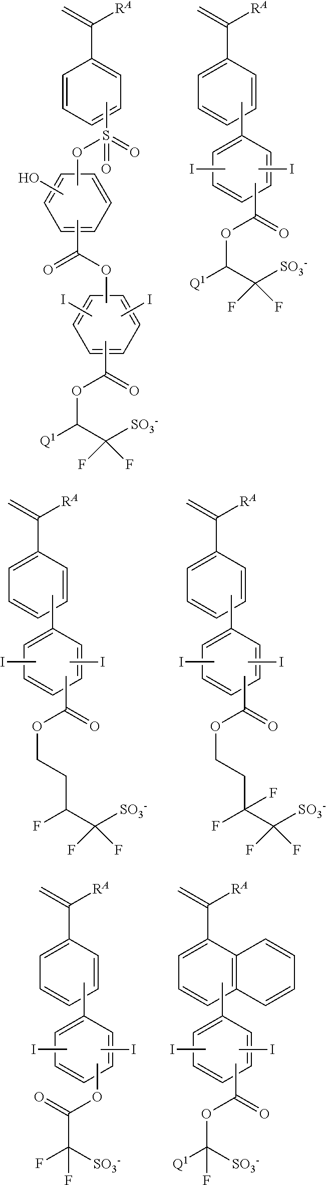

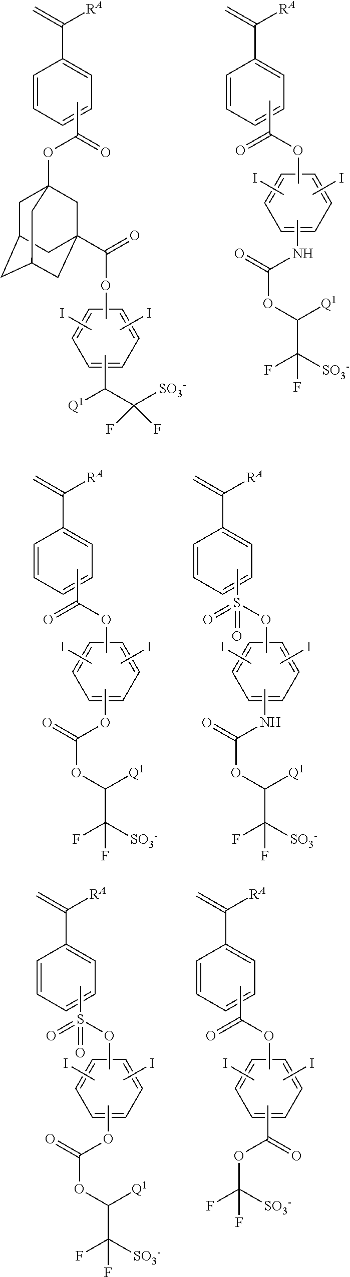

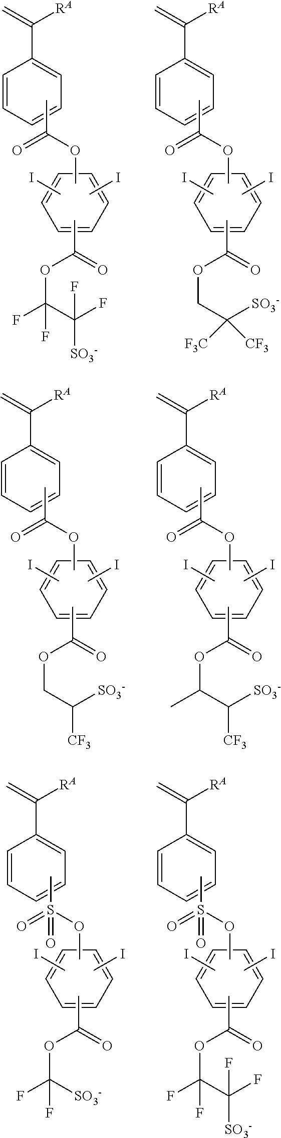

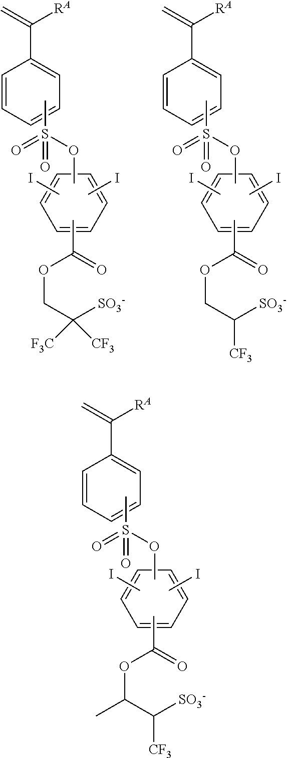

In formula (1), Z⁺ is an onium cation. The onium cation is preferably a sulfonium cation having the following formula (cation-1) or iodonium cation having the following formula (cation-2).

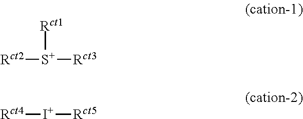

In formulae (cation-1) and (cation-2), R^{ct1} to R^{ct5} are each independently a halogen atom, or a C1-C₃₀ hydrocarbyl group which may contain a heteroatom.

Specific examples of the halogen atom represented by R^{ct1} to R^{ct5} include a fluorine atom, a chlorine atom, a bromine atom, and an iodine atom.

The hydrocarbyl group represented by R^{ct1} to R^{ct5} may be saturated or unsaturated, and may be linear, branched, or cyclic. Examples thereof include C₁-C₃₀ alkyl groups such as methyl, ethyl, n-propyl, isopropyl, n-butyl, sec-butyl and tert-butyl groups; C₃-C₃₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₃₀ alkenyl groups such as vinyl, allyl, propenyl, butenyl and hexenyl groups; C₃-C₃₀ cyclic unsaturated hydrocarbyl groups such as a cyclohexenyl group; C₆-C₃₀ aryl groups such as phenyl, naphthyl and thienyl groups; C₇-C₃₀ aralkyl groups such as benzyl, 1-phenylethyl and 2-phenylethyl groups; and combinations thereof. The aryl groups are preferred. Some or all of hydrogen atoms of the hydrocarbyl group may be replaced by a group containing a heteroatom such as an oxygen atom, a sulfur atom, a nitrogen atom or a halogen atom, some constituent —CH₂— of the hydrocarbyl group may be replaced by a group containing a heteroatom such as an oxygen atom, a sulfur atom or a nitrogen atom, and as a result, the hydrocarbyl group may contain a hydroxy group, a fluorine atom, a chlorine atom, a bromine atom, an iodine atom, a cyano group, a nitro group, a carbonyl group, an ether bond, an ester bond, a sulfonic ester bond, a carbonate bond, a lactone ring, a sultone ring, carboxylic acid anhydride (—C(═O)—O—C(═O)—), a haloalkyl group, or the like.

R^{ct1} and R^{ct2} may bond together to form a ring with the sulfur atom to which they are attached. Examples of the structure of the ring include those represented by the following formula.

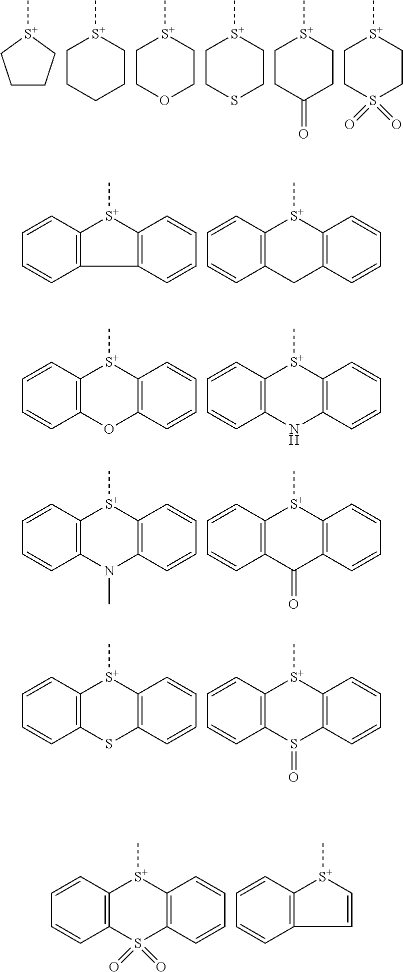

Herein the broken line designates a point of attachment to R^{ct3}.

Examples of the sulfonium cation represented by formula (cation-1) are shown below, but not limited thereto.

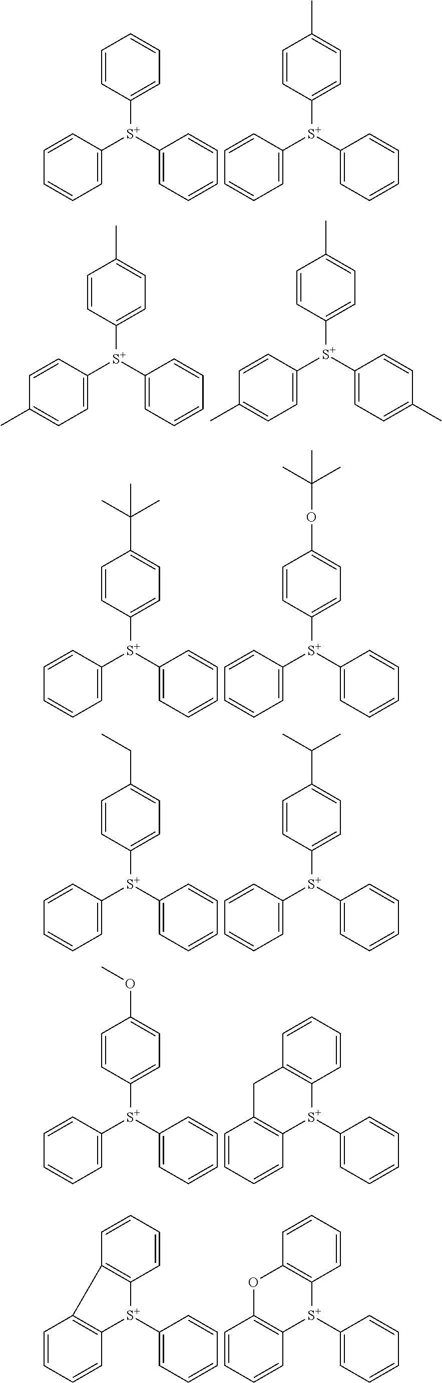

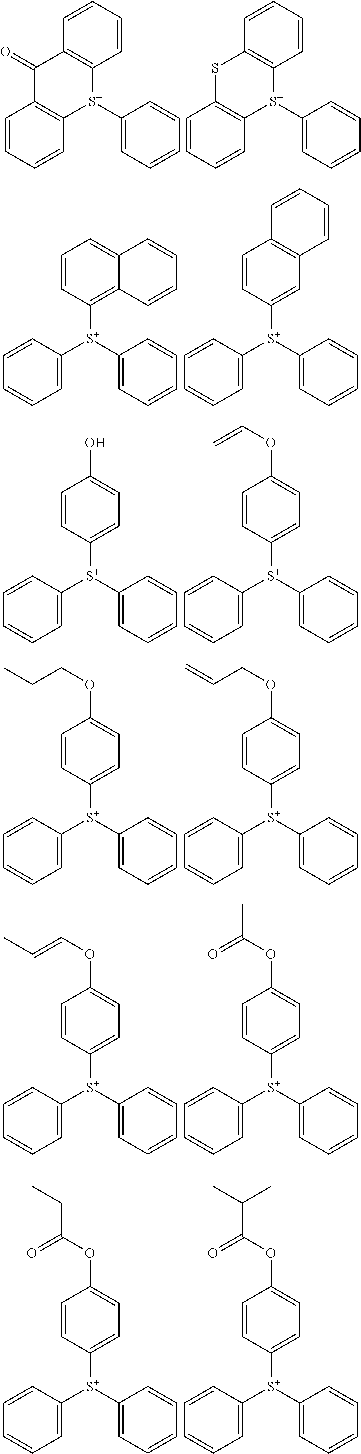

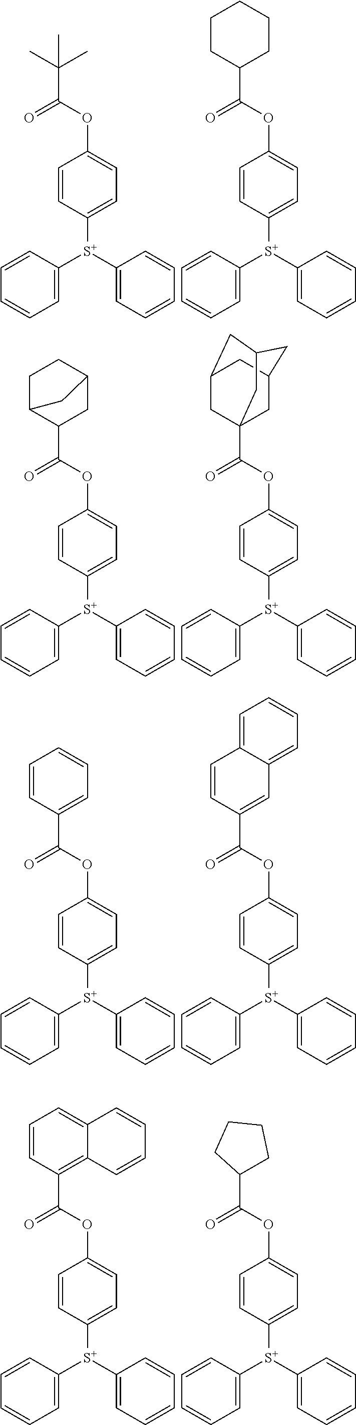

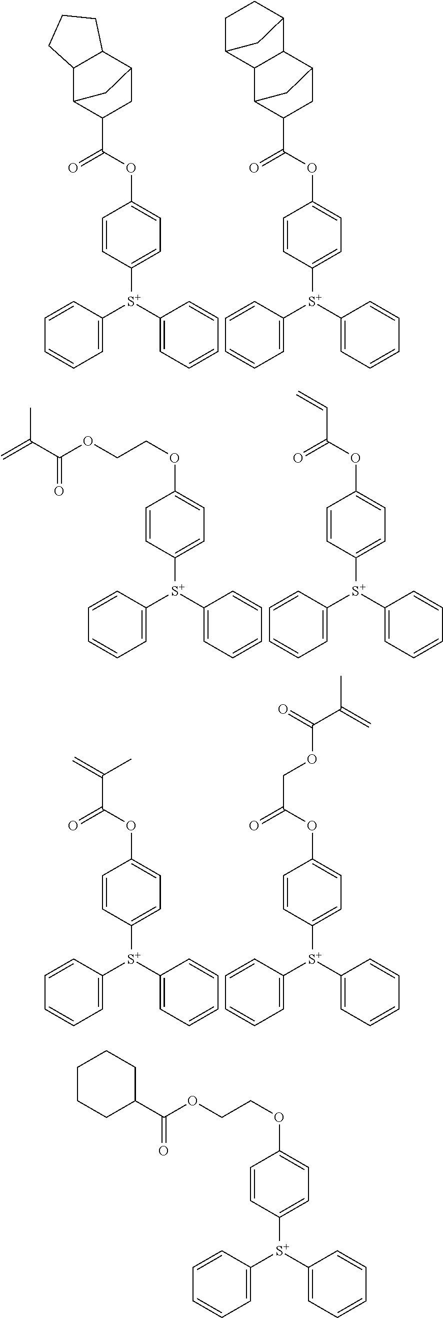

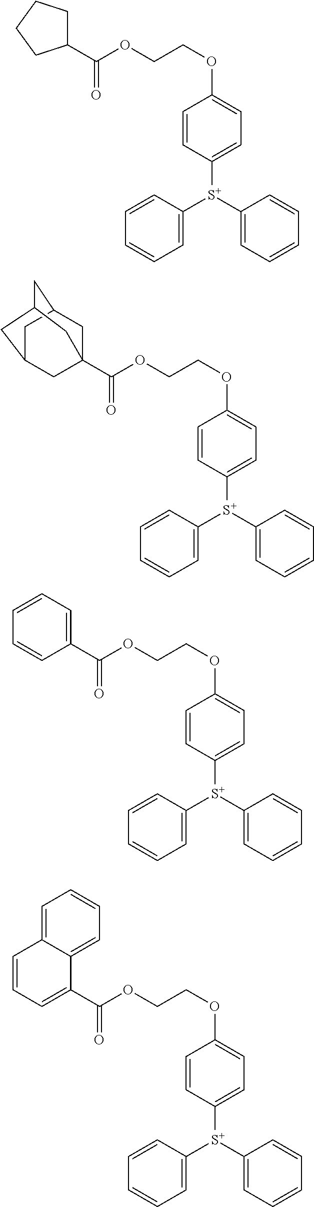 [SMILES: O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2 ; O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12]

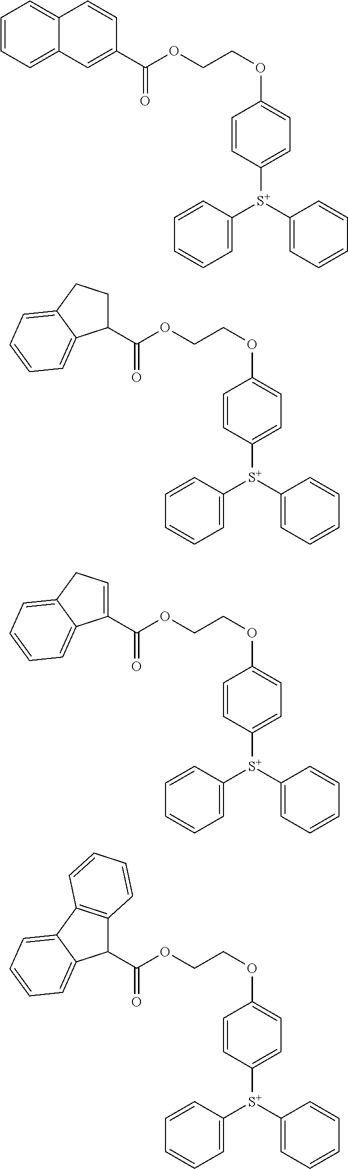 [SMILES: O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21 ; O=C(OCCOc1ccc([S+](c2ccccc2)c2ccccc2)cc1)C1c2ccccc2-c2ccccc21]

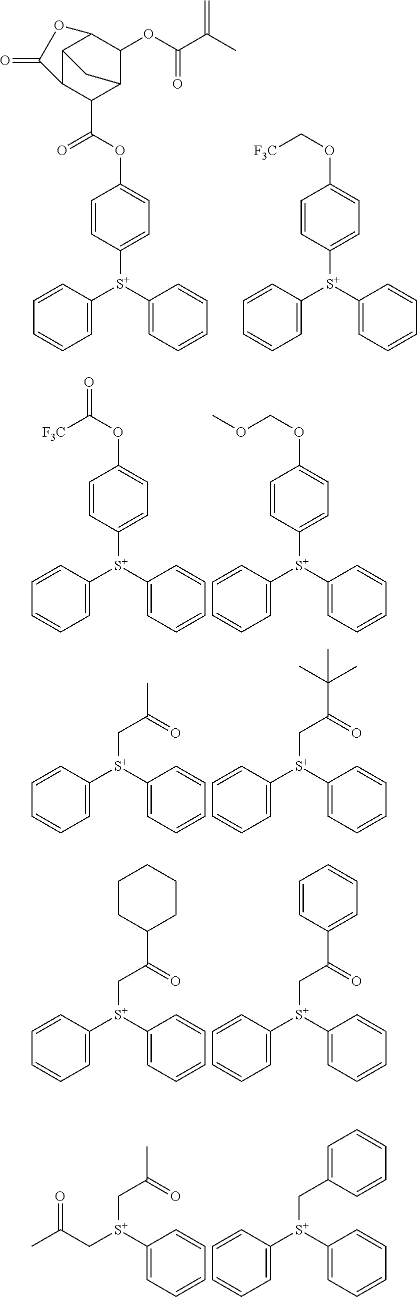

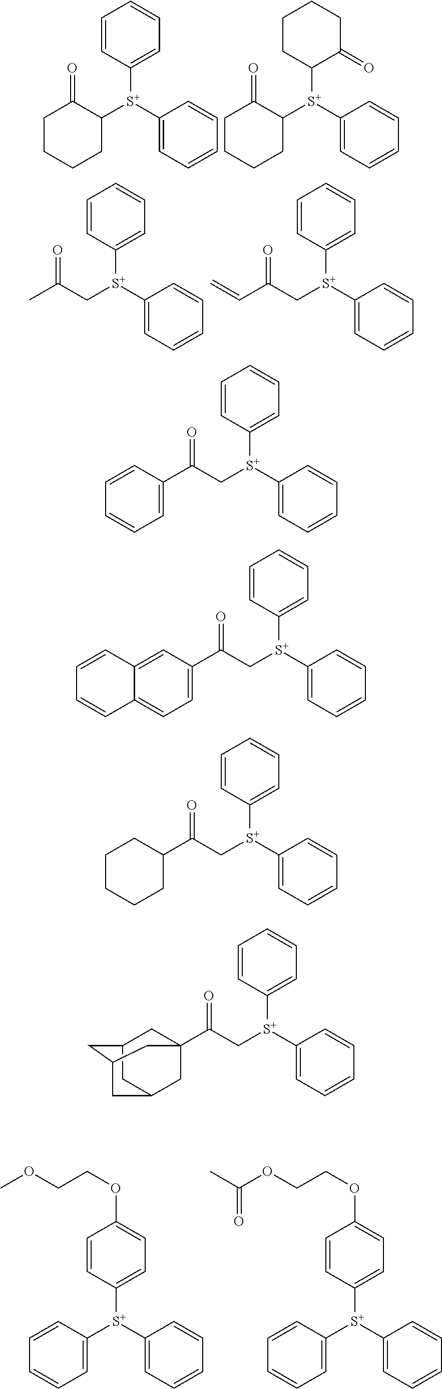

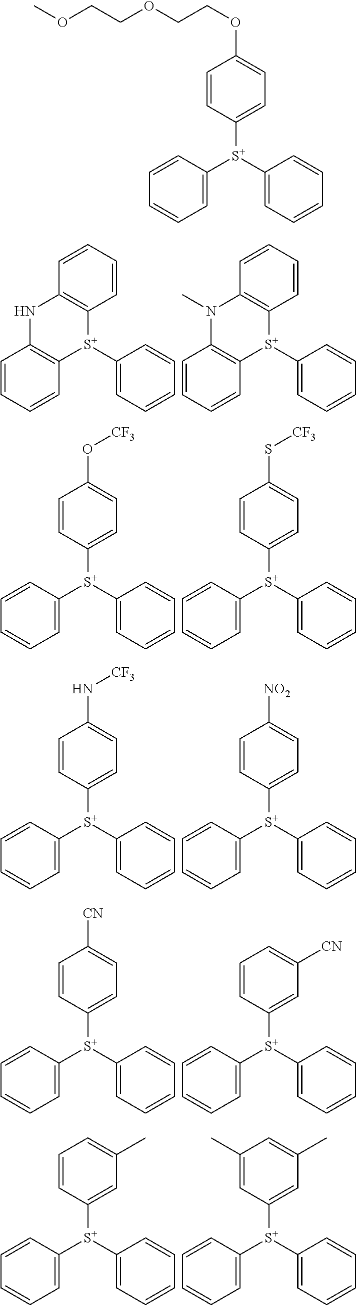

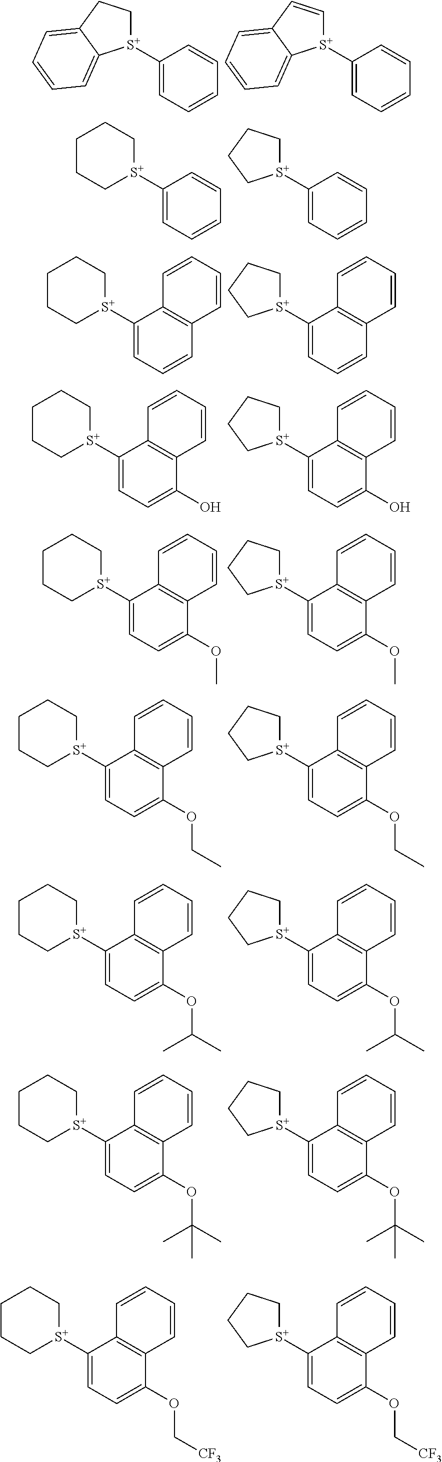

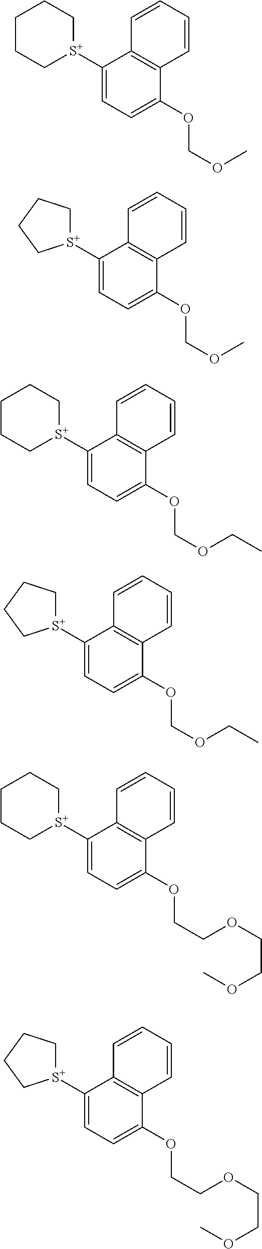 [SMILES: COCOc1ccc([S+]2CCCCC2)c2ccccc12 ; COCOc1ccc([S+]2CCCC2)c2ccccc12 ; CCOCOc1ccc([S+]2CCCCC2)c2ccccc12 ; CCOCOc1ccc([S+]2CCCC2)c2ccccc12 ; COCCOCCOc1ccc([S+]2CCCCC2)c2ccccc12 ; COCCOCCOc1ccc([S+]2CCCC2)c2ccccc12]

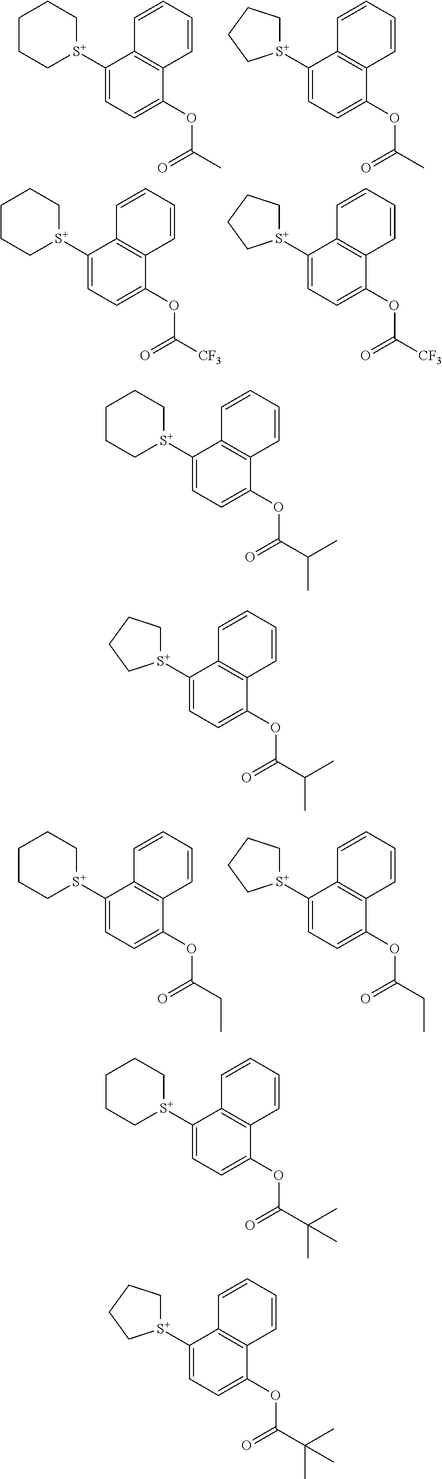

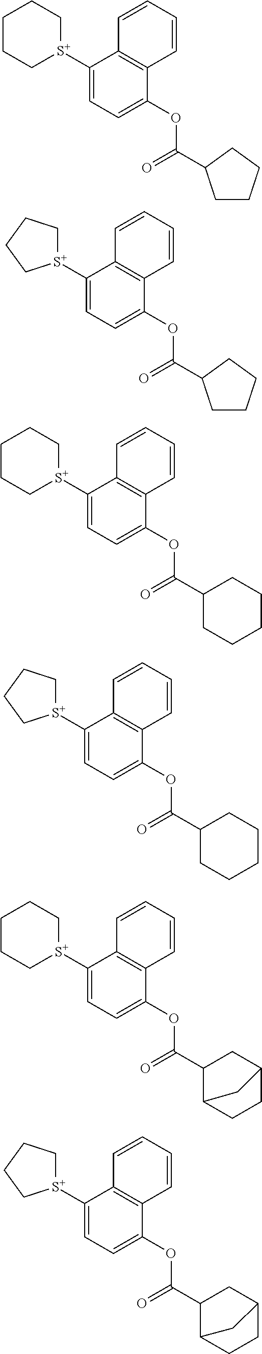 [SMILES: O=C(Oc1ccc([S+]2CCCCC2)c2ccccc12)C1CCCC1 ; O=C(Oc1ccc([S+]2CCCC2)c2ccccc12)C1CCCC1 ; O=C(Oc1ccc([S+]2CCCCC2)c2ccccc12)C1CCCCC1 ; O=C(Oc1ccc([S+]2CCCC2)c2ccccc12)C1CCCCC1 ; O=C(Oc1ccc([S+]2CCCCC2)c2ccccc12)C1CC2CCC1C2 ; O=C(Oc1ccc([S+]2CCCC2)c2ccccc12)C1CC2CCC1C2]

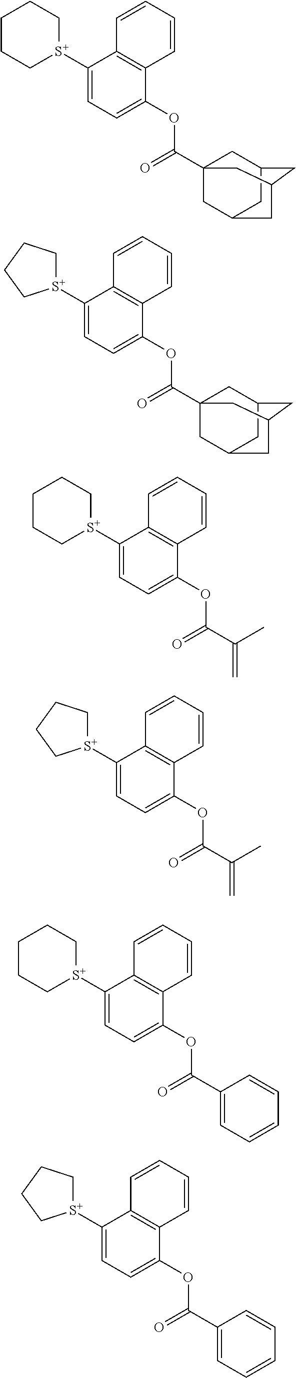 [SMILES: O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12 ; O=C(Oc1ccc([S+]2CCCCC2)c2ccccc12)c1ccccc1 ; O=C(Oc1ccc([S+]2CCCC2)c2ccccc12)c1ccccc1]

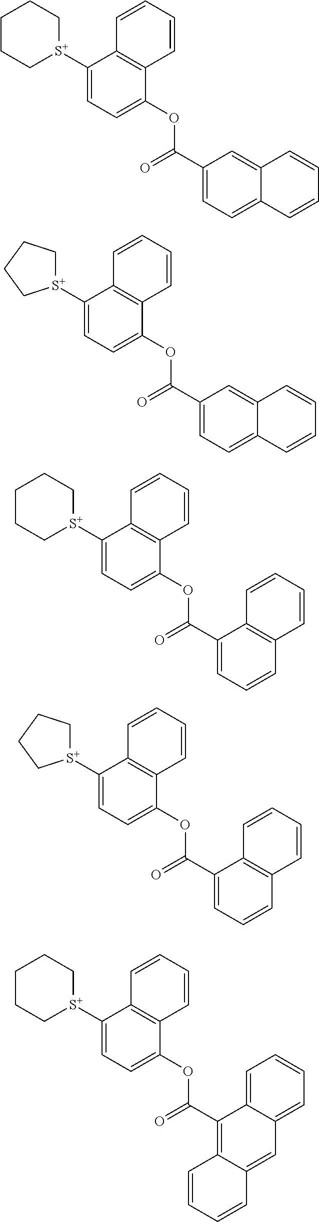 [SMILES: O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12 ; O=C(Oc1ccc([S+]2CCCCC2)c2ccccc12)c1c2ccccc2cc2ccccc12]

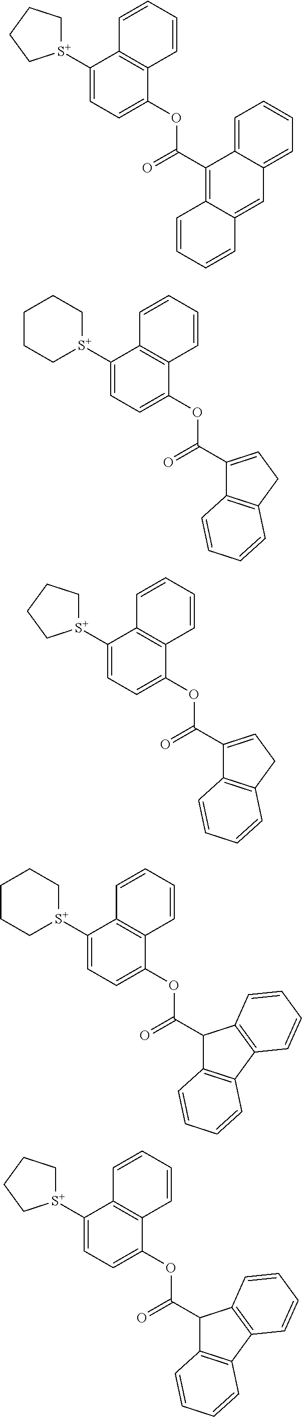 [SMILES: O=C(Oc1ccc([S+]2CCCC2)c2ccccc12)c1c2ccccc2cc2ccccc12 ; O=C(Oc1ccc([S+]2CCCCC2)c2ccccc12)C1=CCc2ccccc21 ; O=C(Oc1ccc([S+]2CCCC2)c2ccccc12)C1=CCc2ccccc21 ; O=C(Oc1ccc([S+]2CCCCC2)c2ccccc12)C1c2ccccc2-c2ccccc21 ; O=C(Oc1ccc([S+]2CCCC2)c2ccccc12)C1c2ccccc2-c2ccccc21]

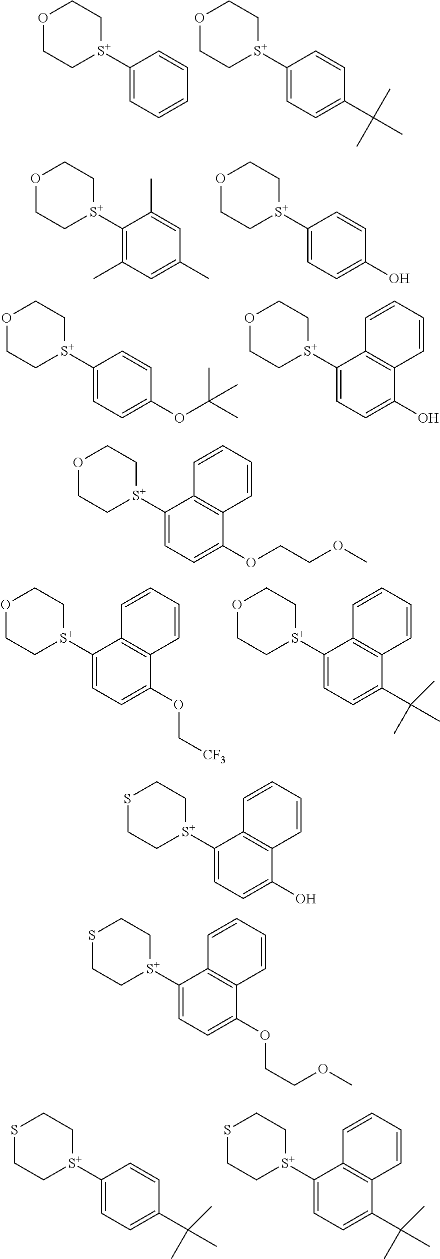

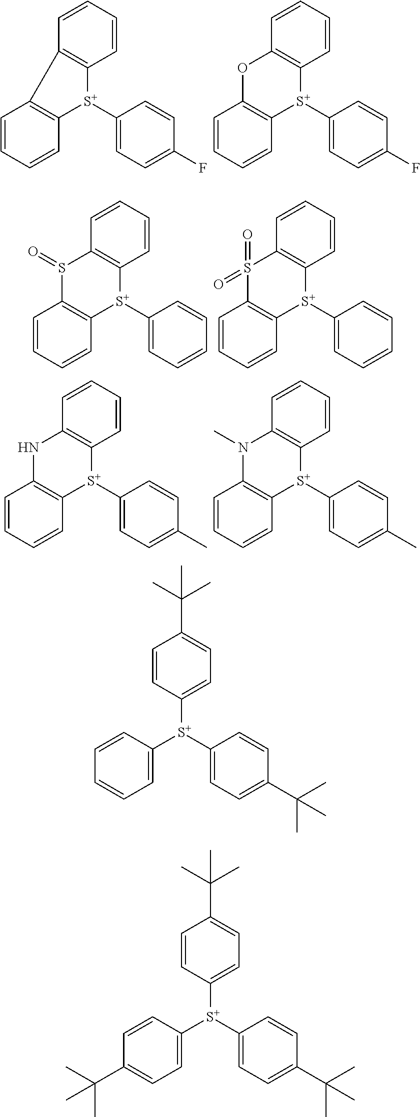

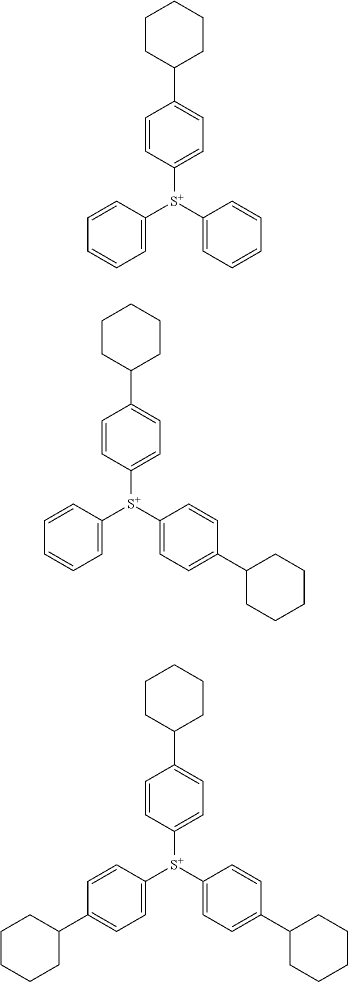 [SMILES: c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1]

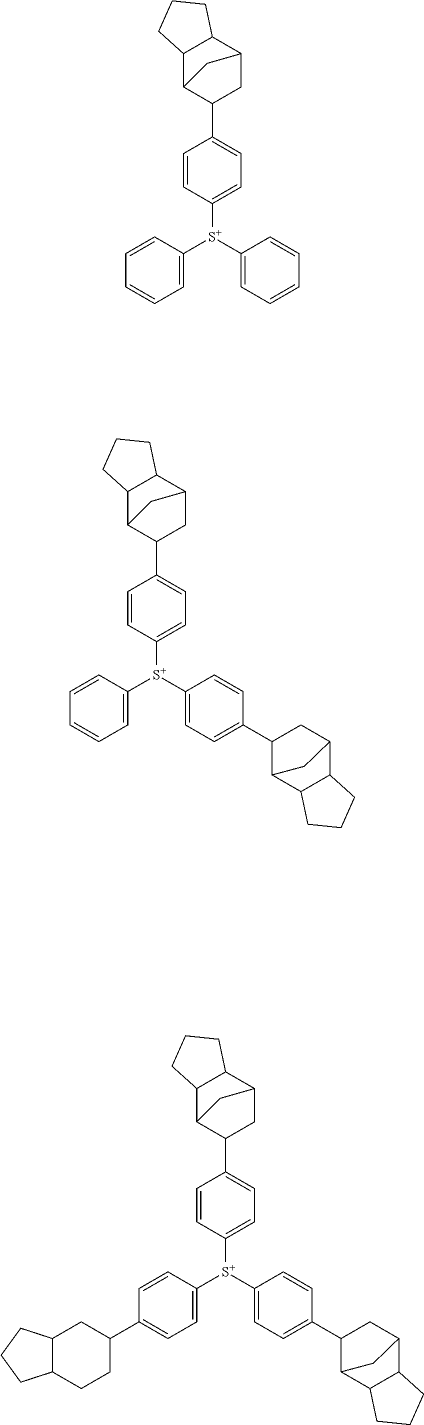 [SMILES: c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CCC2CCCC2C1]

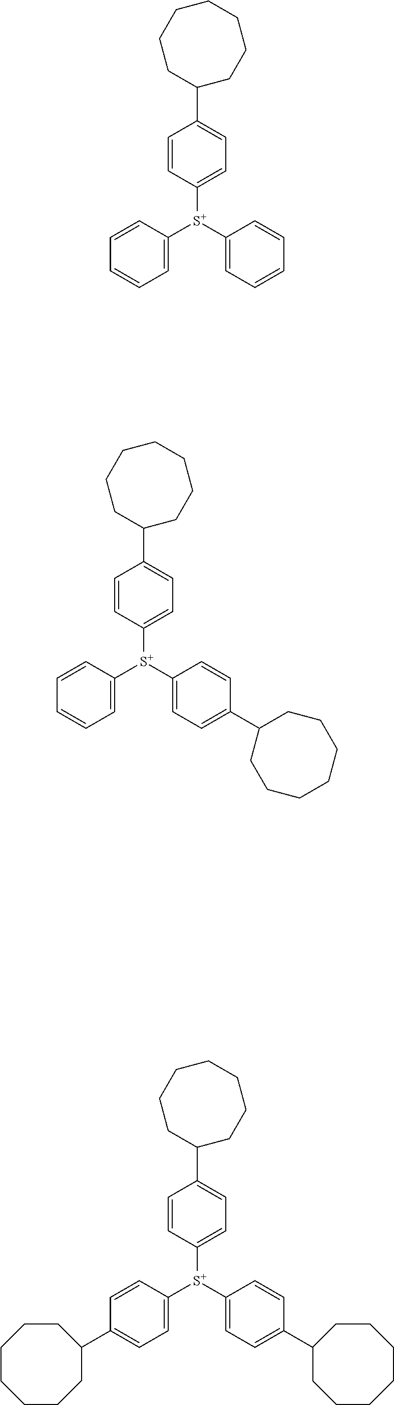 [SMILES: c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1]

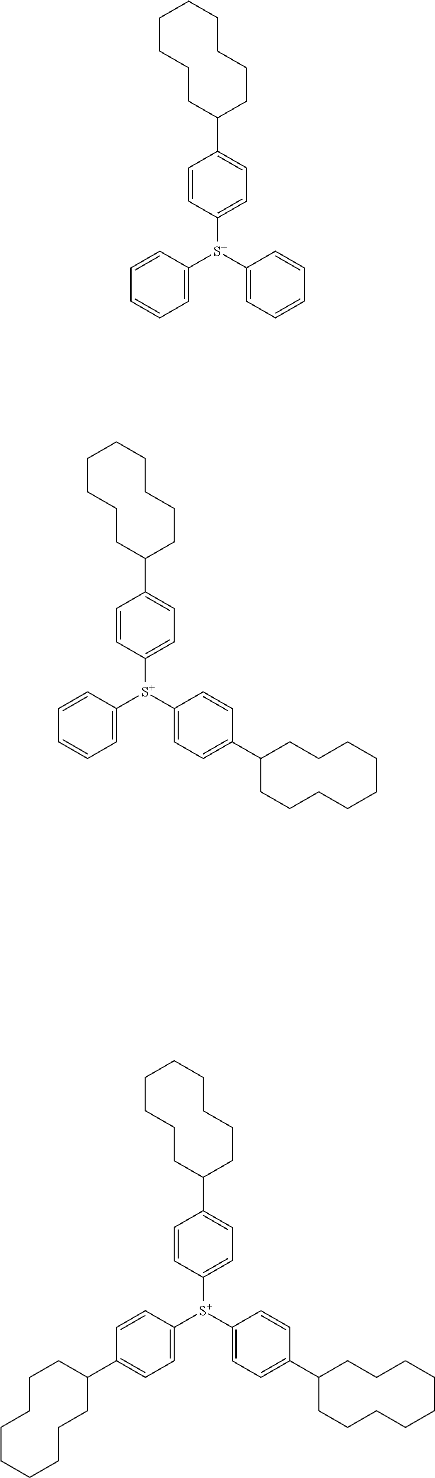 [SMILES: c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1]

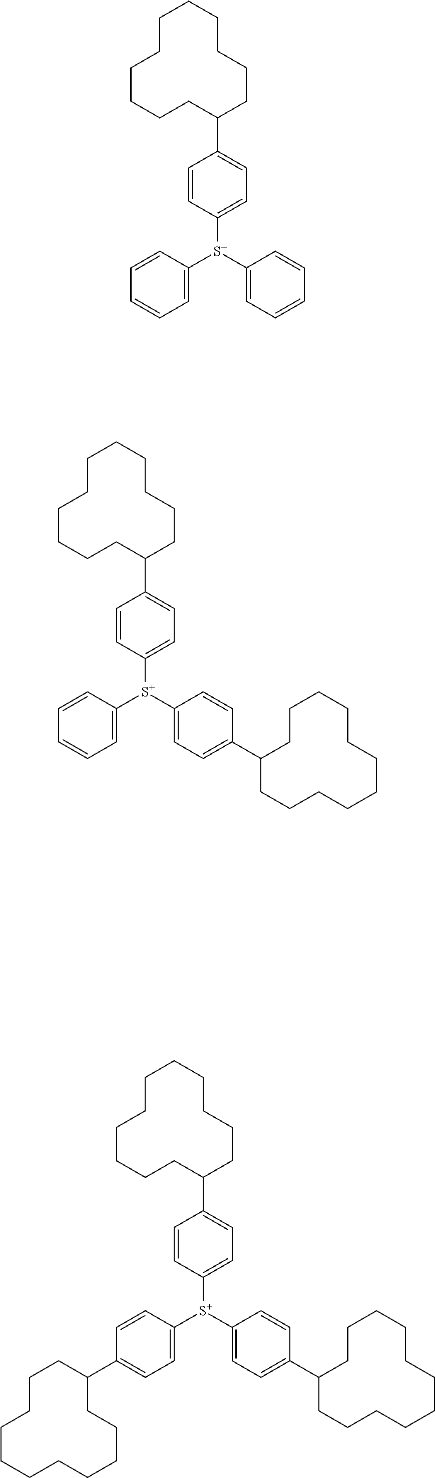 [SMILES: c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1]

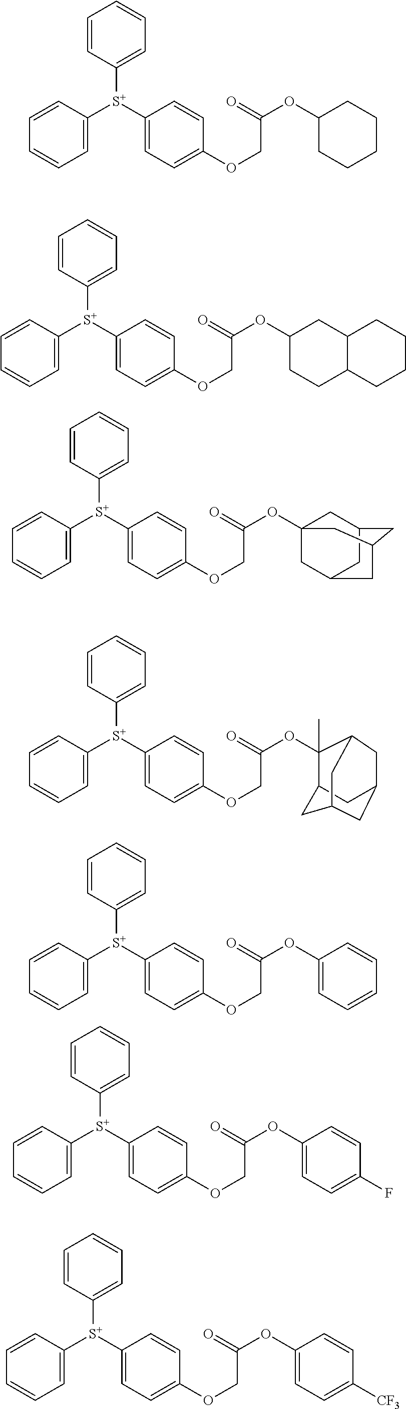

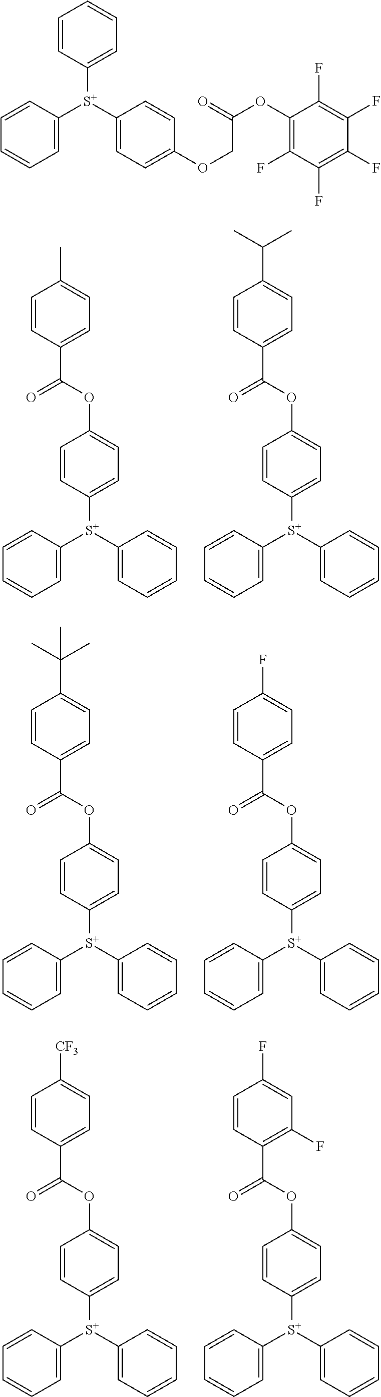

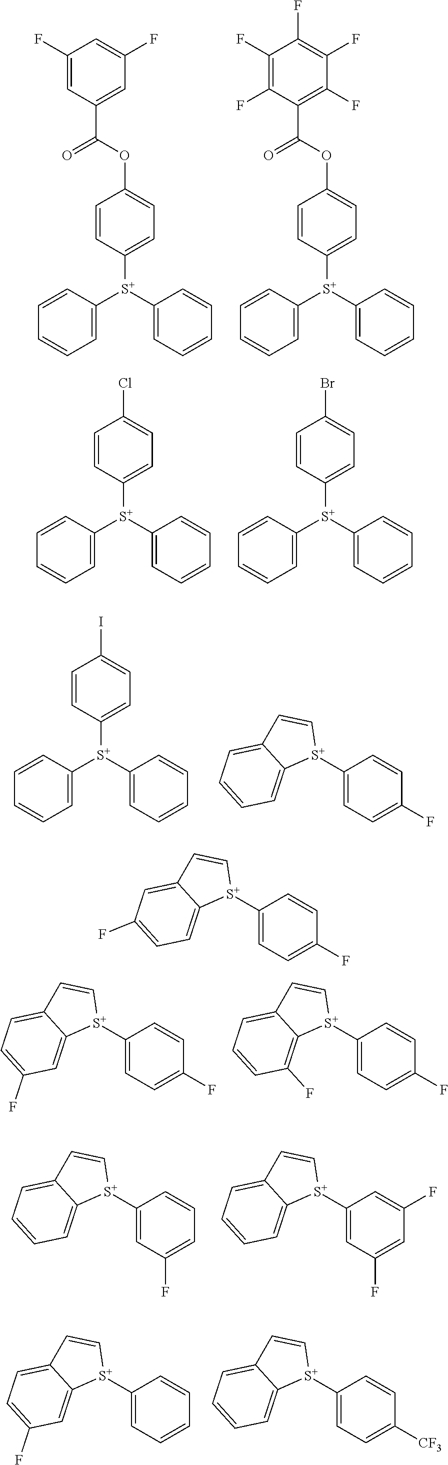

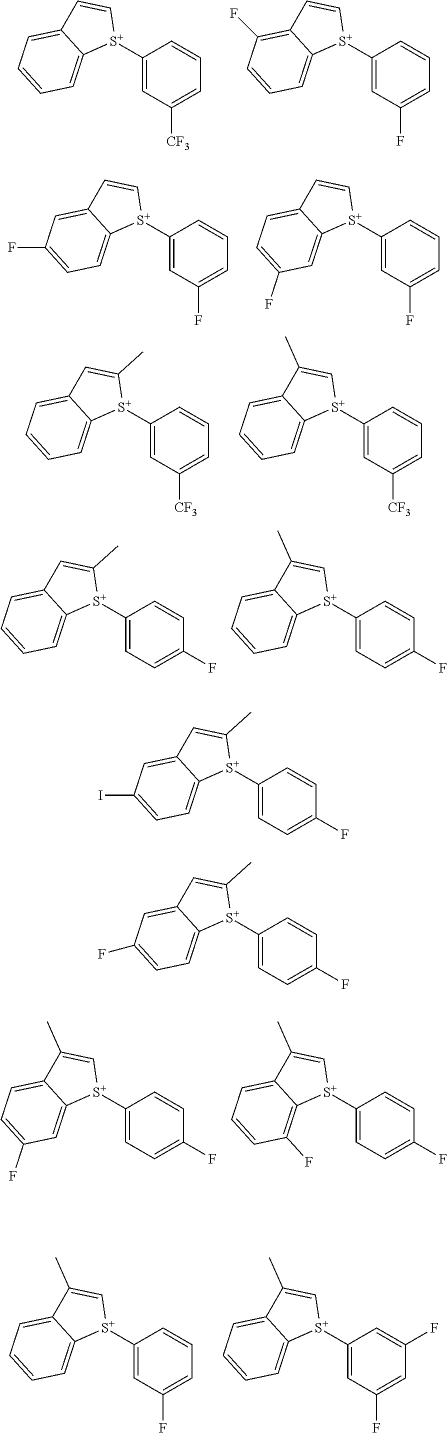

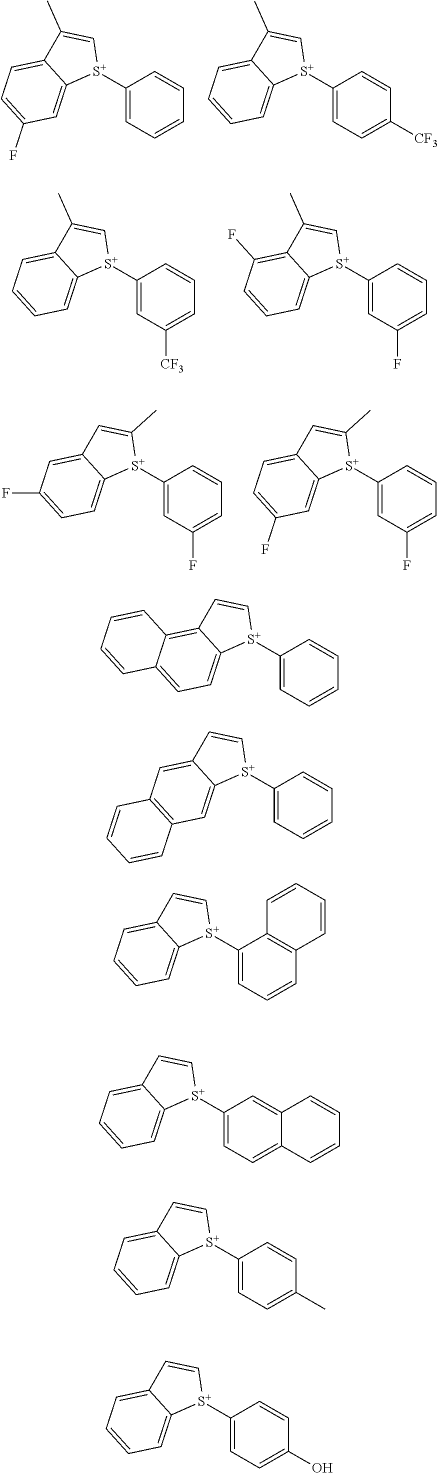

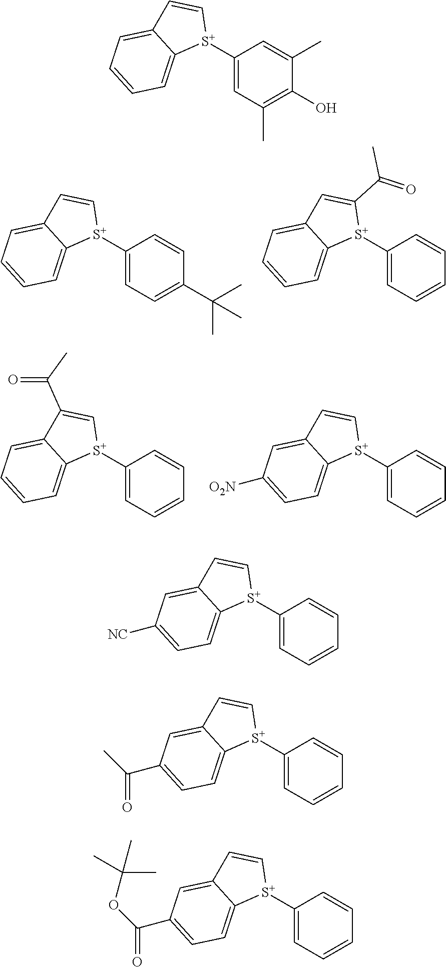

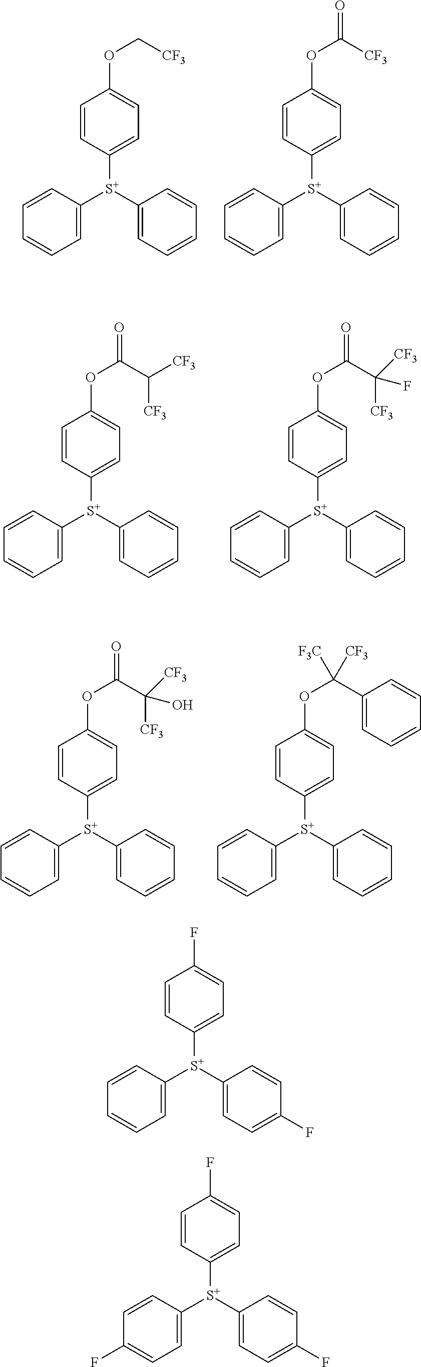

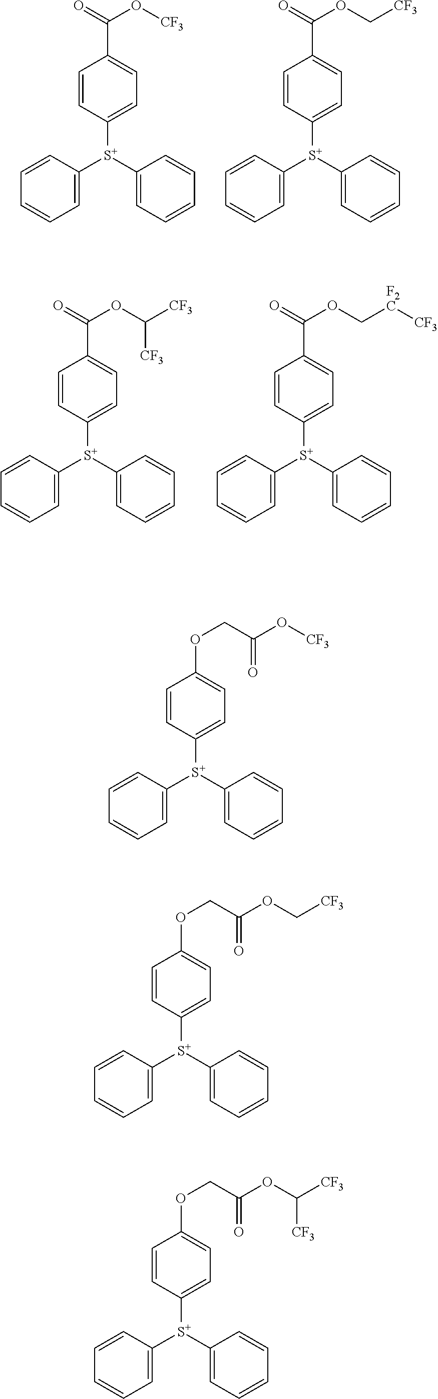

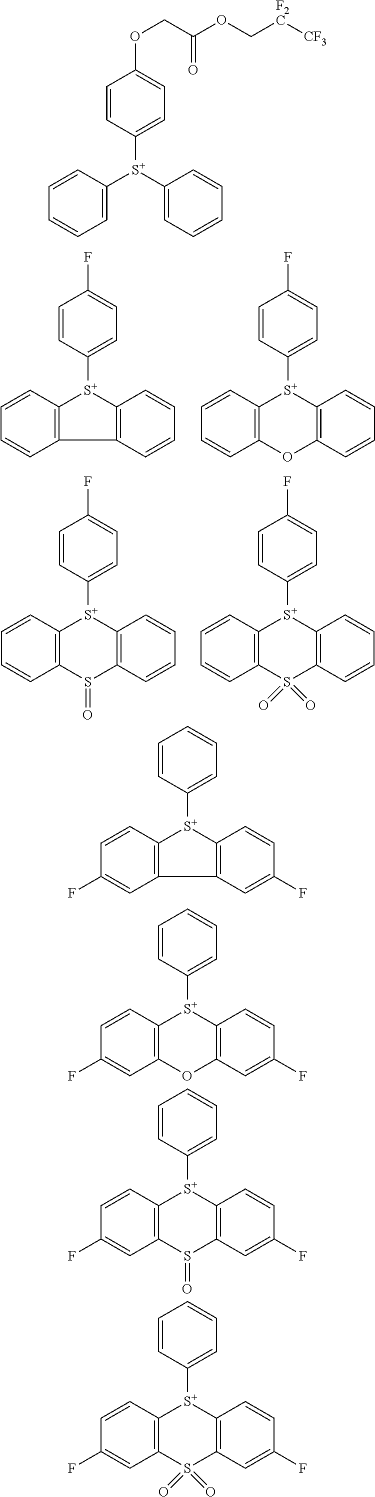

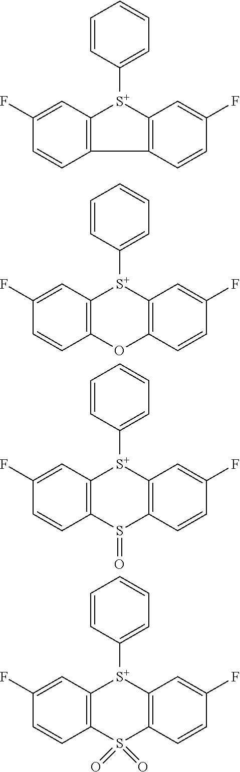 [SMILES: Fc1ccc2c3ccc(F)cc3[s+](-c3ccccc3)c2c1 ; Fc1ccc2c(c1)[S+](c1ccccc1)c1cc(F)ccc1O2 ; O=S1c2ccc(F)cc2[S+](c2ccccc2)c2cc(F)ccc21 ; O=S1(=O)c2ccc(F)cc2[S+](c2ccccc2)c2cc(F)ccc21]

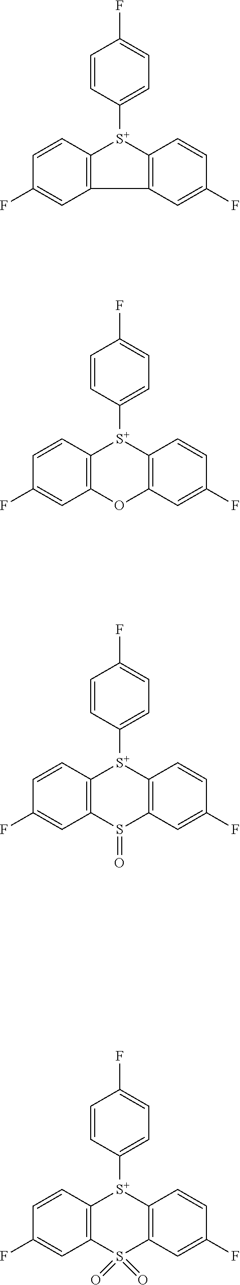 [SMILES: Fc1ccc(-[s+]2c3ccc(F)cc3c3cc(F)ccc32)cc1 ; Fc1ccc([S+]2c3ccc(F)cc3Oc3cc(F)ccc32)cc1 ; O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21]

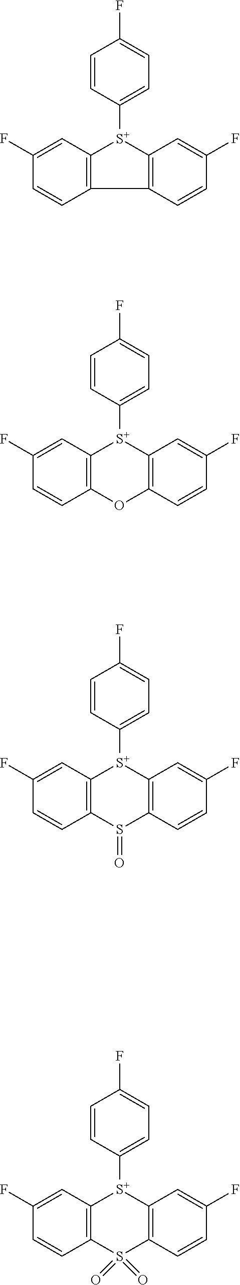 [SMILES: Fc1ccc(-[s+]2c3cc(F)ccc3c3ccc(F)cc32)cc1 ; Fc1ccc([S+]2c3cc(F)ccc3Oc3ccc(F)cc32)cc1 ; O=S1c2ccc(F)cc2[S+](c2ccc(F)cc2)c2cc(F)ccc21 ; O=S1(=O)c2ccc(F)cc2[S+](c2ccc(F)cc2)c2cc(F)ccc21]

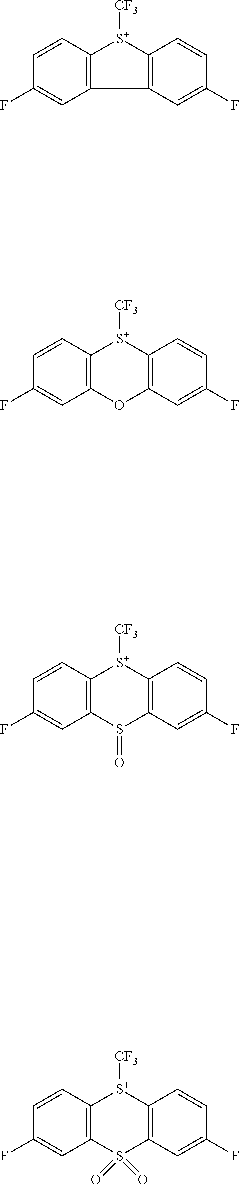 [SMILES: Fc1ccc2c(c1)c1cc(F)ccc1[s+]2C(F)(F)F ; Fc1ccc2c(c1)Oc1cc(F)ccc1[S+]2C(F)(F)F ; O=S1c2cc(F)ccc2[S+](C(F)(F)F)c2ccc(F)cc21 ; O=S1(=O)c2cc(F)ccc2[S+](C(F)(F)F)c2ccc(F)cc21]

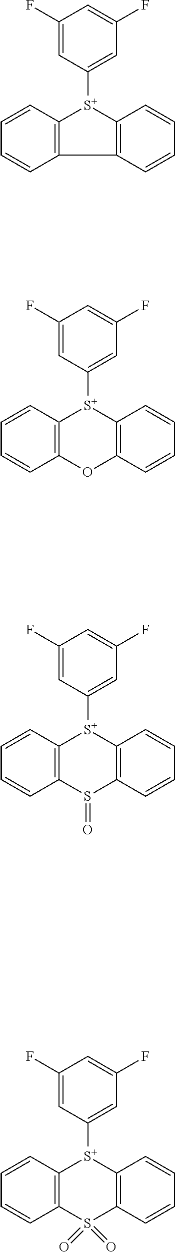 [SMILES: Fc1cc(F)cc(-[s+]2c3ccccc3c3ccccc32)c1 ; Fc1cc(F)cc([S+]2c3ccccc3Oc3ccccc32)c1 ; O=S1c2ccccc2[S+](c2cc(F)cc(F)c2)c2ccccc21 ; O=S1(=O)c2ccccc2[S+](c2cc(F)cc(F)c2)c2ccccc21]

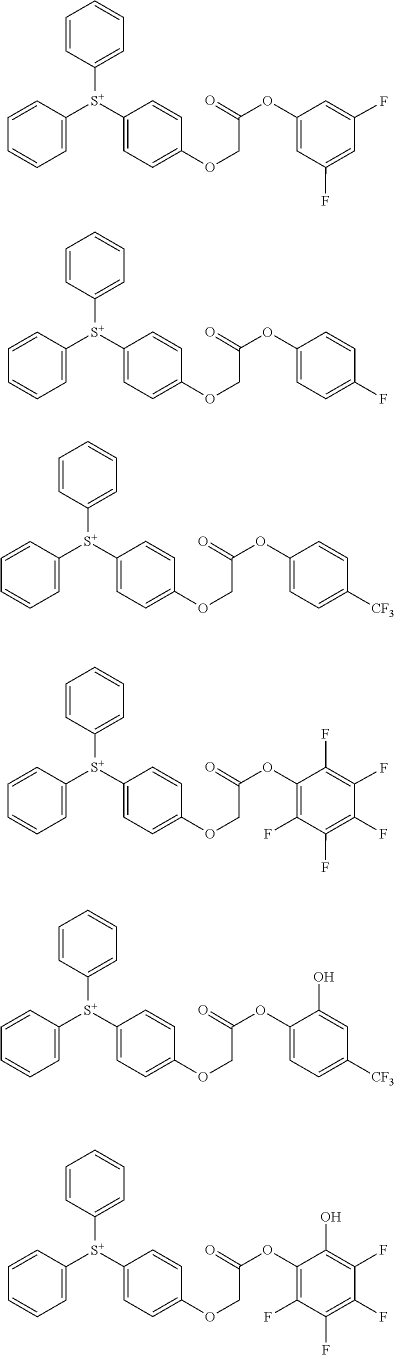 [SMILES: O=C(COc1ccc([S+](c2ccccc2)c2ccccc2)cc1)Oc1cc(F)cc(F)c1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; O=C(COc1ccc([S+](c2ccccc2)c2ccccc2)cc1)Oc1ccc(C(F)(F)F)cc1O ; O=C(COc1ccc([S+](c2ccccc2)c2ccccc2)cc1)Oc1c(O)c(F)c(F)c(F)c1F]

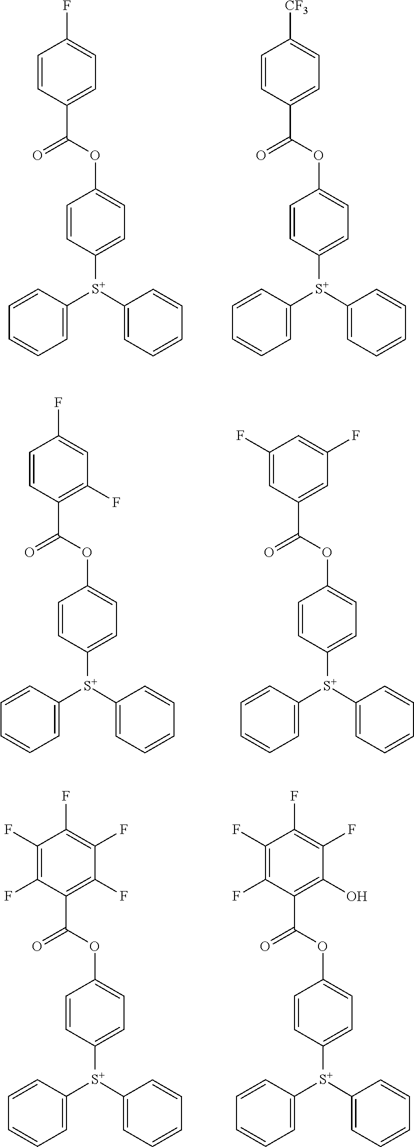 [SMILES: O=C(Oc1ccc([S+](c2ccccc2)c2ccccc2)cc1)c1ccc(F)cc1 ; O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F ; O=C(Oc1ccc([S+](c2ccccc2)c2ccccc2)cc1)c1cc(F)cc(F)c1 ; O=C(Oc1ccc([S+](c2ccccc2)c2ccccc2)cc1)c1c(F)c(F)c(F)c(F)c1F ; O=C(Oc1ccc([S+](c2ccccc2)c2ccccc2)cc1)c1c(O)c(F)c(F)c(F)c1F]

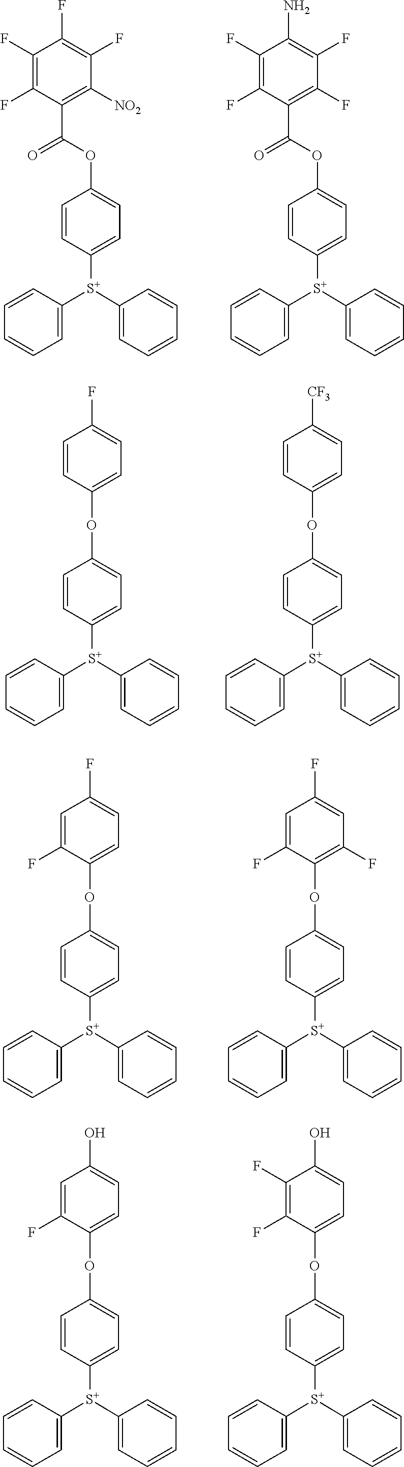

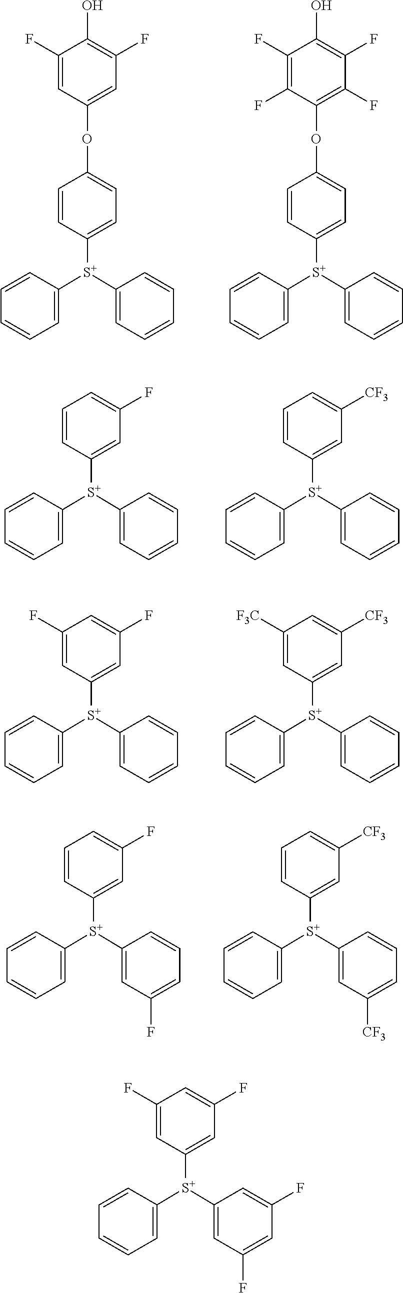

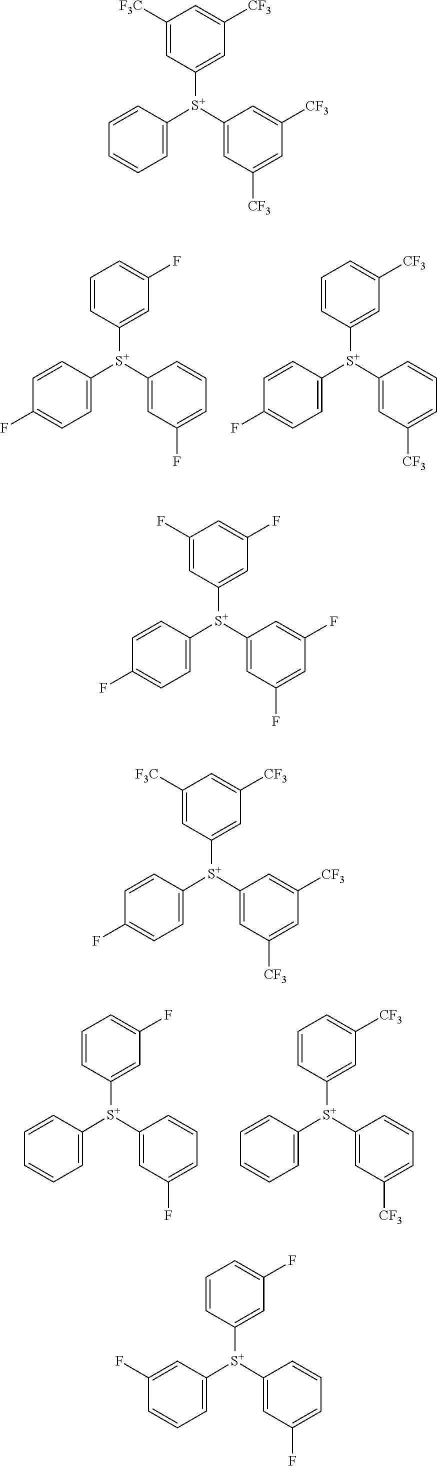

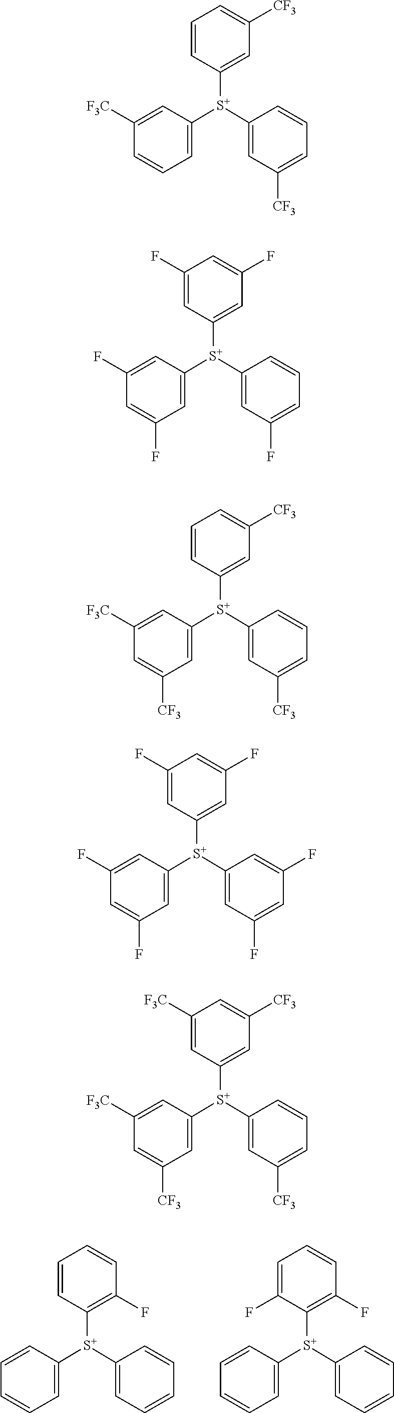

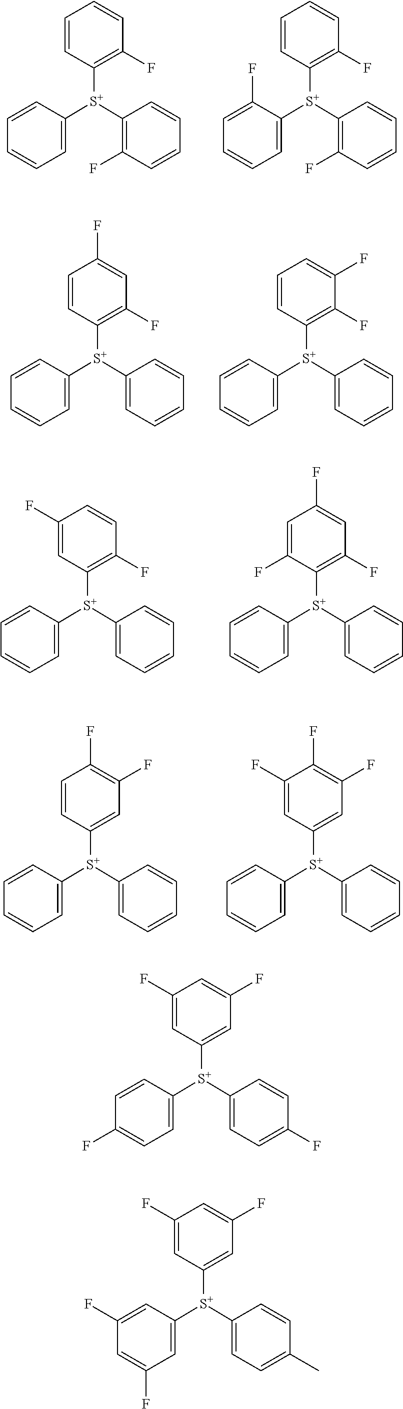

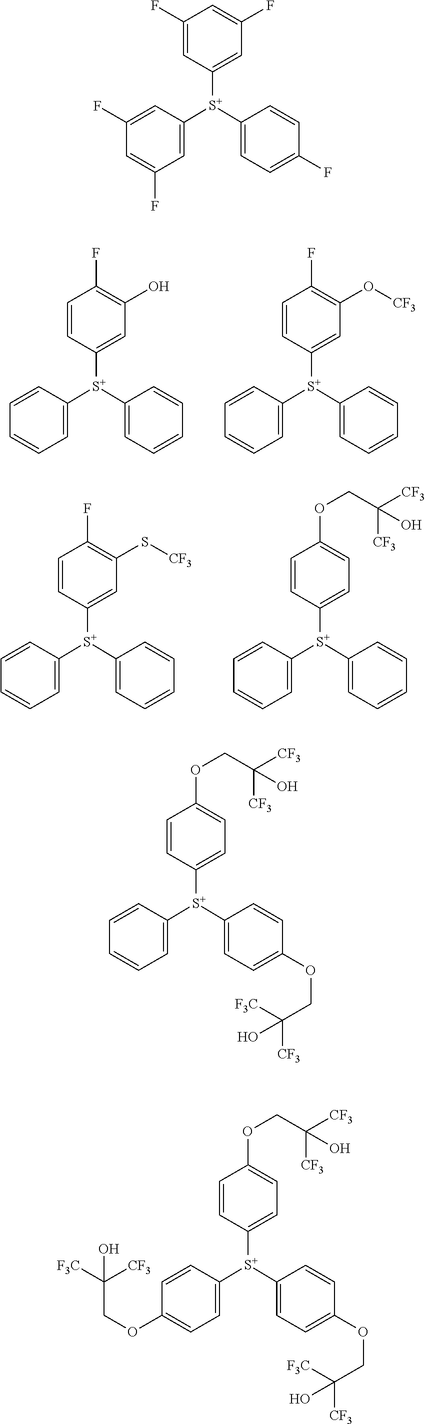

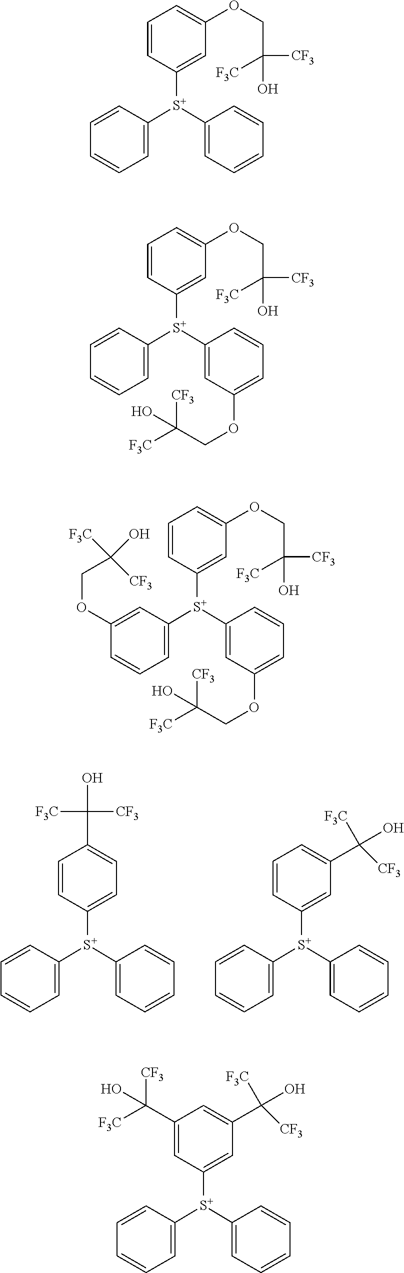 [SMILES: OC(COc1cccc([S+](c2ccccc2)c2ccccc2)c1)(C(F)(F)F)C(F)(F)F ; OC(COc1cccc([S+](c2ccccc2)c2cccc(OCC(O)(C(F)(F)F)C(F)(F)F)c2)c1)(C(F)(F)F)C(F)(F)F ; OC(COc1cccc([S+](c2cccc(OCC(O)(C(F)(F)F)C(F)(F)F)c2)c2cccc(OCC(O)(C(F)(F)F)C(F)(F)F)c2)c1)(C(F)(F)F)C(F)(F)F ; OC(c1ccc([S+](c2ccccc2)c2ccccc2)cc1)(C(F)(F)F)C(F)(F)F ; OC(c1cccc([S+](c2ccccc2)c2ccccc2)c1)(C(F)(F)F)C(F)(F)F ; OC(c1cc([S+](c2ccccc2)c2ccccc2)cc(C(O)(C(F)(F)F)C(F)(F)F)c1)(C(F)(F)F)C(F)(F)F]

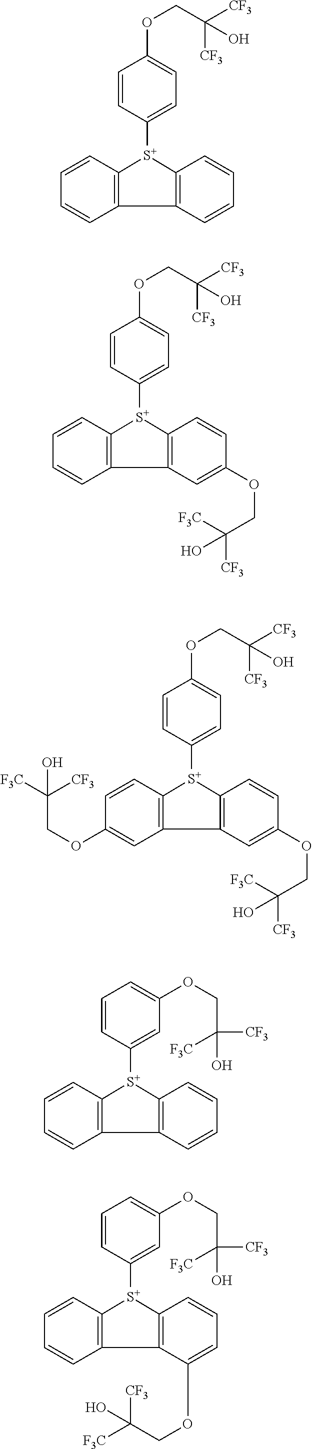 [SMILES: OC(COc1ccc(-[s+]2c3ccccc3c3ccccc32)cc1)(C(F)(F)F)C(F)(F)F ; OC(COc1ccc(-[s+]2c3ccccc3c3cc(OCC(O)(C(F)(F)F)C(F)(F)F)ccc32)cc1)(C(F)(F)F)C(F)(F)F ; OC(COc1ccc(-[s+]2c3ccc(OCC(O)(C(F)(F)F)C(F)(F)F)cc3c3cc(OCC(O)(C(F)(F)F)C(F)(F)F)ccc32)cc1)(C(F)(F)F)C(F)(F)F ; OC(COc1cccc(-[s+]2c3ccccc3c3ccccc32)c1)(C(F)(F)F)C(F)(F)F ; OC(COc1cccc(-[s+]2c3ccccc3c3c(OCC(O)(C(F)(F)F)C(F)(F)F)cccc32)c1)(C(F)(F)F)C(F)(F)F]

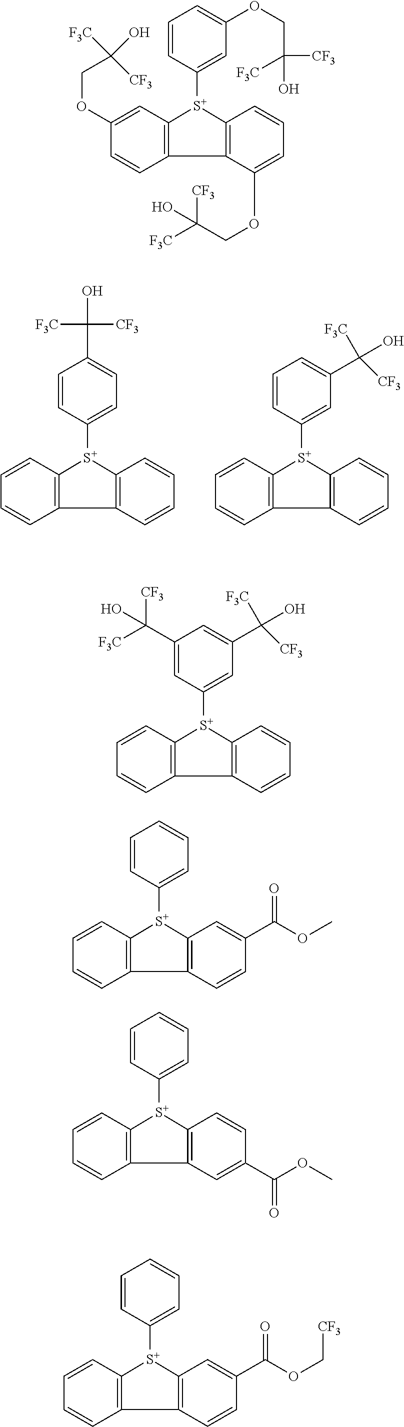

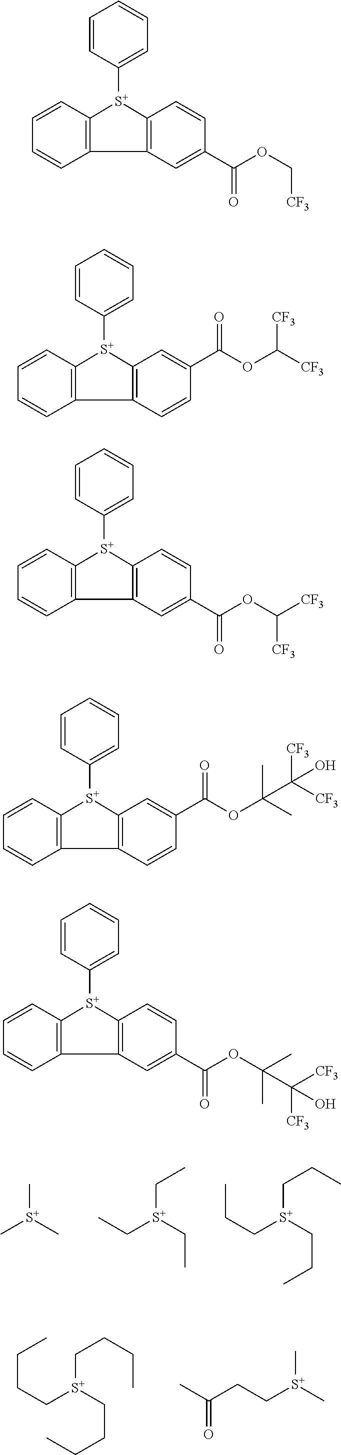

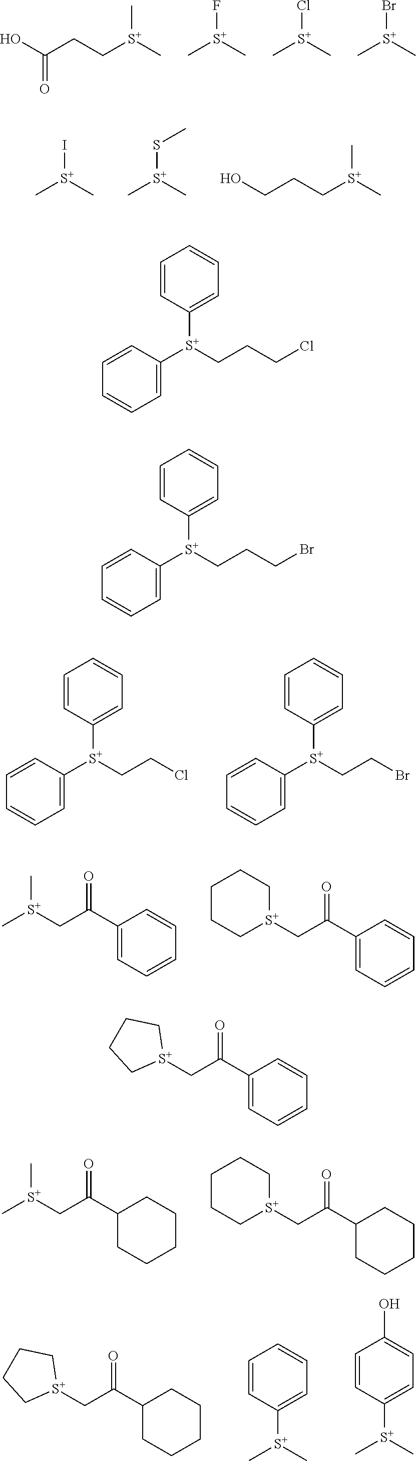

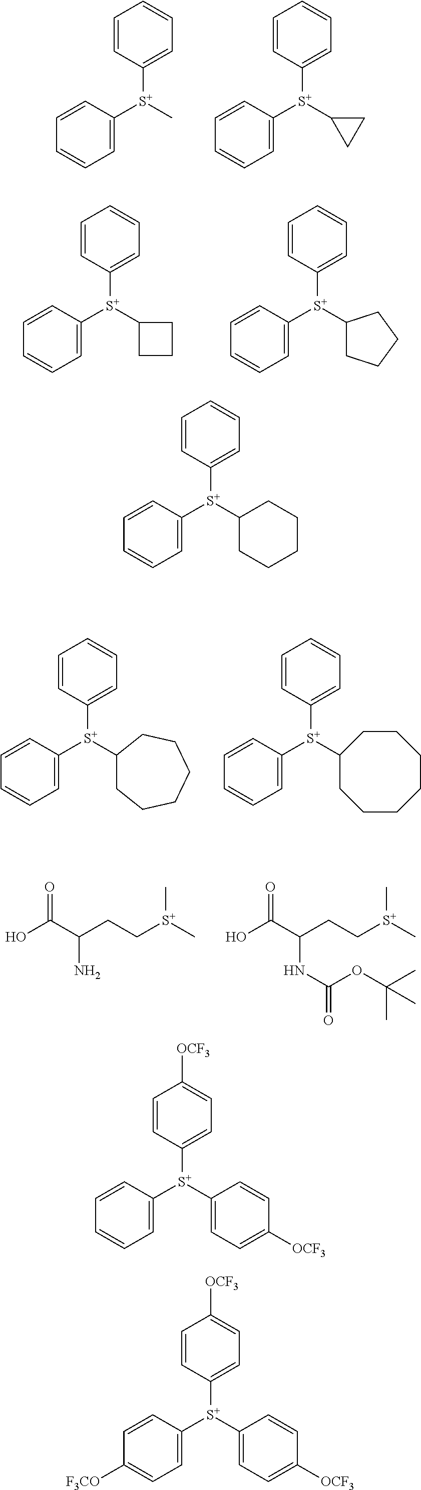

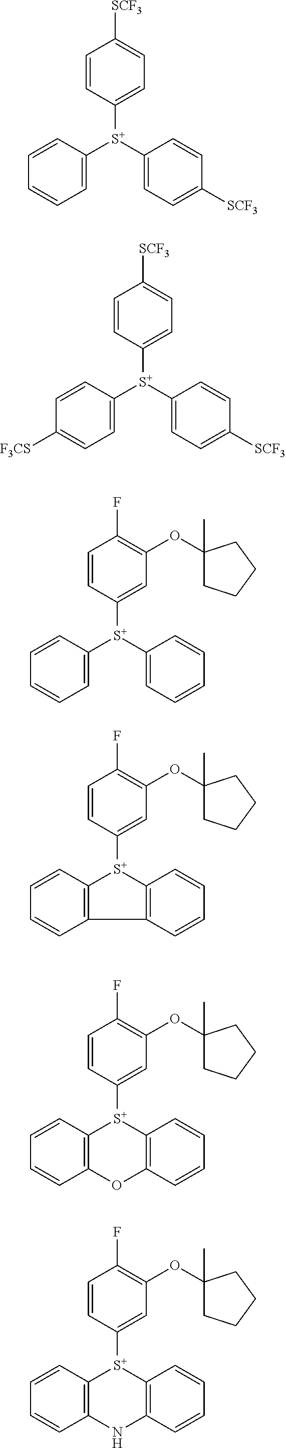

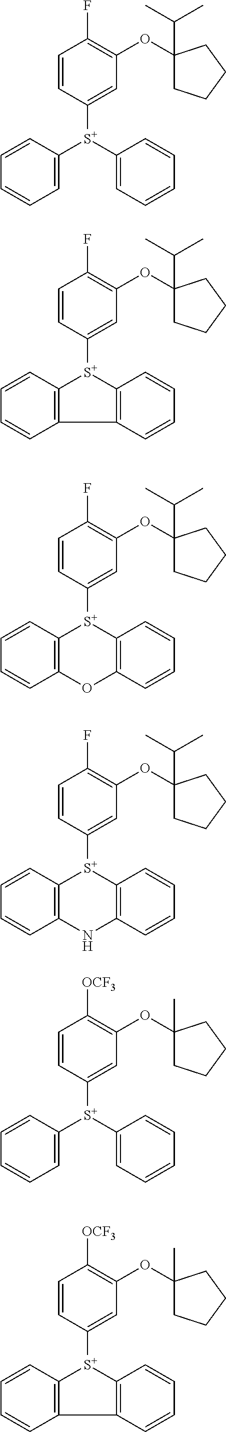 [SMILES: CC(C)C1(Oc2cc([S+](c3ccccc3)c3ccccc3)ccc2F)CCCC1 ; CC(C)C1(Oc2cc(-[s+]3c4ccccc4c4ccccc43)ccc2F)CCCC1 ; CC(C)C1(Oc2cc([S+]3c4ccccc4Oc4ccccc43)ccc2F)CCCC1 ; CC(C)C1(Oc2cc([S+]3c4ccccc4Nc4ccccc43)ccc2F)CCCC1 ; CC1(Oc2cc([S+](c3ccccc3)c3ccccc3)ccc2OC(F)(F)F)CCCC1 ; CC1(Oc2cc(-[s+]3c4ccccc4c4ccccc43)ccc2OC(F)(F)F)CCCC1]

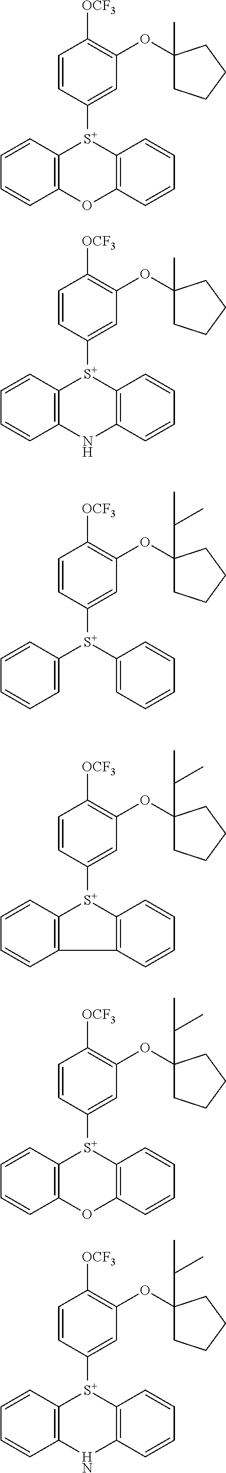 [SMILES: CC1(Oc2cc([S+]3c4ccccc4Oc4ccccc43)ccc2OC(F)(F)F)CCCC1 ; CC1(Oc2cc([S+]3c4ccccc4Nc4ccccc43)ccc2OC(F)(F)F)CCCC1 ; CC(C)C1(Oc2cc([S+](c3ccccc3)c3ccccc3)ccc2OC(F)(F)F)CCCC1 ; CC(C)C1(Oc2cc(-[s+]3c4ccccc4c4ccccc43)ccc2OC(F)(F)F)CCCC1 ; CC(C)C1(Oc2cc([S+]3c4ccccc4Oc4ccccc43)ccc2OC(F)(F)F)CCCC1 ; CC(C)C1(Oc2cc([S+]3c4ccccc4Nc4ccccc43)ccc2OC(F)(F)F)CCCC1]

Examples of the iodonium cation represented by formula (cation-2) are shown below, but not limited thereto.

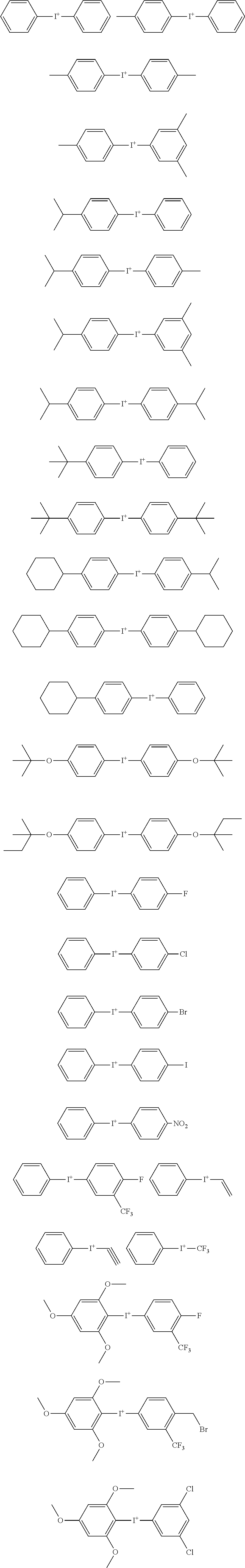

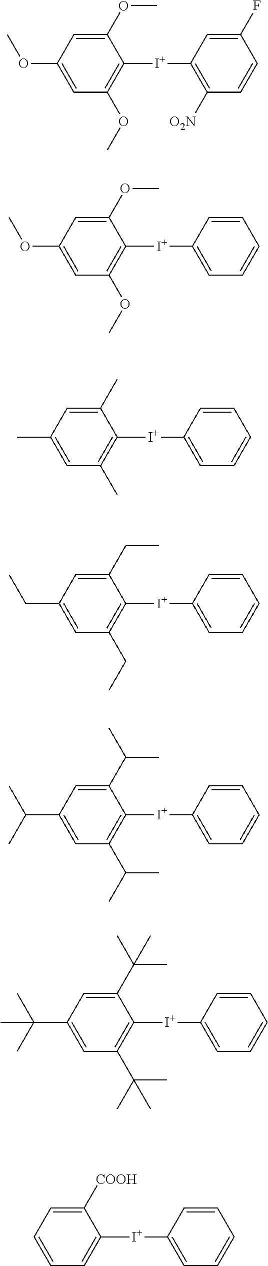

Specific examples of the inventive onium salt type monomer having formula (a) include arbitrary combinations of the anion with the cation.

The onium salt type monomer having formula (a) may be synthesized in the same manner as in synthesis of a sulfonium salt having a polymerizable anion in JP 5201363, but the synthesis method is not limited thereto.

### [Polymer]

The inventive polymer comprises repeat units derived from the onium salt type monomer having formula (a) (hereinafter, also referred to as repeat units (a)).

The inventive polymer is a polymer-bound photoacid generator that functions as a photoacid generator and also as a base polymer in the chemically amplified resist composition. A structural characteristic of the inventive polymer is that the polymer contains repeat units having an onium salt structure that is derived from the inventive onium salt type monomer, has a benzene or naphthalene structure directly bound to a backbone and contains a fluorosulfonate anion having an aromatic ring structure substituted with an iodine atom. Iodine atoms absorb an extremely large amount of EUV having a wavelength 13.5 nm, and generate secondary electrons during exposure, and the energy of the secondary electrons is transferred to the acid generator, thereby increasing the sensitivity. A polymerizable group having a styrene or vinylnaphthalene structure has higher rigidity over a polymerizable group such as a methacrylic acid ester, and improves the glass transition temperature (Tg) of the polymer. It is considered that the aromatic rings in the base polymer or between the base polymers interact with each other (exhibits a π-π stacking effect) to regularly arrange the base polymers, and even in fine pattern formation, resistance to pattern collapse is exhibited against the developer. In an etching step after fine pattern formation, excellent etch resistance is also exhibited because the aromatic ring is directly bound to the backbone. In the aromatic ring substituted with an iodine atom, the iodine atom is preferably bound in an ortho position with respect to a carbon atom to which the polymerizable group is attached. This is expected to suppress the rotation of the bond axis between the aromatic ring of the polymerizable group is attached and the aromatic ring substituted with the iodine atom. This, in turn, suppresses excessive diffusion of generated acid, improves LWR of line patterns and CDU of hole patterns, and enables pattern formation in which pattern collapse hardly occurs. Thus, the inventive polymer is particularly suitable as a material for chemically amplified positive resist compositions.

The polymer may further comprise repeat units having the following formula (b1) (hereinafter, also referred to as repeat units (b1)) or repeat units having the following formula (b2) (hereinafter, also referred to as repeat units (b2)).

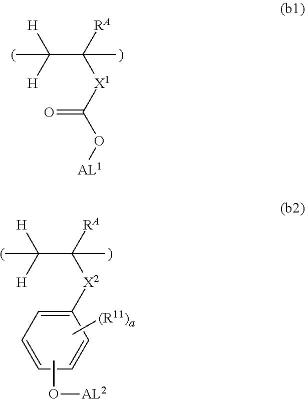

In formulae (b1) and (b2), R^{A} is each independently a hydrogen atom, a fluorine atom, a methyl group, or a trifluoromethyl group.

In formula (b1), X¹ is a single bond, a phenylene group, a naphthylene group, *—C(═O)—O—X¹¹— or *—C(═O)—NH—X¹¹—, and the phenylene group or the naphthylene group may be substituted with a C₁-C₁₀ alkoxy group which may contain a fluorine atom, or a halogen atom. X¹¹ is a C₁-C₁₀ saturated hydrocarbylene group, a phenylene group, or a naphthylene group, and the saturated hydrocarbylene group may contain a hydroxy group, an ether bond, an ester bond or a lactone ring, the asterisk (*) designates a point of attachment to the carbon atom in the backbone,

In the formula (b2), X² is a single bond, *—C(═O)—O— or *—C(═O)—NH—. The asterisk (*) designates a point of attachment to the carbon atom in the backbone, R¹¹ is a halogen atom, a cyano group, a C1-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, and a is an integer of 0 to 4, preferably 0 or 1.

In formulae (b1) and (b2), AL¹ and AL² are each independently an acid labile group. Examples of the acid labile group are as shown in JP-A 2013-80033 and JP-A 2013-83821, but the acid labile group is not limited thereto.

Typical of the acid labile group are groups having the following formulae (AL-1) to (AL-3).

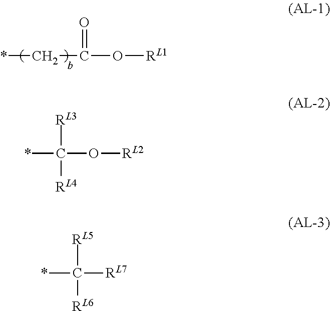

Herein the asterisk (*) designates a point of attachment.

In formulae (AL-1) and (AL-2), R^{L1} and R^{L2} are each independently a C₁-C₄₀ hydrocarbyl group, and may contain a heteroatom such as an oxygen atom, a sulfur atom, a nitrogen atom, a fluorine atom or an iodine atom. The hydrocarbyl group may be straight, branched or cyclic. The hydrocarbyl group is preferably a C₁-C₂₀ hydrocarbyl group.

In formula (AL-1), b is an integer of 0 to 10, preferably an integer of 1 to 5.

In formulae (AL-2), R^{L3} and R^{L4} are each independently a hydrogen atom, a C₁-C₂₀ hydrocarbyl group, and may contain a heteroatom such as an oxygen atom, a sulfur atom, a nitrogen atom, a fluorine atom or an iodine atom. The hydrocarbyl group may be straight, branched or cyclic. Any two of R^{L2}, R^{L3} and R^{L4} may bond together to form a C₃-C₂₀ ring with a carbon atom to which they are attached, or a carbon atom or an oxygen atom. The ring is preferably a ring containing 4 to 16 carbon atoms, and particularly preferably an alicyclic ring.

In formula (AL-3), R^{L5}, R^{L6} and R^{L7} are each independently a C₁-C₂₀ hydrocarbyl group, and may contain a heteroatom such as an oxygen atom, a sulfur atom, a nitrogen atom, a fluorine atom or an iodine atom. The hydrocarbyl group may be straight, branched or cyclic. Any two of R^{L5}, R^{L6} and R^{L7} may bond together to form a C₃-C₂₀ ring with a carbon atom to which they are attached. The ring is preferably a ring containing 4 to 16 carbon atoms, and particularly preferably an alicyclic ring.

Examples of repeat unit b1 are shown below, but not limited thereto. Herein, R^{A} and AL¹ are as defined above.

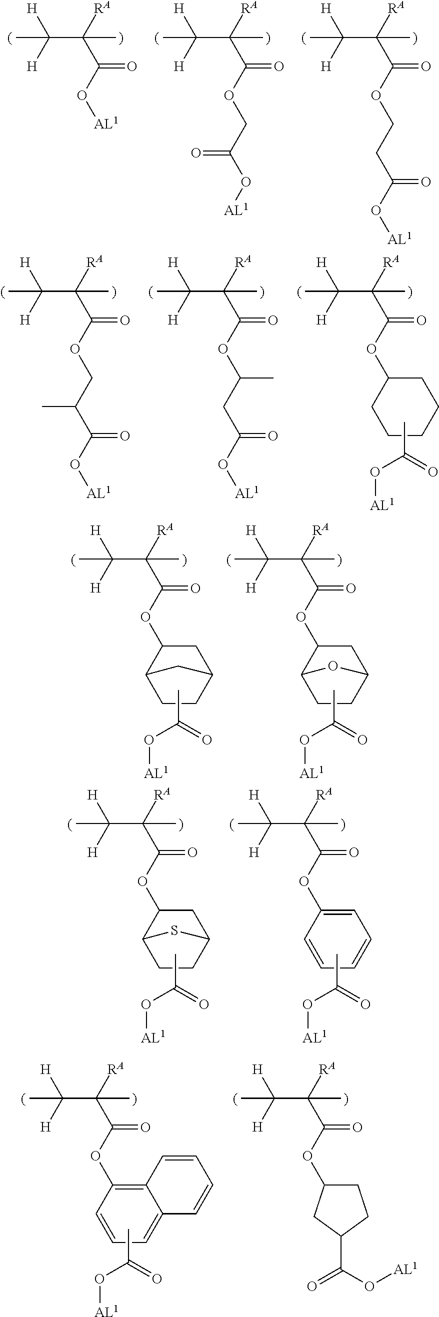

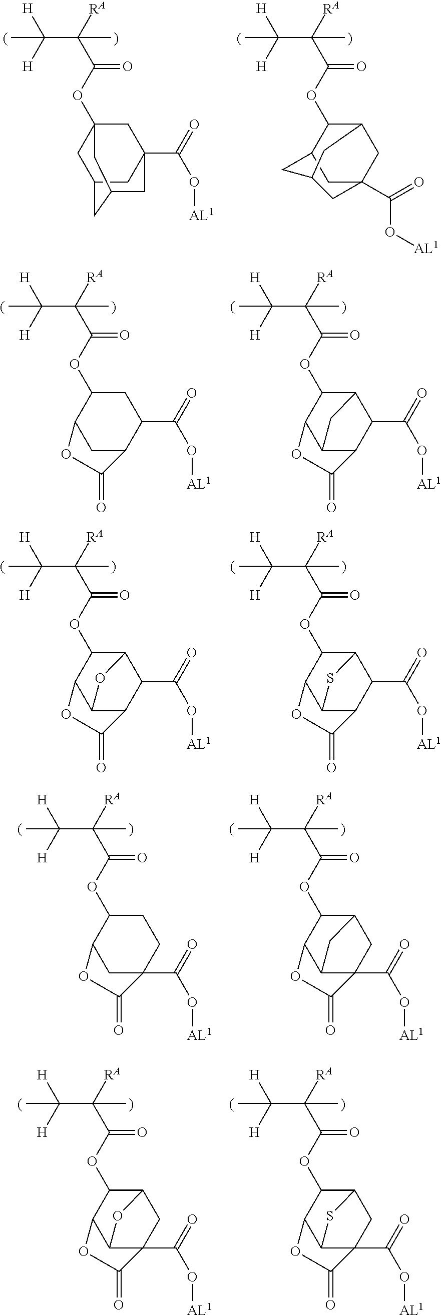

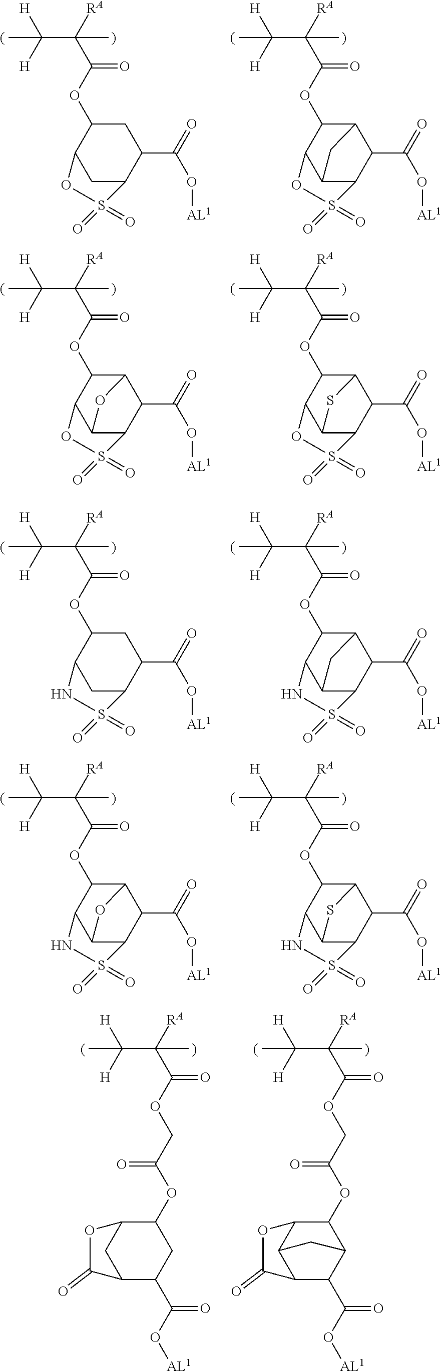

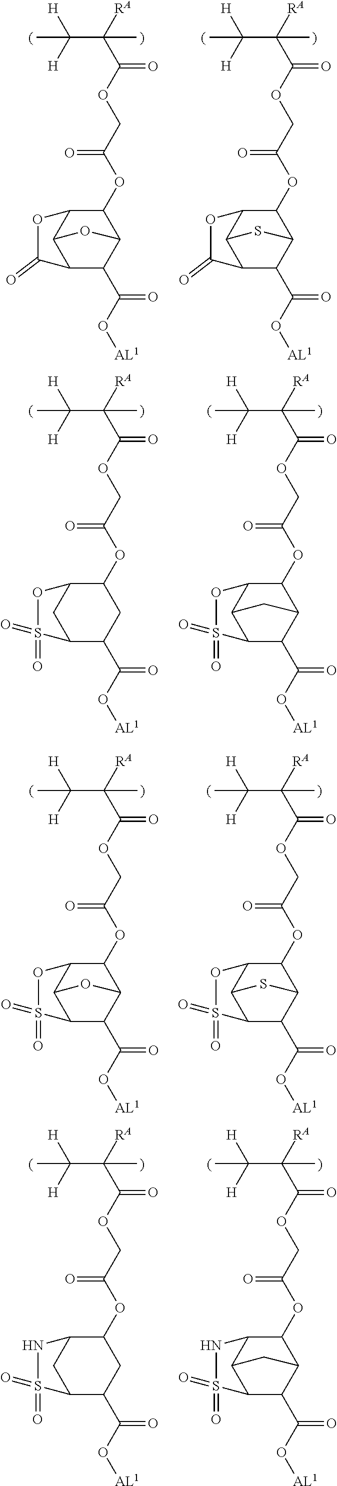

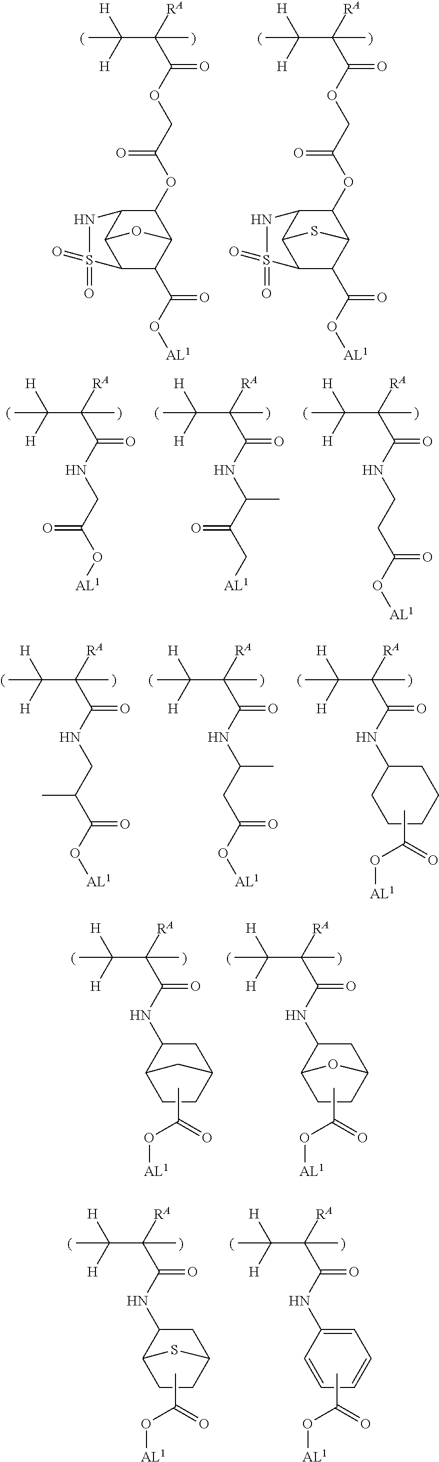

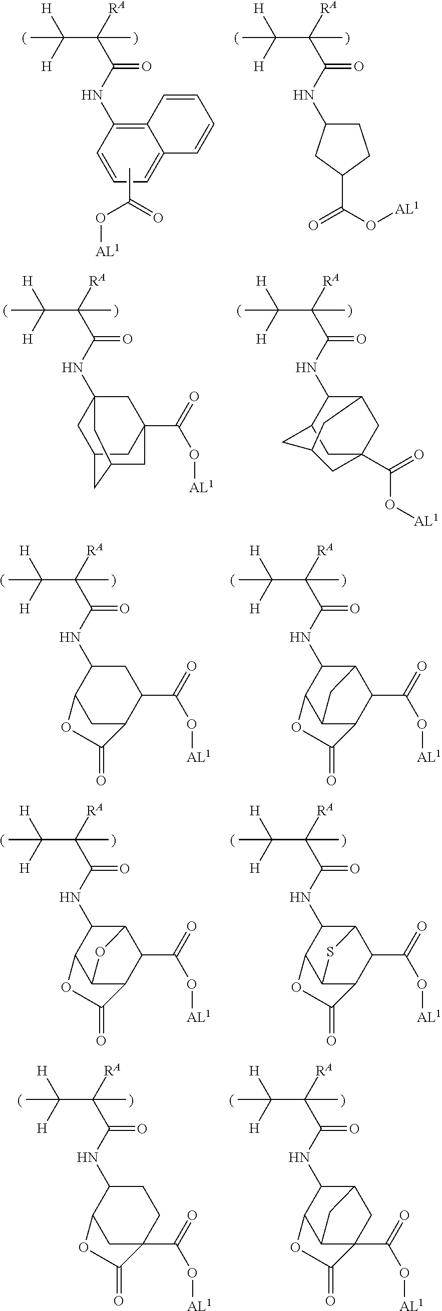

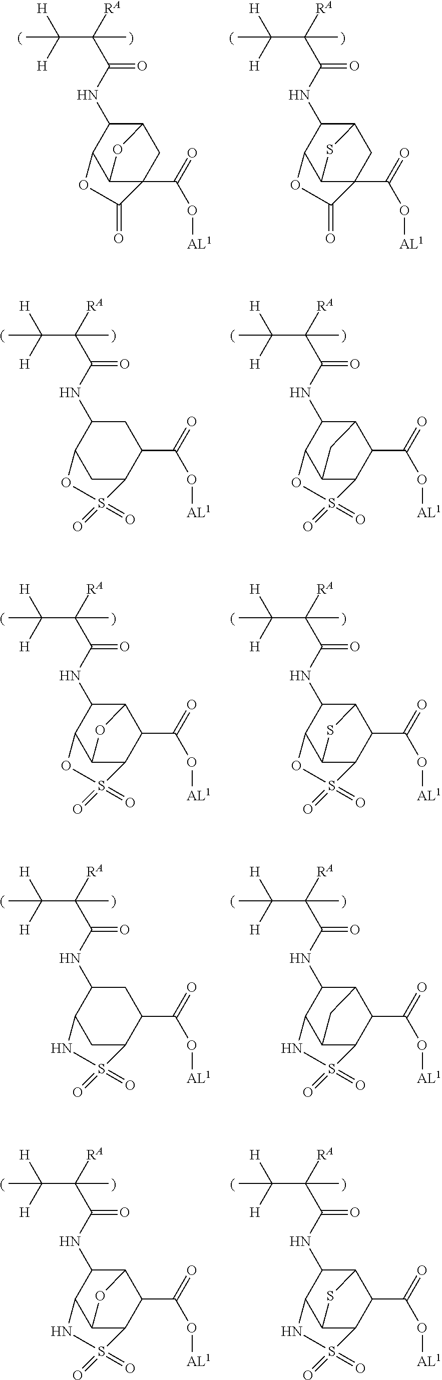

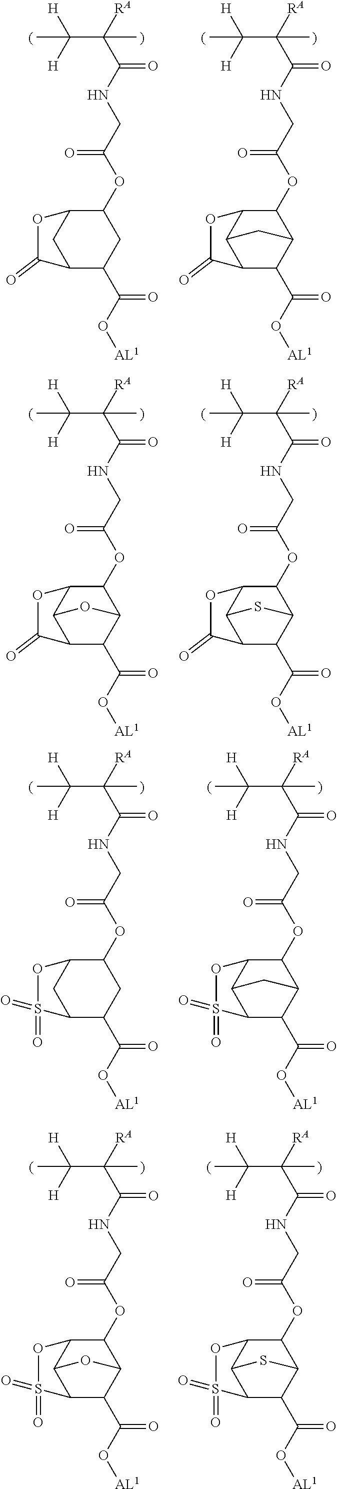

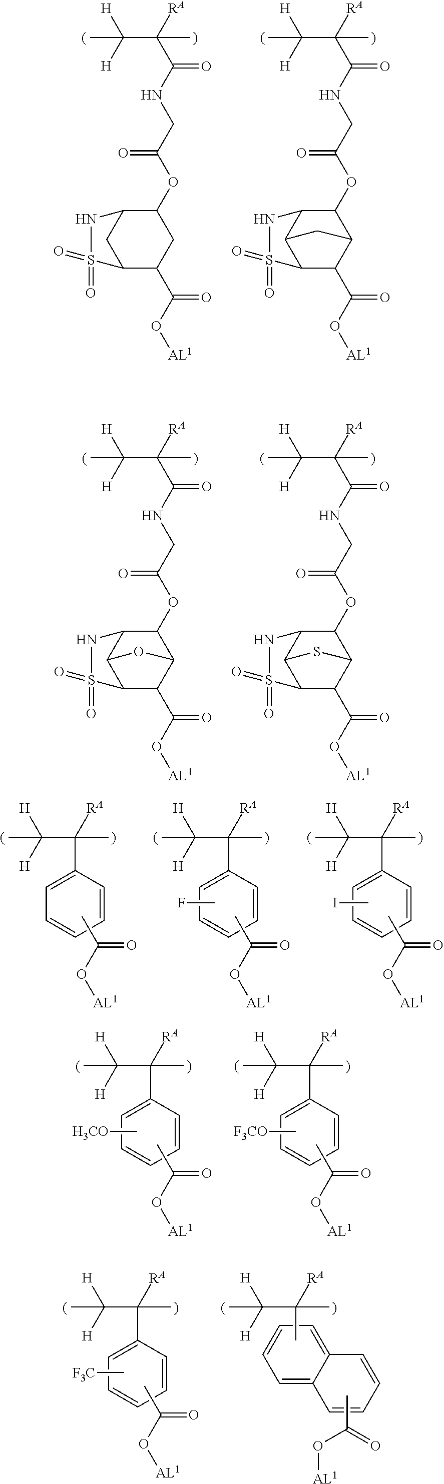

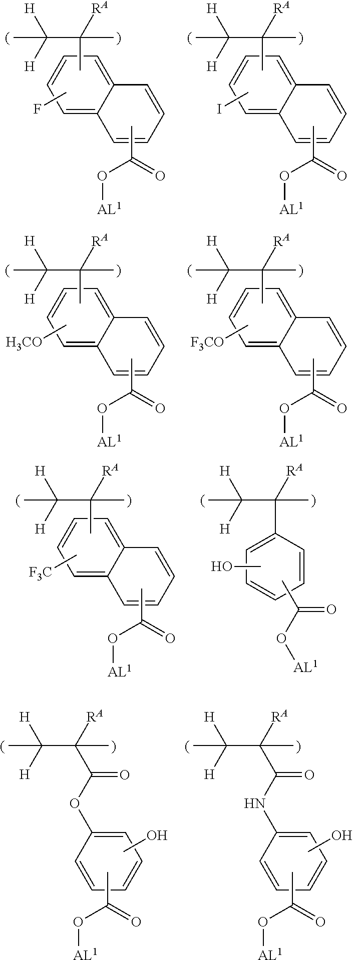

Examples of repeat unit b2 are shown below, but not limited thereto. Herein, R^{A} and AL² are as defined above.

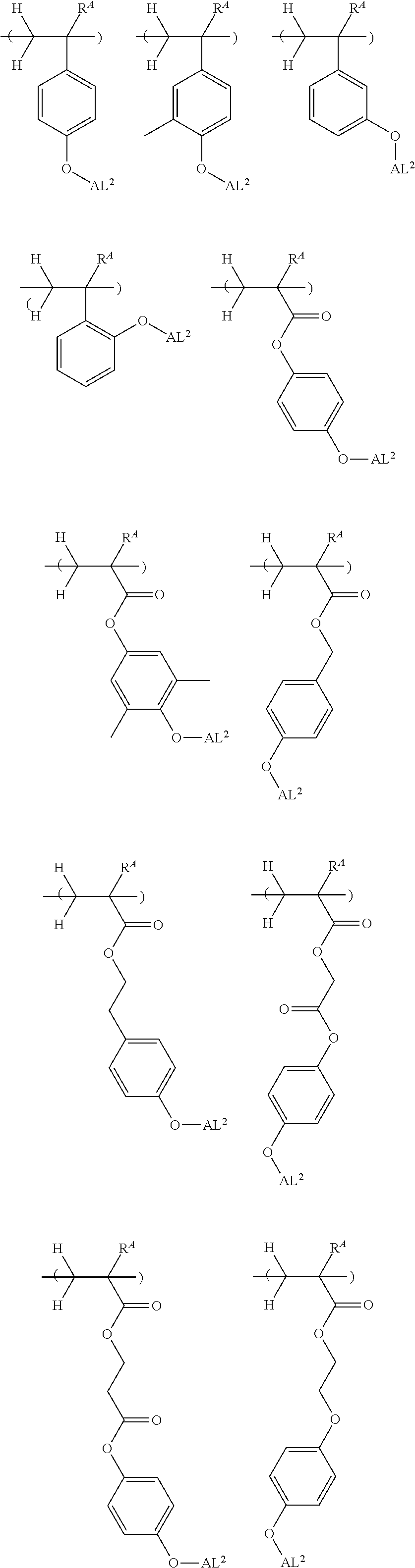

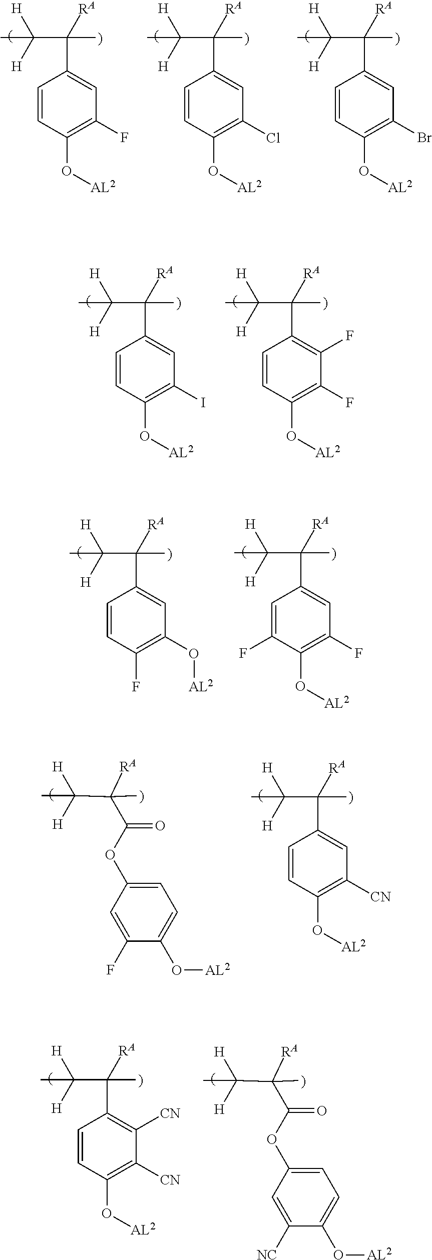

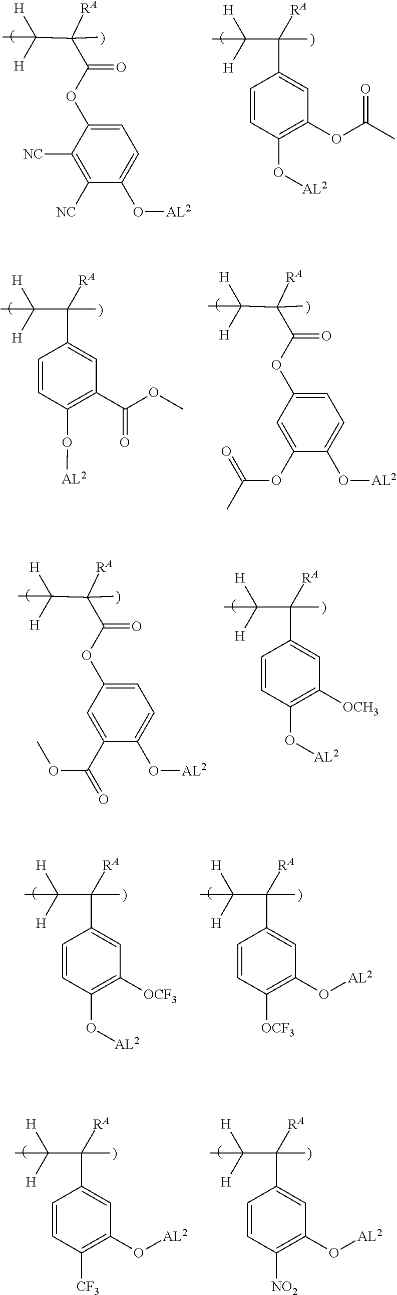

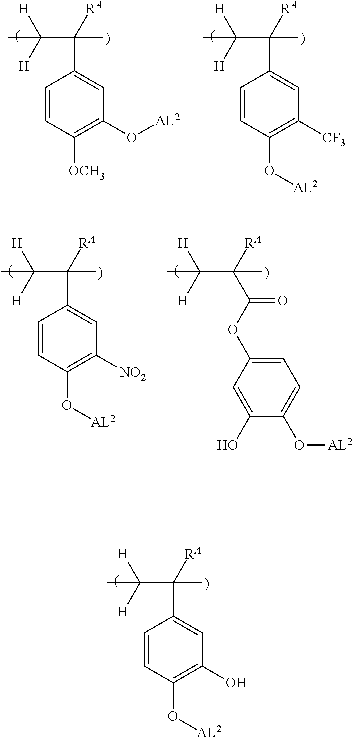

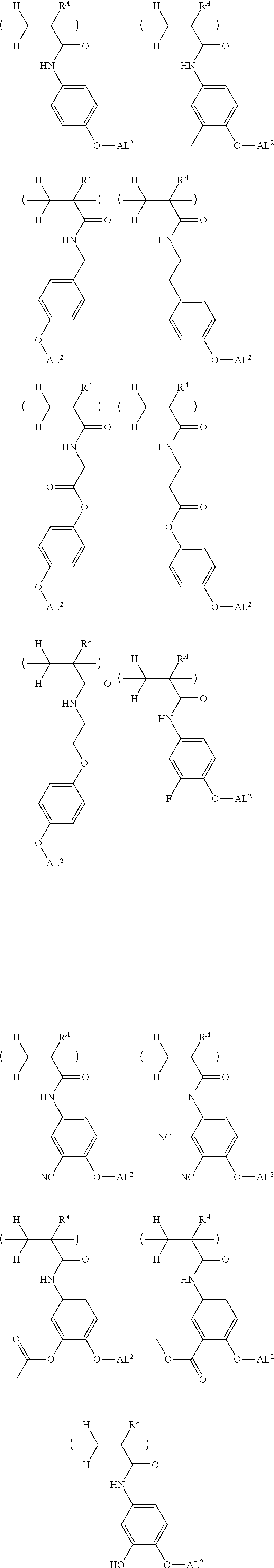

Preferably, the base polymer further comprises repeat units having the following formula (c1) (hereinafter, also referred to as repeat units (c)).

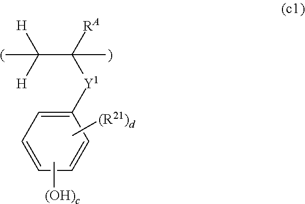

In formula (c1), R^{A} is a hydrogen atom, a fluorine atom, a methyl group, or a trifluoromethyl group. Y¹ is a single bond, *—C(═O)—O— or *—C(═O)—NH—, the asterisk (*) designates a point of attachment to the carbon atom in the backbone, R²¹ is a halogen atom, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, c is an integer of 1 to 4, and d is an integer of 0 to 3, provided that the sum of c+d is 1 or more and 5 or less.

Examples of repeat unit c are shown below, but not limited thereto. Herein, R^{A} is as defined above.

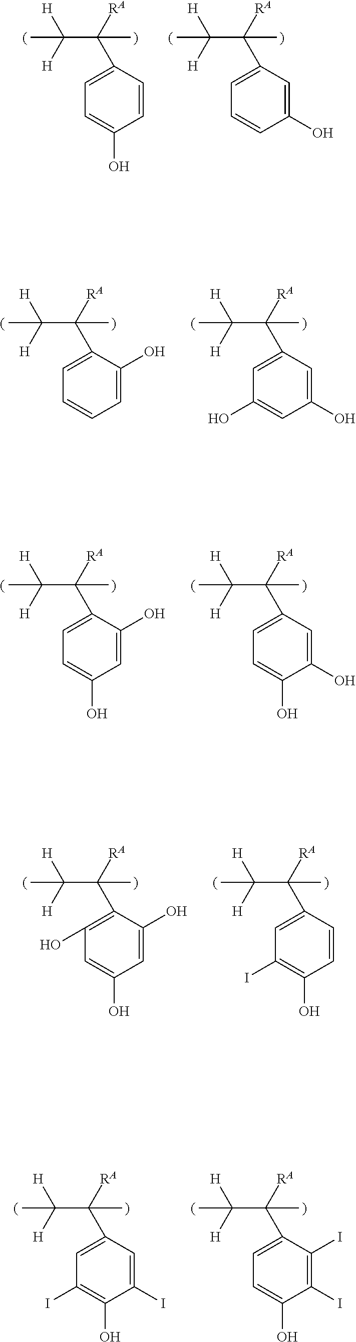

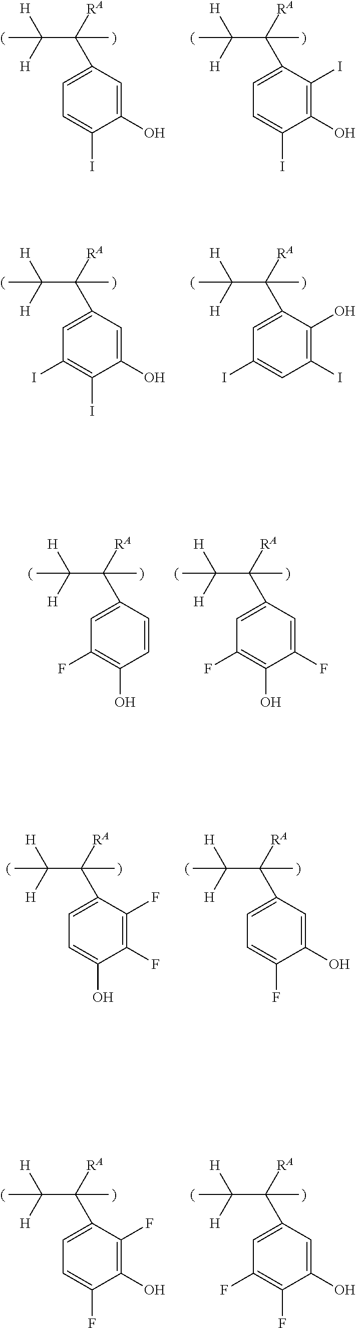

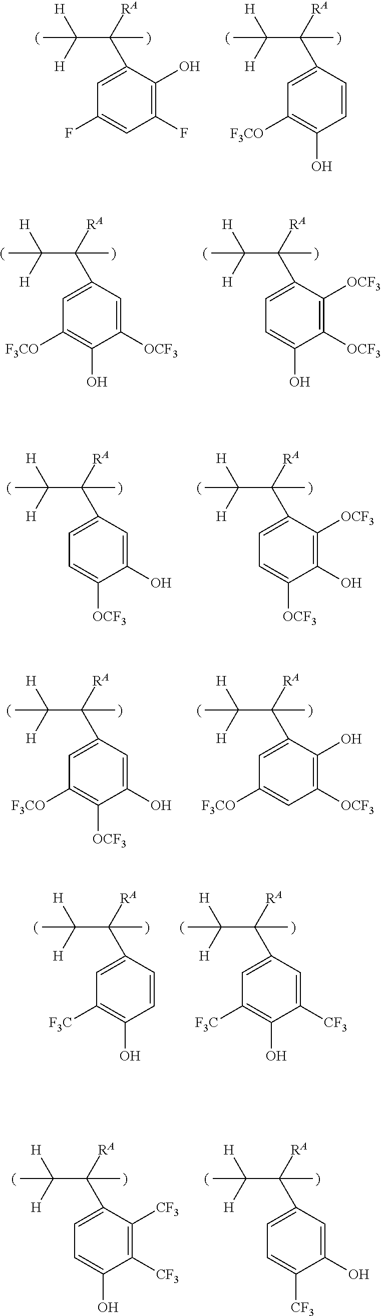

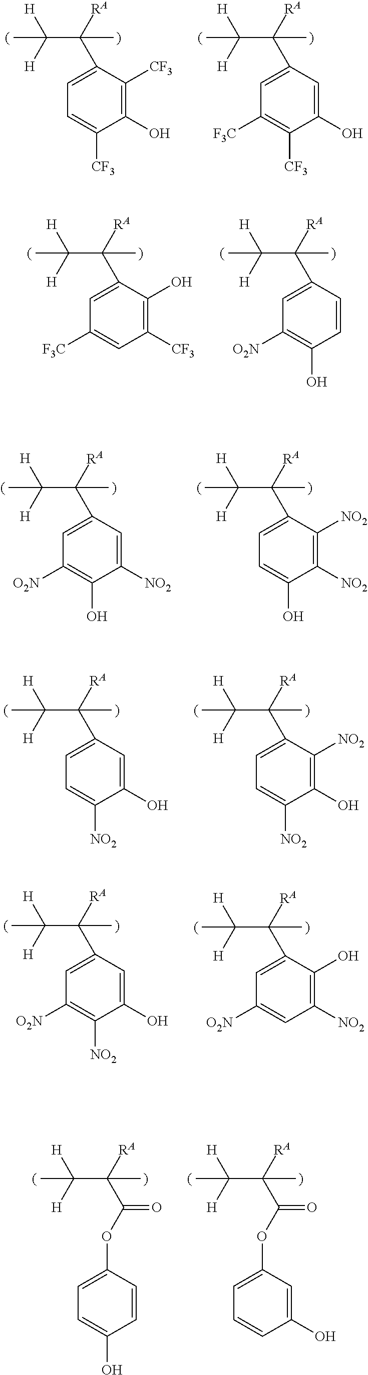

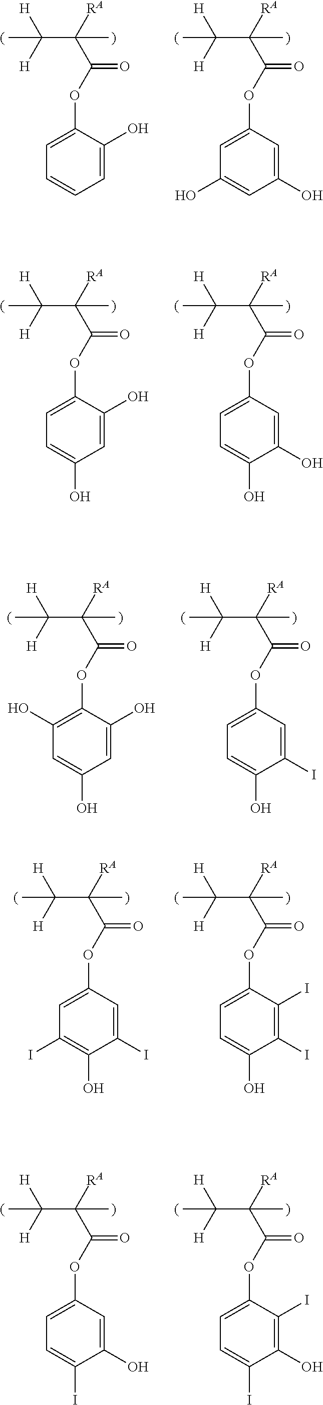

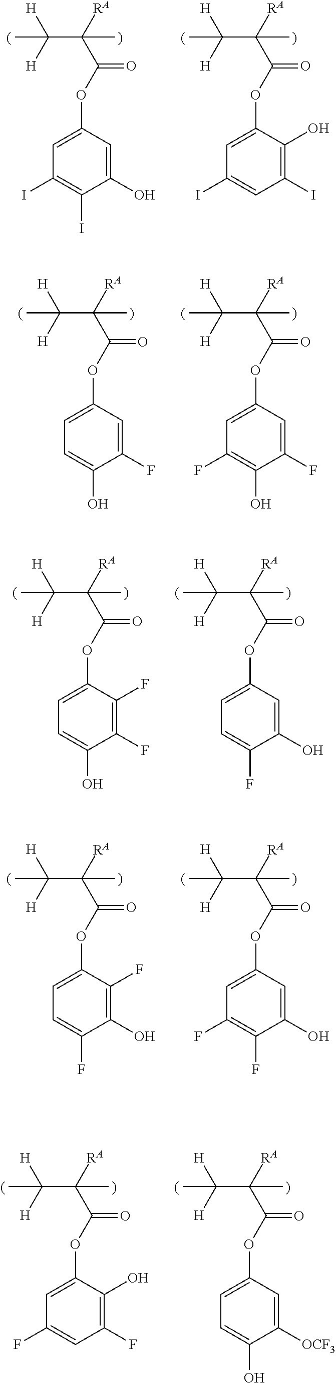

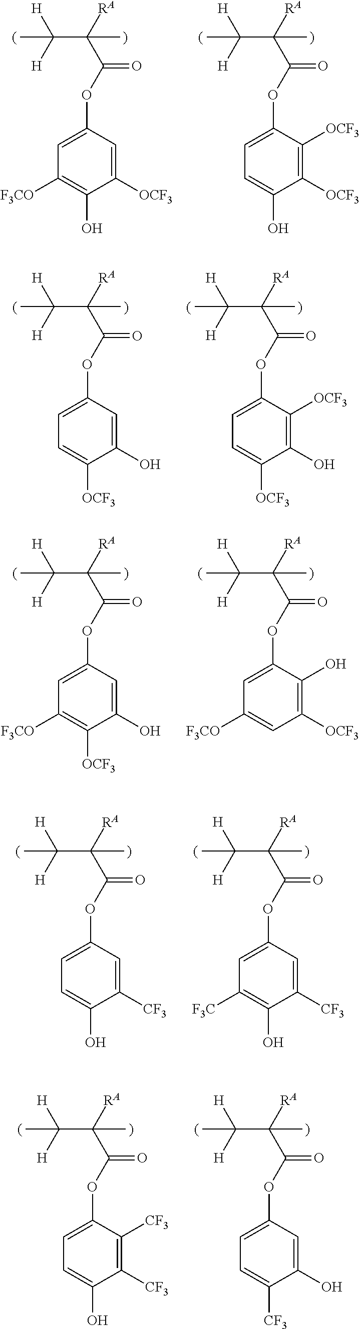

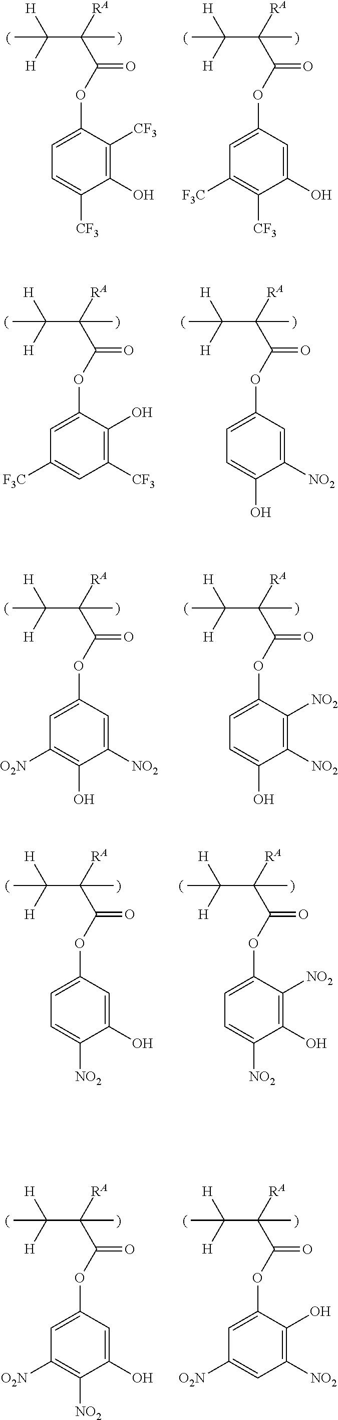

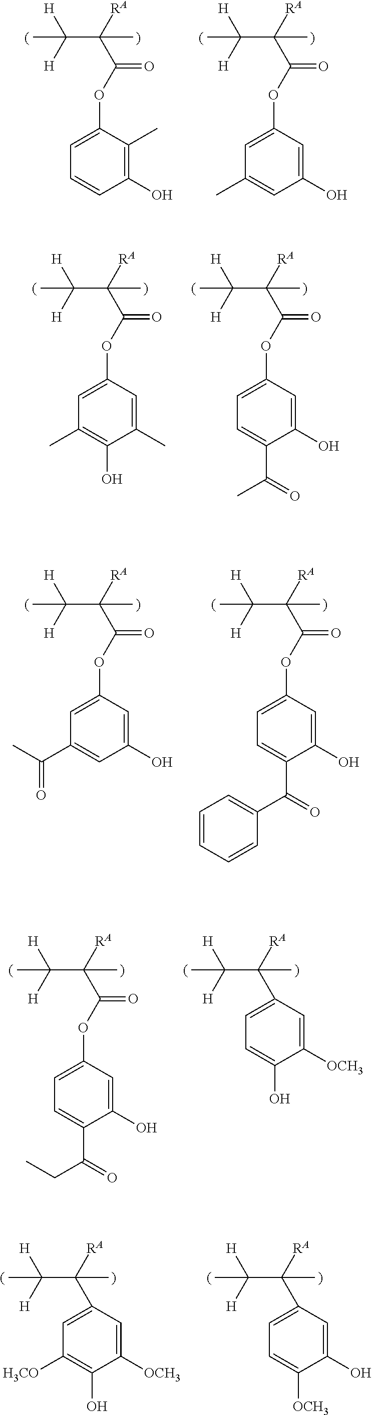

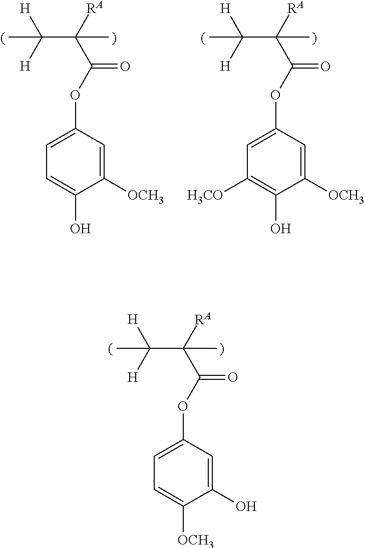

Preferably, the base polymer further comprises repeat units having the following formula (d1) (hereinafter, also referred to as repeat units (d)).

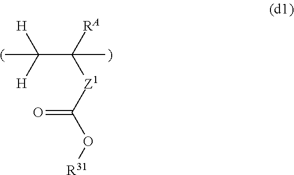

In formula (d1), R^{A} is a hydrogen atom, a fluorine atom, a methyl group, or a trifluoromethyl group. Z¹ is a single bond, a phenylene group, a naphthylene group, *—C(═O)—O—Z¹¹— or *—C(═O)—NH—Z¹¹—, or the phenylene group or the naphthylene group may be substituted with a C₁-C₁₀ alkoxy group which may contain a fluorine atom, or a halogen atom, the asterisk (*) designates a point of attachment to the carbon atom in the backbone, Z¹¹ is a C₁-C₁₀ saturated hydrocarbylene group, a phenylene group, or a naphthylene group, and the saturated hydrocarbylene group may contain a hydroxy group, an ether bond, an ester bond or a lactone ring, and R³¹ is a hydrogen atom, or a C₁-C₂₀ group containing at least one structure selected from a hydroxy group other than a phenolic hydroxy group, a cyano group, a carbonyl group, a carboxy group, an ether bond, an ester bond, a sulfonic ester bond, a carbonate bond, a lactone ring, a sultone ring, and a carboxylic anhydride (—C(═O)—O—C(═O)—).

Examples of repeat unit d are shown below, but not limited thereto. Herein, R^{A} is as defined above.

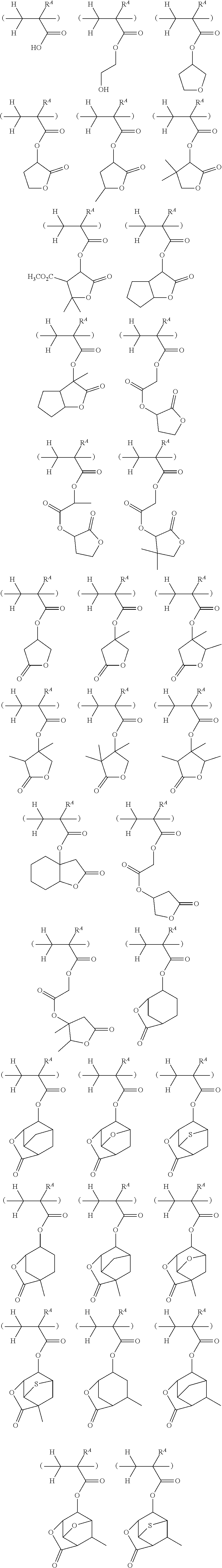

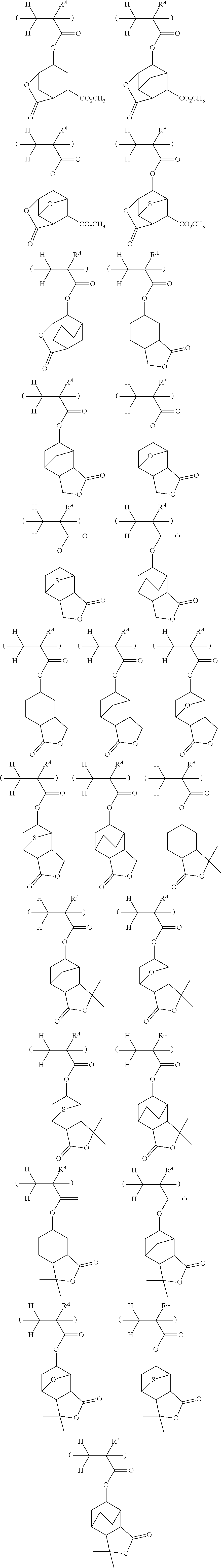

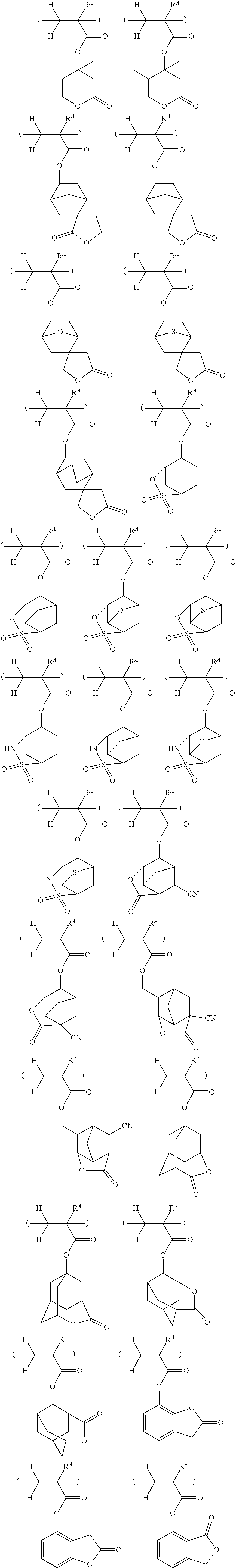

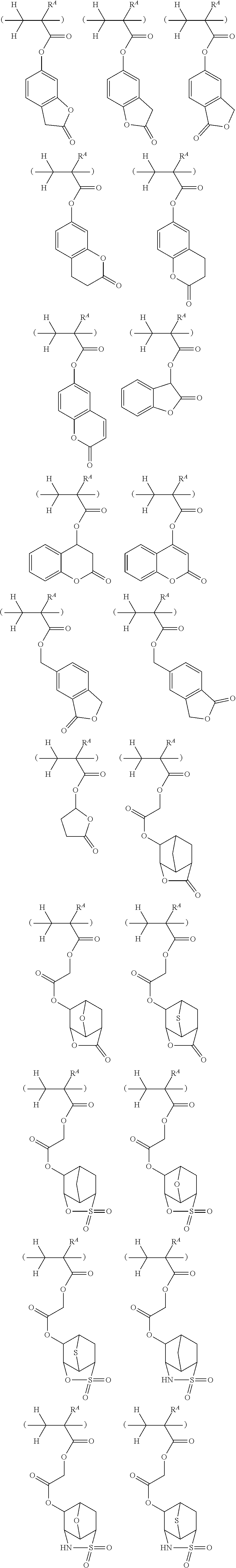

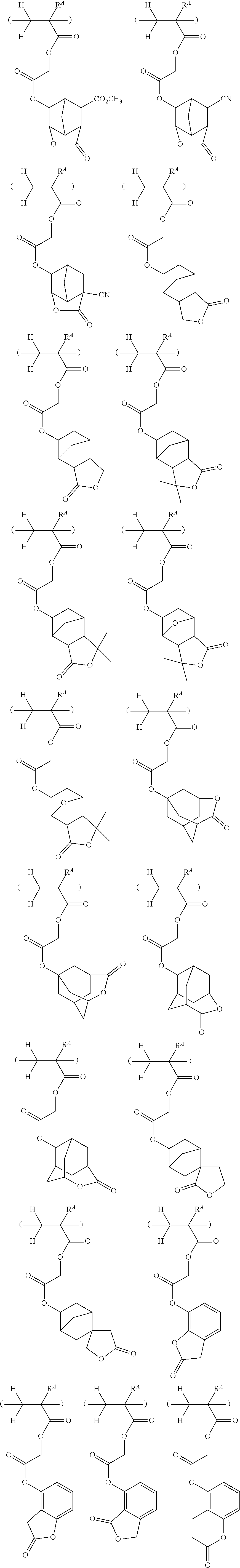

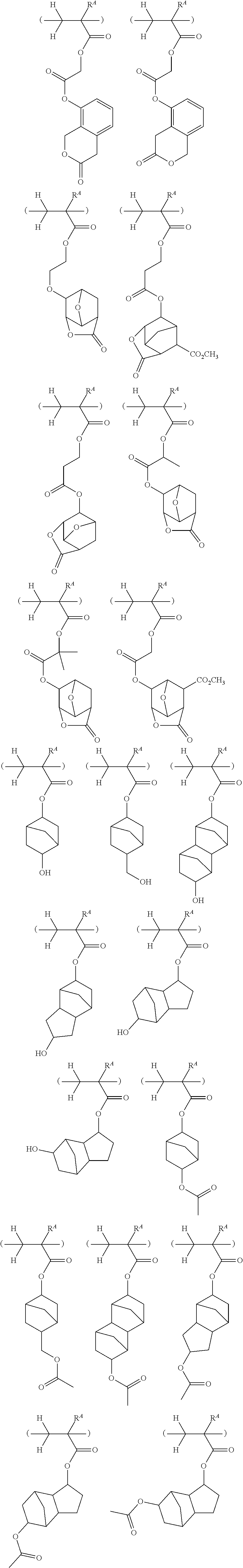

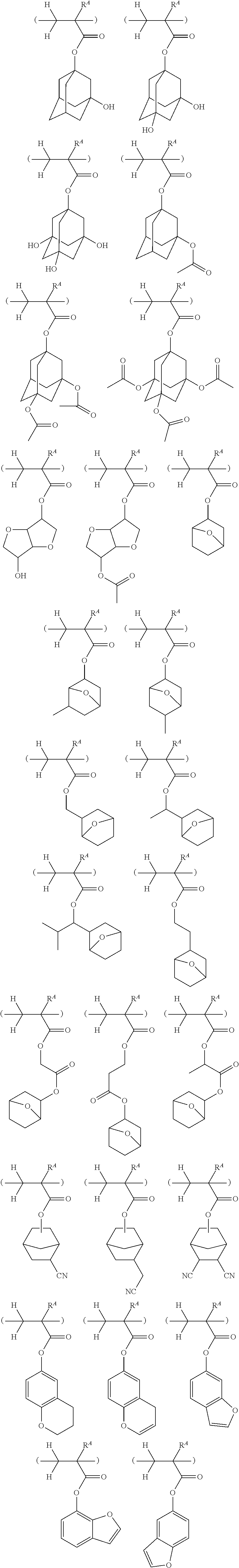

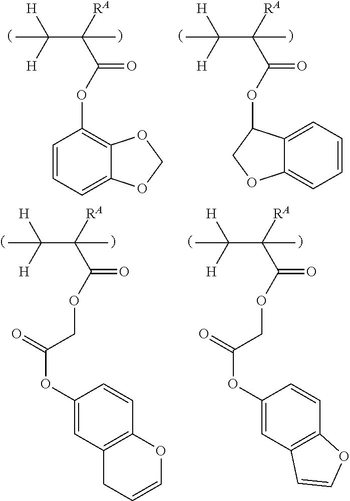

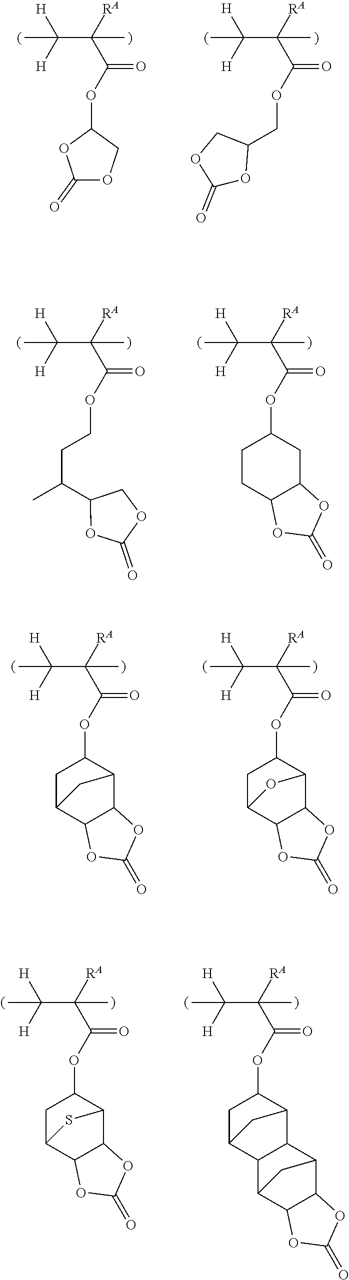

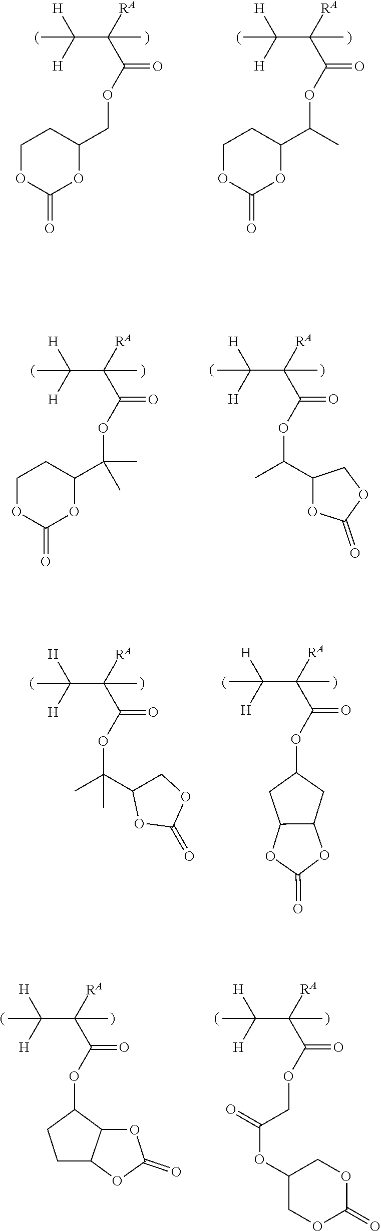

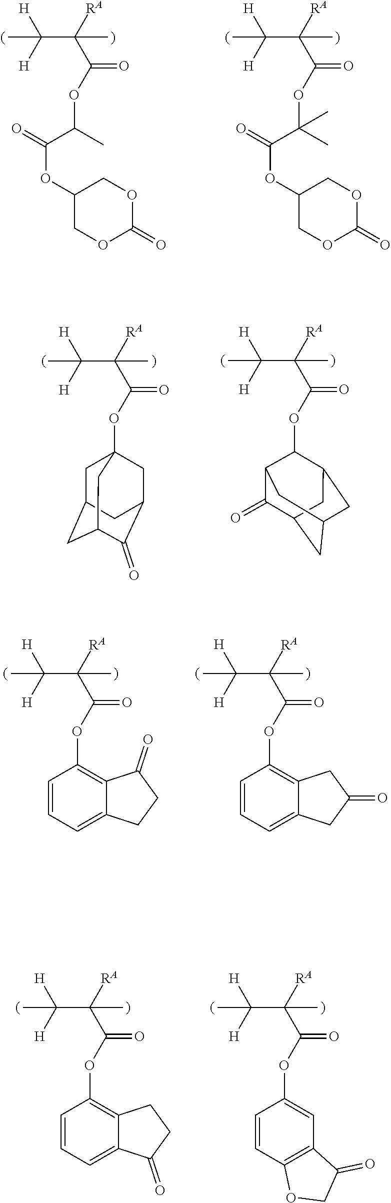

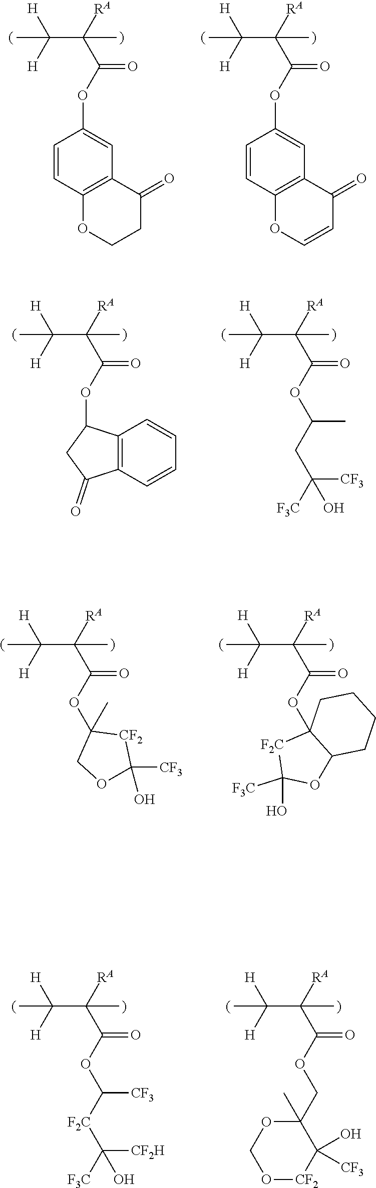

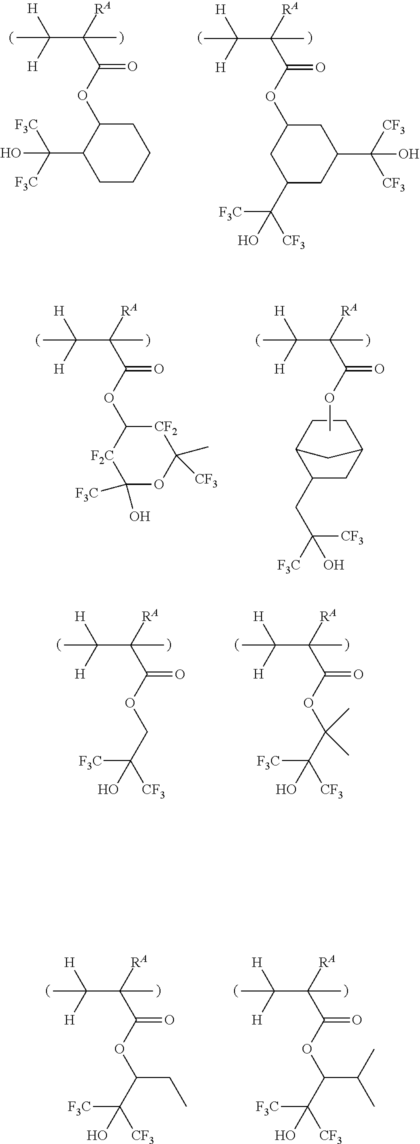

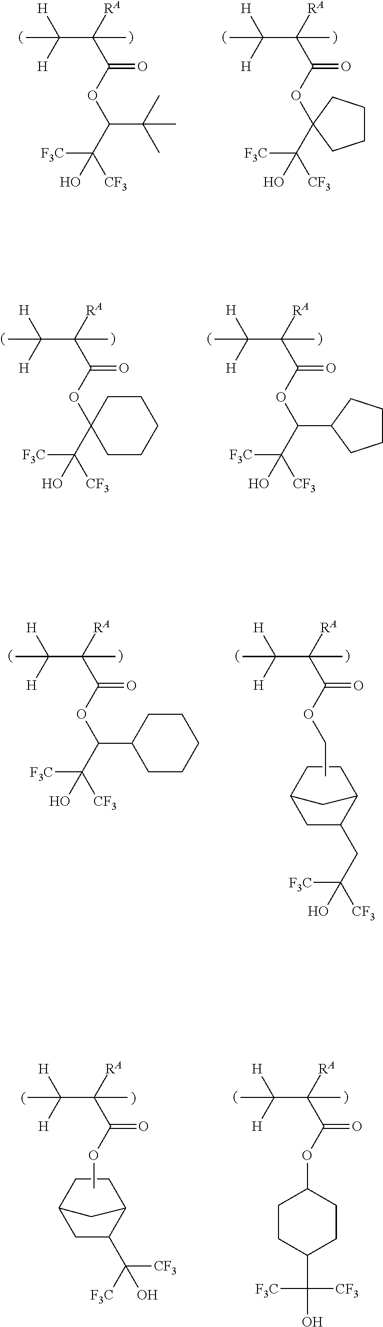

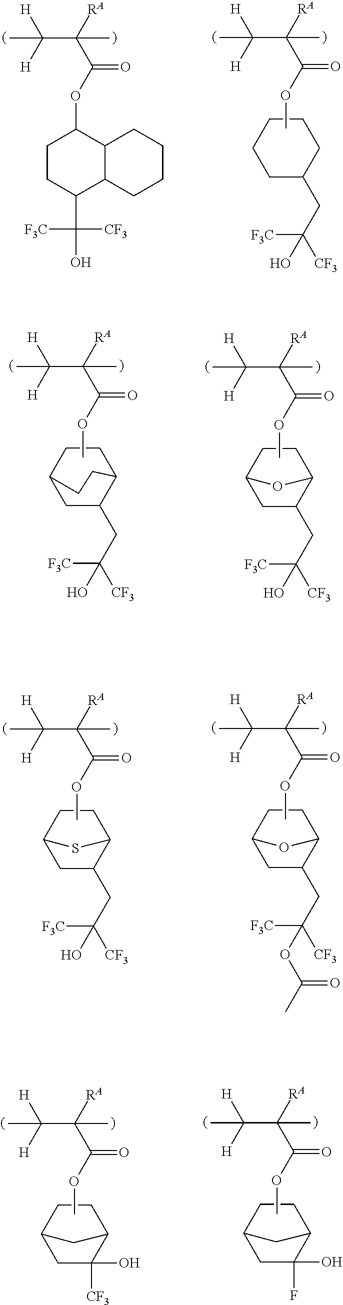

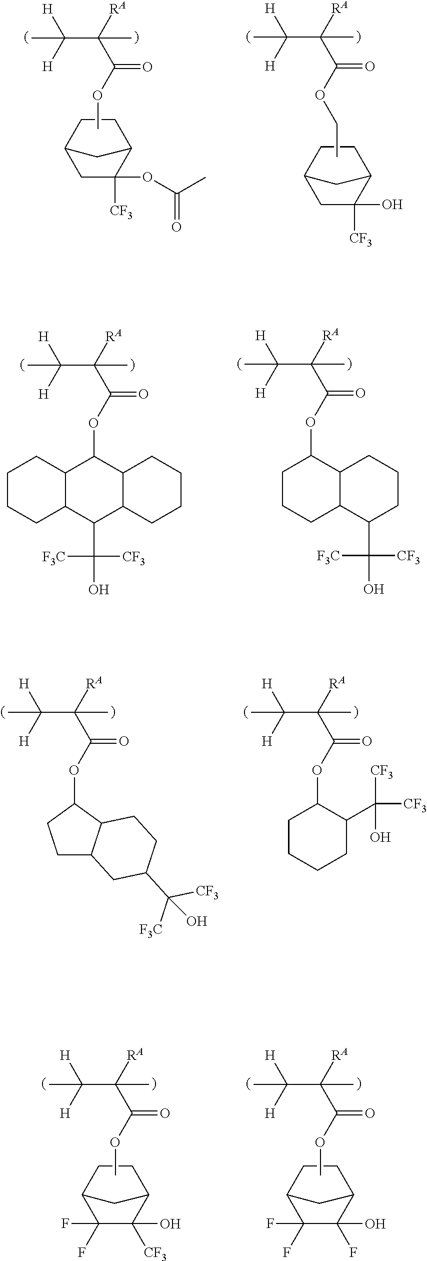

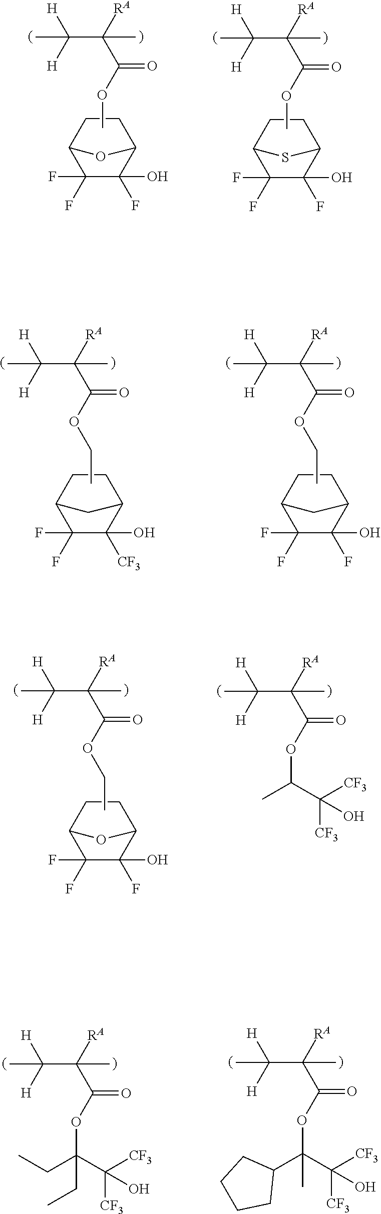

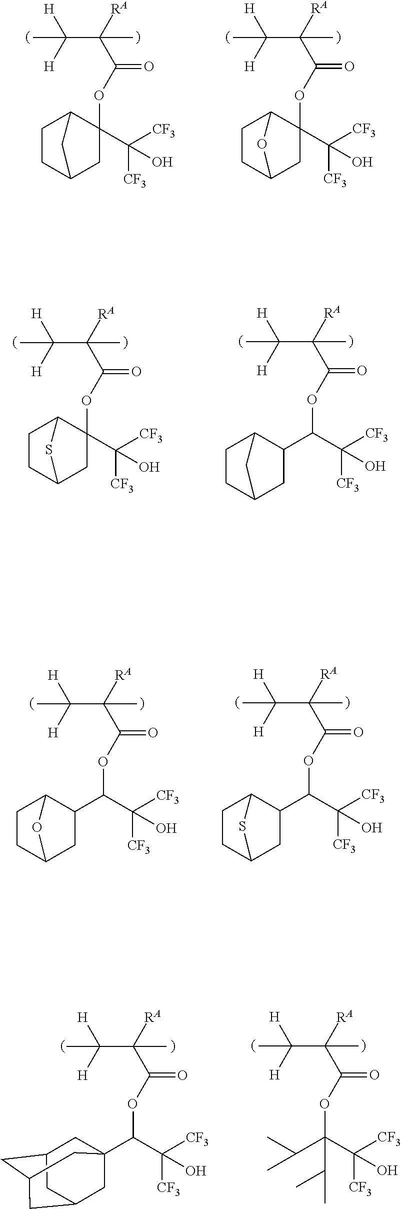

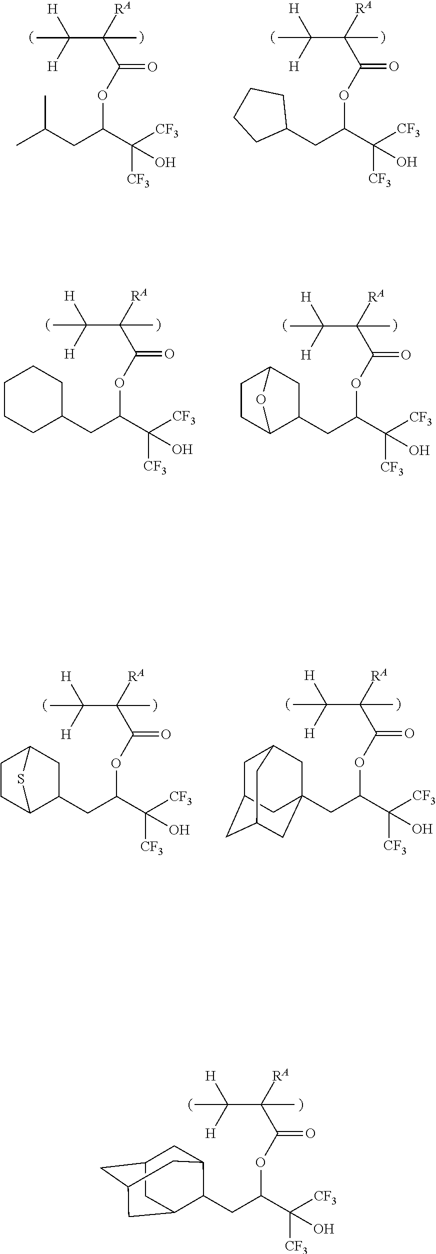

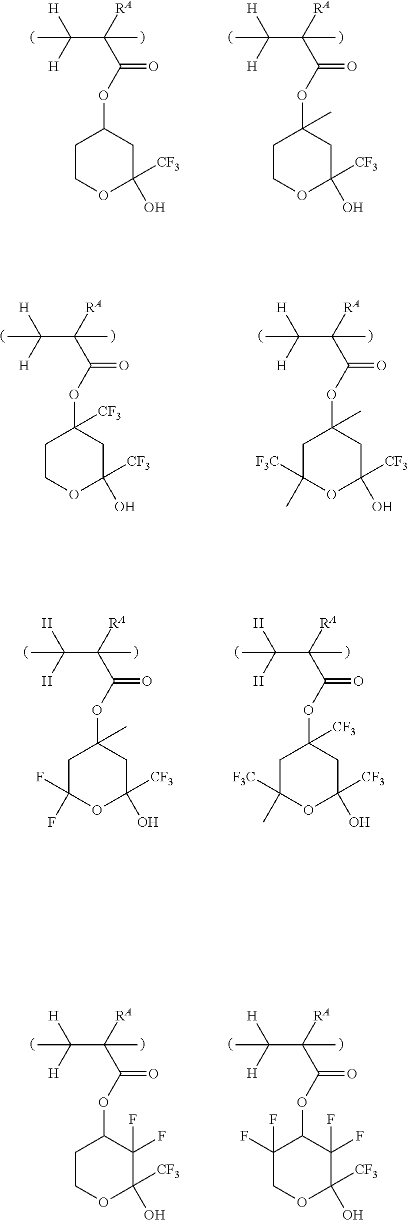

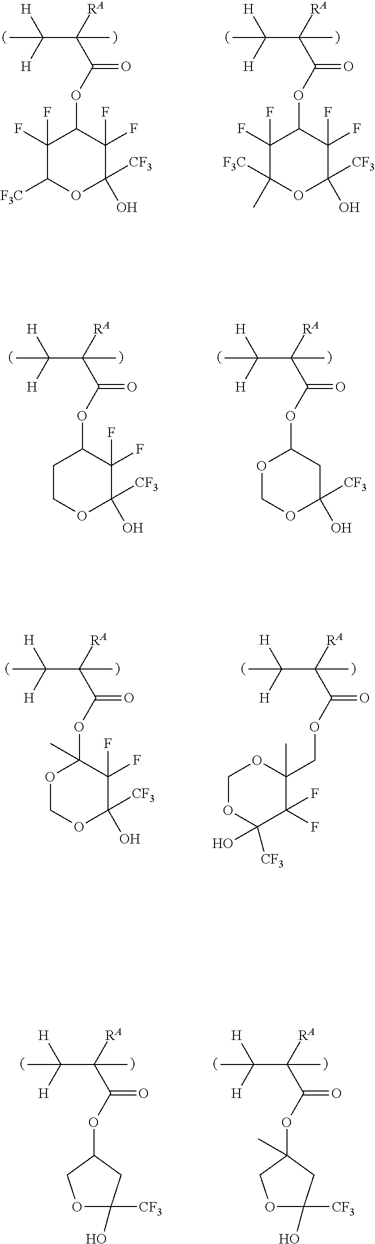

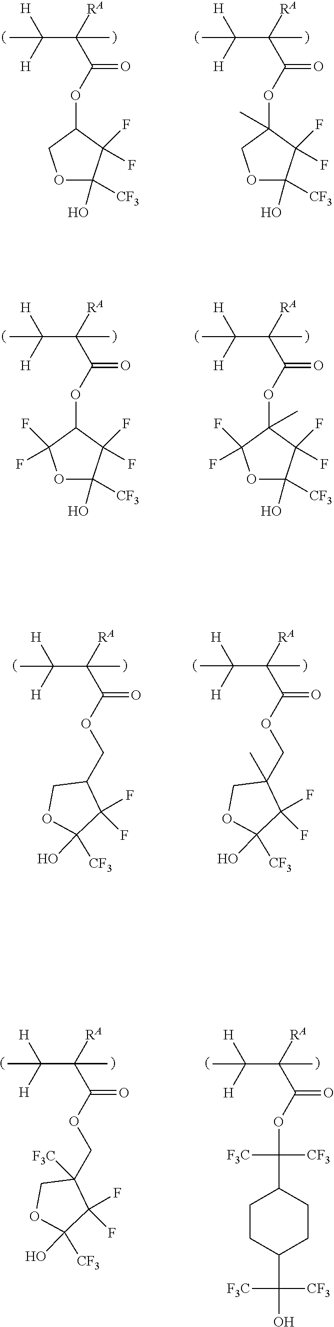

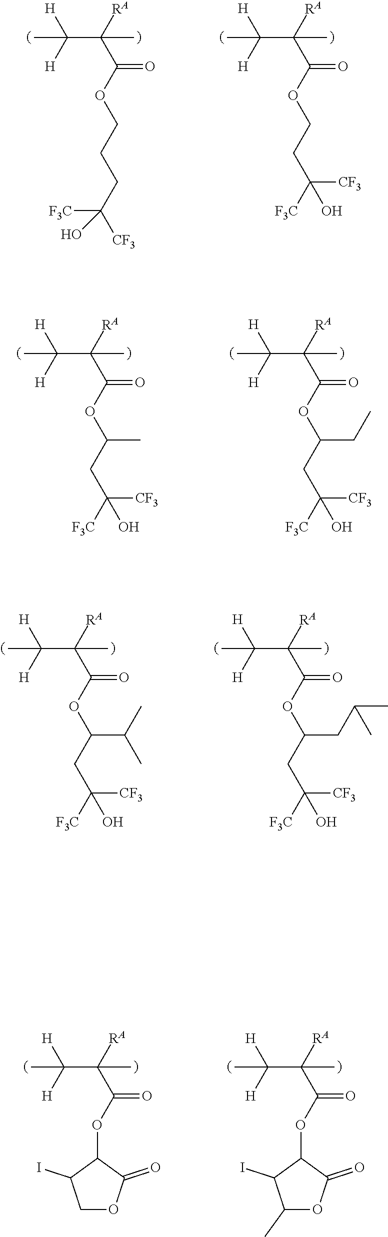

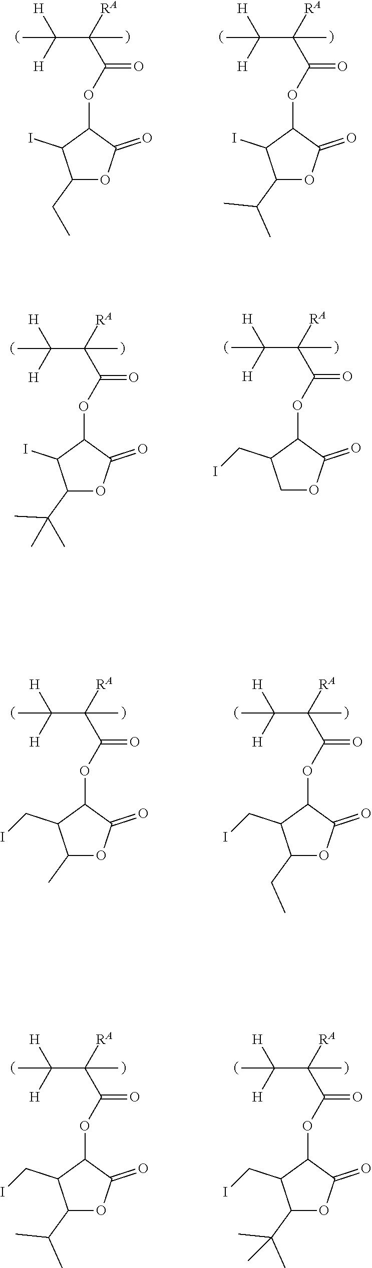

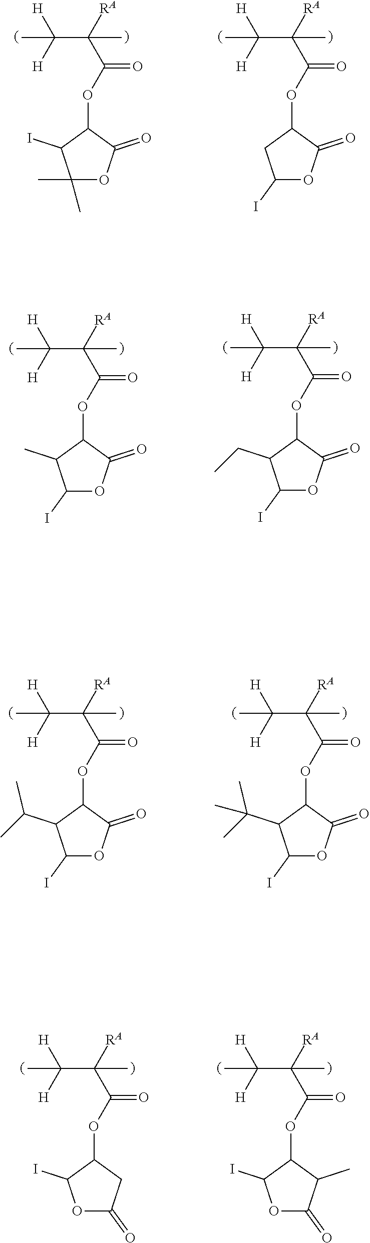

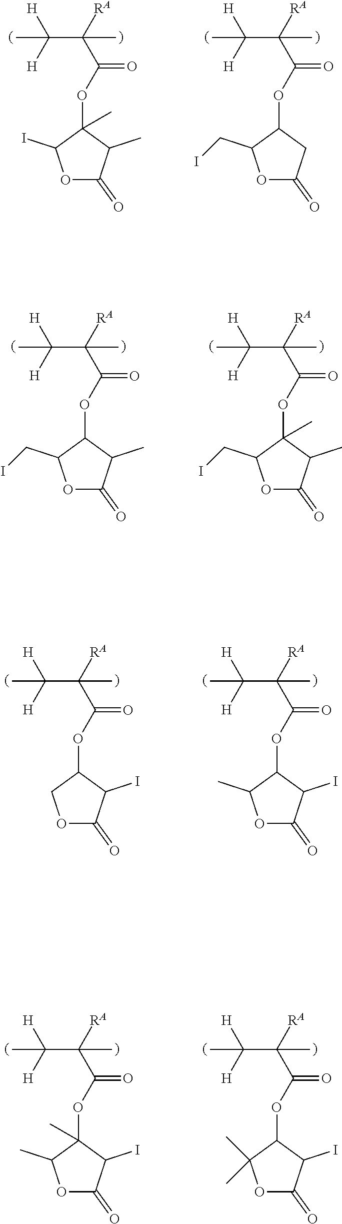

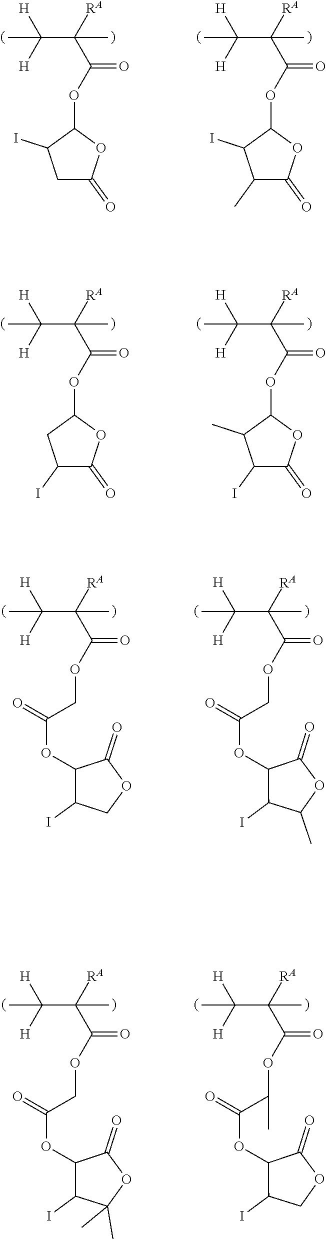

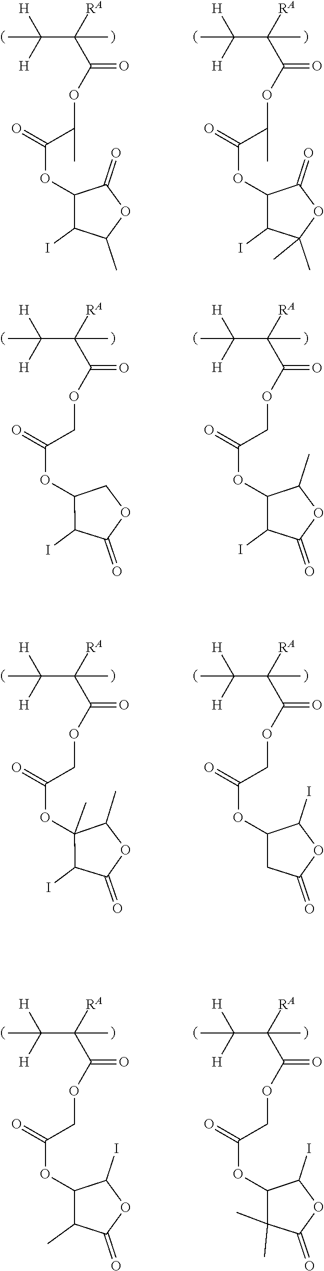

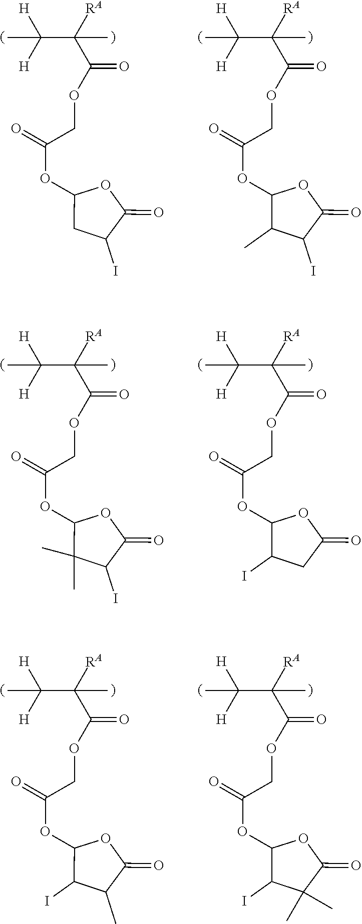

Of the repeat units (c) and (d), those units having a lactone ring as the polar group are preferred in the case of ArF lithography, and those units having a phenol site are preferred in the case of KrF, EB or EUV lithography.

The polymer may further comprise repeat units of a structure having a hydroxy group protected with an acid labile group (hereinafter, also referred to repeat units (e). The repeat unit (e) is not particularly limited as long as the unit includes one or more structures having a hydroxy group protected with a protective group such that the protective group is decomposed to generate a hydroxy group under the action of acid. repeat units having the formula (e1) are preferred.

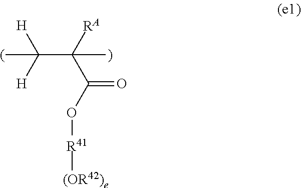

In formula (e1), R^{A} is a hydrogen atom, a fluorine atom, a methyl group, or a trifluoromethyl group. R⁴¹ is a C₁-C₃₀ (e+1)-valent hydrocarbon group which may contain a heteroatom. R⁴² is an acid labile group. e is an integer of 1 to 4.

In formula (e1), the acid labile group R⁴² is deprotected under the action of acid so that a hydroxy group is generated. The structure of R⁴² is not particularly limited, an acetal structure, ketal structure, alkoxycarbonyl group and alkoxymethyl group having the following formula (e2) are preferred, with the alkoxymethyl group having formula (e2) being more preferred.

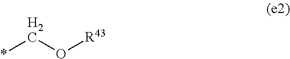

Herein the asterisk (*) designates a point of attachment, and R⁴³ is a C₁-C₁₅ hydrocarbyl group.

Examples of the acid labile group R⁴², the alkoxymethyl group having formula (e2), and the repeat units (e) are as exemplified for the repeat units (d) in JP-A 2020-111564.

In addition to the foregoing units, the base polymer may further comprise repeat units (f) derived from indene, benzofuran, benzothiophene, acenaphthylene, chromone, coumarin, and norbornadiene, or derivatives thereof. Specific examples of the monomer from which repeat units f are derived are shown below, but not limited thereto.

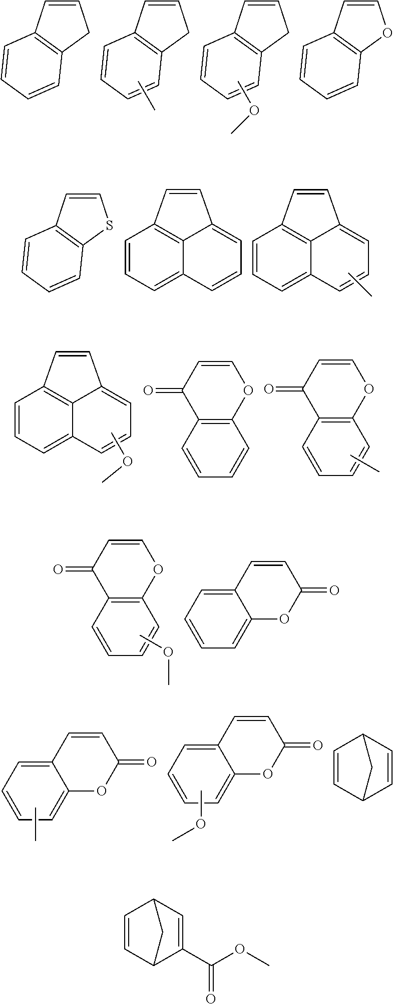

Furthermore, the base polymer may comprise repeat units (g) derived from styrene, indane, vinylpyridine, vinylcarbazole, or derivatives thereof.

In the inventive polymer, a fraction of repeat units (a), (b1), (b2), (c), (d), (e), (f), and (g) are preferably 0<a≤0.4, 0, 0<b1≤0.8, 0≤b2≤0.8, 0<c≤0.6, 0≤d≤0.6, 0≤e≤0.3, 0≤f≤0.3, and 0≤g≤0.3; more preferably 0<a≤0.3, 0<b1≤0.7, 0≤b2≤0.7, 0<c≤0.5, 0≤d≤0.5, 0≤e≤0.2, 0≤f≤0.2, and 0≤g≤0.2, provided that a+b1+b2+c+d+e+f+g≤1.0.

The polymer should preferably have a weight average molecular weight (Mw) in the range of 1,000 to 500,000, and more preferably 3,000 to 100,000. A Mw in the range ensures satisfactory etch resistance and eliminates the risk of resolution being lowered due to a failure to acquire a difference in dissolution rate before and after exposure. In the invention, Mw is a value measured by gel permeation chromatography (GPC) with tetrahydrofuran (THF) or N,N-dimethylformamide (DMF) as a solvent, and calculated as polystyrene.

Since the influence of dispersity (Mw/Mn) becomes stronger as the pattern rule becomes finer, the polymer should preferably have a narrow dispersity (Mw/Mn) of 1.0 to 2.0 in order to provide a resist composition suitable for micropatterning to a small feature size. A Mw/Mn in the range indicates smaller amounts of lower and higher molecular weight fractions and eliminates the risk of leaving foreign matter on the pattern or degrading the pattern profile after exposure and development.

Examples of the method for synthesizing the polymer include a method in which one or more monomers selected from the monomers corresponding to the foregoing repeat units are dissolved in an organic solvent, a radical polymerization initiator is added thereto, and the mixture is heated for polymerization.

Examples of the organic solvent which can be used for polymerization include toluene, benzene, tetrahydrofuran THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator used herein include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the initiator added is preferably 0.01 to 25 mol % based on the total of monomers. The reaction temperature is preferably 50 to 150° C., more preferably 60 to 100° C. The reaction time is preferably 2 to 24 hours, a time of 2 to 12 hours being more preferred in view of production efficiency.

The polymerization initiator may be added to the monomer solution, which is fed to the reactor. Alternatively, a solution of the polymerization initiator is prepared separately from the monomer solution, and the monomer and initiator solutions be independently fed to the reactor. Since there is a possibility that the initiator generates a radical in the standby time, by which polymerization reaction takes place to form an ultrahigh molecular weight compound, it is preferred from the standpoint of quality control that the monomer solution and the initiator solution be independently prepared and added dropwise. The acid labile group that has been incorporated in the monomer may be kept as such, or the polymerization may be followed by protection or partial protection. Any of well-known chain transfer agents such as dodecylmercaptan and 2-mercaptoethanol may be used for the purpose of adjusting molecular weight. An appropriate amount of the chain transfer agent is 0.01 to 20 mol % based on the total of monomers to be polymerized.

Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, hydroxystyrene or hydroxyvinylnaphthalene and other monomers may be dissolved in an organic solvent, a radical polymerization initiator is added thereto, and the mixture is heated for polymerization. Instead, as alternative method, acetoxystyrene or acetoxyvinylnaphthalene may be used and after polymerization, the acetoxy group is deprotected by alkaline hydrolysis, for thereby converting the polymer product to polyhydroxystyrene or hydroxypolyvinylnaphthalene.

Examples of the base that may be used in alkaline hydrolysis include aqueous ammonia and triethylamine. Preferably the reaction temperature is −20° C. to 100° C., more preferably 0° C. to 60° C. The reaction time is 0.2 to 100 hours, more preferably 0.5 to 20 hours.

The amounts of monomers in the monomer solution may be determined appropriate so as to provide the preferred fractions of repeat units as mentioned above.

The reaction solution resulting from polymerization reaction may be used as the final product. Alternatively, the polymer may be recovered in powder form through a purifying step such as re-precipitation step of adding the reaction solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145]. Exemplary solvents include ketones such as cyclohexanone and methyl-2-n-pentyl ketone; alcohols such as 3-methoxybutanol, 3 methyl-3-methoxybutanol, 1-methoxy-2-propanol, 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as diacetone alcohol (DAA); and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30% by weight, more preferably 0.1 to 20% by weight.

Prior to use, the reaction solution or polymer solution is preferably filtered through a filter. Filtration is effective for consistent quality because foreign matter and gel which can cause defects are removed.

Suitable materials of which the filter is made include fluorocarbon, cellulose, nylon, polyester, and hydrocarbon base materials. Preferred for the filtration of a chemically amplified resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

### [Chemically Amplified Resist Composition]

### (A) Base Polymer

The inventive chemically amplified resist composition comprises (A) a base polymer containing the polymer defined above.

The polymer may be used alone or as a mixture of two or more polymers which are different in compositional ratio, Mw and/or Mw/Mn. In addition to the polymer defined above, the base polymer (A) may contain a hydrogenated product of ring-opening metathesis polymerization polymer, which is described in JP-A 2003-66612.

### (B) Organic Solvent

The inventive chemically amplified resist composition may comprise (B) an organic solvent. The (B) organic solvent is not particularly limited as long as the component (A) and components described later are soluble therein. Examples of the organic solvent include ketones such as cyclopentanone, cyclohexanone and methyl-2-n-pentyl ketone; alcohols such as 3-methoxybutanol, 3-methyl-3-methoxybutanol, 1-methoxy-2-propanol, 1-ethoxy-2-propanol; keto-alcohols such as DAA, ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as GBL, which may be used alone or in admixture.

Of the foregoing organic solvents, 1-ethoxy-2-propanol, PGMEA, cyclohexanone, GBL, DAA and mixtures thereof are preferred because the base polymer (A) is most soluble therein.

The content of the organic solvent (B) in the inventive chemically amplified resist composition is preferably 200 to 5,000 parts by weight, more preferably 400 to 3,500 parts by weight per 80 parts by weight of the base polymer (A). The organic solvent (B) may be used alone or in admixture.

### (C) Quencher

The inventive chemically amplified resist composition may comprise (C) a quencher. In the invention, the quencher refers to a compound capable of trapping the acid, which is generated by the photoacid generator in the chemically amplified resist composition upon light exposure, to prevent the acid from diffusing to the unexposed region and to assist in forming the desired pattern.

Examples of the quencher (C) include onium salts having the following formulae (1) and (2).

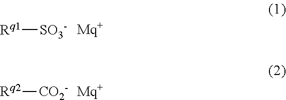

In formula (1), R^{q1} is hydrogen atom or a C₁-C₄₀ hydrocarbyl group which may contain a heteroatom, exclusive of the hydrocarbyl group in which the hydrogen atom bonded to the carbon atom at α-position of the sulfo group is substituted by fluorine atom or fluoroalkyl group. In formula (2), R^{q2} is a hydrogen atom, or a C₁-C₄₀ hydrocarbyl group which may contain a heteroatom.

Examples of the C₁-C₄₀ hydrocarbyl group R^{q1} include C₁-C₄₀ alkyls such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl and n-decyl groups; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2.6}]decyl and adamantyl groups; and C6-C₄₀ aryl groups such as phenyl, naphthyl and anthracenyl groups. Some or all of hydrogen atoms of the hydrocarbyl group may be replaced by a group containing a heteroatom such as an oxygen atom, a sulfur atom, a nitrogen atom or a halogen atom, some constituent —CH₂— of the hydrocarbyl group may be replaced by a group containing a heteroatom such as an oxygen atom, a sulfur atom or a nitrogen atom, and as a result, the hydrocarbyl group may contain a hydroxy group, a fluorine atom, a chlorine atom, a bromine atom, an iodine atom, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

Examples of the hydrocarbyl group R^{q2} include those exemplified above for R^{q1}, fluorinated saturated hydrocarbyl groups such as trifluoromethyl and trifluoroethyl groups, and fluorinated aryl groups such as pentafluorophenyl and 4-trifluoromethylphenyl groups.

Examples of the anion in the onium salt having formula (1) are shown below, but not limited thereto.

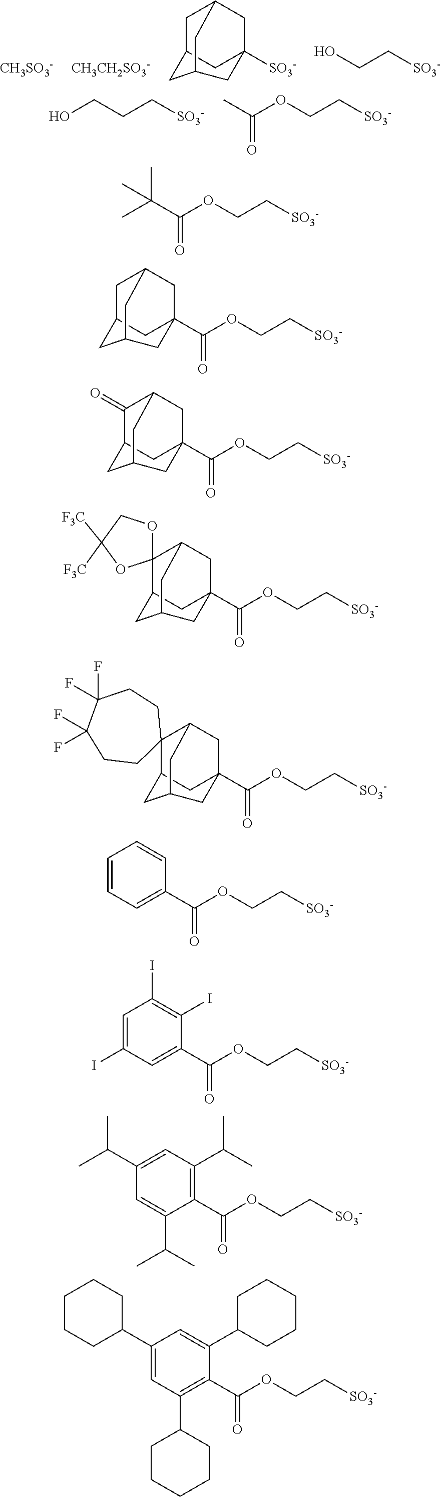

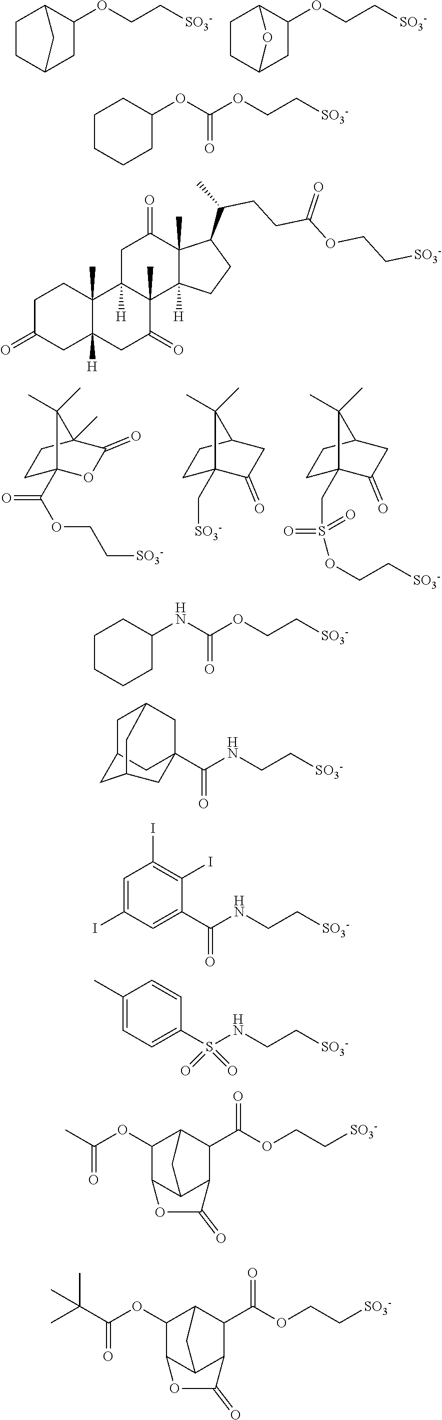

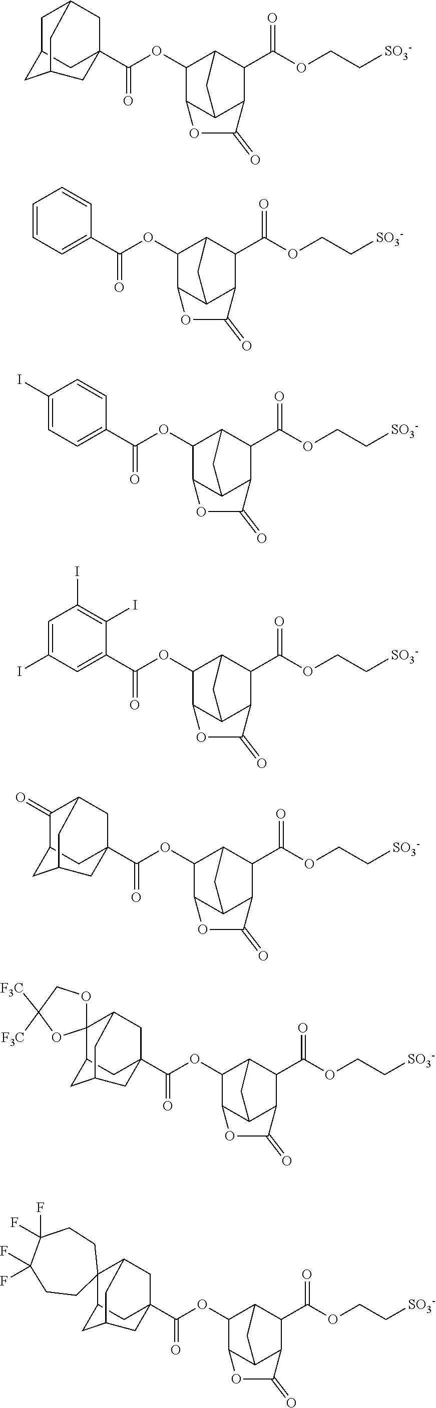

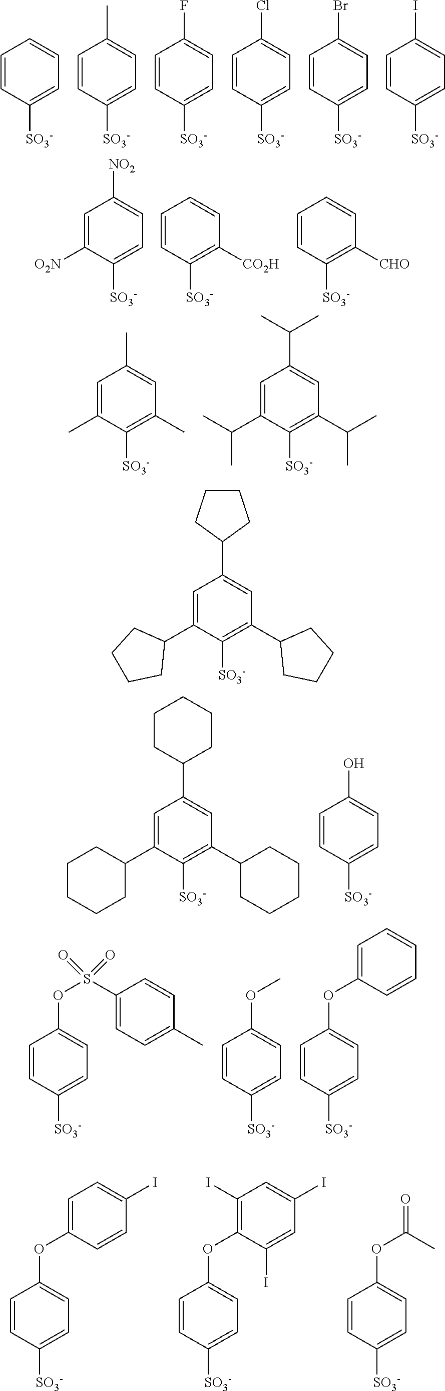

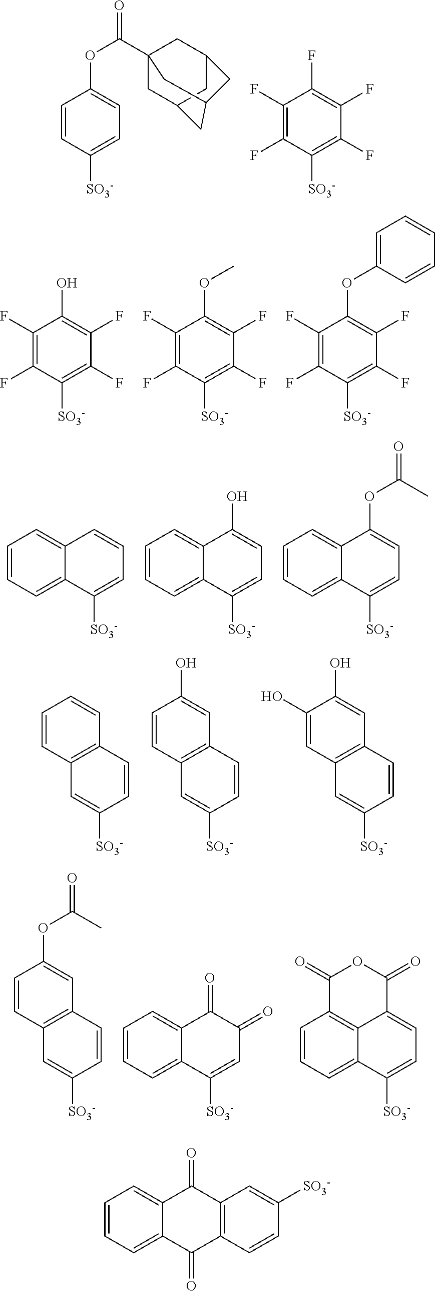

Examples of the anion in the onium salt having formula (2) are shown below, but not limited thereto.

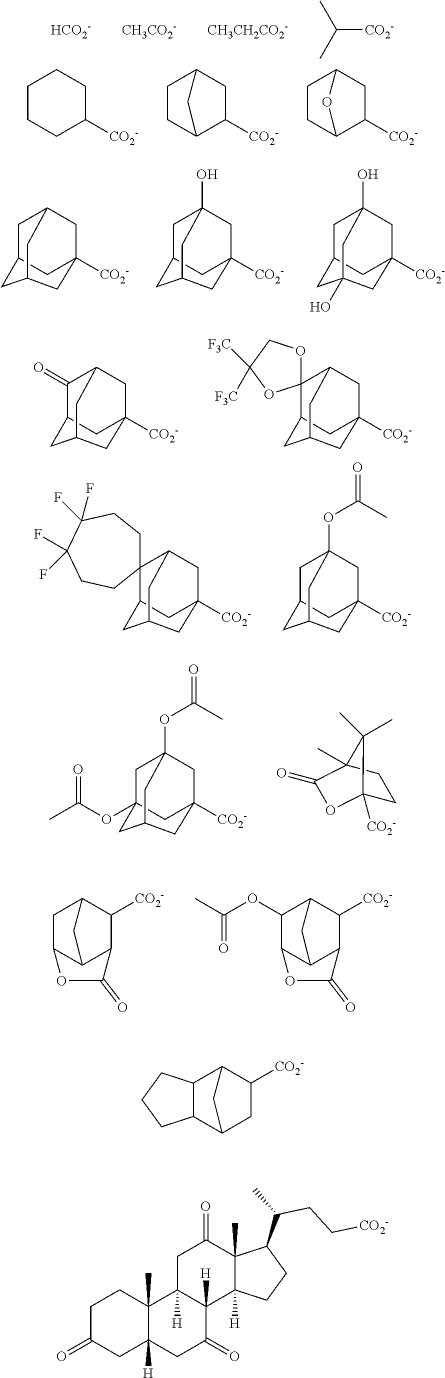

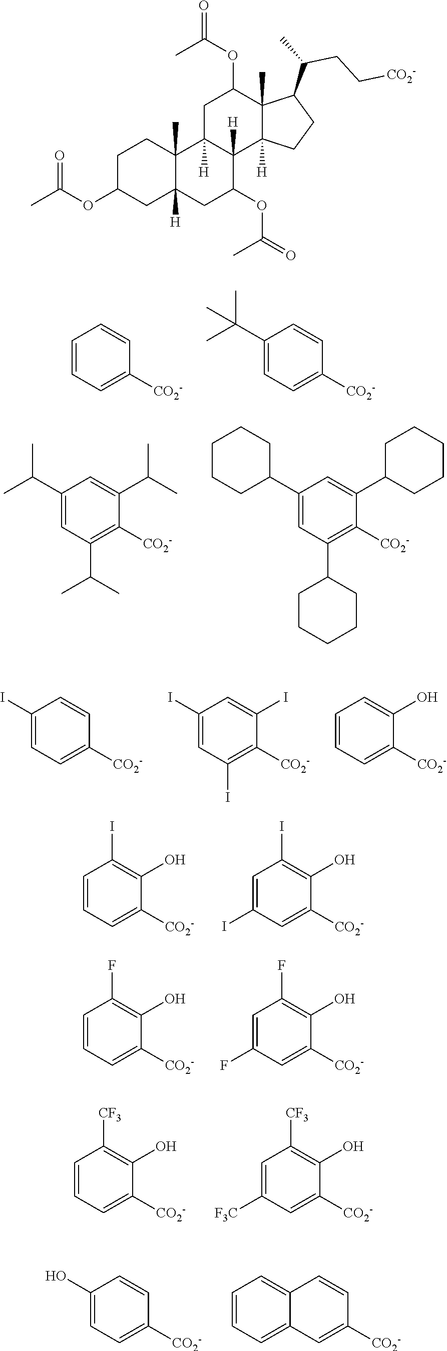

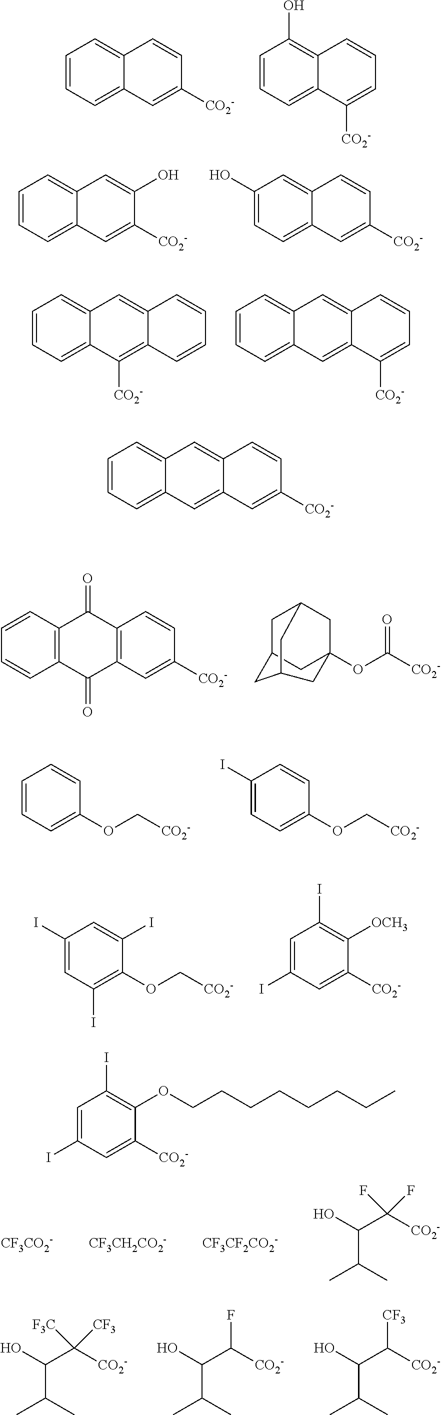

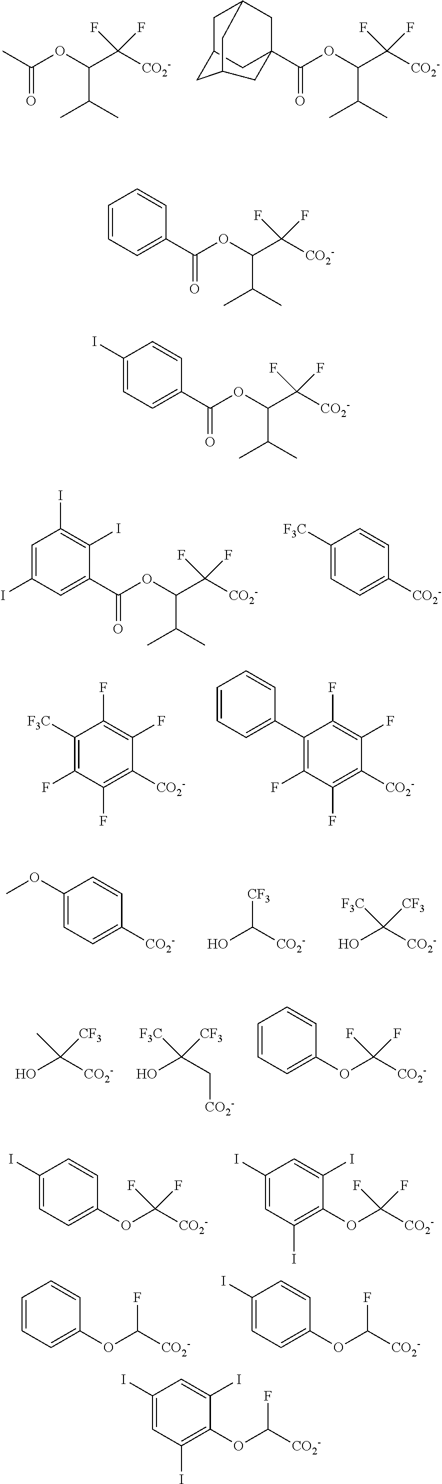

In the formulae (1) and (2), Mq⁺ is an onium cation. The onium cation is preferably a sulfonium cation having the formula (cation-1), iodonium cation having the formula (cation-2) or ammonium cation having the following formula (cation-3).

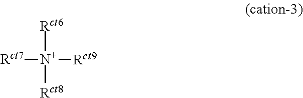

In formula (cation-3), R^{ct6} to R^{ct9} are each independently a C₁-C₄₀ hydrocarbyl group which may contain a heteroatom. R^{ct6} and R^{ct7} may bond together to form a ring with the nitrogen atom to which they are attached. Examples of the hydrocarbyl group are as exemplified above as hydrocarbyl groups R^{ct1} to R^{ct5} in formulae (cation-1) and (cation-2).

Examples of the ammonium cation represented by formula (cation-3) are shown below, but not limited thereto.

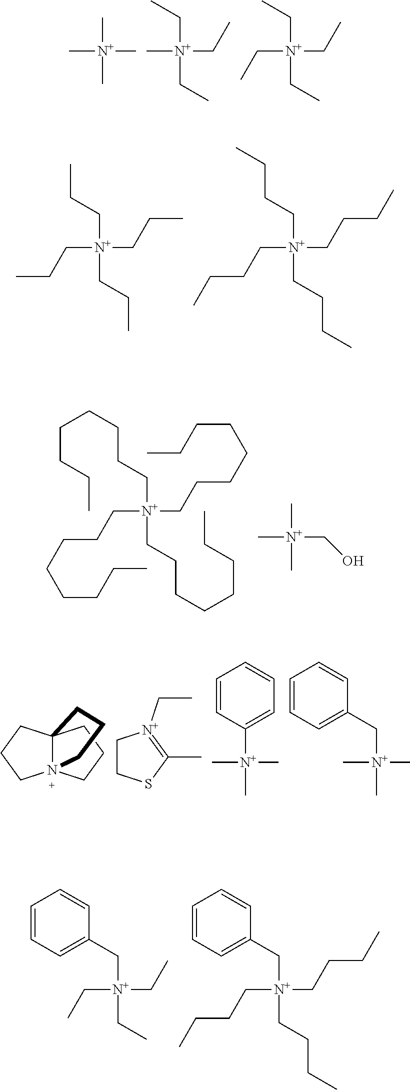

Examples of the onium salt having formula (1) or (2) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt having formula (1) or (2) functions as a quencher in the chemically amplified resist composition because the counter anion of the onium salt is a conjugated base of a weak acid. This is because the counter anion of the onium salt is a conjugated base of a weak acid. As used herein, the weak acid indicates an acidity insufficient to deprotect an acid labile group from an acid labile group-containing unit for the base polymer. The onium salt having formula (1) or (2) functions as a quencher when used in combination with an onium salt type photoacid generator having a conjugated base of a strong acid (typically a sulfonic acid which is fluorinated at α-position) as the counter anion. In a system using a mixture of an onium salt capable of generating a strong acid (e.g., α-position fluorinated sulfonic acid) and an onium salt capable of generating a weak acid (e.g., non-fluorinated sulfonic acid or carboxylic acid), if the strong acid generated from the photoacid generator upon exposure to high-energy radiation collides with the unreacted onium salt having a weak acid anion, then a salt exchange occurs whereby the weak acid is released and an onium salt having a strong acid anion is formed. In this course, the strong acid is exchanged into an acid having a low catalysis, incurring apparent deactivation of the acid for enabling to control acid diffusion.

JP 6848776 discloses an onium salt having sulfonium cation and phenoxide anion sites in the same molecule, JP 6583136 and JP-A 2020-200311 disclose an onium salt having sulfonium cation and carboxylate anion sites in the same molecule, and JP 6274755 discloses an onium salt having iodonium cation and carboxylate anion sites in the same molecule. These onium salts may also be used as the quencher (C).

If a photoacid generator capable of generating a strong acid is an onium salt, an exchange from the strong acid generated upon exposure to high-energy radiation to a weak acid as above can take place, but it rarely happens that the weak acid generated upon exposure to high-energy radiation collides with the unreacted onium salt capable of generating a strong acid to induce a salt exchange. This is because of a likelihood of an onium cation forming an ion pair with a stronger acid anion.

When the inventive chemically amplified resist composition comprises an onium salt of formula (1) or (2) as the quencher (C), the amount of the onium salt used is preferably 0.1 to 20 parts by weight, more preferably 0.1 to 10 parts by weight per 80 parts by weight of the base polymer (A). As long as the amount of component (C) is in the range, a satisfactory resolution is available without a substantial lowering of sensitivity. The onium salt having formula (1) or (2) may be used alone or in admixture.

The inventive chemically amplified resist composition may comprise a nitrogen-containing compound as the quencher (C). Examples of the nitrogen-containing compound (C) include primary, secondary and tertiary amine compounds, specifically amine compounds having a hydroxy group, ether bond, ester bond, lactone ring, cyano group or sulfonic ester bond, as described in JP A 2008-111103, paragraphs [0146]-[0164]. Primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-46501, for example.

When the inventive chemically amplified resist composition comprises a nitrogen-containing compound as the quencher (C), the amount of the nitrogen-containing compound used is preferably 0.001 to 12 parts by weight, more preferably 0.01 to 8 parts by weight per 80 parts by weight of the base polymer (A). The nitrogen-containing compound may be used alone or in admixture.

### (D) Acid Generator

The inventive chemically amplified resist composition may comprise an acid generator as long as the benefits of the present invention is not compromised. The acid generator is typically a compound (photoacid generator) capable of generating an acid upon exposure to actinic ray or radiation. Although the photoacid generator used herein is not particularly limited as long as it is capable of generating an acid upon exposure to high-energy radiation, those compounds capable of generating sulfonic acid, imide acid (imidic acid) or methide acid are preferred. Suitable photoacid generators include sulfonium salts, iodonium salts, sulfonyldiazomethane, N-sulfonyloxyimide, and oxime-O-sulfonate acid generators. Exemplary acid generators are described in JP-A 2008-111103, paragraphs [0122]-[0142].

Also, a sulfonium salt having the following formula (3-1) and an iodonium salt having the following formula (3-2) are advantageously used as the other acid generator.

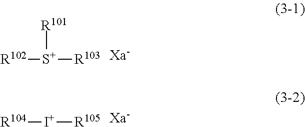

In formulae (3-1) and (3-2), R¹⁰¹ to R¹⁰⁵ are each independently a halogen atom, or a C1-C₂₀ hydrocarbyl group which may contain a heteroatom. Examples of the halogen atom and the hydrocarbyl group are as exemplified above as halogen atoms and hydrocarbyl groups R^{ct1} to R⁰¹⁵ in formulae (cation-1) and (cation-2). Some or all of hydrogen atoms of the hydrocarbyl group may be replaced by a group containing a heteroatom such as an oxygen atom, a sulfur atom, a nitrogen atom or a halogen atom, some constituent —CH₂— of the hydrocarbyl group may be replaced by a group containing a heteroatom such as an oxygen atom, a sulfur atom or a nitrogen atom, and as a result, the hydrocarbyl group may contain a hydroxy group, a fluorine atom, a chlorine atom, a bromine atom, an iodine atom, a cyano group, a nitro group, a carbonyl group, an ether bond, an ester bond, a sulfonic ester bond, a carbonate bond, a lactone ring, a sultone ring, carboxylic acid anhydride (—C(═O)—O—C(═O)—), a haloalkyl group, or the like. R¹⁰¹ and R¹⁰² may bond together to form a ring with a sulfur atom to which they are attached. Examples of the ring formed here are as exemplified above for formula (cation-) where R^{ct1} and R^{ct2} bond together to form a ring with a sulfur atom to which they are attached.

Examples of the sulfonium salt cation represented by formula (3-1) are as exemplified above for the sulfonium cation represented by formula (cation-1). Examples of the iodonium salt cation represented by formula (4-2) are as exemplified above for the iodonium cation represented by formula (cation-2).

In formulae (3-1) and (3-2), Xa⁻ is an anion of the following formula (3A), (3B), (3C) or (3D).

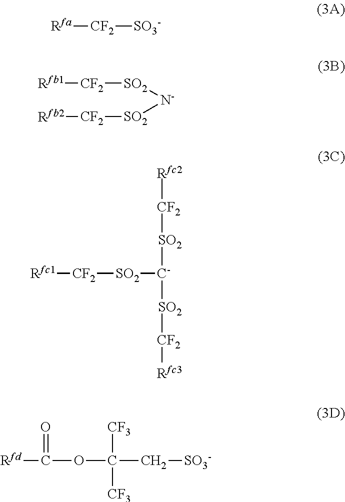

In formula (3A), R^{fa} is a fluorine atom, or a C₁-C₄₀ hydrocarbyl group which may contain a heteroatom. The hydrocarbyl group may be saturated or unsaturated, and may be linear, branched, or cyclic. Examples thereof are as will be exemplified later for hydrocarbyl group R^{fa} in formula (3A′).

Of the anions of formula (3A), a structure having formula (3A′) is preferred.

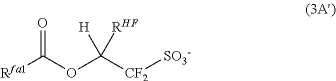

In formula (3A′), R^{HF} is a hydrogen atom or a trifluoromethyl group, preferably a trifluoromethyl group.

In formula (3A′), R^{fa1} is a C₁-C₃₈ hydrocarbyl group which may contain a heteroatom. Suitable heteroatoms include oxygen, nitrogen, sulfur and halogen atoms, with oxygen being preferred. Of the hydrocarbyl groups, those of 6 to 30 carbon atoms are preferred because a high resolution is available in fine pattern formation.

The C₁-C₃₈ hydrocarbyl group R^{fa1} may be saturated or unsaturated and straight, branched or cyclic. Examples thereof include C₁-C₃₈ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl and icocyl groups; C₃-C₃₈ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclodecyl, tetracyclodecylmethyl and dicyclohexylmethyl groups; C₂-C₃₈ unsaturated aliphatic hydrocarbyl groups such as allyl and 3-cyclohexenyl groups; C₆-C₃₈ aryl groups such as phenyl, 1-naphthyl and 2-naphthyl groups; C₇-C₃₈ aralkyl groups such as benzyl and diphenylmethyl groups; and combinations thereof.

Some or all of hydrogen atoms of the hydrocarbyl group may be replaced by a group containing a heteroatom such as an oxygen atom, a sulfur atom, a nitrogen atom or a halogen atom, some constituent —CH₂— of the hydrocarbyl group may be replaced by a group containing a heteroatom such as an oxygen atom, a sulfur atom or a nitrogen atom, and as a result, the hydrocarbyl group may contain a hydroxy group, a fluorine atom, a chlorine atom, a bromine atom, an iodine atom, a cyano group, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like. The heteroatom is preferably an oxygen atom. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl groups.

With respect to the synthesis of the sulfonium salt having an anion of formula (3A′), reference is made to JP-A 2007-145797, JP-A 2008-106045, JP-A 2009-7327, and JP-A 2009-258695. Also useful are the sulfonium salts described in JP-A 2010-215608, JP-A 2012-41320, JP-A 2012-106986, and JP-A 2012-153644.

Examples of the anion having formula (3A) are shown below, but not limited thereto. In the following formulae, Ac is an acetyl group.

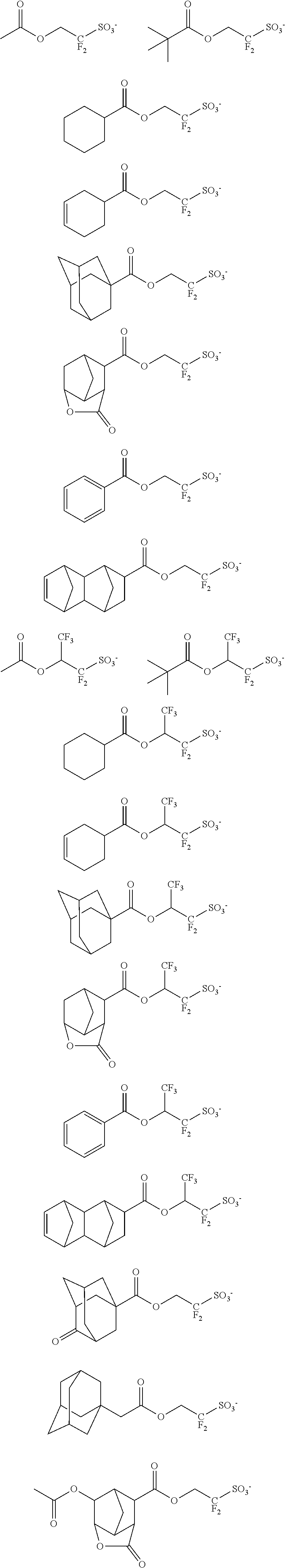

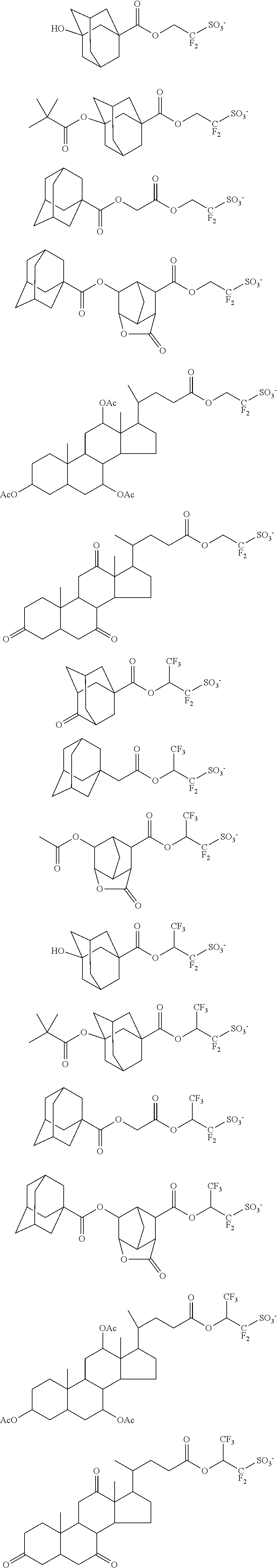

In formula (3B), R^{fb1} and R^{fb2} are each independently a fluorine atom, or a C₁-C₄₀ hydrocarbyl group which may contain a heteroatom. The hydrocarbyl group may be saturated or unsaturated, and may be linear, branched, or cyclic. Examples of the hydrocarbyl group are as exemplified above as a hydrocarbyl group R^{fa1} in formula (3A′). Preferably R^{fb1} and R^{fb2} each are a fluorine atom or a straight C₁-C₄ fluorinated alkyl group. A pair of R^{fb1} and R^{fb2} may bond together to form a ring with the linkage (—CF₂—SO₂—N⁻—SO₂—CF₂—) to which they are attached, and the ring-forming pair is preferably a fluorinated ethylene or fluorinated propylene group.

In formula (3C), R^{fc1}, R^{fc2} and R^{fc3} are each independently a fluorine atom, or a C₁-C₄₀ hydrocarbyl group which may contain a heteroatom. The hydrocarbyl group may be saturated or unsaturated, and may be linear, branched, or cyclic. Examples of the hydrocarbyl group are as exemplified above as a hydrocarbyl group R^{fa1} in formula (3A′). Preferably R^{fc1}, R^{fc2} and R^{fc3} each are fluorine atom or a straight C₁-C₄ fluorinated alkyl group. A pair of R^{fc1} and R^{fc2} may bond together to form a ring with the linkage (—CF₂—SO₂—C⁻—SO₂—CF₂—) to which they are attached, and the ring-forming pair is preferably a fluorinated ethylene or fluorinated propylene group.

In formula (3D), R^{fd} is a C₁-C₄₀ hydrocarbyl group which may contain a heteroatom. The hydrocarbyl group may be saturated or unsaturated, and may be linear, branched, or cyclic. Examples of the hydrocarbyl group are as exemplified above as a hydrocarbyl group R^{fa1} in formula (3A′).

With respect to the synthesis of the sulfonium salt having an anion of formula (3D), reference is made to JP-A 2010-215608, and JP-A 2014-133723.

Examples of the anion having formula (3D) are shown below, but not limited thereto.

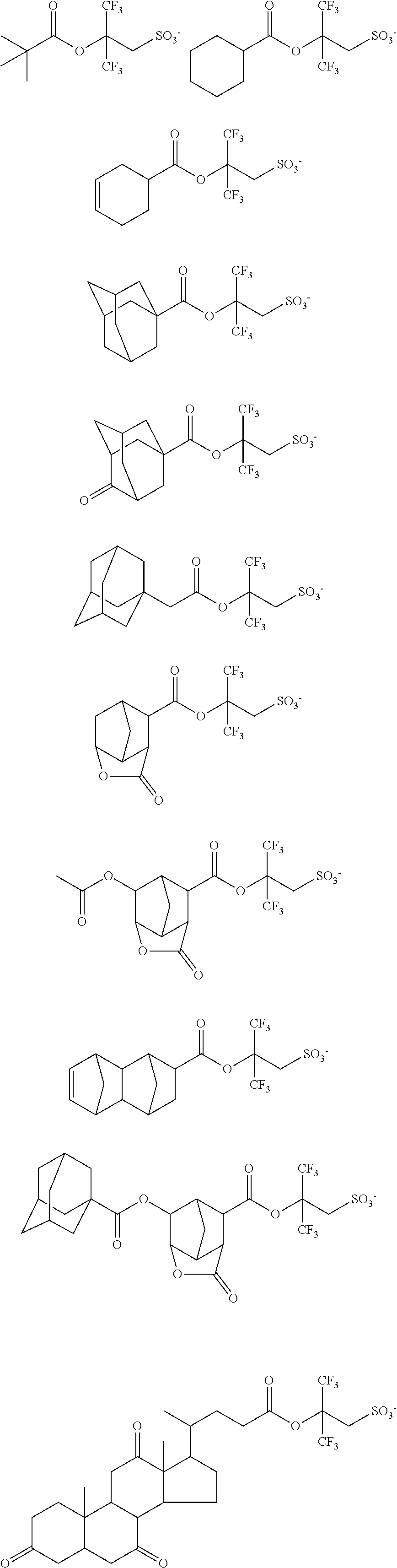

The photoacid generator having the anion of formula (3D)) does not have a fluorine atom at the α-position relative to the sulfo group, but two trifluoromethyl groups at the β-position. For this reason, it has a sufficient acidity to sever the acid labile groups in the base polymer. Thus the compound is an effective photoacid generator.

Also, a compound having the formula (4) can be suitably used as a photoacid generator.

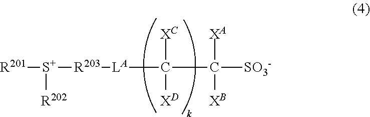

In formula (4), R²⁰¹ to R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹ and R²⁰² and R²⁰³ may bond together to form a ring with a sulfur atom to which they are attached. Examples of the ring are as exemplified above for formula (cation-1) where R^{ct1} and R^{ct2} bond together to form a ring with a sulfur atom to which they are attached.

The C₁-C₃₀ hydrocarbyl groups R²⁰¹ and R²⁰² may be saturated or unsaturated and straight, branched or cyclic. Examples thereof include C1-C₃₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl and n-decyl groups; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl and adamantyl groups; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl and anthracenyl groups; and combinations thereof. Some or all of hydrogen atoms of the hydrocarbyl group may be replaced by a group containing a heteroatom such as an oxygen atom, a sulfur atom, a nitrogen atom or a halogen atom, some constituent —CH₂— of the hydrocarbyl group may be replaced by a group containing a heteroatom such as an oxygen atom, a sulfur atom or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonic ester bond, a carbonate bond, a lactone ring, a sultone ring, carboxylic anhydride (—C(═O)—O—C(═O)—), a haloalkyl group, or the like.

The C₁-C₃₀ hydrocarbylene group R²⁰³ may be saturated or unsaturated and straight, branched or cyclic. Examples thereof include C1-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl and heptadecane-1,17-diyl groups; C₃-C₃₀ cyclic saturated hydrocarbylene groups such as cyclopentanediyl, cyclohexanediyl, norbornanediyl and adamantanediyl groups; and C₆-C₃₀ arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene and tert-butylnaphthylene groups; and combinations thereof. Some or all of hydrogen atoms of the hydrocarbylene group may be replaced by a group containing a heteroatom such as an oxygen atom, a sulfur atom, a nitrogen atom or a halogen atom, some constituent —CH₂— of the hydrocarbylene group may be replaced by a group containing a heteroatom such as an oxygen atom, a sulfur atom or a nitrogen atom, and as a result, the hydrocarbyl group may contain a hydroxy group, a cyano group, a fluorine atom, a chlorine atom, a bromine atom, an iodine atom, a carbonyl group, an ether bond, an ester bond, a sulfonic ester bond, a carbonate bond, a lactone ring, a sultone ring, carboxylic acid anhydride (—C(═O)—O—C(═O)—), a haloalkyl group, or the like. The heteroatom is preferably an oxygen atom.

In formula (4), L^{A} is a single bond, an ether bond, or a C₁-C₂₀ hydrocarbylene group which may contain a heteroatom. The hydrocarbylene group may be saturated or unsaturated and straight, branched or cyclic. Examples thereof are as exemplified above as a hydrocarbylene group R²⁰³.

In formula (4), X^{A}, X^{B}, X^{C} and X^{D} are each independently a hydrogen atom, a fluorine atom or a trifluoromethyl group. It is to be noted that at least one of X^{A}, X^{B}, X^{C} and X^{D} a fluorine atom or a trifluoromethyl group.

In formula (4), k is an integer of 0 to 3.

Preferably, the photoacid generator of formula (4) has the following formula (4′).

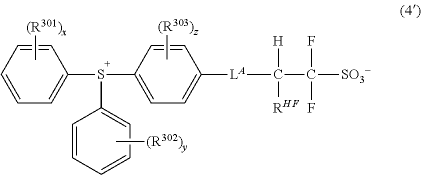

In formula (4′), L^{A} is as defined above. R^{HF} is a hydrogen atom or a trifluoromethyl group, preferably a trifluoromethyl group. R³⁰¹, R³⁰² and R³⁰³ each independently a C₁-C₂₀ hydrocarbyl group which may contain a heteroatom. The hydrocarbyl group may be saturated or unsaturated, and may be linear, branched, or cyclic. Examples of the hydrocarbyl group are as exemplified above as a hydrocarbyl group R^{fa1} in formula (3A′). x and y are each independently an integer of 0 to 5. z is an integer of 0 to 4.

Examples of the photoacid generator having formula (4) are as exemplified as for the photoacid generator having formula (2) in JP-A 2017-26980.

Of the foregoing photoacid generators, those having an anion of formula (3A′) or (3D) are especially preferred because of reduced acid diffusion and high solubility in the resist solvent. Also those having formula (4′) are especially preferred because of extremely reduced acid diffusion.

As other acid generators, sulfonium salts and iodonium salts having the following formula (5-1) or (5-2) may also be used. These salts contain an anion having an aromatic ring substituted with an iodine atom.

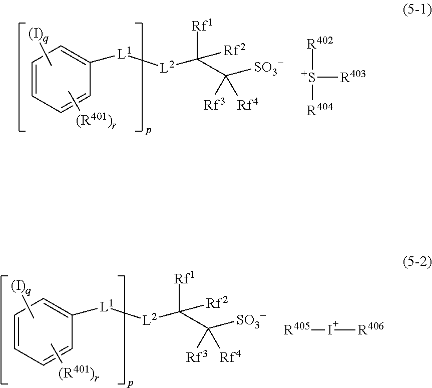

In formulae (5-1) and (5-2), p is 1, 2 or 3. q is an integer of 1 to 5, r is an integer of 0 to 3, and the sum of q+r is an integer of 1 to 5. q is preferably 1, 2 or 3, more preferably 2 or 3. r is preferably 0, 1 or 2.

In formulae (5-1) and (5-2), L¹ is a single bond, an ether bond, an ester bond, or a C₁-C₆ saturated hydrocarbylene group which may contain an ether bond or an ester bond. The saturated hydrocarbylene group may be straight, branched or cyclic.

In formulae (5-1) and (5-2), L² is a single bond or a C₁-C₂₀ divalent linking group when p=1, and a C₁-C₂₀ (p+1)-valent linking group which may contain oxygen atom, sulfur atom or nitrogen atom when p=2 or 3.

In formulae (5-1) and (5-2), R⁴⁰¹ is a hydroxy group, a carboxy group, a fluorine atom, a chlorine atom, a bromine atom, or an amino group, or a C₁-C₂₀ hydrocarbyl group, C₁-C₂₀ hydrocarbyloxy group, C₂-C₂₀ hydrocarbylcarbonyl group, C₂-C₂₀ hydrocarbyloxycarbonyl group, C₂-C₂₀ hydrocarbylcarbonyloxy group, or C₁-C₂₀ hydrocarbylsulfonyloxy group, which may contain a fluorine atom, a chlorine atom, a bromine atom, a hydroxy group, an amino group or an ether bond, or —N(R^{401A})(R^{401B}), —N(R^{401C})—C(═O)—R^{401D} or —N(R^{401C})—C(═O)—O—R^{401D}. R^{401A} and R^{401B} are each independently a hydrogen atom, or a C₁-C₆ saturated hydrocarbyl group. R^{401C} is a hydrogen atom, or a C₁-C₆ saturated hydrocarbyl group which may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. R^{401D} is a C₁-C₁₆ aliphatic hydrocarbyl group, a C₆-C₁₄ aryl group, or a C₇-C₁₅ aralkyl group, a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy and hydrocarbylsulfonyloxy groups may be straight, branched or cyclic. Groups R⁴⁰¹ may be the same or different when p and/or r is 2 or more.

Of these, R⁴⁰¹ is preferably hydroxy group, —N(R^{401C})—C(═O)—R^{401D}, —N(R^{401C})—C(═O)—O—R^{401D}, a fluorine atom, a chlorine atom, a bromine atom, a methyl group, or a methoxy group.

In formula (5-1) and (5-2), Rf^{f11} to Rf¹⁴ are each independently a hydrogen atom, a fluorine atom or a trifluoromethyl group, and at least one of Rf¹¹ to Rf¹⁴ is fluorine atom or trifluoromethyl group. Rf¹¹ and Rf¹², taken together, may form a carbonyl group. Particularly, both Rf¹³ and Rf¹⁴ are preferably fluorine atoms.

In formulae (5-1) and (5-2), R⁴⁰² to R⁴⁰⁶ are each independently a halogen atom, or a C₁-C₂₀ hydrocarbyl group which may contain a heteroatom. The hydrocarbyl group may be saturated or unsaturated, and may be linear, branched, or cyclic. Examples thereof are as exemplified above as hydrocarbyl groups R^{ct1} to R^{ct5} in formulae (cation-1) and (cation-2). Some or all of hydrogen atoms of the hydrocarbyl group may be replaced by a hydroxy group, a carboxy group, a halogen atom, a cyano group, a nitro group, a mercapto group, a sultone ring, a sulfo group or a sulfonium salt-containing group, and some constituent —CH₂— of the hydrocarbyl group may be replaced by an ether bond, an ester bond, a carbonyl group, an amide bond, a carbonate bond, or a sulfonic ester bond. R⁴⁰² and R⁴⁰³ may bond together to form a ring with a sulfur atom to which they are attached. Examples of the ring are as exemplified above for formula (cation-1) where R^{ct1} and R^{ct2} bond together to form a ring with a sulfur atom to which they are attached.

Examples of the sulfonium salt cation represented by formula (5-1) are as exemplified above for the sulfonium cation represented by formula (cation-1). Examples of the iodonium salt cation represented by formula (5-2) are as exemplified above for the iodonium cation represented by formula (cation-2).

Examples of the anion in the onium salt having formula (5-1) or (5-2) are shown below, but not limited thereto.

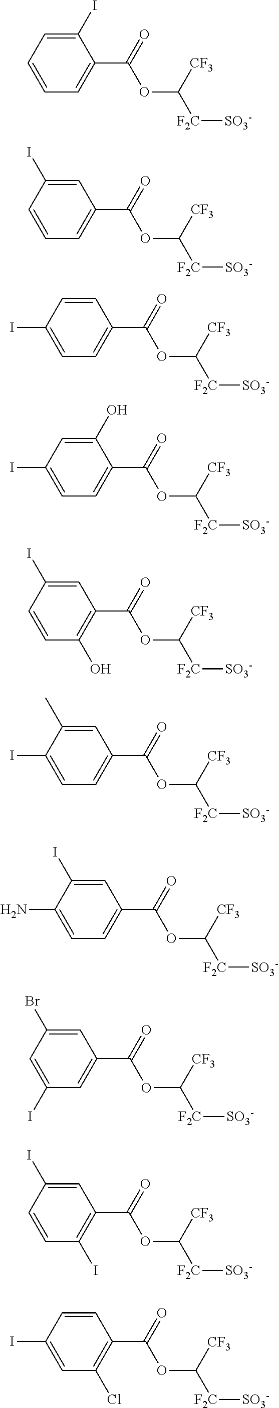

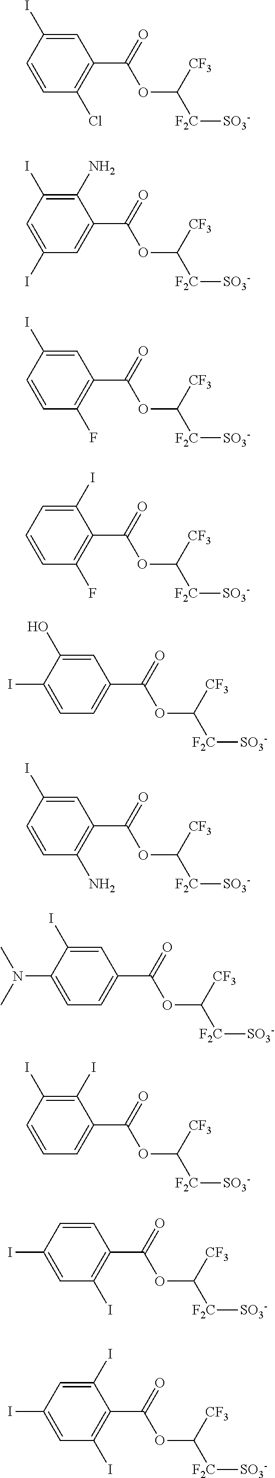

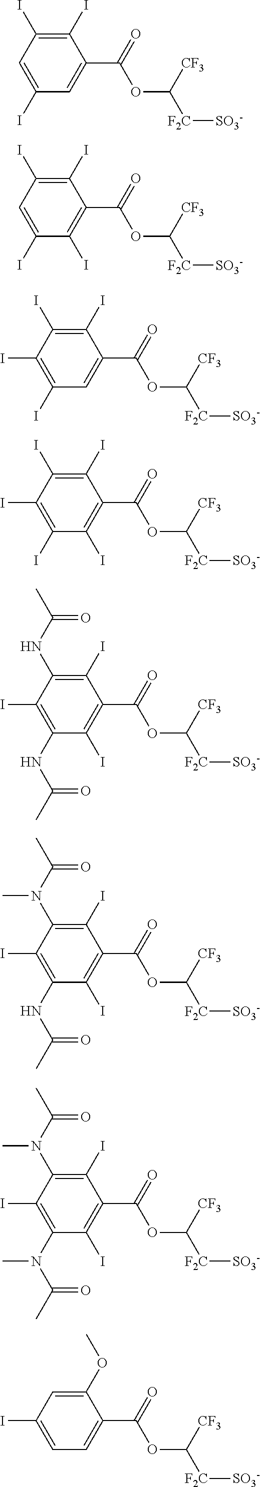

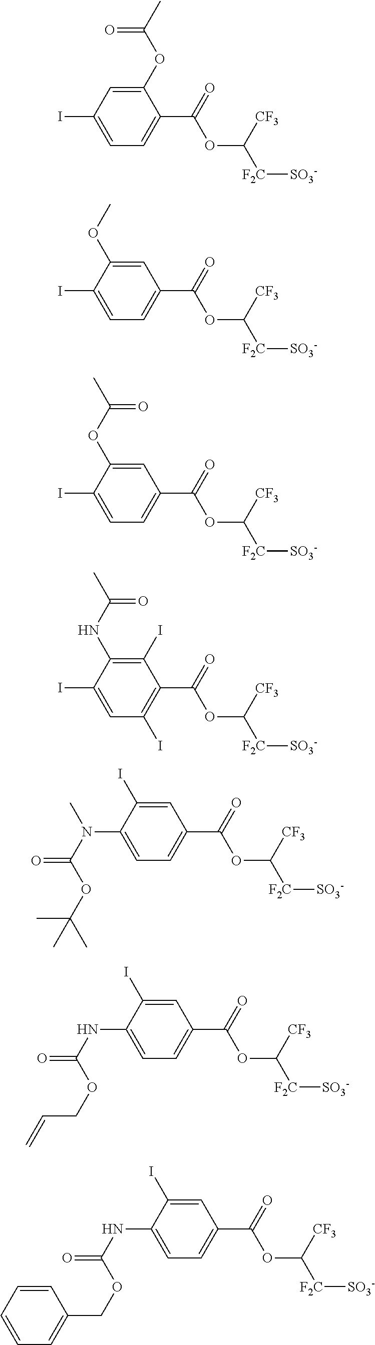

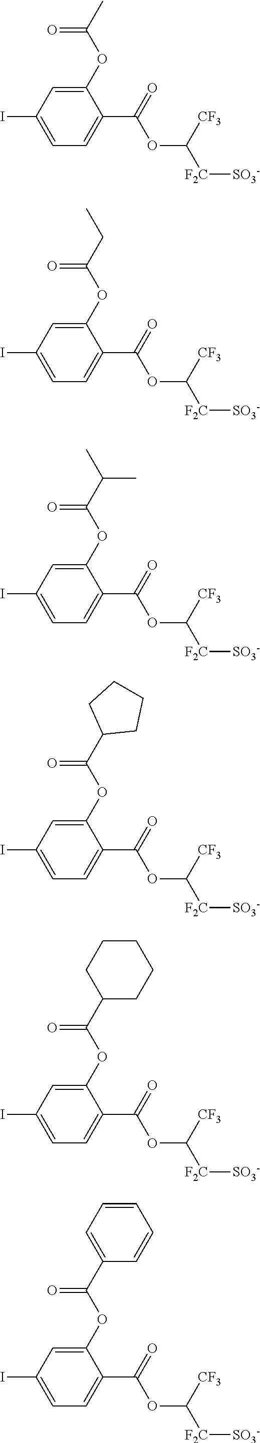

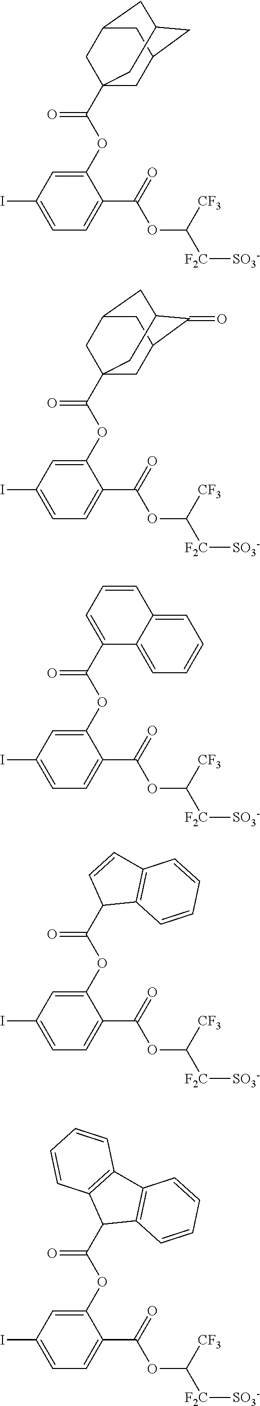

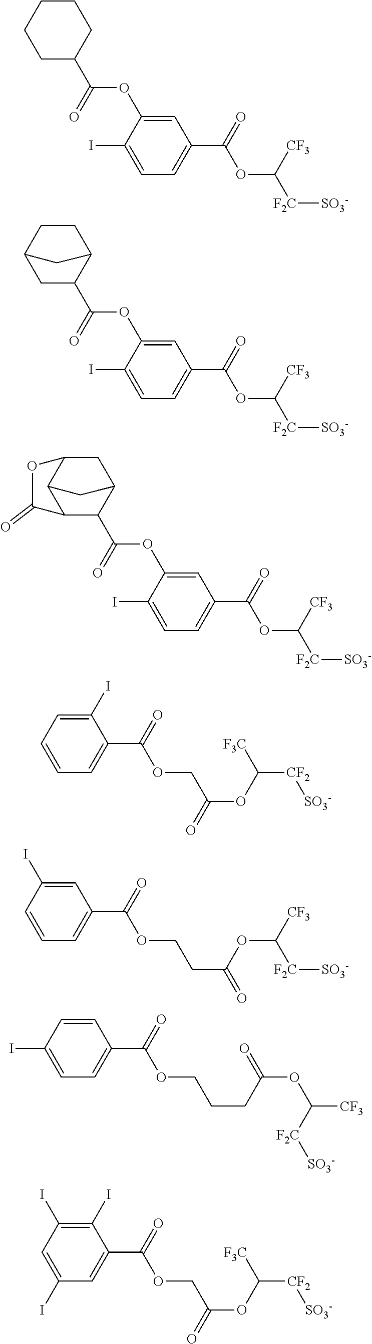

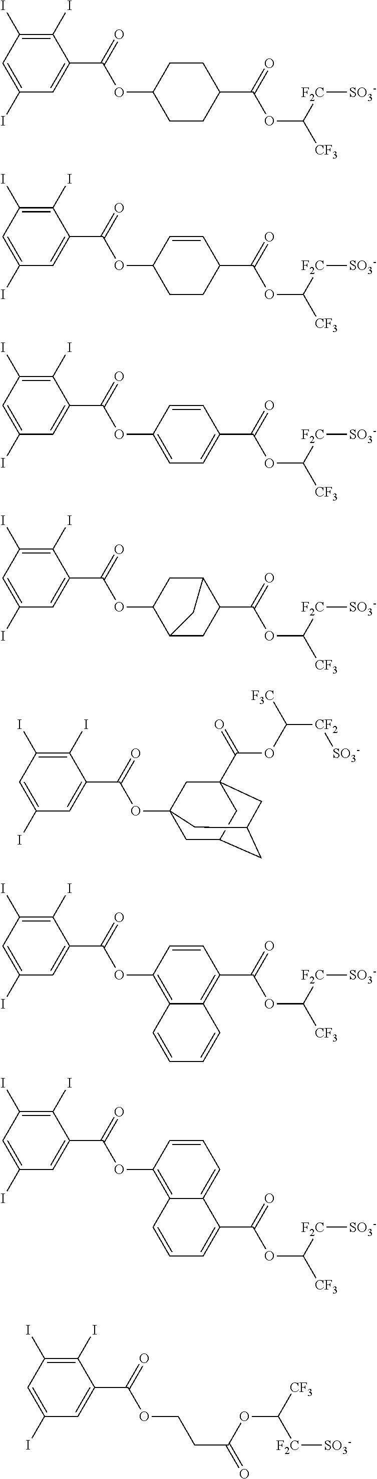

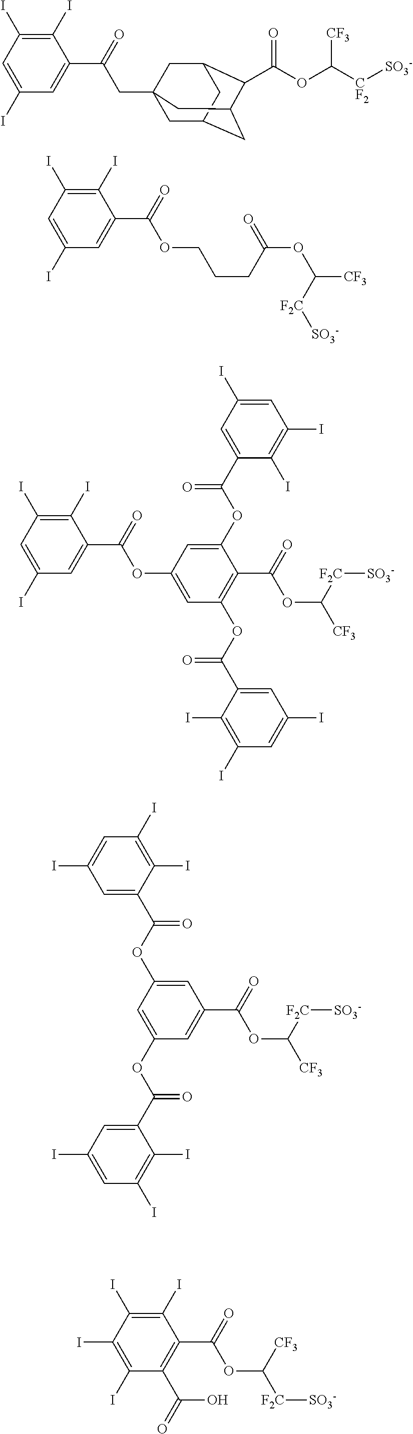

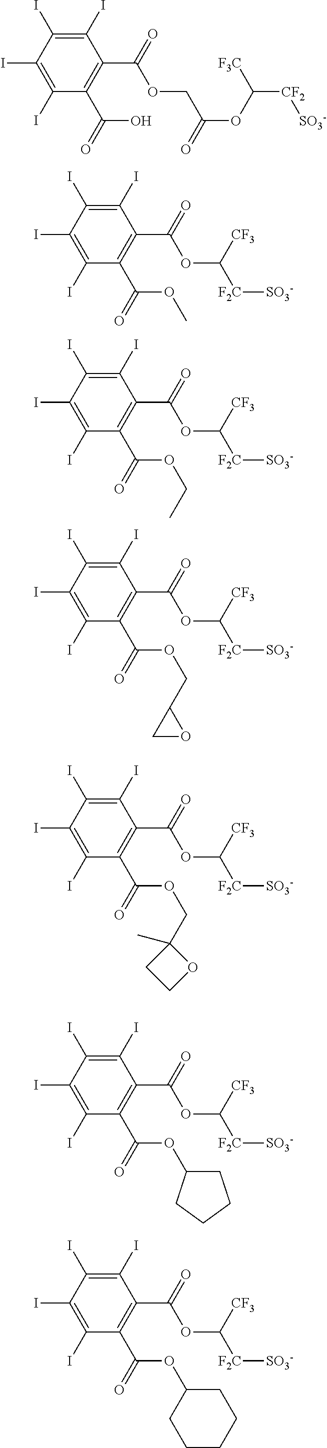

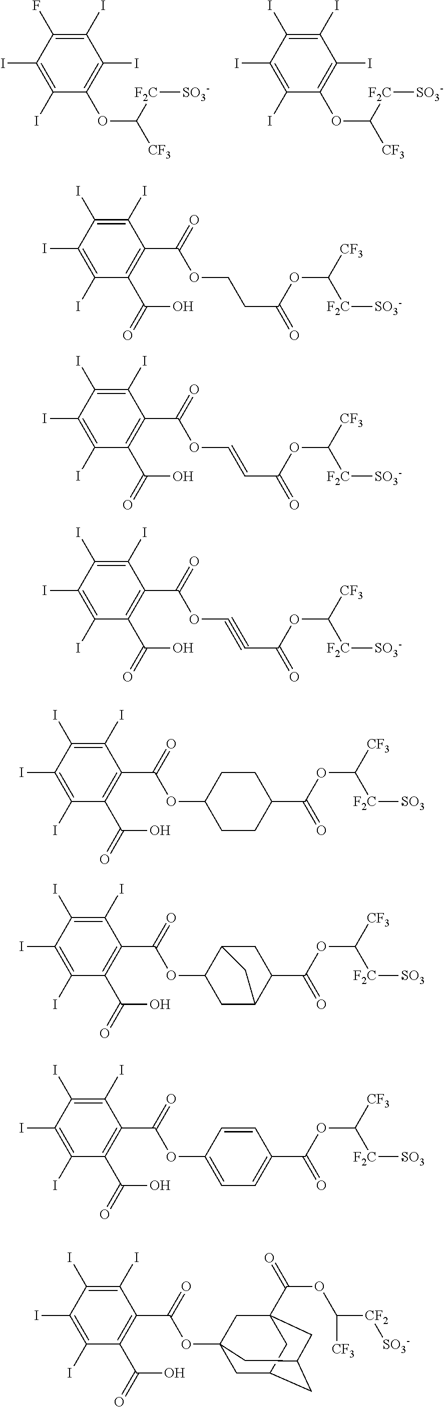

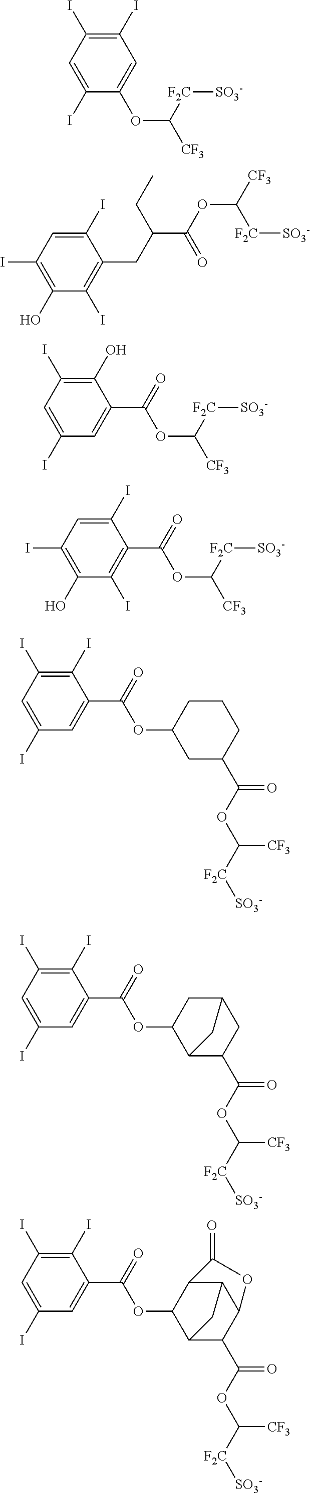

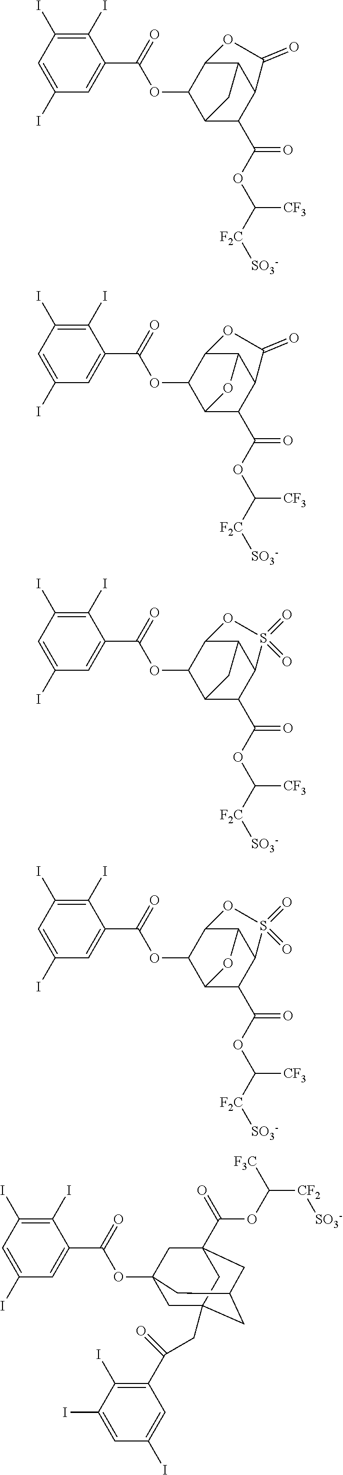

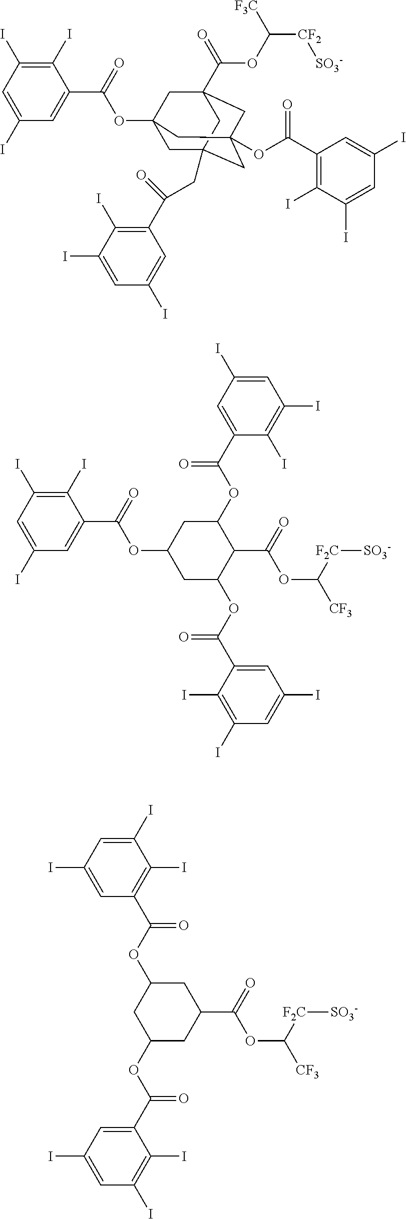

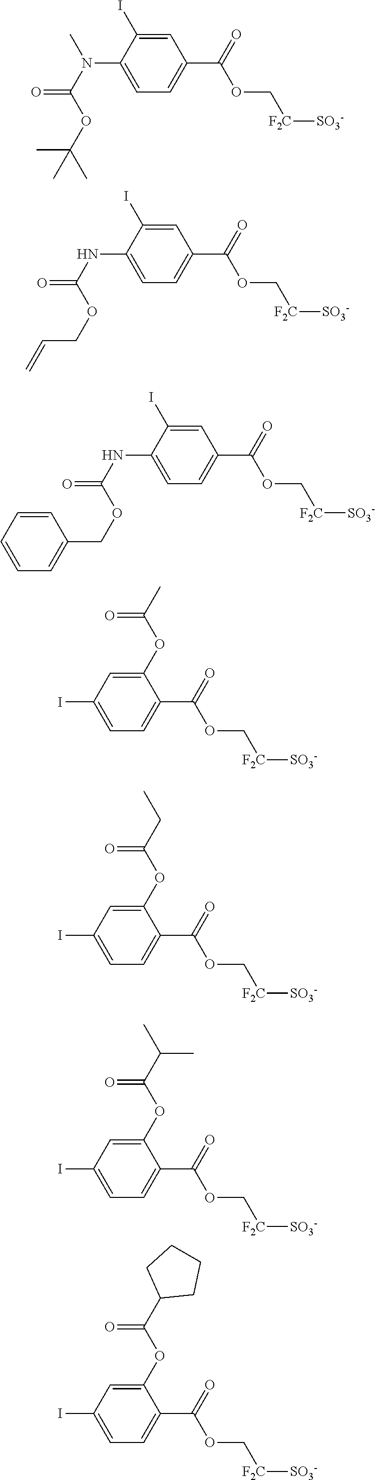

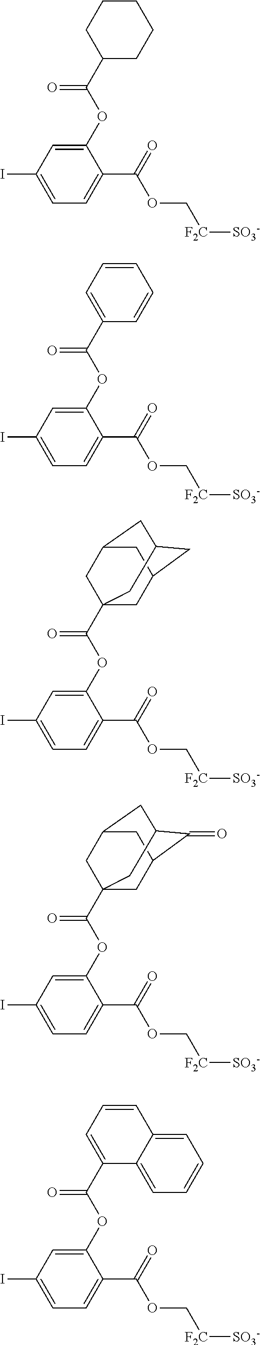

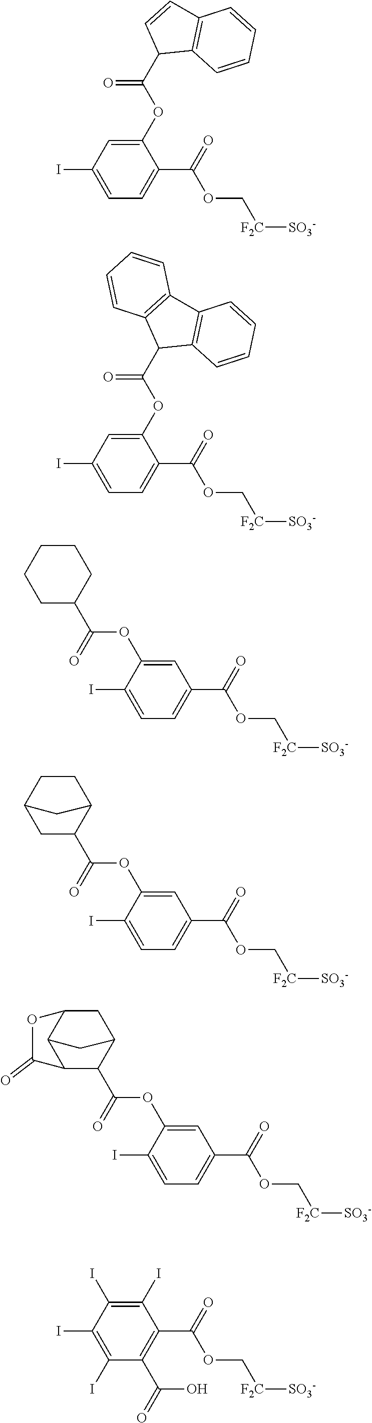

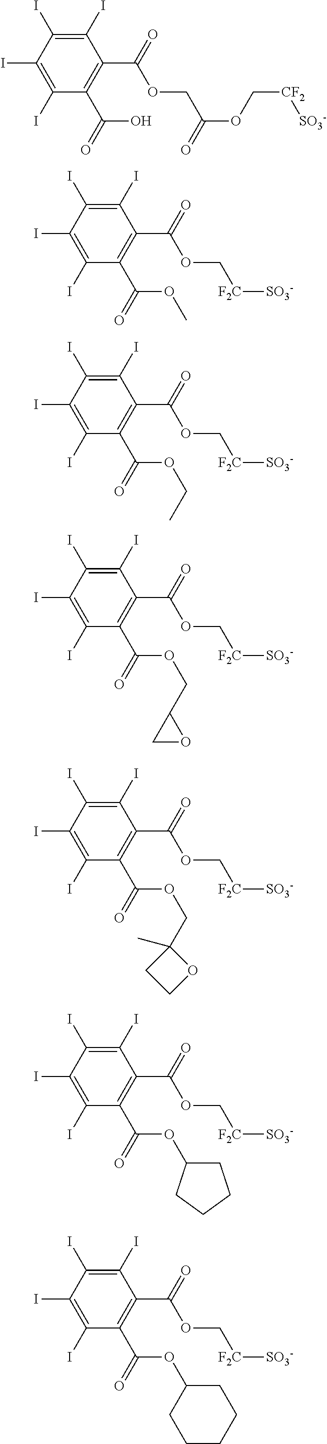

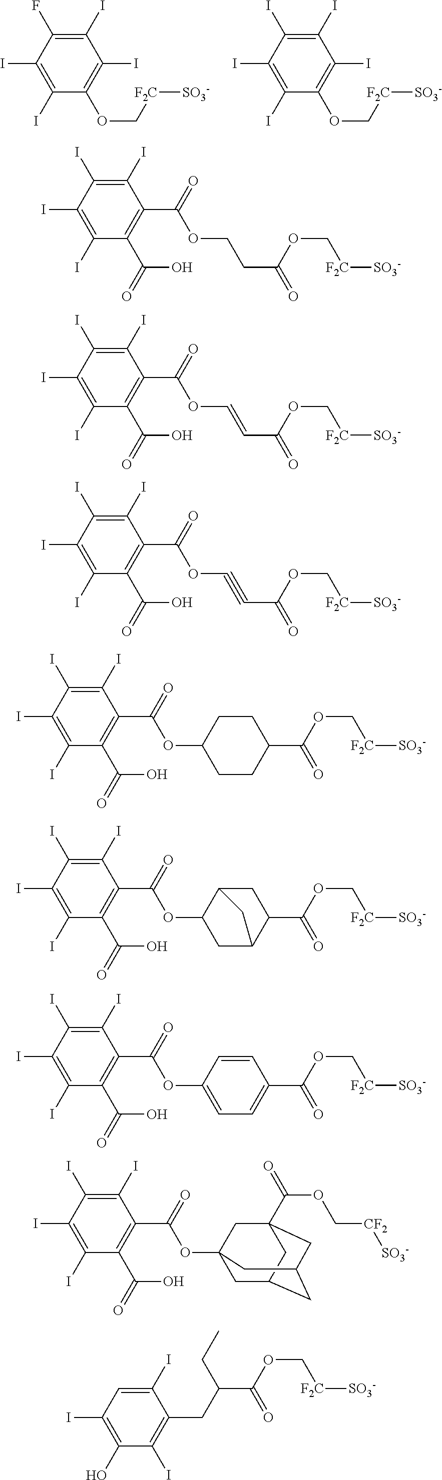

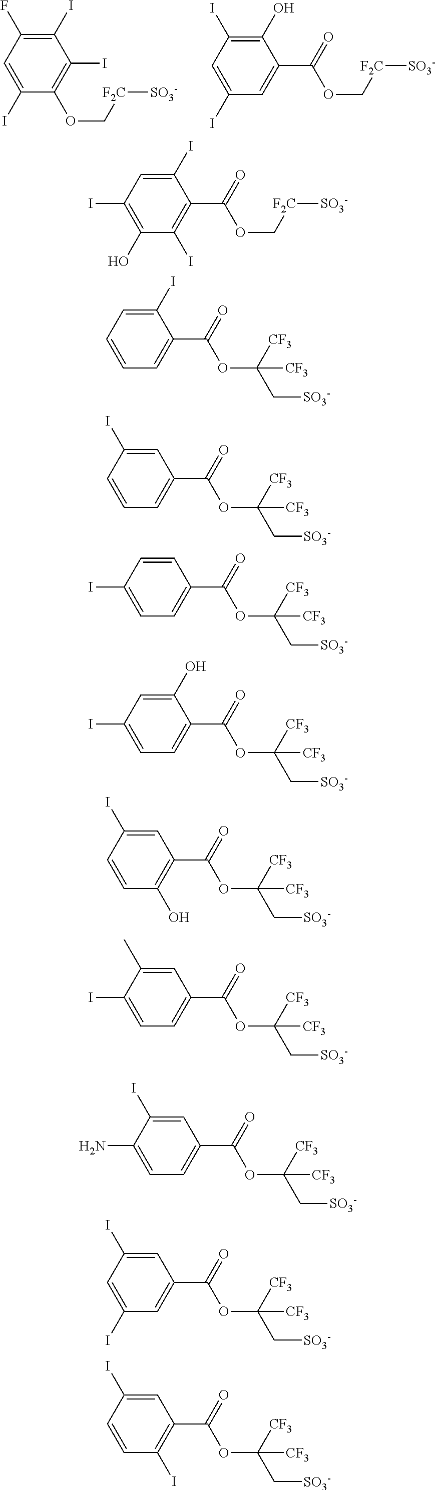

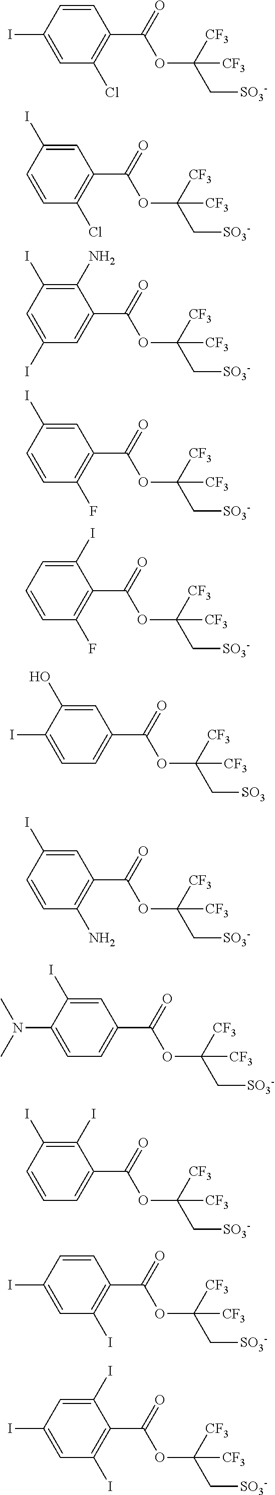

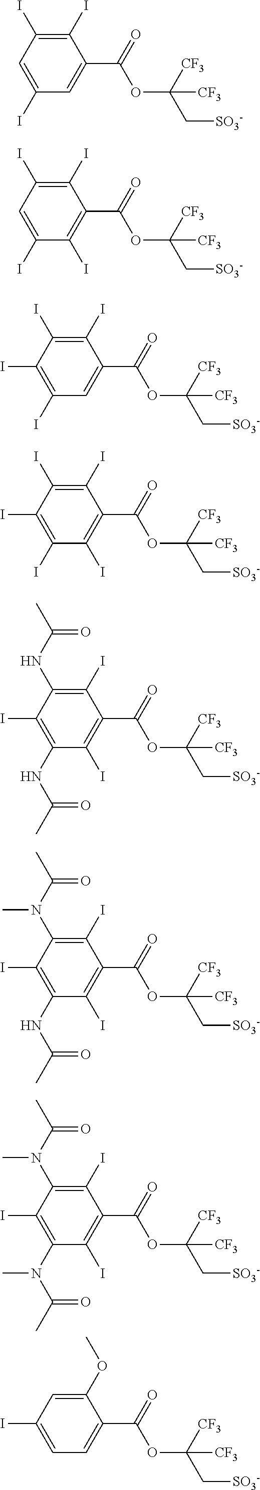

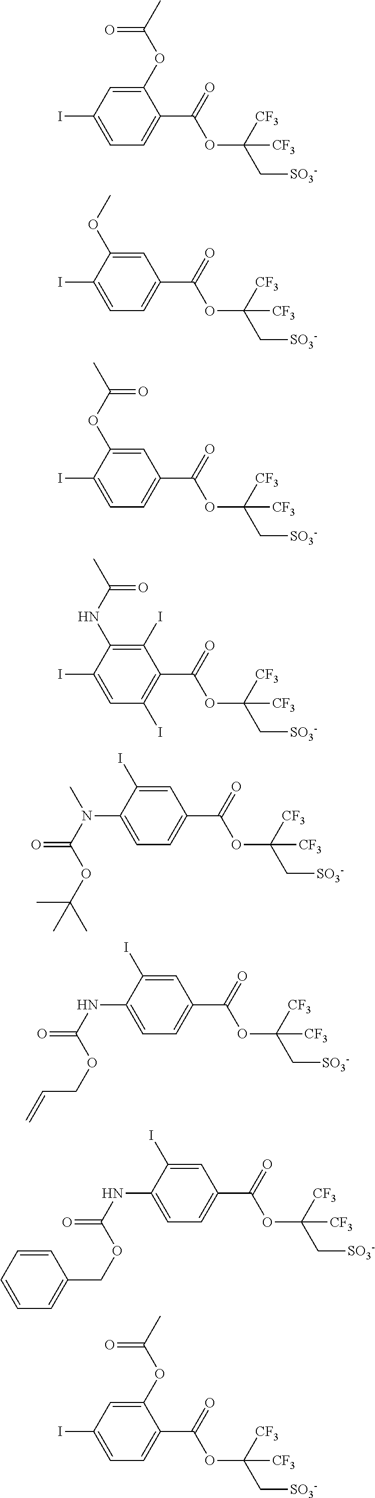

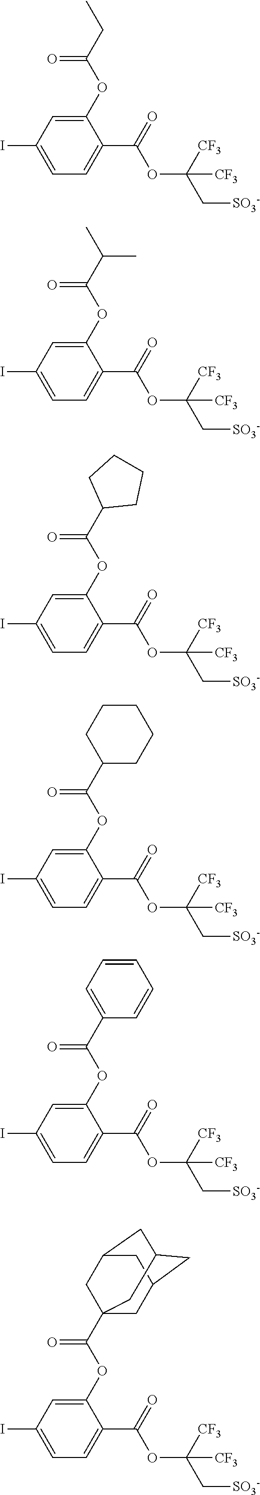

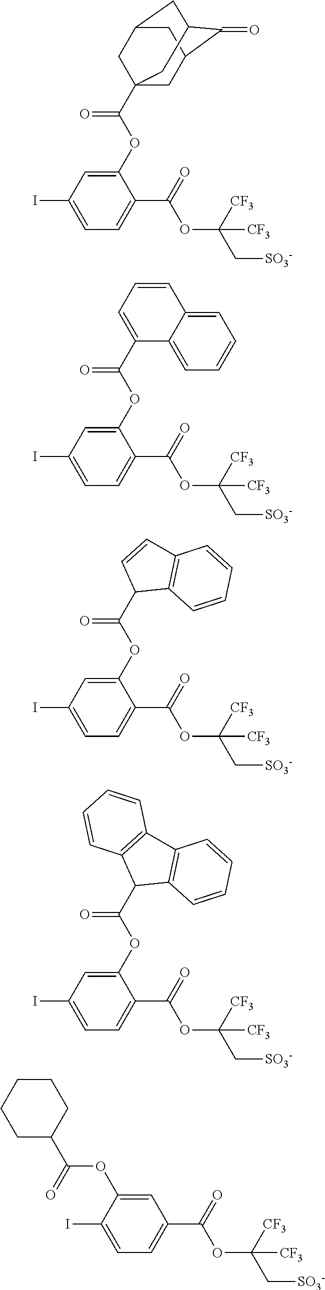

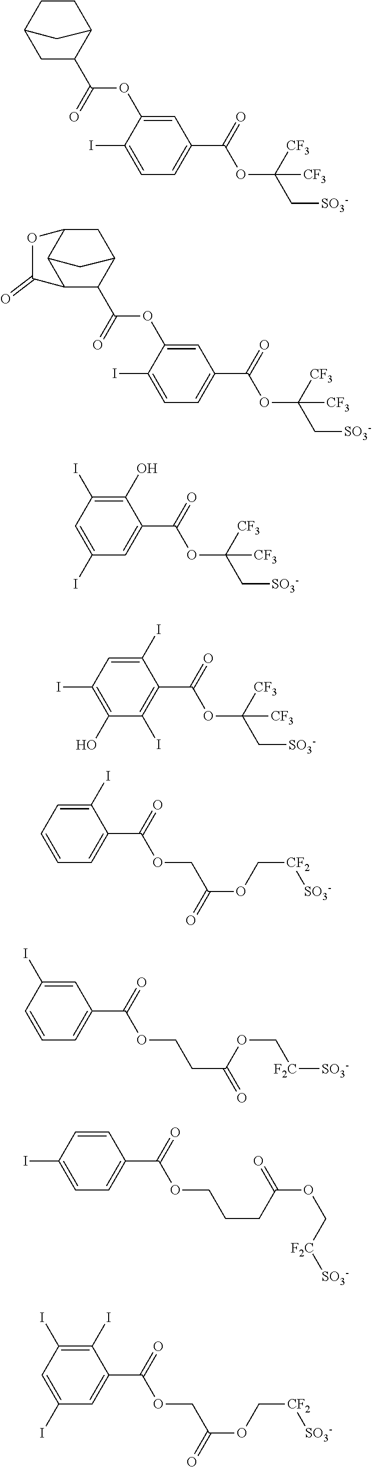

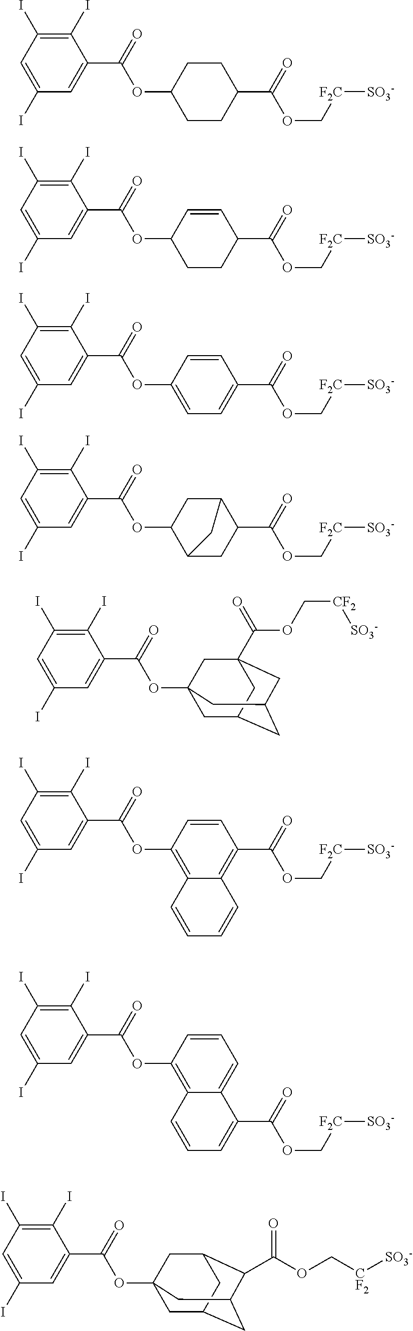

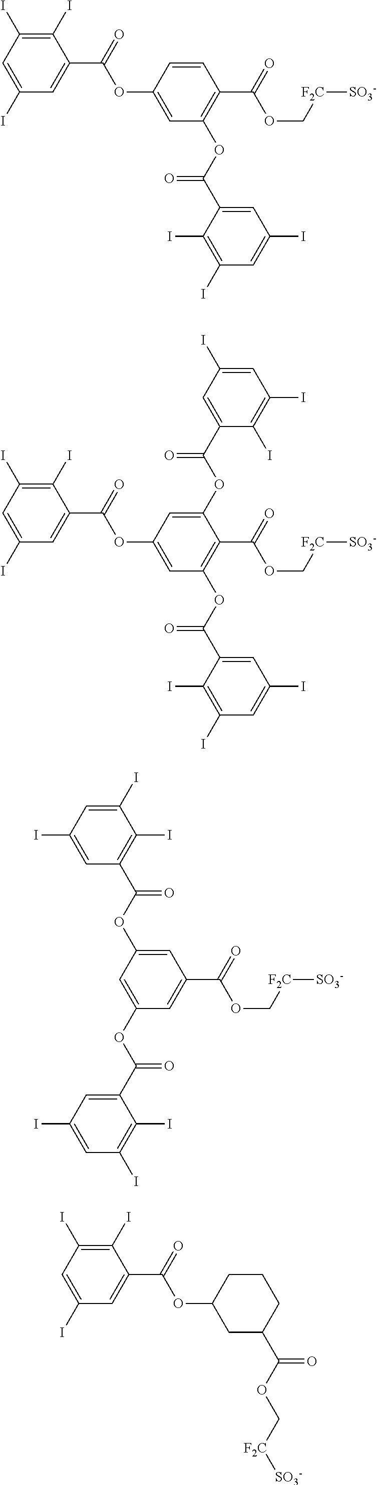

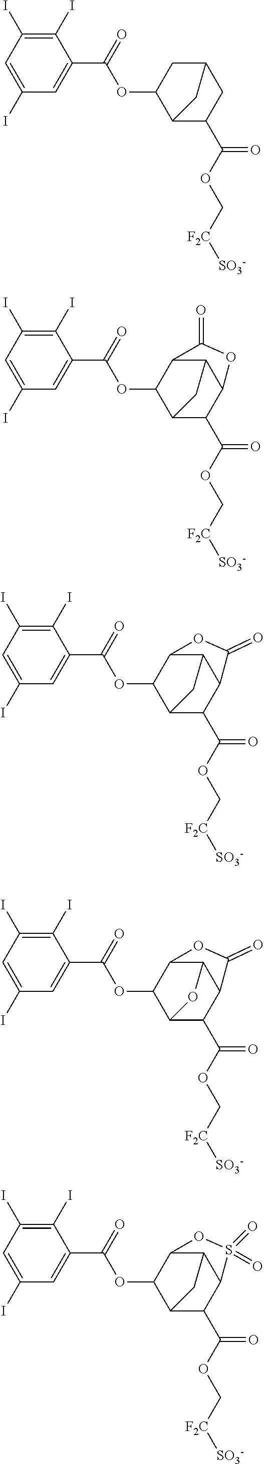

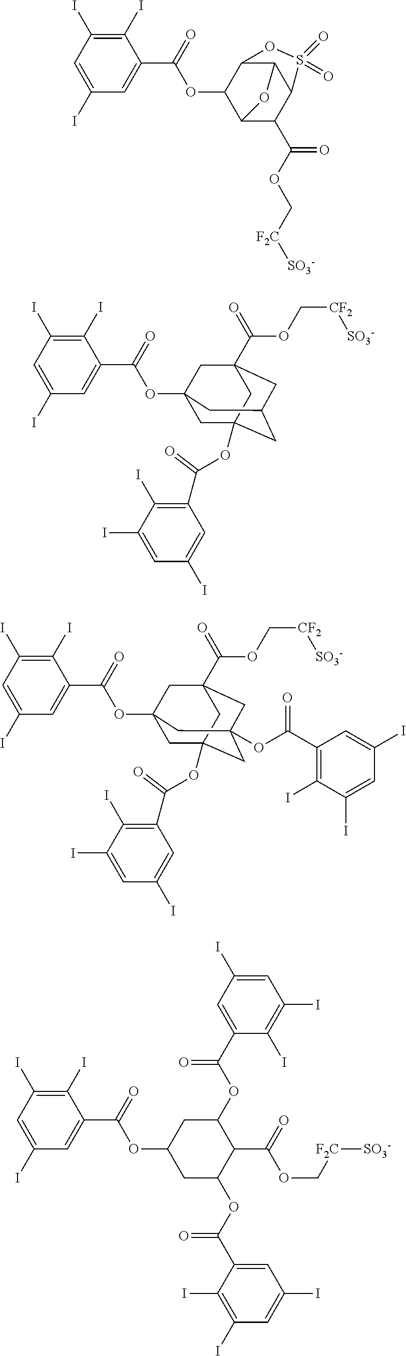

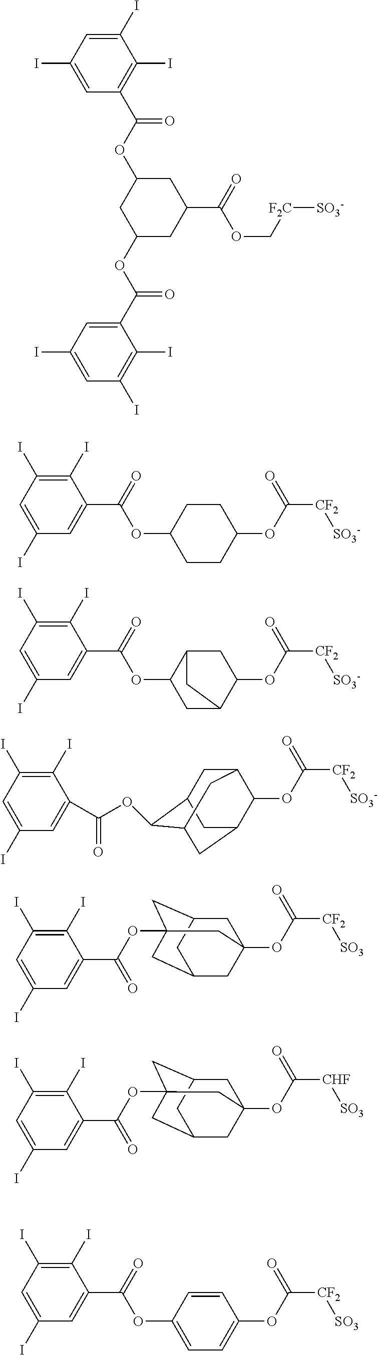

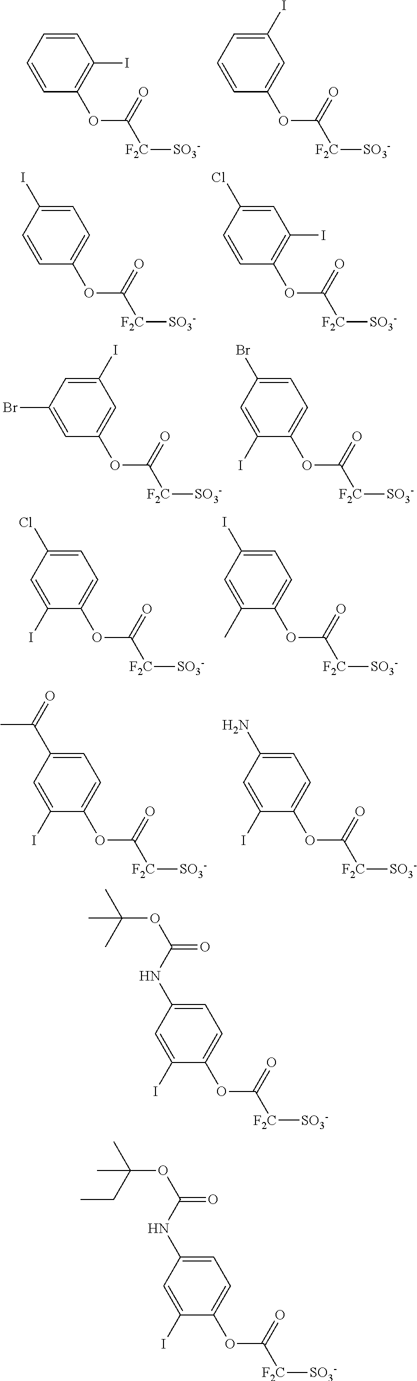

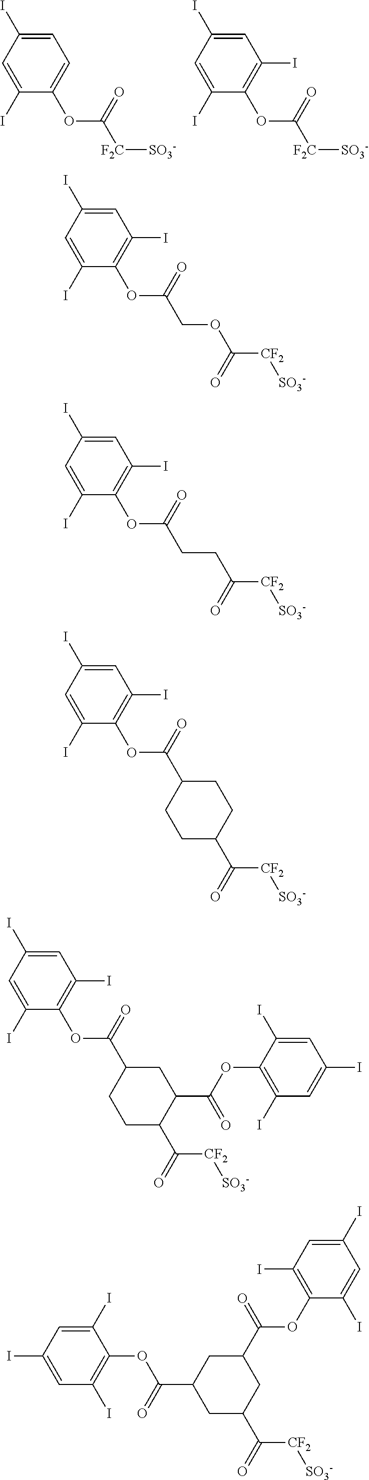

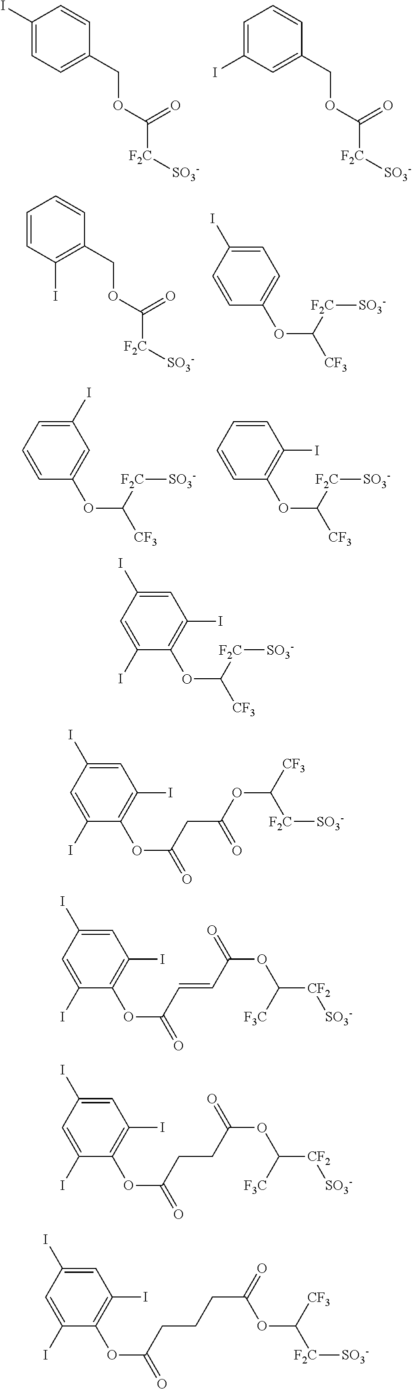

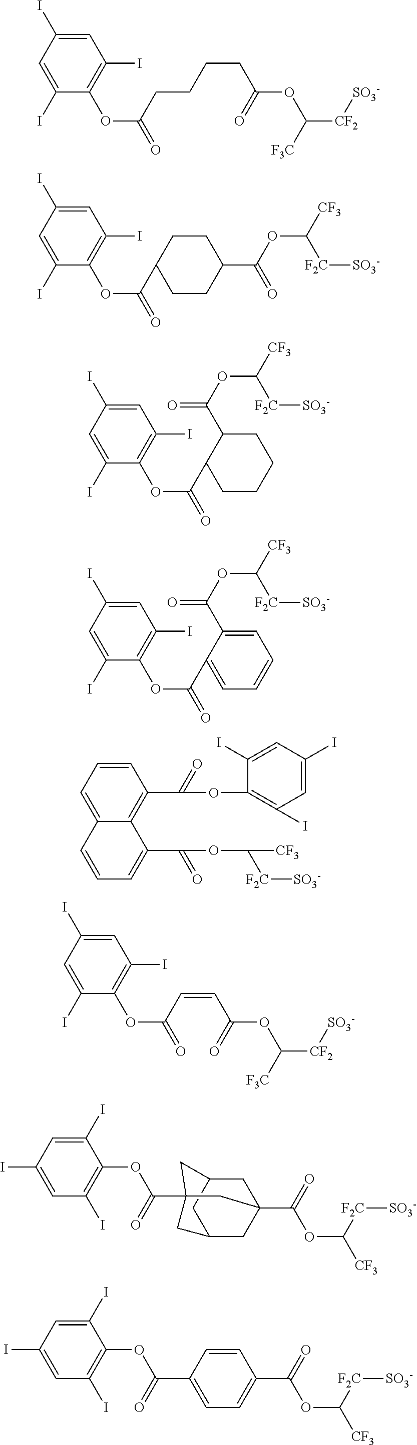

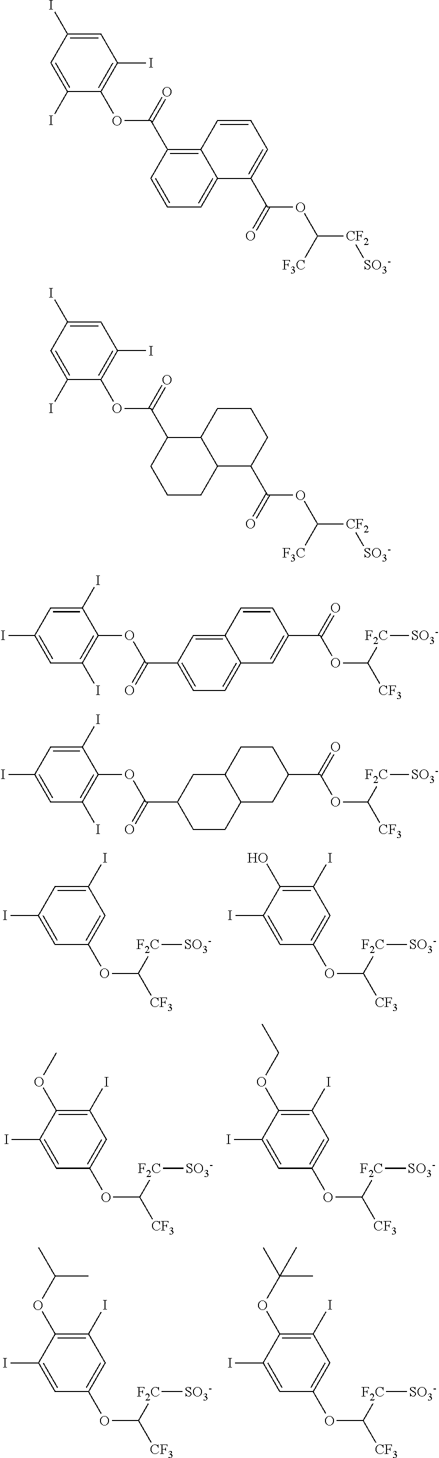

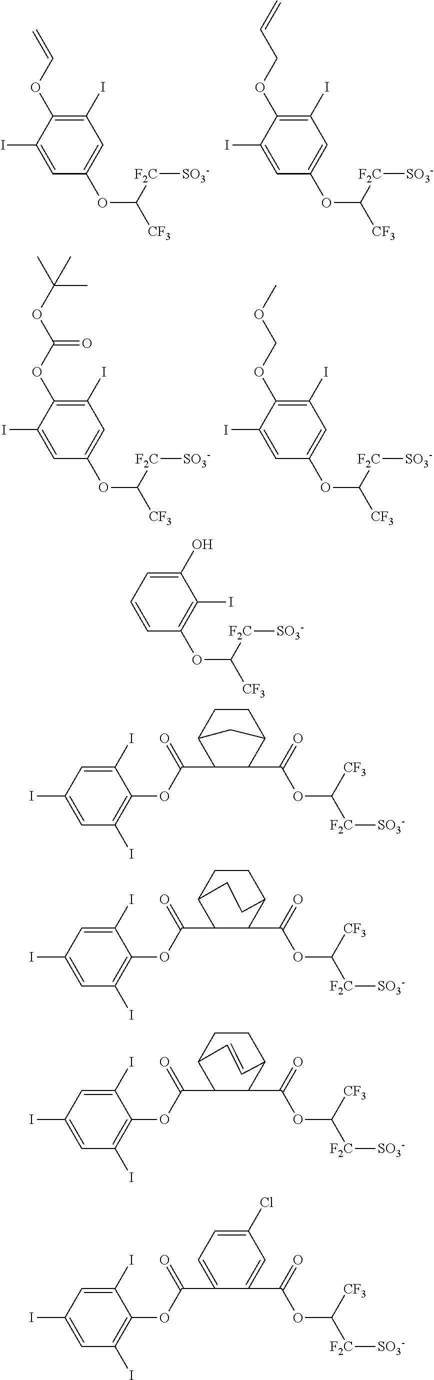

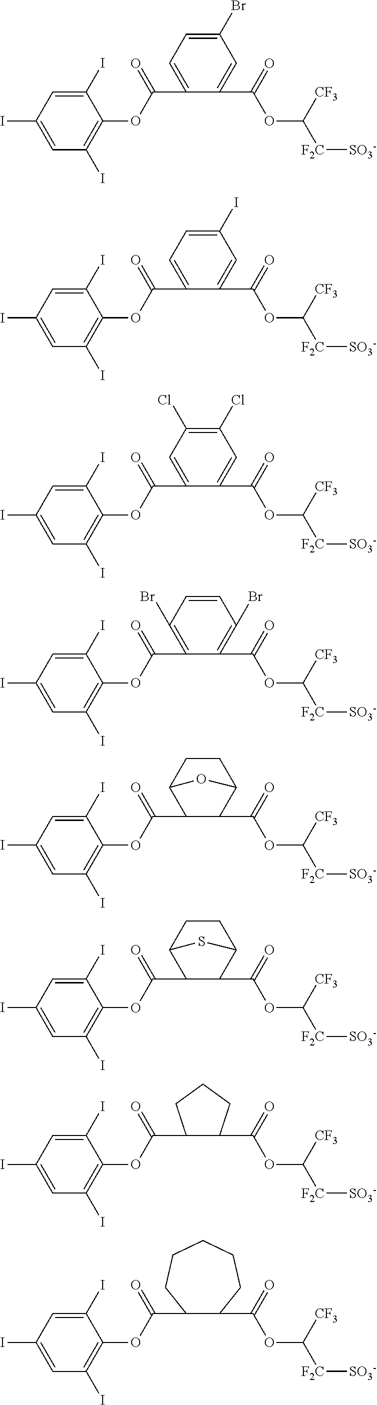

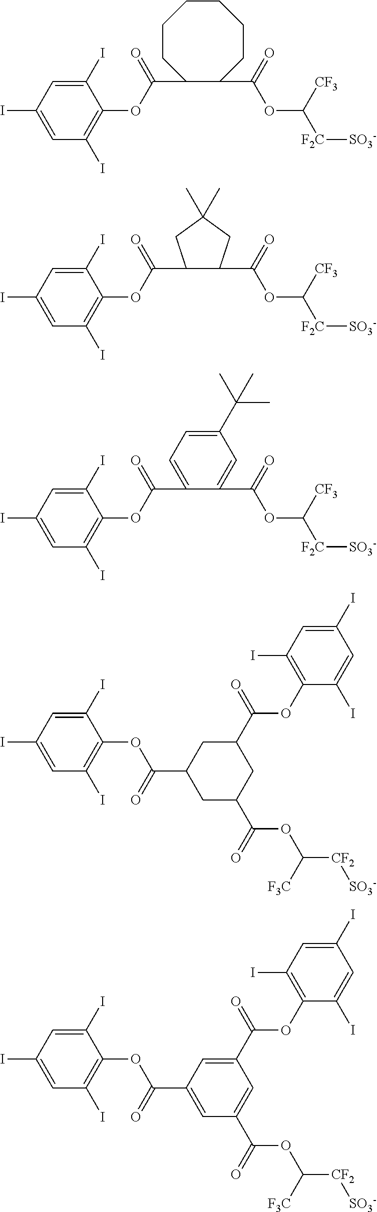

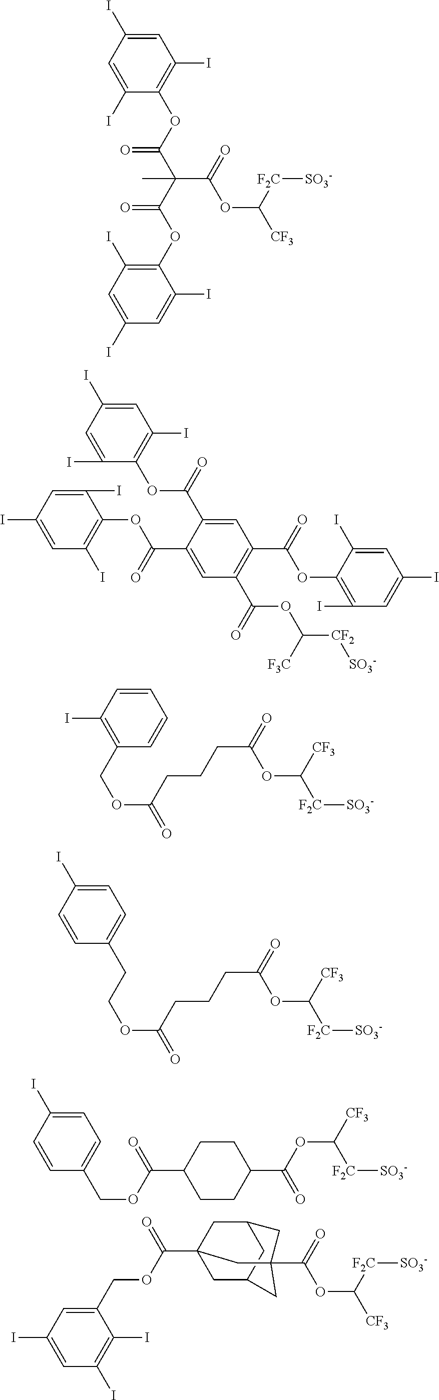

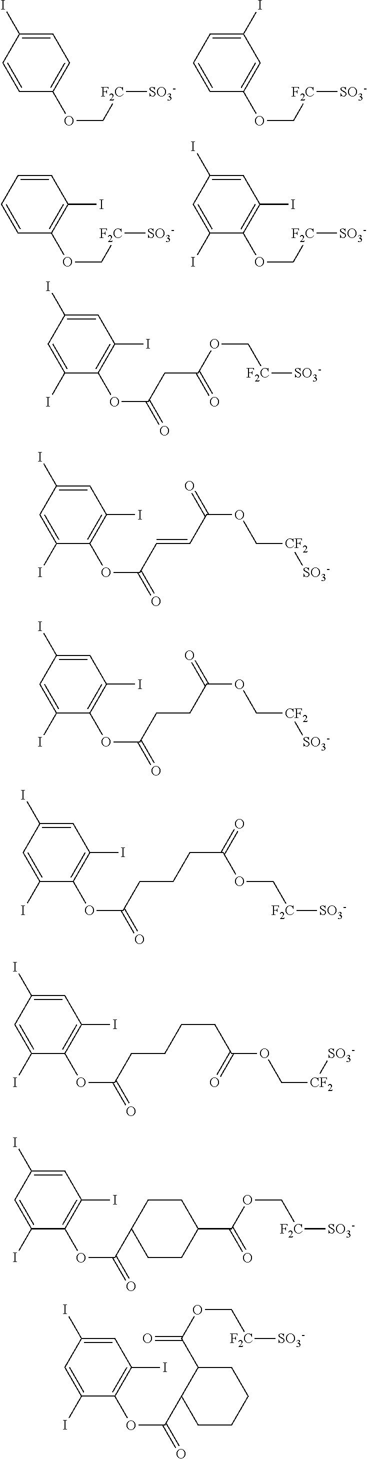

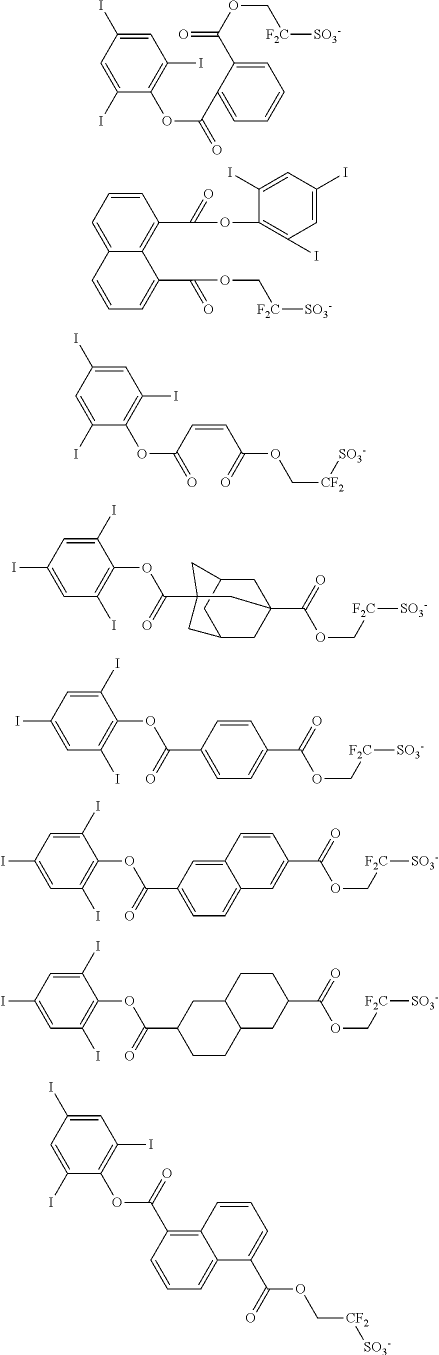

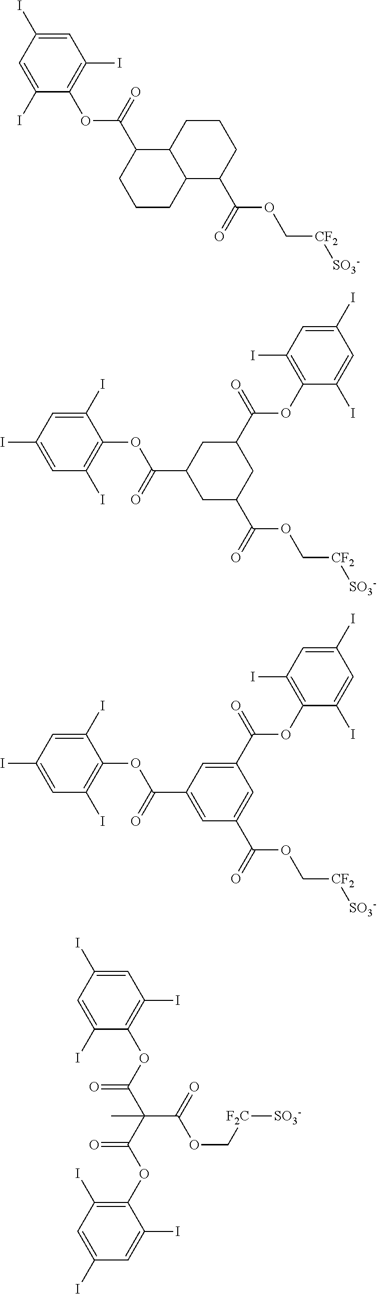

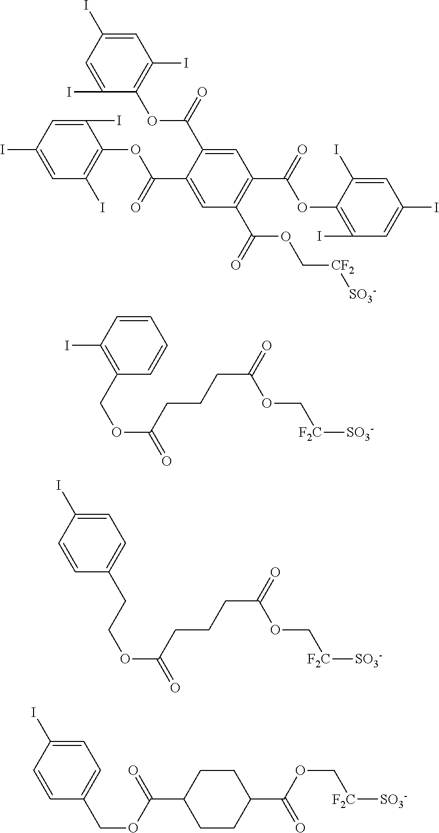

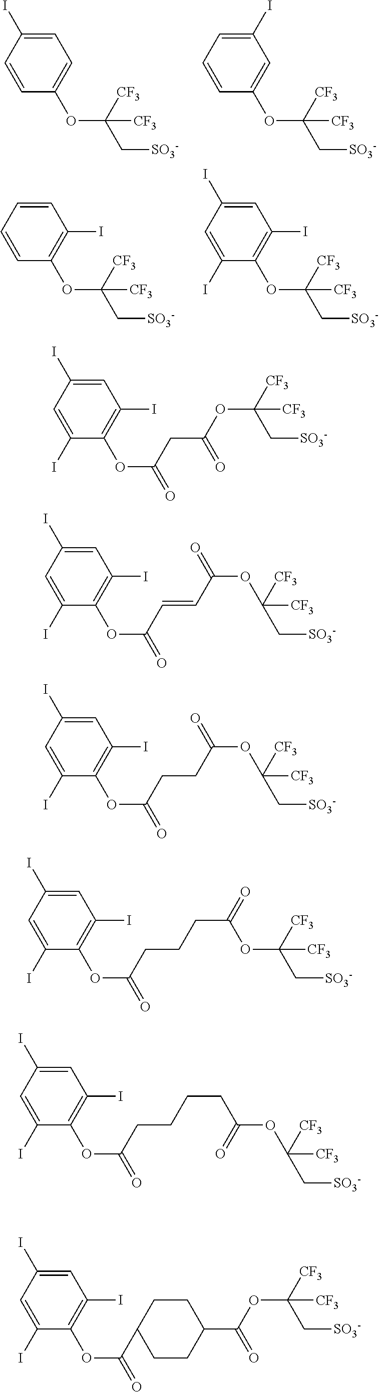

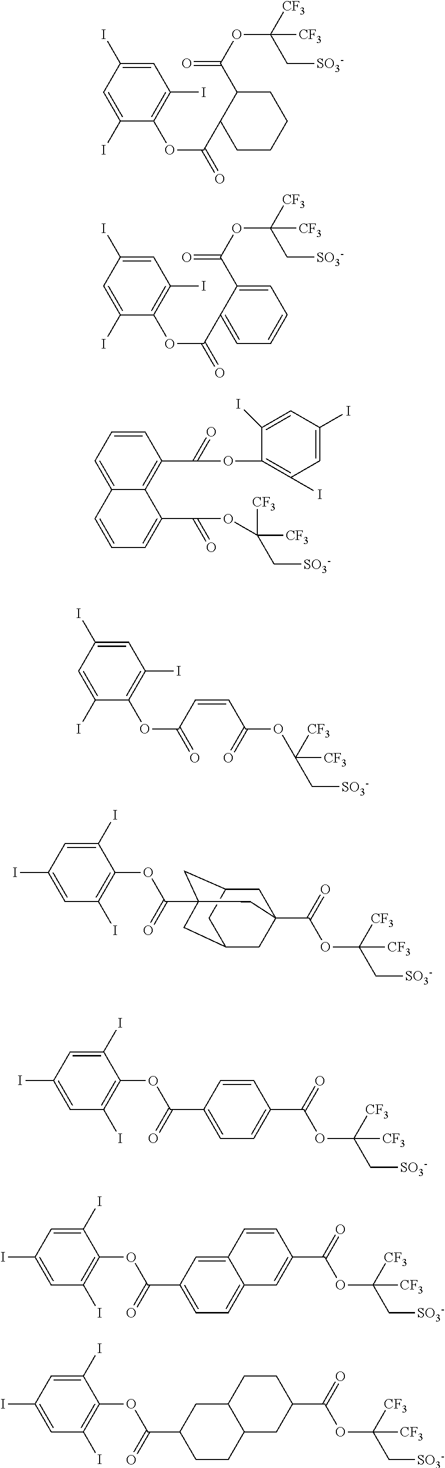

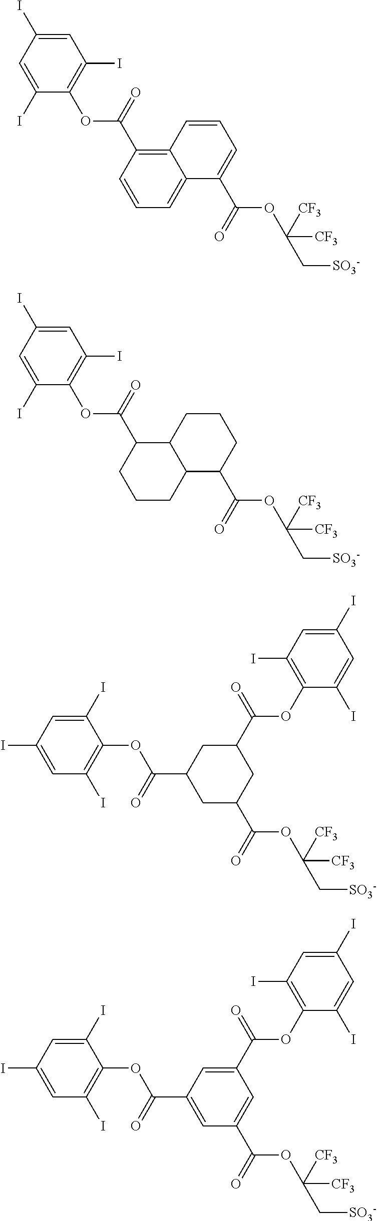

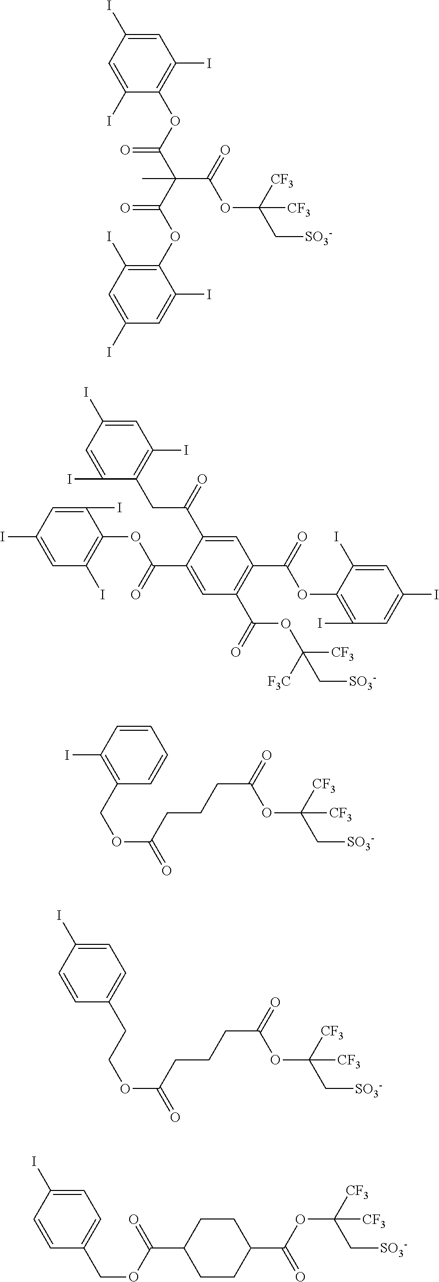

When the inventive chemically amplified resist composition comprises acid generator (D), the amount of the acid generator (D) used is preferably 0.1 to 40 parts by weight, more preferably 0.5 to 20 parts by weight per 80 parts by weight of the base polymer (A). An amount of the acid generator (D) in the range ensures good resolution and eliminates the risk of leaving foreign matter after development or during separation of resist film. The acid generator (D) may be used alone or in admixture.

### (E) Surfactant

The inventive chemically amplified resist composition may further comprise (E) a surfactant. It is typically (E) a surfactant which is insoluble or substantially insoluble in water and alkaline developer, or a surfactant which is insoluble or substantially insoluble in water and soluble in alkaline developer. For the surfactant, reference should be made to those compounds described in JP-A 2010-215608 and JP-A 2011-16746.

While many examples of the surfactant which is insoluble or substantially insoluble in water and alkaline developer are described in the patent documents cited herein, preferred examples are fluorochemical surfactants FC-4430 (3M), Olfine® E1004 (Nissin Chemical Co., Ltd.), Surflon® S-381, KH-20 and KH-30 (AGC Seimi Chemical Co., Ltd.). Partially fluorinated oxetane ring-opened polymers having the formula (surf-1) are also useful.

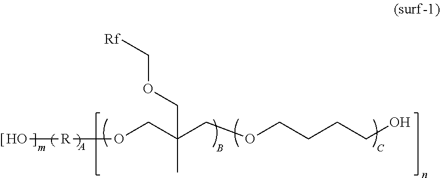

It is provided herein that R, Rf, A, B, C, m, and n are applied to only formula (surf-1), independent of the above descriptions. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

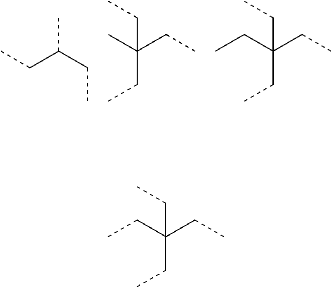

Herein the broken line denotes a valence bond. These formulae are partial structures derived from glycerol, trimethylol ethane, trimethylol propane, and pentaerythritol, respectively.

Of these, 1,4-butylene and 2,2-dimethyl-1,3-propylene are preferred.

Rf is trifluoromethyl group or pentafluoroethyl group, preferably trifluoromethyl group. The subscript m is an integer of 0 to 3, n is an integer of 1 to 4, and the sum of m and n, which represents the valence of R, is an integer of 2 to 4. A is 1. B is an integer of 2 to 25, preferably an integer of 4 to 20. C is an integer of 0 to 10, preferably 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the surface of a resist film for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign matter which becomes defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Examples of the polymeric surfactant include those containing repeat units of at least one type selected from the formulae (6A) to (6E).

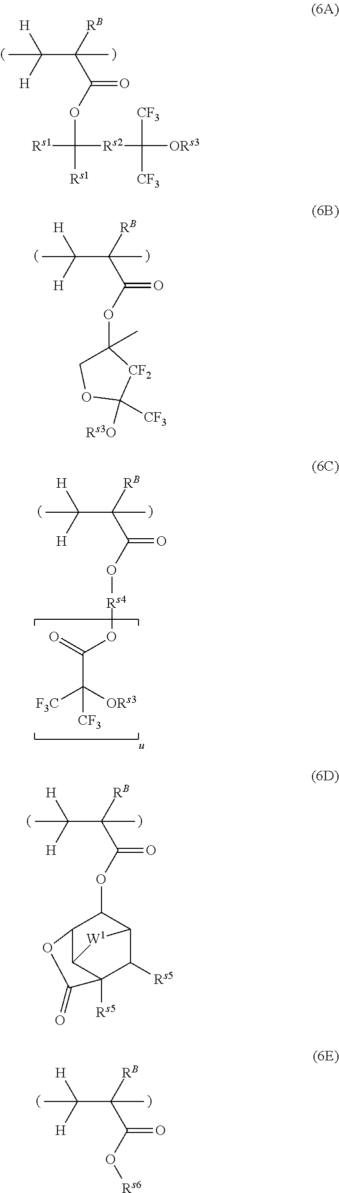

In formulae (6A) to (6E), R^{B} is a hydrogen atom, a fluorine atom, a methyl group, or a trifluoromethyl group. W¹ is —CH₂—, —CH₂CH₂— or —O—, or two separate —H. R^{s1} is each independently a hydrogen atom or a C₁-C₁₀ hydrocarbyl group. R^{s2} is a single bond or a C₁-C₅ straight or branched hydrocarbylene group. R^{s3} is each independently a hydrogen atom, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group. u is an integer of 1 to 3. R^{s5} is each independently hydrogen atom or a group: —C(═O)—O—R^{sa}. R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The C₁-C₁₀ hydrocarbyl group R^{s1} is preferably saturated while it may be straight, branched or cyclic. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl and n-decyl, and C₃-C₁₀ cyclic saturated hydrocarbyl groups such as cyclopropyl, cyclobutyl, cyclopentyl, cyclohexyl, adamantyl and norbornyl. Inter alia, C₁-C₆ hydrocarbyl groups are preferred.

The hydrocarbylene group R^{s2} is preferably saturated while it may be straight, branched or cyclic. Examples thereof include methylene, ethylene, propylene, butylene and pentylene groups.

The hydrocarbyl group R^{s3} or R^{s6} may be saturated or unsaturated and straight, branched or cyclic. Examples thereof include saturated hydrocarbyl groups and aliphatic unsaturated hydrocarbyl groups such as alkenyl and alkynyl groups, with the saturated hydrocarbyl groups being preferred. Examples of the saturated hydrocarbyl groups include those exemplified for the hydrocarbyl group R^{s1} as well as undecyl, dodecyl, tridecyl, tetradecyl, and pentadecyl groups. Examples of the fluorinated hydrocarbyl group R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group R^{s3} include the groups of formulae (AL-3) to (AL-5), trialkylsilyl groups in which each alkyl group is a C₁-C₆ alkyl group, and C₄-C₂₀ oxoalkyl groups.

The (u+1)-valent hydrocarbon or fluorinated hydrocarbon group R^{s4} may be straight, branched or cyclic, and examples thereof include the foregoing hydrocarbyl or fluorinated hydrocarbyl groups from which “u” number of hydrogen atoms are eliminated.

The fluorinated hydrocarbyl group R^{sa} is preferably saturated while it may be straight, branched or cyclic. Examples thereof include the foregoing hydrocarbyl groups in which some or all hydrogen atoms are substituted by fluorine atoms. Illustrative examples of the groups include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units of formulae (6A) to (6E) are shown below, but not limited thereto. Herein R^{B} is as defined above.

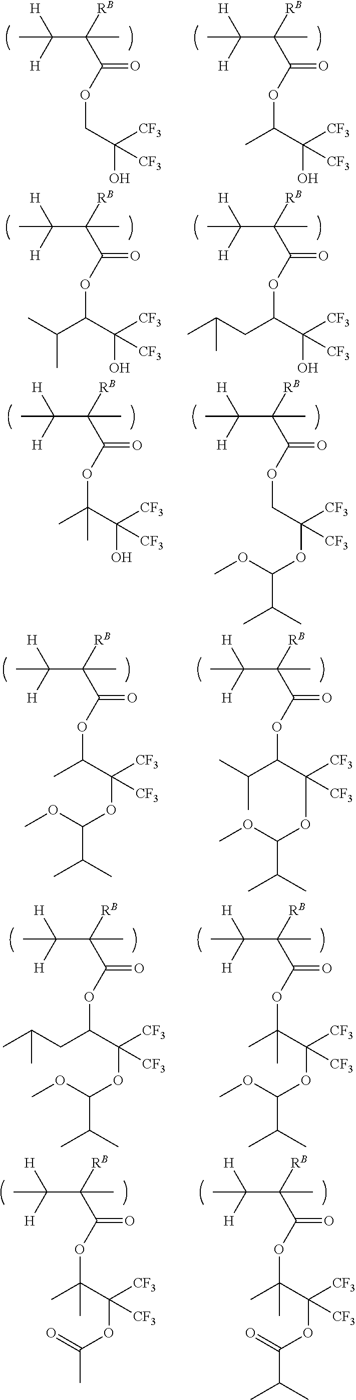

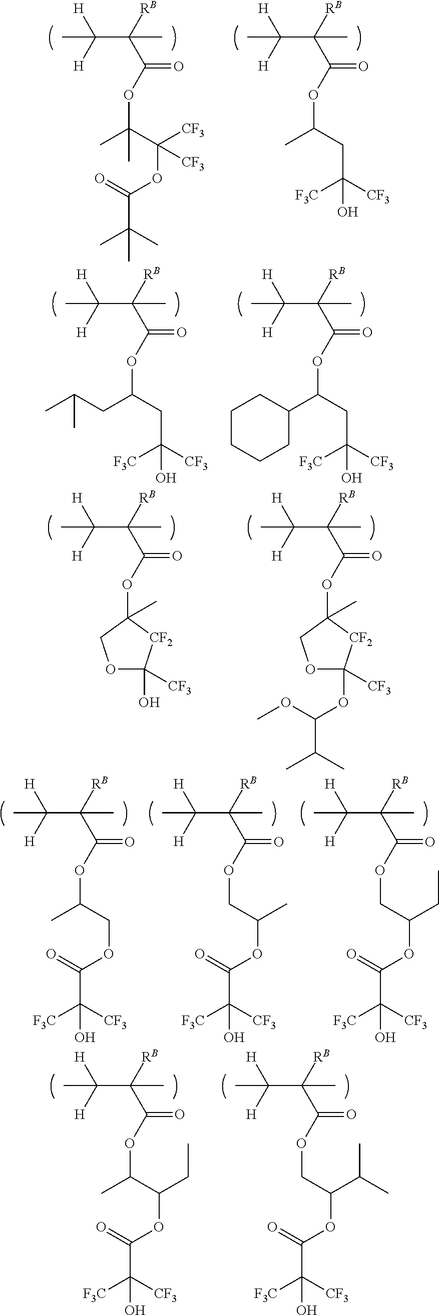

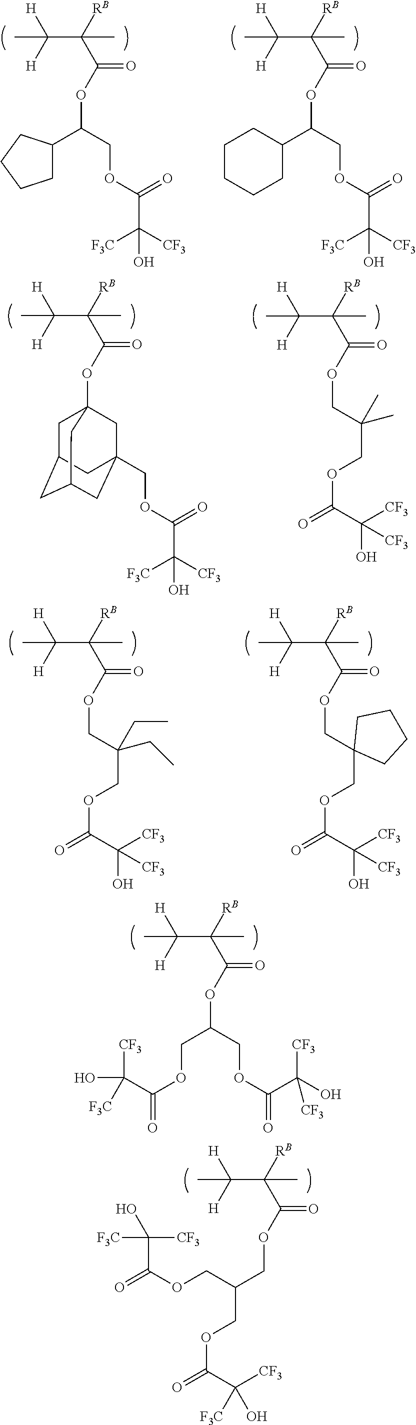

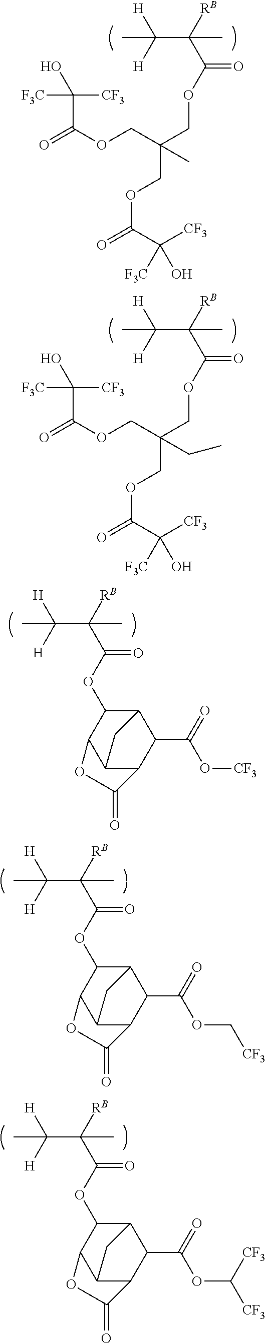

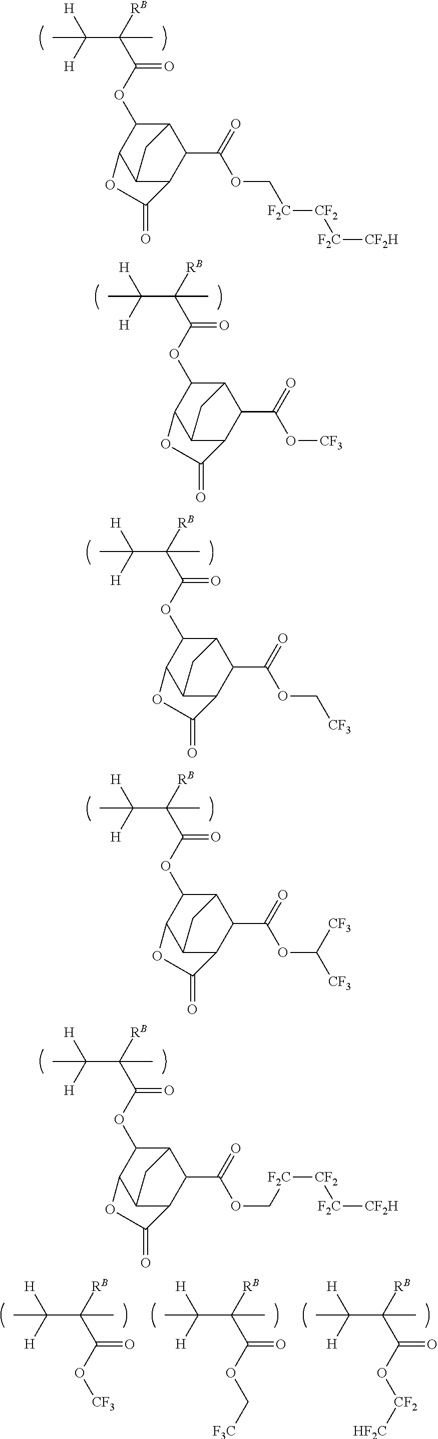

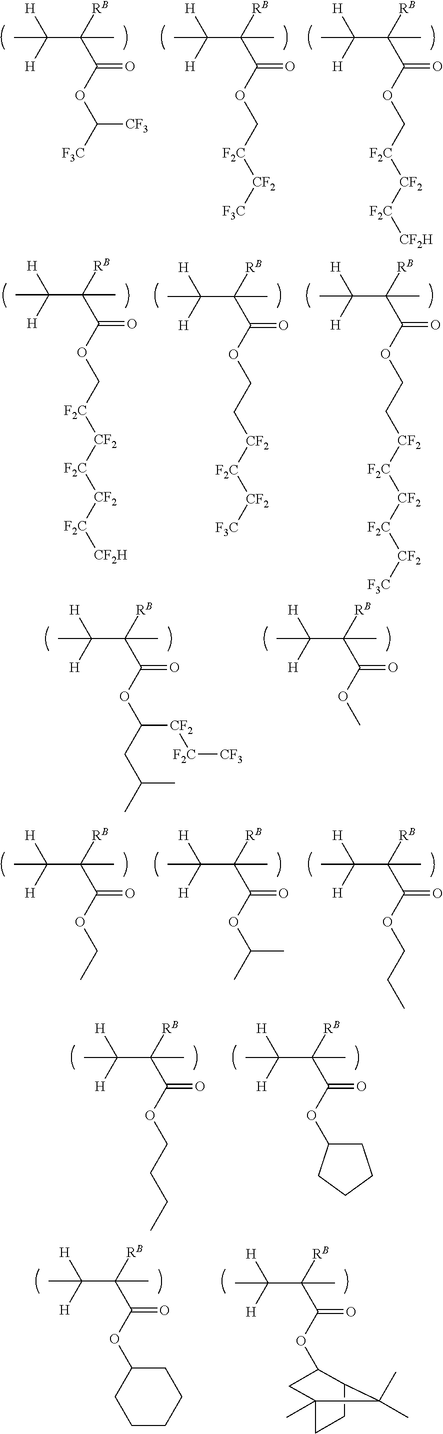

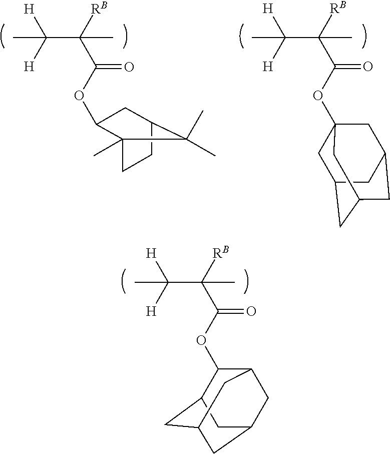

The polymeric surfactant may further contain repeat units other than the repeat units having formulae (6A) to (6E). Typical other repeat units are, for example, those derived from methacrylic acid and α-trifluoromethylacrylic acid derivatives. In the polymeric surfactant, the content of the repeat units having formulae (6A) to (6E) is preferably at least 20 mol %, more preferably at least 60 mol %, most preferably 100 mol % of the overall repeat units.

Mw of the polymeric surfactant is preferably 1,000 to 500,000, more preferably 3,000 to 100,000. Mw/Mn is preferably 1.0 to 2.0, more preferably 1.0 to 1.6.

The polymeric surfactant may be synthesized, for example, by dissolving an unsaturated bond-containing monomer or monomers, from which repeat units having formulae (6A) to (6E) and optional other repeat units are derived, in an organic solvent, adding a radical initiator, and heating for polymerization. Examples of the suitable organic solvent used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such, or the polymerization may be followed by protection or partial protection.

During the synthesis of the polymeric surfactant, any of well-known chain transfer agents such as dodecylmercaptan and 2-mercaptoethanol may be used for the purpose of adjusting molecular weight. An appropriate amount of the chain transfer agent is 0.01 to 10 mol % based on the total moles of monomers to be polymerized.

When the chemically amplified resist composition contains the surfactant (E), the amount of the surfactant (E) used is 0.1 to 50 parts by weight, more preferably 0.5 to 10 parts by weight per 80 parts by weight of the base polymer (A). As long as the amount of the surfactant (E) is at least 0.1 parts by weight, the receding contact angle of resist film surface with water is fully improved. As long as the amount of the surfactant (E) is up to 50 parts by weight, the dissolution rate of resist film surface in developer is so low that the resulting small-size pattern may maintain a sufficient height. The surfactant (E) may be used alone or in admixture.

### (F) Dissolution Inhibitor

The inventive chemically amplified resist composition may further comprise (F) a dissolution inhibitor. The inclusion of a dissolution inhibitor in the positive chemically amplified resist composition of the present invention may lead to an increased difference in dissolution rate between exposed and unexposed areas and a further improvement in resolution.

The dissolution inhibitor which can be used herein is, for example, a compound having at least two phenolic hydroxy groups on the molecule, in which an average of from 0 to 100 mol % of all the hydrogen atoms on the phenolic hydroxy groups are replaced by acid labile groups or a compound having at least one carboxy group on the molecule, in which an average of 50 to 100 mol % of all the hydrogen atoms on the carboxy groups are replaced by acid labile groups, both the compounds having a molecular weight of 100 to 1,000, and preferably 150 to 800. Typical are bisphenol A, trisphenol, phenolphthalein, cresol novolac, naphthalenecarboxylic acid, adamantanecarboxylic acid, and cholic acid derivatives in which the hydrogen atom on the hydroxy or carboxy group is substituted by an acid labile group, as described in JP-A 2008-122932, paragraphs [0155]-[0178].

When the inventive chemically amplified resist composition comprises the dissolution inhibitor (F), the amount of the dissolution inhibitor (F) used is preferably 0 to 50 parts by weight, more preferably 5 to 40 parts by weight per 80 parts by weight of the base polymer (A).

### (G) Other Components

The inventive chemically amplified resist composition may further comprise (G) other components, for example, a compound which is decomposed with an acid to generate another acid (i.e., acid amplifier compound), an organic acid derivative, fluorinated alcohol, and a water repellency improver. The acid amplifier compound is described in JP-A 2009-269953 and JP-A 2010-215608. The acid amplifier compound is preferably used in an amount of 0 to 5 parts, more preferably 0 to 3 parts by weight per 80 parts by weight of the base polymer (A). An extra amount of the acid amplifier compound can make the acid diffusion control difficult and cause degradations to resolution and pattern profile. With respect to the organic acid derivative and fluorinated alcohol, reference should be made to JP-A 2009-269953 and JP-A 2010-215608.

The water repellency improver may be used in the topcoatless immersion lithography. Suitable water repellency improvers include polymers having a fluoroalkyl group and polymers having a specific structure with a 1,1,1,3,3,3-hexafluoro-2-propanol residue and are described in JP-A 2007-297590 and JP A 2008-111103, for example. The water repellency improver should be soluble in alkaline developers and organic solvent developers. The water repellency improver of specific structure with a 1,1,1,3,3,3-hexafluoro-2-propanol residue is well soluble in the developer. A polymer having an amino group or amine salt copolymerized as repeat units may serve as the water repellency improver and is effective for preventing evaporation of acid during PEB, thus preventing any hole pattern opening failure after development. When the inventive chemically amplified resist composition comprises the water repellency improved, the amount of the water repellency improver used is preferably 0 to 20 parts by weight, more preferably 0.5 to 10 parts by weight per 80 parts by weight of the base polymer (A).

### [Pattern Forming Process]

The inventive chemically amplified resist composition is used in the fabrication of various integrated circuits. Pattern formation using the chemically amplified resist composition may be performed by well-known lithography processes. The process generally involves the steps of applying the chemically amplified resist composition onto a substrate to form a resist film thereon, exposing the resist film to high-energy radiation, and developing the exposed resist film in a developer.

The inventive chemically amplified resist composition is first applied onto a substrate on which an integrated circuit is to be formed (e.g., Si, SiO₂, SiN, SiON, TiN, WSi, BPSG, SOG, or organic antireflective coating) or a substrate on which a mask circuit is to be formed (e.g., Cr, CrO, CrON, MoSi₂, or SiO₂) by a suitable coating technique such as spin coating, roll coating, flow coating, dipping, spraying or doctor coating. The resulting resist film is generally 0.01 to 2.0 μm thick. The coating is prebaked on a hotplate preferably at a temperature of 60 to 150° C. for 10 seconds to 30 minutes, and more preferably at 80 to 120° C. for 30 seconds to 20 minutes to form a resist film.

The resist film is then exposed to high-energy radiation. Examples of the high-energy radiation include UV, deep-UV, EB, EUV of wavelength 3-15 nm, i-line, x-ray, soft x-ray, excimer laser light, γ-ray or synchrotron radiation. When UV, deep-UV, EUV, x-ray, soft x-ray, excimer laser light, γ-ray or synchrotron radiation is used as the high-energy radiation, the resist film is exposed thereto directly or through a mask having a desired pattern in a dose of preferably about 1 to 200 mJ/cm², more preferably about 10 to 100 mJ/cm². When EB is used as the high-energy radiation, the resist film is exposed thereto directly or through a mask having a desired pattern in a dose of preferably about 0.1 to 100 μC/cm², more preferably about 0.5 to 50 μC/cm². It is appreciated that the inventive chemically amplified resist composition is suited in micropatterning using ArF excimer laser with a wavelength 193 nm, KrF excimer laser with a wavelength 248 nm, EB, EUV with a wavelength 3 to 15 nm, x-ray, soft x ray, γ-ray or synchrotron radiation as the high-energy radiation.

After the exposure, the resist film may be baked (PEB) on a hotplate preferably at 60 to 150° C. for 10 seconds to 30 minutes, more preferably at 80 to 120° C. for 30 seconds to 20 minutes.

After the exposure or PEB, the resist film is developed in a developer in the form of an aqueous base solution for 3 seconds to 3 minutes, preferably 5 seconds to 2 minutes by conventional techniques such as dip, puddle and spray techniques. A typical developer is a 0.1 to 10 wt %, preferably 2 to 5 wt % aqueous solution of tetramethylammonium hydroxide (TMAH), tetraethylammonium hydroxide, tetrapropylammonium hydroxide or tetrabutylammonium hydroxide. The resist film in the exposed area is dissolved in the developer whereas the resist film in the unexposed area is not dissolved. In this way, the desired positive pattern is formed on the substrate.

In formation of the negative pattern, an organic solvent developer may be used instead of the aqueous base solution. Specific examples of the developer used herein include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. The organic solvents may be used alone or in admixture.

At the end of development, the resist film may be rinsed. As the rinsing liquid, a solvent which is miscible with the developer and does not dissolve the resist film is preferred. Suitable solvents include C₃-C₁₀ alcohols, C₈-C₁₂ ether compounds, and C₆-C₁₂ alkanes, alkenes and alkynes, and aromatic solvents.

Specific examples of the alcohols having 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol.

Specific examples of the ether compounds having 8 to 12 carbon atoms include di-n-butyl ether, diisobutyl ether, di-sec-butyl ether, di-n-pentyl ether, diisopentyl ether, di-sec-pentyl ether, di-tert-pentyl ether, and di-n-hexyl ether.

Specific examples of the alkanes having 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Specific examples of the C₆-C₁₂ alkene include hexene, heptene, octene, cyclohexene, methylcyclohexene, dimethylcyclohexene, cycloheptene, and cyclooctene. Specific examples of the C₆-C₁₂ alkyne include hexyne, heptyne, and octyne.

Specific examples of the aromatic solvents include toluene, xylene, ethylbenzene, isopropylbenzene, tert-butylbenzene and mesitylene.

Rinsing is effective for reducing the resist pattern collapse and defect formation. However, rinsing is not essential. If rinsing is omitted, the amount of solvent used may be reduced.

A hole or trench pattern after development may be shrunk by the thermal flow, RELACS®, or DSA process. A hole pattern is shrunk by applying a shrink agent thereto, and baking the resist composition such that the shrink agent may undergo crosslinking at the resist film surface due to diffusion of the acid catalyst from the resist film during baking, and the shrink agent may attach to the sidewall of the hole pattern. The baking temperature is preferably 70 to 180° C., more preferably 80 to 170° C., and the baking time is preferably 10 to 300 seconds to remove the excess shrink agent and shrink the hole pattern.

### EXAMPLES

Hereinafter, the present invention is specifically described with reference to a Synthesis Example, Examples, and Comparative Examples, but the present invention is not limited to the following Examples. The apparatuses used are as follows.
MALDI TOF-MS: S3000 manufactured by JEOL Ltd.

### [1] Synthesis of Onium Salt Type Monomers

### [Example 1-1] Synthesis of Onium Salt Type Monomer a-1

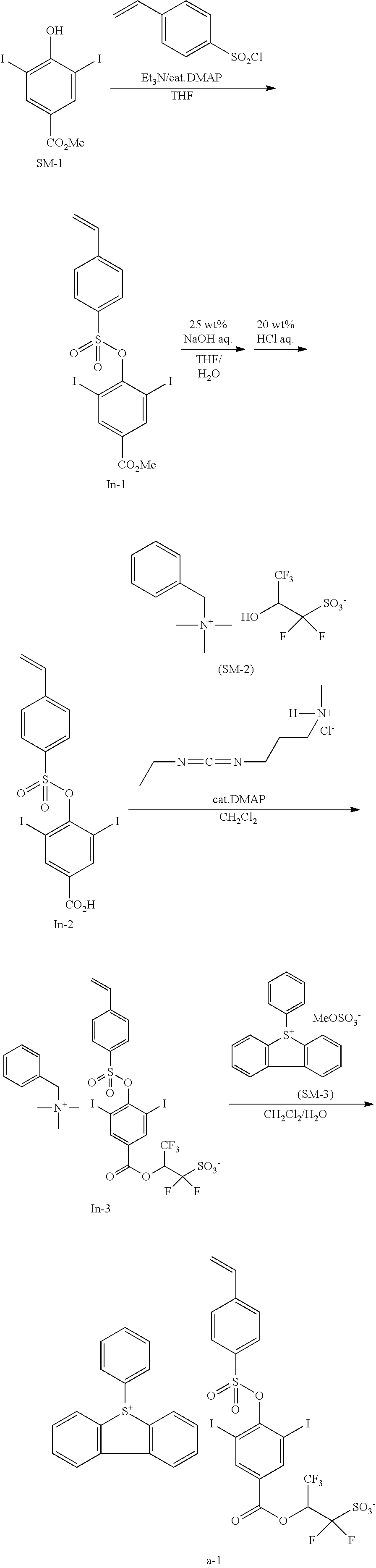

### (1) Synthesis of Intermediate In-1

In a reactor under nitrogen atmosphere, 37.2 g of reactant SM-1, 18.0 g of triethylamine and 1.50 g of 4-dimethylaminopyridine were dissolved or suspended in 200 g of THF, followed by cooling in an ice bath. Subsequently, a solution obtained by dissolving 16.4 g of p-styrenesulfonyl chloride in 50 g of THF was added dropwise. After addition, the reactor was warmed up to an internal temperature of 50° C., and the reaction solution was aged for 6 hours. Subsequently, the reaction solution was cooled, and 100 g of saturated sodium hydrogencarbonate was added to quench the reaction. The reaction product was extracted twice with 200 g of ethyl acetate, followed by ordinary aqueous work-up and solvent distillation. Subsequent purification by silica gel chromatography gave 45.8 g (yield 97%) of Intermediate In-1 as oily matter.

### (2) Synthesis of Intermediate In-2

Under nitrogen atmosphere, 45.8 g of Intermediate In-1 was dissolved in 200 g of THF. 15.5 g of a 25 wt % sodium hydroxide aqueous solution was added dropwise while the reactor was cooled in an ice bath. After addition, the reactor was warmed up to an internal temperature of 30° C., and the reaction solution was aged for 12 hours. After aging, the reaction solution was cooled, and 18.2 g of 20 wt % hydrochloric acid was added dropwise to quench the reaction. The reaction product was extracted twice with 200 g of ethyl acetate, followed by ordinary aqueous work-up and solvent distillation. Subsequent recrystallization with hexane gave 37.5 g (yield 90%) of Intermediate In-2 as white crystals.

### (3) Synthesis of Intermediate In-3

Under nitrogen atmosphere, a reactor was charged with 37.5 g of Intermediate In-2, 34.8 g of reactant SM-2, 1.1 g of 4-dimethylaminopyridine and 150 g of methylene chloride, and cooled in an ice bath. 20.1 g of 1-Ethyl-3-(3-dimethylaminopropyl)carbodiimide hydrochloride powder was added while the reactor was kept at an internal temperature of 20° C. or lower. After addition, the reaction mixture was warmed up to room temperature, and aged for 12 hours. After aging, 100 g of water was added to quench the reaction, followed by ordinary aqueous work-up and solvent distillation. Subsequent recrystallization by addition of diisopropyl ether gave 61.5 g (yield 89%) of Intermediate In-3 as oily matter.

### (4) Synthesis of Onium Salt Type Monomer a-1

Under nitrogen atmosphere, a reactor was charged with 61.5 g of Intermediate In-3, 31.8 g of reactant SM-3, 200 g of methylene chloride and 150 g of water, and the mixture was stirred for 30 minutes. Subsequently, the organic layer was taken out, washed with water, and then concentrated under reduced pressure. Purification of the concentrate by silica gel chromatography gave 61.0 g (yield 87%) of the titled onium salt type monomer a-1 as oily matter.
MALDI TOF-MS:
POSITIVE M⁺261 (corresponding to C₁₈H₁₃S⁺)
NEGATIVE M-766 (corresponding to C₁₈H₁₀F₅I₂O₈S₂⁻)

### [Examples 1-2 to 1-7] Synthesis of Onium Salt Type Monomers a-2 to a-10

Onium Salt Type Monomers a-2 to a-10 of the following formulae were synthesized using the corresponding reactants and well-known organic chemistry reaction.

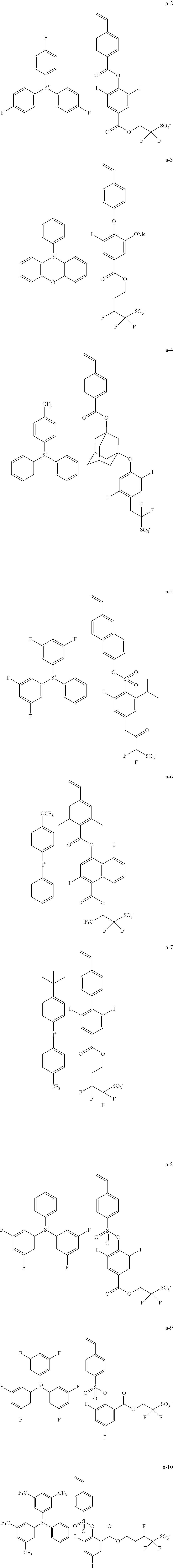

### [Comparative Examples 1-1 to 1-4] Synthesis of Comparative Onium Salt Type Monomers ca-1 to ca-4

Comparative onium salt type monomers ca-1 to ca-4 of the following formulae were synthesized using the corresponding reactants and well-known organic chemistry reaction.

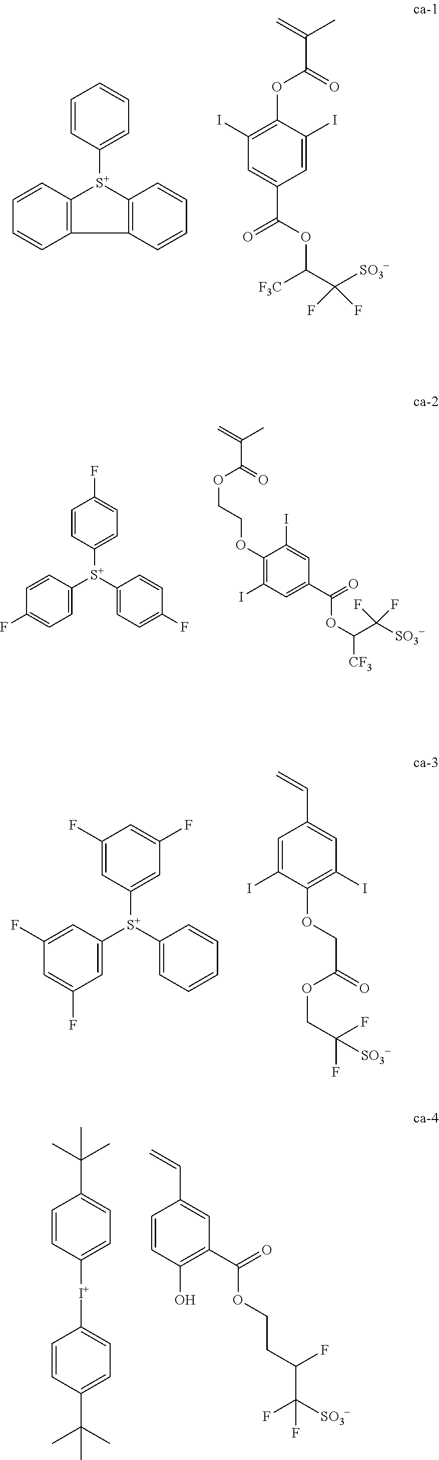

### [2] Synthesis of Base Polymer

Monomers a-1 to a-10, comparative monomers ca-1 to ca-4, and the monomers shown below were used in the synthesis of base polymers.

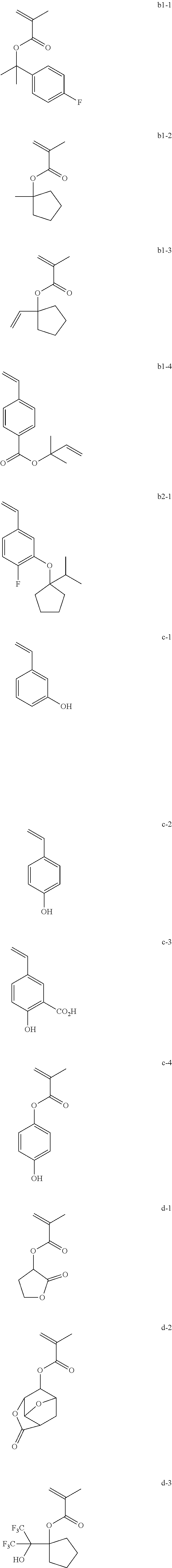

### [Example 2-1] Synthesis of Polymer P-1

A flask under nitrogen atmosphere was charged with 49.4 g of Monomer a-1, 39.3 g of Monomer b1-1, 11.5 g of Monomer c-1, 3.68 g of V-601 (manufactured by Fujifilm Wako Pure Chemical Corp.), and 139 g of MEK to prepare a monomer/initiator solution. Another flask under nitrogen atmosphere was charged with 46 g of MEK, which was heated to 80° C. with stirring. The monomer/initiator solution was added dropwise to the MEK over 4 hours. After the completion of dropwise addition, the polymerization solution was continuously stirred for 2 hours while maintaining the temperature of 80° C., and then cooled to room temperature. The obtained polymerization solution was added dropwise to 3,000 g of hexane with vigorous stirring, and the precipitated polymer was separated by filtration. The precipitate was washed twice with 600 g of hexane and vacuum dried at 50° C. for 20 hours to obtain Polymer P-1 as white powder (amount 97.1 g, yield 97%). Polymer P-1 had a Mw of 9,400 and a Mw/Mn of 1.75. It is noted that Mw is as measured by GPC versus polystyrene standards using DMF solvent.

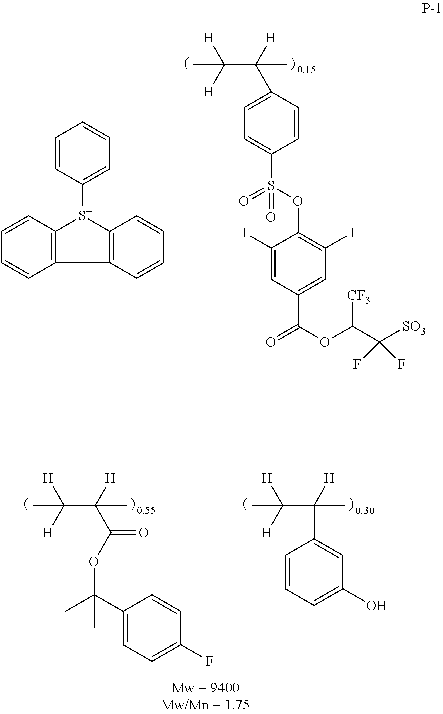

### [Examples 2-2 to 2-23 and Comparative Examples 2-1 to 2-10] Synthesis of Polymers P-2 to P-23 and Comparative Polymers CP-1 to CP-10

Polymers shown in Tables 1 and 2 were prepared by the same procedure as in Example of synthesis 2-1 except that the type and blending ratio of monomers were changed.

TABLE 1

| | | | | Incorpo- | | Incorpo- | | Incorpo- | | Incorpo- | | Incorpo- | | |
|---|---|---|---|---|---|---|---|---|---|---|---|---|---|---|
| | | | | ration | | ration | | ration | | ration | | ration | | |
| | | | Unit | ratio | Unit | ratio | Unit | ratio | Unit | ratio | Unit | ratio | | |
| | | Polymer | a | (mol %) | b1 | (mol %) | b2 | (mol %) | c | (mol %) | d | (mol %) | Mw | Mw/Mn |
| Example | 2-1 | P-1 | a-1 | 15 | b1-1 | 55 | — | — | c-1 | 30 | — | — | 9,400 | 1.75 |
| | 2-2 | P-2 | a-2 | 15 | b1-1 | 55 | — | — | c-1 | 30 | — | — | 9,200 | 1.76 |
| | 2-3 | P-3 | a-3 | 15 | b1-1 | 55 | — | — | c-1 | 30 | — | — | 9,000 | 1.77 |
| | 2-4 | P-4 | a-4 | 15 | b1-1 | 55 | — | — | c-1 | 30 | — | — | 9,100 | 1.76 |
| | 2-5 | P-5 | a-5 | 15 | b1-1 | 55 | — | — | c-1 | 30 | — | — | 8,900 | 1.74 |
| | 2-6 | P-6 | a-6 | 15 | b1-1 | 55 | — | — | c-1 | 30 | — | — | 9,000 | 1.75 |
| | 2-7 | P-7 | a-7 | 15 | b1-1 | 55 | — | — | c-1 | 30 | — | — | 9,200 | 1.73 |
| | 2-8 | P-8 | a-1 | 15 | b1-2 | 55 | — | — | c-1 | 30 | — | — | 9,100 | 1.75 |
| | 2-9 | P-9 | a-1 | 15 | b1-3 | 55 | — | — | c-1 | 30 | — | — | 8,800 | 1.76 |
| | 2-10 | P-10 | a-2 | 15 | b1-4 | 55 | — | — | c-1 | 30 | — | — | 9,100 | 1.75 |
| | 2-11 | P-11 | a-3 | 10 | b1-3 | 35 | b2-1 | 20 | c-2 | 25 | d-1 | 10 | 9,200 | 1.75 |
| | 2-12 | P-12 | a-4 | 15 | b1-1 | 25 | — | — | c-2 | 35 | — | — | 9,300 | 1.73 |
| | | | | | b1-2 | 25 | | | | | | | | |
| | 2-13 | P-13 | a-5 | 15 | b1-3 | 50 | — | — | c-3 | 25 | d-2 | 10 | 9,200 | 1.74 |
| | 2-14 | P-14 | a-6 | 15 | b1-4 | 25 | b2-1 | 25 | c-4 | 35 | — | — | 9,100 | 1.75 |
| | 2-15 | P-15 | a-7 | 10 | b1-2 | 35 | b2-1 | 15 | c-2 | 30 | d-3 | 10 | 9,000 | 1.73 |
| | 2-16 | P-16 | a-1 | 15 | b1-1 | 35 | — | — | c-4 | 35 | — | — | 9,500 | 1.74 |
| | | | | | b1-3 | 15 | | | | | | | | |
| | 2-17 | P-17 | a-2 | 15 | b1-1 | 25 | b2-1 | 25 | c-2 | 20 | d-1 | 15 | 9,200 | 1.75 |
| | 2-18 | P-18 | a-3 | 20 | b1-2 | 45 | — | — | c-2 | 30 | d-3 | 5 | 9,100 | 1.74 |
| | 2-19 | P-19 | a-1 | 5 | b1-1 | 50 | — | — | c-2 | 25 | d-2 | 10 | 9,200 | 1.75 |
| | | | | | | | | | | | d-3 | 10 | | |
| | 2-20 | P-20 | a-1 | 15 | b1-2 | 30 | — | — | c-2 | 35 | — | — | 9,200 | 1.74 |
| | | | | | b1-3 | 20 | | | | | | | | |
| | 2-21 | P-21 | a-8 | 15 | b1-1 | 55 | — | — | c-1 | 30 | — | — | 9,400 | 1.73 |
| | 2-22 | P-22 | a-9 | 15 | b1-1 | 55 | — | — | c-1 | 30 | — | — | 9,500 | 1.74 |
| | 2-23 | P-23 | a-10 | 15 | b1-1 | 55 | — | — | c-1 | 30 | — | — | 9,200 | 1.76 |

TABLE 2

| | | | | Incorpo- | | Incorpo- | | Incorpo- | | Incorpo- | | Incorpo- | | |
|---|---|---|---|---|---|---|---|---|---|---|---|---|---|---|
| | | | | ration | | ration | | ration | | ration | | ration | | |
| | | | Unit | ratio | Unit | ratio | Unit | ratio | Unit | ratio | Unit | ratio | | |
| | | Polymer | a | (mol %) | b1 | (mol %) | b2 | (mol %) | c | (mol %) | d | (mol %) | Mw | Mw/Mn |
| Comparative | 2-1 | CP-1 | ca-1 | 15 | b1-1 | 55 | — | — | c-1 | 30 | — | — | 9,500 | 1.74 |
| Example | 2-2 | CP-2 | ca-2 | 15 | b1-1 | 55 | — | — | c-1 | 30 | — | — | 9,100 | 1.75 |
| | 2-3 | CP-3 | ca-3 | 15 | b1-1 | 55 | — | — | c-1 | 30 | — | — | 9,200 | 1.73 |
| | 2-4 | CP-4 | ca-4 | 15 | b1-1 | 55 | — | — | c-1 | 30 | — | — | 8,900 | 1.76 |
| | 2-5 | CP-5 | ca-1 | 10 | b1-3 | 35 | b2-1 | 20 | c-2 | 20 | d-1 | 20 | 9,000 | 1.74 |
| | 2-6 | CP-6 | ca-2 | 10 | b1-2 | 35 | b2-1 | 15 | c-2 | 30 | d-3 | 10 | 9,200 | 1.75 |
| | 2-7 | CP-7 | ca-3 | 15 | b1-1 | 35 | — | — | c-4 | 35 | — | — | 9,100 | 1.74 |
| | | | | | b1-3 | 15 | | | | | | | | |
| | 2-8 | CP-8 | ca-4 | 15 | b1-3 | 50 | — | — | c-3 | 25 | d-2 | 10 | 9,300 | 1.73 |
| | 2-9 | CP-9 | ca-2 | 15 | b1-1 | 25 | b2-1 | 25 | c-2 | 20 | d-1 | 15 | 9,200 | 1.76 |
| | 2-10 | CP-10 | ca-3 | 5 | b1-1 | 50 | — | — | c-2 | 25 | d-2 | 10 | 9,200 | 1.74 |
| | | | | | | | | | | | d-3 | 10 | | |

### [3] Preparation of Chemically Amplified Resist Composition

### Examples 3-1 to 3-26 and Comparative Examples 3-1 to 3-10

A chemically amplified resist composition (R-1 to R-26 and CR-1 to CR-10) was prepared by dissolving a component selected from base polymers (P-1 to P-23), a component selected from comparative base polymers (CP-1 to CP-10), a component selected from acid generators (PAG-1 and PAG-2) and a component selected from quenchers (SQ-1 to SQ-3 and AQ-1) in a solvent containing 0.01 wt % of surfactant FC-4430 (3M Company) in accordance with the formulation shown in Tables 3 and 4 below, and filtering the solution through a Teflon® filter with a pore size of 0.2 μm.

TABLE 3

| | | Resist | Base polymer | Quencher | Acid generator | Solvent 1 | Solvent 2 |
|---|---|---|---|---|---|---|---|
| | | composition | (pbw) | (pbw) | (pbw) | (pbw) | (pbw) |
| Example | 3-1 | R-1 | P-1 (80) | SQ-1 (8.0) | — | PGMEA (2200) | DAA (900) |
| | 3-2 | R-2 | P-2 (80) | SQ-1 (7.8) | — | PGMEA (2200) | DAA (900) |
| | 3-3 | R-3 | P-3 (80) | SQ-1 (7.4) | — | PGMEA (2200) | DAA (900) |
| | 3-4 | R-4 | P-4 (80) | SQ-1 (8.0) | — | PGMEA (2200) | DAA (900) |
| | 3-5 | R-5 | P-5 (80) | SQ-1 (8.0) | — | PGMEA (2200) | DAA (900) |
| | 3-6 | R-6 | P-6 (80) | SQ-1 (8.0) | — | PGMEA (2200) | DAA (900) |
| | 3-7 | R-7 | P-7 (80) | SQ-1 (7.8) | — | PGMEA (2200) | DAA (900) |
| | 3-8 | R-8 | P-8 (80) | SQ-2 (8.0) | — | PGMEA (2200) | DAA (900) |
| | 3-9 | R-9 | P-9 (80) | SQ-2 (8.0) | — | PGMEA (2200) | DAA (900) |
| | 3-10 | R-10 | P-10 (80) | SQ-1 (4.0) | — | PGMEA (2200) | DAA (900) |
| | | | | AQ-1 (4.0) | | | |
| | 3-11 | R-11 | P-11 (80) | SQ-3 (7.8) | — | PGMEA (2200) | DAA (900) |
| | 3-12 | R-12 | P-12 (80) | SQ-3 (7.6) | — | PGMEA (2200) | DAA (900) |
| | 3-13 | R-13 | P-13 (80) | SQ-1 (7.8) | — | PGMEA (2200) | DAA (900) |
| | 3-14 | R-14 | P-14 (80) | SQ-3 (8.0) | — | PGMEA (2200) | DAA (900) |
| | 3-15 | R-15 | P-15 (80) | SQ-2 (4.0) | PAG-1 (10) | PGMEA (2200) | DAA (900) |
| | | | | AQ-1 (4.0) | | | |
| | 3-16 | R-16 | P-16 (80) | SQ-1 (8.2) | — | PGMEA (2200) | DAA (900) |
| | 3-17 | R-17 | P-17 (80) | SQ-1 (7.6) | — | PGMEA (2200) | DAA (900) |
| | 3-18 | R-18 | P-18 (80) | SQ-3 (8.0) | — | PGMEA (2200) | DAA (900) |
| | 3-19 | R-19 | P-19 (80) | SQ-3 (4.0) | PAG-2 (15) | PGMEA (2200) | DAA (900) |
| | | | | AQ-1 (4.0) | | | |
| | 3-20 | R-20 | P-20 (80) | SQ-2 (7.6) | — | PGMEA (2200) | DAA (900) |
| | 3-21 | R-21 | P-21 (80) | SQ-1 (7.8) | — | PGMEA (2200) | DAA (900) |
| | 3-22 | R-22 | P-22 (80) | SQ-1 (7.6) | — | PGMEA (2200) | DAA (900) |
| | 3-23 | R-23 | P-23 (80) | SQ-1 (8.0) | — | PGMEA (2200) | DAA (900) |
| | 3-24 | R-24 | P-21 (80) | SQ-4 (7.8) | — | PGMEA (2200) | DAA (900) |
| | 3-25 | R-25 | P-22 (80) | SQ-4 (7.6) | — | PGMEA (2200) | DAA (900) |
| | 3-26 | R-26 | P-23 (80) | SQ-4 (8.0) | — | PGMEA (2200) | DAA (900) |

TABLE 4

| | | Resist | Base polymer | Quencher | Acid generator | Solvent 1 | Solvent 2 |
|---|---|---|---|---|---|---|---|
| | | composition | (pbw) | (pbw) | (pbw) | (pbw) | (pbw) |
| Comparative | 3-1 | CR-1 | P-1 (80) | SQ-1 (8.0) | — | PGMEA (2200) | DAA (900) |
| Example | 3-2 | CR-2 | P-2 (80) | SQ-1 (7.8) | — | PGMEA (2200) | DAA (900) |
| | 3-3 | CR-3 | P-3 (80) | SQ-1 (7.8) | — | PGMEA (2200) | DAA (900) |
| | 3-4 | CR-4 | P-4 (80) | SQ-1 (8.2) | — | PGMEA (2200) | DAA (900) |
| | 3-5 | CR-5 | P-5 (80) | SQ-3 (7.8) | — | PGMEA (2200) | DAA (900) |
| | 3-6 | CR-6 | P-6 (80) | SQ-2 (4.0) | PAG-1 (10) | PGMEA (2200) | DAA (900) |
| | | | | AQ-1 (4.0) | | | |
| | 3-7 | CR-7 | P-7 (80) | SQ-1 (8.2) | — | PGMEA (2200) | DAA (900) |
| | 3-8 | CR-8 | P-8 (80) | SQ-2 (7.8) | — | PGMEA (2200) | DAA (900) |
| | 3-9 | CR-9 | P-9 (80) | SQ-3 (7.6) | — | PGMEA (2200) | DAA (900) |
| | 3-10 | CR-10 | P-10 (80) | SQ-3 (4.0) | PAG-2 (15) | PGMEA (2200) | DAA (900) |
| | | | | AQ-1 (4.0) | | | |

The solvents, quenchers (SQ-1 to SQ-4, AQ-1) and acid generators (PAG-1, PAG-2) in Tables 3 and 4 are as identified below.

### Solvent:

PGMEA (propylene glycol monomethyl ether acetate)
DAA (diacetone alcohol)
Quencher: SQ-1 to SQ-4, AQ-1

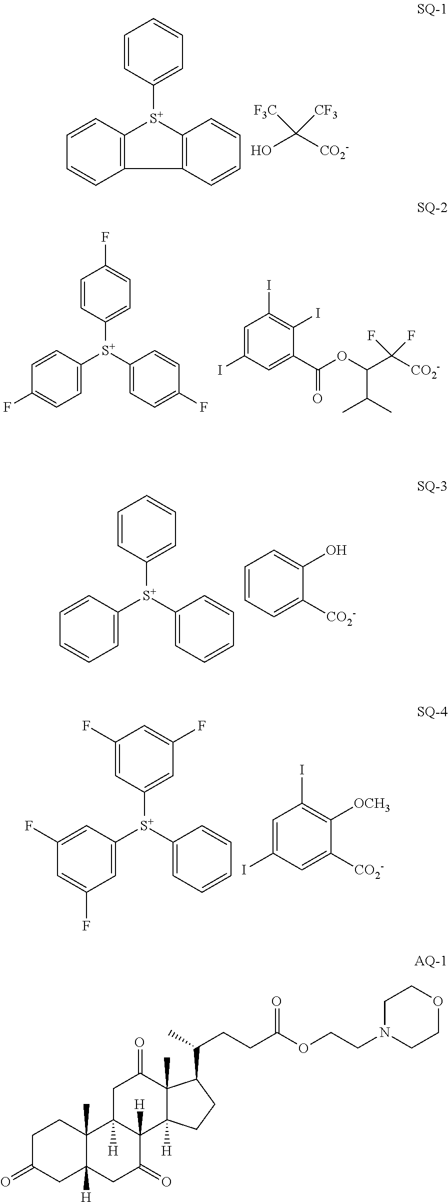

Acid generator: PAG-1, PAG-2

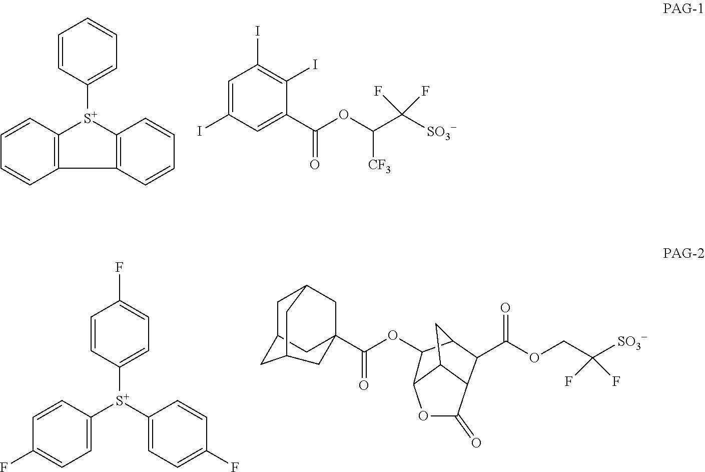

### [4] EUV Lithography Test (1)

### Examples 4-1 to 4-26 and Comparative Examples 4-1 to 4-10

Each of the chemically amplified resist compositions (R-1 to R-26, CR-1 to CR-10) shown in Tables 3 and 4 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3300 manufactured by ASML (NA 0.33, a 0.9/0.6, dipole illumination), the resist film was exposed to EUV through a mask bearing a line-and-space (LS) pattern having a width of 18 nm (on-wafer size) and a pitch of 36 nm while changing the dose at a pitch of 1 mJ/cm² and the focus at a pitch of 0.020 μm. The resist film was baked (PEB) at the temperature shown in Tables 5 and 6 for 60 seconds. This was followed by puddle development in a 2.38 wt % TMAH aqueous solution for 30 seconds, rinsing with a surfactant-containing rinse fluid, and spin drying. A positive LS pattern was obtained.

The obtained LS pattern was observed under CD-SEM (CG6300, Hitachi High-Technologies Corp.) and evaluated for sensitivity, exposure latitude (EL), LWR, depth of focus (DOF), and collapse limit by the following methods. The results are shown in Tables 5 and 6.

### [Evaluation of Sensitivity]

The optimum dose Eop (mJ/cm²) which provided a LS pattern with a line width of 18 nm and a pitch of 36 nm was determined as an index of sensitivity. A smaller value indicates a higher sensitivity.

### [Evaluation of EL]

The exposure dose which provided a LS pattern with a space width of 18 nm±10% (i.e., 16.2 to 19.8 nm) was determined. EL (%) is calculated from the exposure doses according to the following equation: A greater value indicates better performance.

$$EL ( \% ) = ( \vert {E}_{{1}^{-}} {E}_{2} \vert / Eop ) \times 100$$

wherein E₁ is an optimum exposure dose which provides a LS pattern with a line width of 16.2 nm and a pitch of 36 nm, E₂ is an optimum exposure dose which provides a LS pattern with a line width of 19.8 nm and a pitch of 36 nm, and Eop is an optimum exposure dose which provides a LS pattern with a line width of 18 nm and a pitch of 36 nm.

### [Evaluation of LWR]

For the LS pattern formed by exposure at the optimum dose Eop, the line width was measured at 10 longitudinally spaced apart points, from which a 3-fold value (3σ) of standard deviation (σ) was determined and reported as LWR. A smaller value of 3σ indicates a pattern having small roughness and uniform line width.

### [Evaluation of DOF]

As an index of DOF, a range of focus which provided a LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 5

| | | Resist | PEB temp. | Optimal exposure dose | EL | LWR | DOF | Collapse limit |
|---|---|---|---|---|---|---|---|---|
| | | composition | (° C.) | (mJ/cm²) | (%) | (nm) | (nm) | (nm) |
| Example | 4-1 | R-1 | 95 | 35 | 19 | 2.5 | 120 | 10.6 |
| | 4-2 | R-2 | 100 | 36 | 17 | 2.6 | 110 | 10.8 |
| | 4-3 | R-3 | 100 | 37 | 18 | 2.7 | 120 | 10.9 |
| | 4-4 | R-4 | 95 | 35 | 19 | 2.6 | 110 | 11.1 |
| | 4-5 | R-5 | 105 | 36 | 17 | 2.7 | 100 | 11.2 |
| | 4-6 | R-6 | 100 | 35 | 18 | 2.6 | 120 | 10.9 |
| | 4-7 | R-7 | 95 | 37 | 18 | 2.6 | 110 | 11.2 |
| | 4-8 | R-8 | 95 | 36 | 19 | 2.7 | 100 | 11.1 |
| | 4-9 | R-9 | 100 | 35 | 19 | 2.7 | 110 | 11.5 |
| | 4-10 | R-10 | 100 | 36 | 18 | 2.6 | 120 | 11.3 |
| | 4-11 | R-11 | 100 | 35 | 19 | 2.7 | 120 | 10.8 |
| | 4-12 | R-12 | 95 | 36 | 17 | 2.6 | 110 | 11.3 |
| | 4-13 | R-13 | 105 | 36 | 18 | 2.8 | 120 | 11.2 |
| | 4-14 | R-14 | 100 | 35 | 17 | 2.7 | 100 | 11.4 |
| | 4-15 | R-15 | 95 | 36 | 19 | 2.6 | 110 | 11.1 |
| | 4-16 | R-16 | 95 | 37 | 18 | 2.7 | 120 | 10.9 |
| | 4-17 | R-17 | 100 | 35 | 17 | 2.8 | 110 | 10.8 |
| | 4-18 | R-18 | 95 | 37 | 19 | 2.6 | 110 | 11.3 |
| | 4-19 | R-19 | 95 | 36 | 18 | 2.7 | 120 | 11.3 |
| | 4-20 | R-20 | 100 | 35 | 17 | 2.7 | 110 | 11.2 |
| | 4-2 1 | R-21 | 100 | 34 | 18 | 2.4 | 120 | 11.1 |
| | 4-22 | R-22 | 105 | 35 | 19 | 2.5 | 110 | 10.9 |
| | 4-23 | R-23 | 100 | 34 | 17 | 2.4 | 120 | 11.4 |
| | 4-24 | R-24 | 100 | 33 | 18 | 2.4 | 110 | 10.9 |
| | 4-25 | R-25 | 105 | 33 | 19 | 2.4 | 120 | 10.8 |
| | 4-26 | R-26 | 100 | 34 | 17 | 2.4 | 120 | 11.1 |

TABLE 6

| | | Resist | PEB temp. | Optimal exposure dose | EL | LWR | DOF | Collapse limit |
|---|---|---|---|---|---|---|---|---|
| | | composition | (° C.) | (mJ/cm²) | (%) | (nm) | (nm) | (nm) |
| Comparative | 4-1 | CR-1 | 95 | 39 | 13 | 3.5 | 90 | 13.2 |
| Example | 4-2 | CR-2 | 100 | 38 | 14 | 3.4 | 80 | 13.9 |
| | 4-3 | CR-3 | 100 | 40 | 14 | 3.3 | 80 | 12.1 |
| | 4-4 | CR-4 | 100 | 41 | 14 | 3.2 | 90 | 12.5 |
| | 4-5 | CR-5 | 95 | 40 | 14 | 3.5 | 80 | 13.5 |
| | 4-6 | CR-6 | 100 | 39 | 15 | 3.4 | 90 | 13.6 |
| | 4-7 | CR-7 | 100 | 40 | 13 | 3.3 | 90 | 12.3 |
| | 4-8 | CR-8 | 100 | 41 | 14 | 3.2 | 80 | 12.5 |
| | 4-9 | CR-9 | 95 | 40 | 14 | 3.4 | 90 | 13.9 |
| | 4-10 | CR-10 | 100 | 41 | 14 | 3.3 | 80 | 12.4 |

From the results shown in Tables 5 and 6, it is revealed that the inventive chemically amplified resist composition comprising a base polymer containing repeat units derived from an onium salt type monomer has a high sensitivity, and excellent EL, LWR and DOF. The resist composition is also confirmed to have a low collapse resistance value, and resistance to pattern collapse in fine pattern formation. This demonstrates that chemically amplified resist compositions are suitable as materials for EUV lithography.

### [5] EUV Lithography Test (2)

### Examples 5-1 to 5-26 and Comparative Examples 5-1 to 5-10

Each of the chemically amplified resist compositions (R-1 to R-26, CR-1 to CR-10) shown in Tables 3 and 4 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 manufactured by ASML (NA 0.33, a 0.9/0.6, quadrupole illumination), the resist film was exposed to EUV through a mask bearing a hole pattern having a pitch (on-wafer size) of 46 nm+20% bias. The resist film was baked (PEB) on a hotplate at the temperature shown in Tables 7 and 8 for 60 seconds and developed in a 2.38 wt % TMAH aqueous solution for 30 seconds to form a hole pattern having a size of 23 nm.

Using CD-SEM (CG6300) manufactured by Hitachi High-Technologies Corp., the exposure dose which provided a hole pattern of 23 nm size was measured and reported as sensitivity, and the size of 50 holes was measured, from which a 3-fold value (36) of standard deviation (6) was calculated and reported as critical dimension uniformity (CDU). The results are shown in Tables 7 and 8.

TABLE 7

| | | | | Optimal | |
|---|---|---|---|---|---|
| | | Resist | PEB temp. | exposure dose | CDU |
| | | composition | (° C.) | (mJ/cm²) | (nm) |
| Example | 5-1 | R-1 | 95 | 23 | 2.2 |
| | 5-2 | R-2 | 95 | 24 | 2.3 |
| | 5-3 | R-3 | 90 | 24 | 2.5 |
| | 5-4 | R-4 | 90 | 24 | 2.3 |
| | 5-5 | R-5 | 90 | 25 | 2.4 |
| | 5-6 | R-6 | 95 | 23 | 2.5 |
| | 5-7 | R-7 | 95 | 25 | 2.4 |
| | 5-8 | R-8 | 90 | 24 | 2.5 |
| | 5-9 | R-9 | 95 | 24 | 2.4 |
| | 5-10 | R-10 | 95 | 25 | 2.5 |
| | 5-11 | R-11 | 95 | 23 | 2.5 |
| | 5-12 | R-12 | 90 | 23 | 2.6 |
| | 5-13 | R-13 | 90 | 25 | 2.4 |
| | 5-14 | R-14 | 90 | 24 | 2.5 |
| | 5-15 | R-15 | 90 | 25 | 2.4 |
| | 5-16 | R-16 | 85 | 23 | 2.5 |
| | 5-17 | R-17 | 95 | 23 | 2.6 |
| | 5-18 | R-18 | 95 | 24 | 2.4 |
| | 5-19 | R-19 | 90 | 25 | 2.5 |
| | 5-20 | R-20 | 95 | 24 | 2.4 |
| | 5-21 | R-21 | 95 | 23 | 2.2 |
| | 5-22 | R-22 | 90 | 24 | 2.3 |
| | 5-23 | R-23 | 95 | 23 | 2.2 |
| | 5-24 | R-24 | 95 | 23 | 2.3 |
| | 5-25 | R-25 | 90 | 23 | 2.2 |
| | 5-26 | R-26 | 95 | 24 | 2.2 |

TABLE 8

| | | | | Optimal | |
|---|---|---|---|---|---|
| | | Resist | PEB temp. | exposure dose | CDU |
| | | composition | (° C.) | (mJ/cm²) | (nm) |
| Comparative | 5-1 | CR-1 | 95 | 29 | 3.1 |
| Example | 5-2 | CR-2 | 95 | 20 | 3.0 |
| | 5-3 | CR-3 | 95 | 31 | 3.1 |
| | 5-4 | CR-4 | 90 | 32 | 3.2 |
| | 5-5 | CR-5 | 90 | 28 | 2.9 |
| | 5-6 | CR-6 | 95 | 29 | 3.1 |
| | 5-7 | CR-7 | 90 | 20 | 3.2 |
| | 5-8 | CR-8 | 90 | 29 | 3.2 |
| | 5-9 | CR-9 | 90 | 31 | 2.9 |
| | 5-10 | CR-10 | 95 | 30 | 3.3 |

It is demonstrated in Tables 7 and 8 that chemically amplified resist compositions within the scope of the invention exhibit a high sensitivity and improved CDU.

### [6] Evaluation of Dry Etch Resistance

### Examples 6-1 to 6-23 and Comparative Examples 6-1 to 6-10

2 g of each of the polymers shown in Tables 1 and 2 (Polymers P-1 to P-23 and Comparative Polymers CP-1 to CP-10) was dissolved in 10 g of cyclohexanone. The solution was filtered through a filter with a pore size of 0.2 μm, and spin-coated on a Si substrate to form a 300 nm-thick film thereon, and the film was tested under the following conditions.

[Etching Test with CHF₃/CF₄-Based Gas]

A polymer film thickness difference before and after etching was determined using a dry etching apparatus TE-8500 P manufactured by Tokyo Electron Limited.

The etching conditions are shown below.
Chamber pressure: 40 Pa
RF power: 1000 W
Gap: 9 mm
CHF₃ gas flow rate: 30 mL/min
CF₄ gas flow rate: 30 mL/min
Ar gas flow rate: 100 mL/min
Time: 60 sec

In this evaluation, a smaller film thickness difference, i.e. a smaller amount of decrease, indicates higher etch resistance.

The results of dry etch resistance evaluation are shown in Tables 9 and 10.

TABLE 9

| | | | CHF₃/CF₄-based gas |
|---|---|---|---|
| | | Polymer | etching rate (nm/min) |
| Example | 6-1 | P-1 | 95 |
| | 6-2 | P-2 | 96 |
| | 6-3 | P-3 | 97 |
| | 6-4 | P-4 | 95 |
| | 6-5 | P-5 | 96 |
| | 6-6 | P-6 | 96 |
| | 6-7 | P-7 | 97 |
| | 6-8 | P-8 | 95 |
| | 6-9 | P-9 | 94 |
| | 6-10 | P-10 | 95 |
| | 6-11 | P-11 | 97 |
| | 6-12 | P-12 | 96 |
| | 6-13 | P-13 | 95 |
| | 6-14 | P-14 | 98 |
| | 6-15 | P-15 | 96 |
| | 6-16 | P-16 | 95 |
| | 6-17 | P-17 | 96 |
| | 6-18 | P-18 | 96 |
| | 6-19 | P-19 | 95 |
| | 6-20 | P-20 | 96 |
| | 6-21 | P-21 | 94 |
| | 6-22 | P-22 | 95 |
| | 6-23 | P-23 | 95 |

TABLE 10

| | | | CHF₃/CF₄-based gas |
|---|---|---|---|
| | | Polymer | etching rate (nm/min) |
| Comparative | 6-1 | CP-1 | 114 |
| Example | 6-2 | CP-2 | 118 |
| | 6-3 | CP-3 | 101 |
| | 6-4 | CP-4 | 100 |
| | 6-5 | CP-5 | 112 |
| | 6-6 | CP-6 | 115 |
| | 6-7 | CP-7 | 102 |
| | 6-8 | CP-8 | 100 |
| | 6-9 | CP-9 | 115 |
| | 6-10 | CP-10 | 103 |

From the results shown in Tables 9 and 10, it is confirmed that the inventive polymer has excellent dry etch resistance to a CHF₃/CF₄-based gas.

Japanese Patent Application No. 2023-178545 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. An onium salt type monomer having the following formula (a): 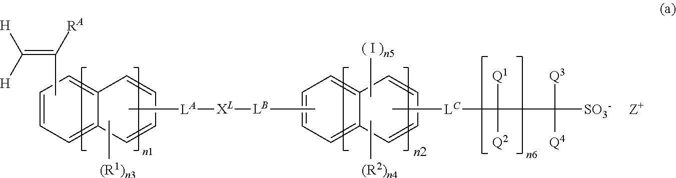
wherein n1 is 0 or 1, n2 is 0 or 1, n3 is an integer of 0 to 4, n4 is an integer of 0 to 4, n5 is an integer of 1 to 6, provided that the sum of n4+n5 is 1 or more and 4 or less when n2 is 0, and the sum of n4+n5 is 1 or more and 6 or less when n2 is 1, n6 is an integer of 0 to 4,
R^{A} is a hydrogen atom, a fluorine atom, a methyl group, or a trifluoromethyl group,
R¹ is a halogen atom, or a C₁-C₂₀ hydrocarbyl group which may contain a heteroatom,
a plurality of R¹ moieties may bond together to form a ring with the carbon atom to which they are attached when n3 is 2, 3 or 4,
R² is a halogen atom other than an iodine atom, or a C₁-C₂₀ hydrocarbyl group which may contain a heteroatom,
a plurality of R² moieties may bond together to form a ring with the carbon atom to which they are attached when n4 is 2, 3 or 4,
L^{A}, L^{B} and L^{C} are each independently a single bond, an ether bond, an ester bond, a sulfonic ester bond, a sulfonic amide bond, a carbonate bond or a carbamate bond,
X^{L} is a single bond, or a C₁-C₄₀ hydrocarbylene group which may contain a heteroatom,
Q¹ and Q² are each independently a hydrogen atom, a fluorine atom, or a C₁-C₆ fluorinated saturated hydrocarbyl group,
Q³ and Q⁴ are each independently a fluorine atom, or a C1-C₆ fluorinated saturated hydrocarbyl group, and
Z⁺ is an onium cation.

2. The onium salt type monomer according to claim 1 which has the following formula (a1): 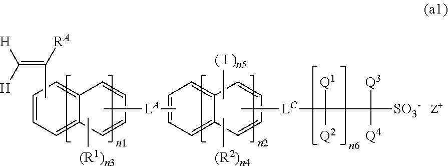
wherein n1 to n6, R^{A}, R¹, R², L^{A}, L^{C}, Q¹ to Q⁴, and Z⁺ are as defined above.

3. The onium salt type monomer according to claim 2 which has the following formula (a2): 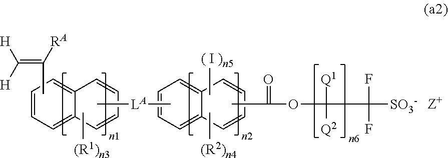
wherein n1 to n6, R^{A}, R¹, R², L^{A}, Q¹, Q², and Z⁺ are as defined above.

4. The onium salt type monomer according to claim 1, wherein Z⁺ is a sulfonium cation having the following formula (cation-1) or iodonium cation having the following formula (cation-2): 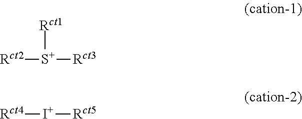
wherein R^{ct1} to R^{ct5} are each independently a halogen atom, or a C₁-C₃₀ hydrocarbyl group which may contain a heteroatom, and R^{ct1} and R^{ct2} may bond together to form a ring with the sulfur atom to which they are attached.

5. A polymer comprising repeat units derived from the onium salt type monomer of claim 1.

6. The polymer according to claim 5, further comprising repeat units having the following formula (b1) or (b2): 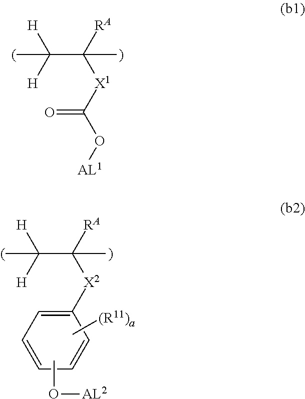
wherein R^{A} is each independently a hydrogen atom, a fluorine atom, a methyl group, or a trifluoromethyl group;
X¹ is a single bond, a phenylene group, a naphthylene group, *—C(═O)—O—X¹¹—, or *—C(═O)—NH—X¹¹—, where the phenylene group or naphthylene group may be substituted with a C₁-C₁₀ alkoxy group which may contain a fluorine atom, or a halogen atom, and X¹¹ is a C₁-C₁₀ saturated hydrocarbylene group, a phenylene group or a naphthylene group, where the saturated hydrocarbylene group may contain a hydroxy group, an ether bond, an ester bond, or a lactone ring;
X² is a single bond, *—C(═O)—O—, or *—C(═O)—NH—;
the asterisk (*) designates a point of attachment to the carbon atom in the backbone;
AL¹ and AL² are each independently an acid labile group;
R¹¹ is a halogen atom, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom; and
a is an integer of 0 to 4.

7. The polymer according to claim 5, further comprising repeat units having the following formula (c1): 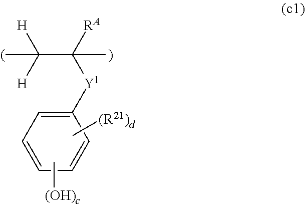
wherein R^{A} is a hydrogen atom, a fluorine atom, a methyl group, or a trifluoromethyl group;
Y¹ is a single bond, *—C(═O)—O— or *—C(═O)—NH—, and the asterisk (*) designates a point of attachment to the carbon atom in the backbone;
R²¹ is a halogen atom, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom;
c is an integer of 1 to 4, and d is an integer of 0 to 3, provided that the sum of c+d is 1 or more and 5 or less.

8. The polymer according to claim 5, further comprising repeat units having the following formula (d1): 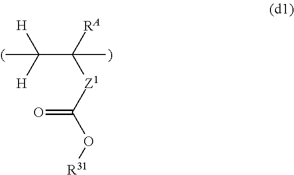
wherein R^{A} is a hydrogen atom, a fluorine atom, a methyl group, or a trifluoromethyl group;
Z¹ is a single bond, a phenylene group, a naphthylene group, *—C(═O)—O—Z¹¹—, or *—C(═O)—NH—Z¹¹—, where the phenylene group or naphthylene group may be substituted with a C₁-C₁₀ alkoxy group which may contain a fluorine atom, or halogen atom, the asterisk (*) designates a point of attachment to the carbon atom in the backbone, and Z¹¹ is a C₁-C₁₀ saturated hydrocarbylene group, a phenylene group or a naphthylene group, where the saturated hydrocarbylene group may contain a hydroxy group, an ether bond, an ester bond, or a lactone ring; and
R³¹ is a hydrogen atom, or a C₁-C₂₀ group containing at least one structure selected from a hydroxy group other than a phenolic hydroxy group, a cyano group, a carbonyl group, a carboxy group, an ether bond, an ester bond, a sulfonic ester bond, a carbonate bond, a lactone ring, a sultone ring, and carboxylic anhydride (—C(═O)—O—C(═O)—).

9. A chemically amplified resist composition comprising (A) a base polymer containing the polymer of claim 5.

10. The chemically amplified resist composition according to claim 9, further comprising (B) an organic solvent.

11. The chemically amplified resist composition according to claim 9, further comprising (C) a quencher.

12. The chemically amplified resist composition according to claim 9, further comprising (D) an acid generator.

13. The chemically amplified resist composition according to claim 9, further comprising (E) a surfactant.

14. The chemically amplified resist composition according to claim 9, further comprising (F) a dissolution inhibitor.

15. A pattern forming process comprising the steps of applying the chemically amplified resist composition of claim 9 onto a substrate to form a resist film thereon, exposing the resist film to high-energy radiation, and developing the exposed resist film in a developer.

16. The pattern forming process according to claim 15, wherein the high-energy radiation is ArF excimer laser having a wavelength 193 nm, KrF excimer laser having a wavelength 248 nm, an electron beam, or EUV having a wavelength 3 to 15 nm.
